(19) United States
(12) Patent Application Publication
(10) Pub. No.: US 2012/0129763 A1
(43) Pub. Date: May 24, 2012

(54) NOVEL SEMI-SYNTHETIC GLYCOPEPTIDES AS ANTIBACTERIAL AGENTS

(75) Inventors: Daniel Chu, Santa Clara, CA (US); Tao Ye, Kowloon (CN)

(73) Assignee: BIOMARIN PHARMACEUTICAL INC., Novato, CA (US)

(21) Appl. No.: 13/358,374

(22) Filed: Jan. 25, 2012

Related U.S. Application Data
(63) Continuation of application No. 12/332,268, filed on Dec. 10, 2008, now abandoned.
(60) Provisional application No. 61/016,783, filed on Dec. 26, 2007.

(51) Int. Cl. A61K 38/14; A61P 31/04; C07K 9/00

(52) U.S. Cl. 514/2.9; 530/322

(57) ABSTRACT

Semi-synthetic glycopeptides having antibacterial activity are described, in particular, the semi-synthetic glycopeptides described herein are made by chemical modification of a glycopeptide (Compound A, Compound B, Compound H or Compound C) or monosaccharide made by hydrolyzing the disaccharide moiety of the amino acid-4 of the parent glycopeptide in acidic medium to give the amino acid-4 monosaccharide; conversion of the monosaccharide to the amino-sugar derivative; acylation of the amino substituent on the amino acid-4 amino-substituted sugar moiety on these scaffolds with certain acyl groups; and conversion of the acid moiety on the macrocyclic ring of these scaffolds to certain substituted amides. Key reaction is the treatment of properly protected intermediate compound with isocyanate or carrying a Hofmann degradation of the primary amide of the 3^{rd} amino acid asparagines with phenyl-bis-trifluoroacetate to give the primary amine. Also provided are methods for the synthesis of the compounds, pharmaceutical compositions containing the compounds, and methods of use of the compounds for the treatment and/or prophylaxis of diseases, especially bacterial infections.

### RELATED APPLICATIONS

This application is a continuation of U.S. patent application Ser. No. 12/332,268, filed Dec. 10, 2008, which claims the benefit of U.S. Provisional Patent Application No. 61/016,783, filed Dec. 26, 2007, the contents of both of which applications are hereby incorporated by reference in their entireties.

### FIELD OF THE INVENTION

Described herein are semi-synthetic glycopeptides having antibacterial activity, pharmaceutical compositions comprising these compounds, and methods of treatment using semi-synthetic glycopeptides.

### BACKGROUND OF THE INVENTION

The emergence of drug resistant bacterial strains has highlighted the need for synthesizing and identifying antibiotics with improved activity. Naturally occurring and semi-synthetic glycopeptide antibiotics used to combat bacterial infections include compounds such as vancomycin, desmethylvancomycin, eremomycin, teicoplanin (complex of five compounds), dalbavancin, oritavancin, telavancin, and A82846B (LY264826) having structures A, B, C, D, E, F, G and H:

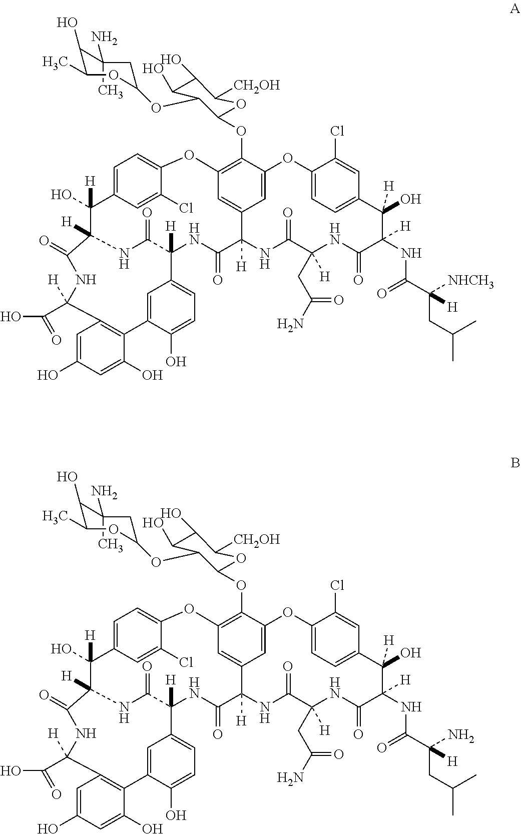

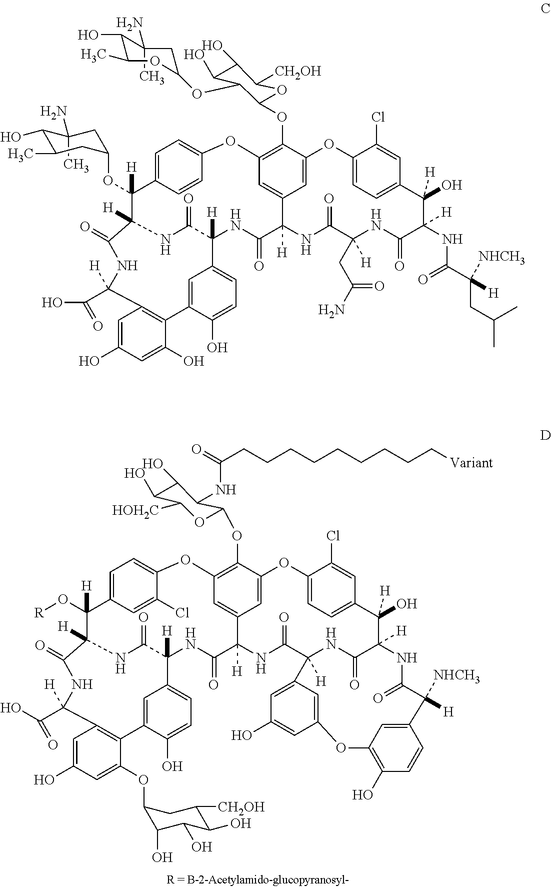

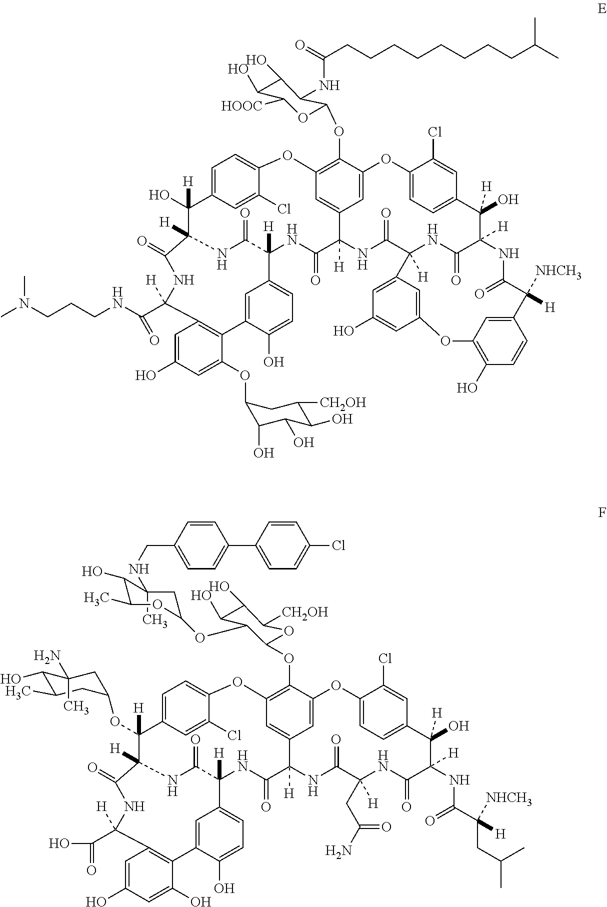

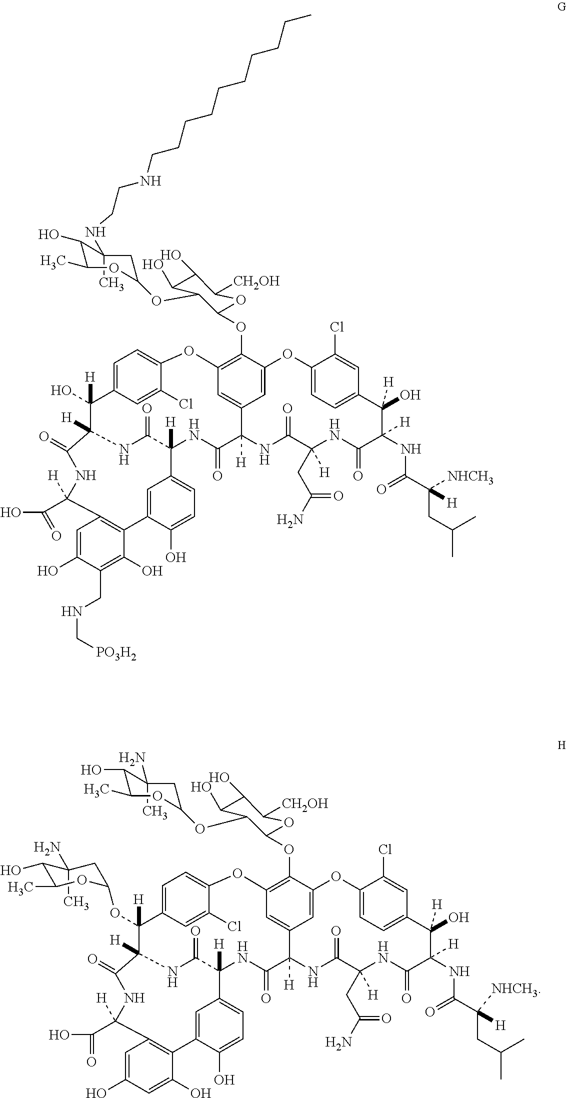

These compounds are used to treat and prevent bacterial infection, but as with other antibacterial agents, bacterial strains having resistance or insufficient susceptibility to these compounds have been identified, and these compounds have been found to have limited effect against certain bacterial infections e.g., against pulmonary S. aureus infections caused by Compound A intermediate-resistant S. aureus or infections due to Compound A resistant-enterococci.

### SUMMARY OF THE INVENTION

Described herein are semi-synthetic glycopeptides that have antibacterial activity. Also provided are methods for synthesis of the compounds, pharmaceutical compositions containing the compounds, and methods of use of the compounds for the treatment and/or prophylaxis of diseases, especially bacterial infections.

In one aspect described herein are compounds formed by modification of Compound A, Compound B, Compound C or Compound H scaffolds to provide semi-synthetic glycopeptides that have antibacterial activity, as well as their pharmaceutical acceptable salts, esters, solvates, alkylated quaternary ammonium salts, stereoisomers, tautomers or prodrugs thereof, and which are used, in some embodiments, as antibacterial agents for the treatment of bacterial infections with superior microbiology and pharmacokinetic properties than currently available glycopeptide antibacterial agents.

In one aspect described herein are compounds having a structure selected from the group consisting of Formulas (I-XII):

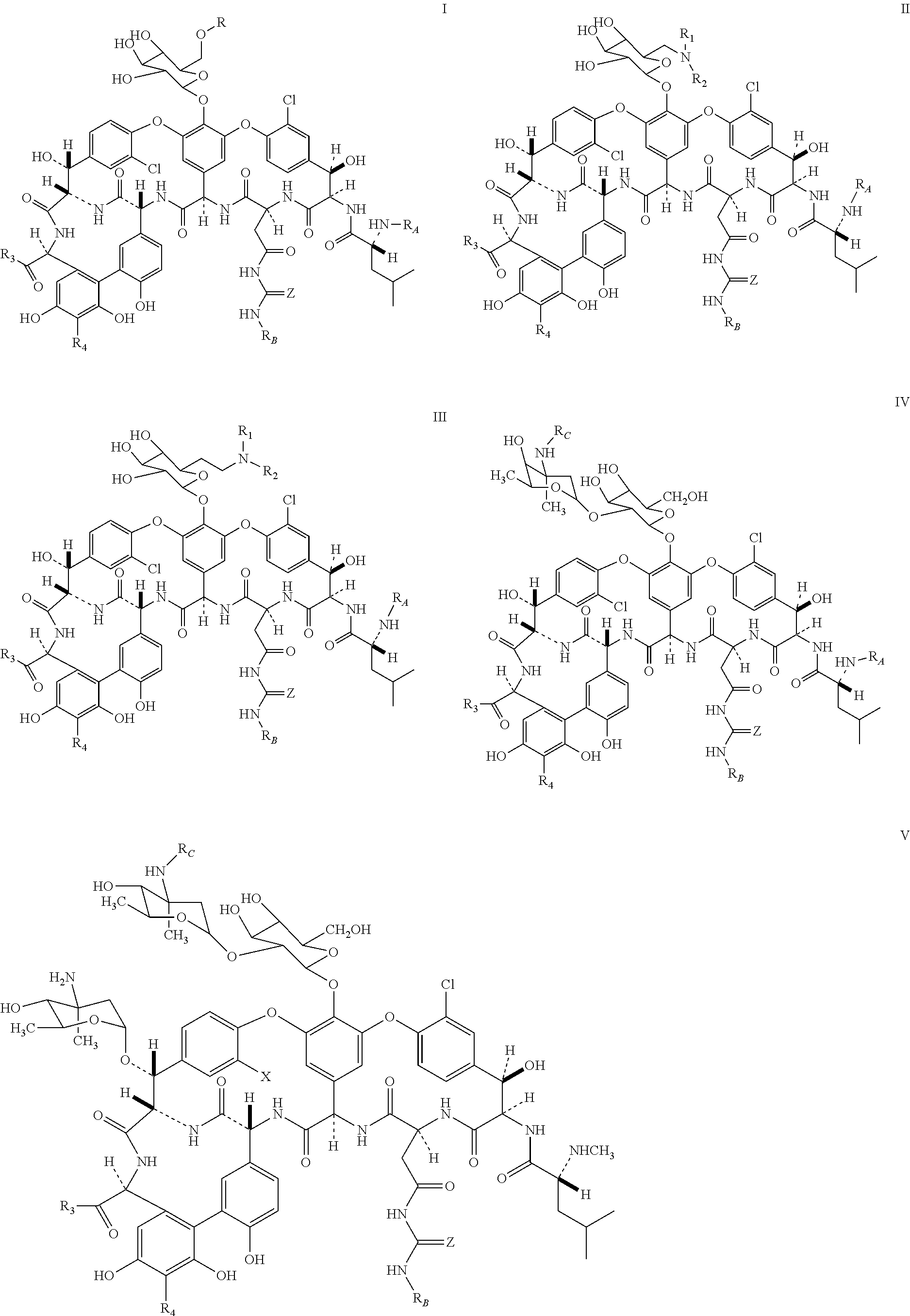

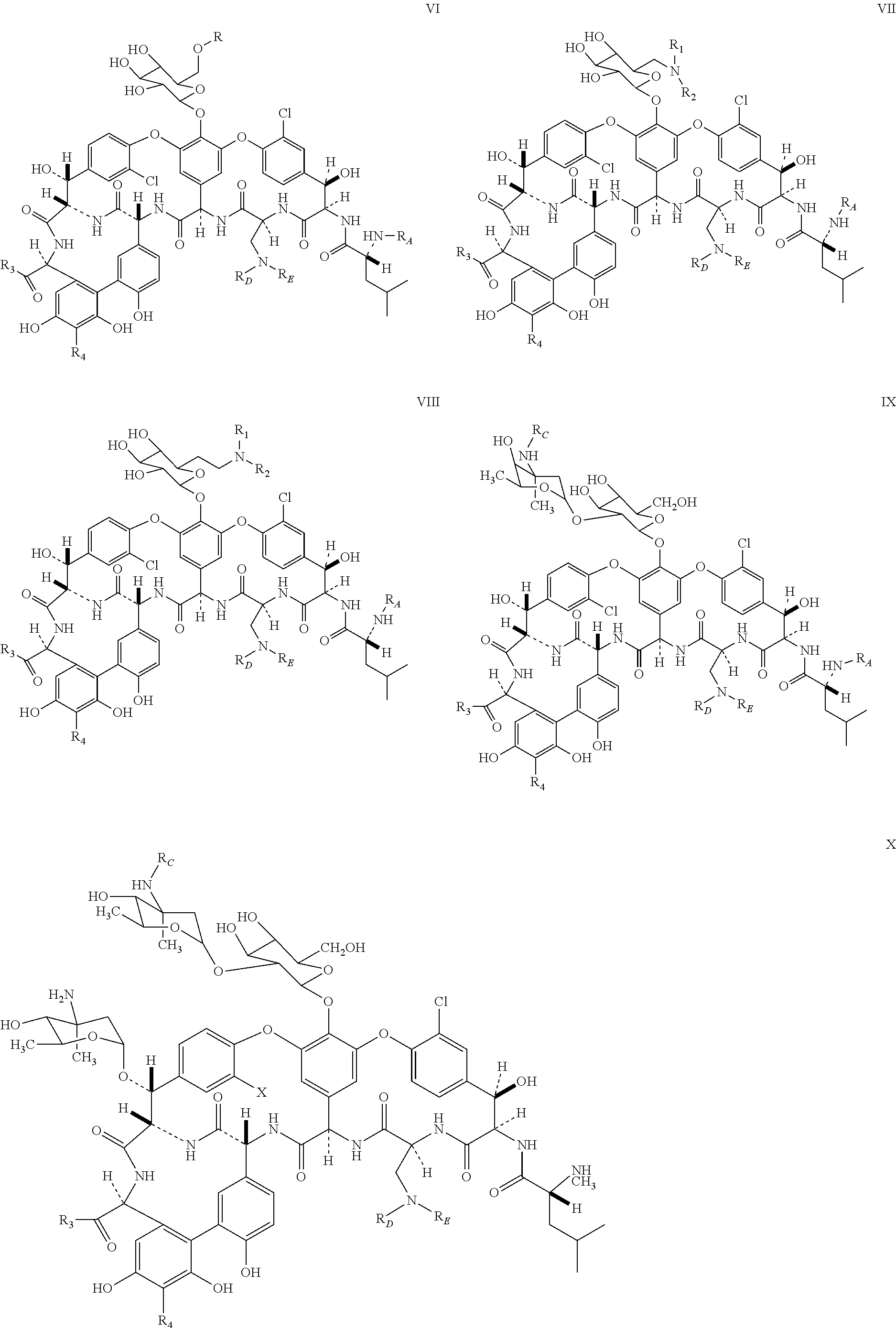

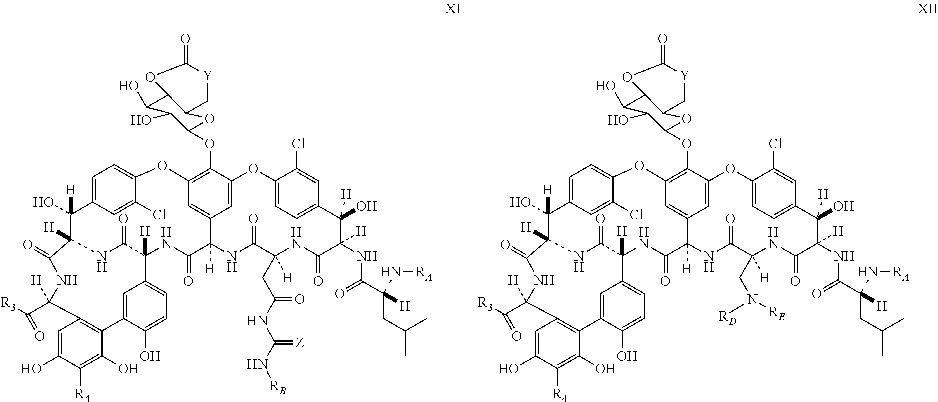

wherein,

R_{A} is selected from the group consisting of
a) hydrogen,
b) methyl,
c) C₂-C₁₂-alkyl;

R₁ and R₂ are each independently selected from the group consisting of
a) hydrogen,
b) C₁-C₁₂-alkyl,
c) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) amino,
   (f) C₁-C₁₂-alkylamino,
   (g) C₁-C₁₂-dialkylamino,
   (h) alkenyl,
   (i) alkynyl,
   C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
   or
   R₁ and R₂ taken together with the atom to which they are attached form a substituted heteroaryl or 3-10 membered heterocycloalkyl ring which optionally contains one to two hetero functionalities selected from the group consisting of —O—, —N—, —NH, —N(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)ₙ— wherein n is 1 or 2 and the 3-10 membered heterocycloalkyl ring is optionally substituted with one or more substituents independently selected from the group consisting of
   (a) halogen,
   (b) hydroxyl,
   (c) C₁-C₃-alkoxy,
   (d) C₁-C₃-alkoxy-C₁-C₃-alkoxy,
   (e) oxo,
   (f) C₁-C₃-alkyl,
   (g) halo-C₁-C₃-alkyl,
   (h) C₁-C₃-alkoxy-C₁-C₃-alkyl,
and
k) C(═O) R₇,
l) C(═O) CH R₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl,
   or
   R₈ and R₁₀ or R₉ and R₁₀ taken together with the atom to which they are attached form a 3-10 membered heterocycloalkyl ring which is optionally substituted with one or more substituents independently selected from the group consisting of
   (a) halogen,
   (b) hydroxyl,
   (c) C₁-C₃-alkoxy,
   (d) C₁-C₃-alkoxy-C₁-C₃-alkoxy,
   (e) oxo,
   (f) C₁-C₃-alkyl,
   (g) halo-C₁-C₃-alkyl,
   (h) C₁-C₃-alkoxy-C₁-C₃-alkyl;

R₇ is selected from the group consisting of
a) hydrogen,
b) C₁-C₁₂-alkyl,
c) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) amino,
   (f) C₁-C₁₂-alkylamino,
   (g) C₁-C₁₂-dialkylamino,
   (h) alkenyl,
   (i) alkynyl,
   (j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
k) C₁-C₁₂-alkylamino;

X is selected from the group consisting of
(1) hydrogen,
(2) chlorine;

Y is selected from the group consisting of
(1) oxygen,
(2) NR₁, wherein R₁ is as previously defined;

Z is selected from the group consisting of
(1) oxygen,
(2) sulfur;

R is selected from the group consisting of
(1) hydrogen,
(2) cycloalkyl,
(3) cycloalkenyl,
(4) C₁-C₁₂-alkyl,
(5) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) —COOR₅ wherein R₅ is hydrogen or loweralkyl,
   (f) —C(O)NR₅R₆ wherein R₅ is as previously defined and R₆ is hydrogen or loweralkyl,
   (g) amino,
   (h) —NR₅R₆ wherein R₅ and R₆ are as previously defined,
      or
      R₅ and R₆ are taken together with the atom to which they are attached form a 3-10 membered heterocycloalkyl ring which is optionally substituted with one or more substituents independently selected from the group consisting of
      (i) halogen,
      (ii) hydroxy,
      (iii) C₁-C₃-alkoxy,
      (iv) C₁-C₃-alkoxy-C₁-C₃-alkoxy,
      (v) oxo,
      (vi) C₁-C₁₂-alkyl,
      (vii) halo-C₁-C₁₂-alkyl,
      and
      (viii) C₁-C₃-alkoxy-C₁-C₁₂-alkyl,
   (i) aryl,
   (j) substituted aryl,
   (k) heteroaryl,
   (l) substituted heteroaryl,
   (m) mercapto,
   (n) C₁-C₁₂-thioalkoxy,
(6) C(═O)O R₁₁, wherein R₁₁ is hydrogen, loweralkyl, substituted loweralkyl, aryl, substituted aryl, heteroaryl or substituted heteroaryl,
(7) C(═O)N R₁₁ R₁₂, wherein R₁₁ is as previously defined and R₁₂ is hydrogen, loweralkyl, substituted loweralkyl, aryl, substituted aryl, heteroaryl or substituted heteroaryl,
   or
   R₁₁ and R₁₂ together with the atom to which they are attached form a 3-10 membered heterocycloalkyl ring, which is optionally substituted with one or more substituents independently selected from the group consisting of
      (a) halogen,
      (b) hydroxy,
      (c) C₁-C₃-alkoxy,
      (d) C₁-C₃-alkoxy-C₁-C₃-alkoxy,
      (e) oxo,
      (f) C₁-C₁₂-alkyl,
      (g) substituted loweralkyl,
      (h) halo-C₁-C₁₂-alkyl,
      (i) amino,
      (j) alkylamino,
      (k) dialkylamino
   and
      (l) C₁-C₃-alkoxy-C₁-C₁₂-alkyl,
   or
   R and its connected oxygen atom taken together is halogen;

R₃ is selected from the group consisting of
(1) OH,
(2) 1-adamantanamino,
(3) 2-adamantanamino,
(4) 3-amino-1-adamantanamino,
(5) 1-amino-3-adamantanamino,
(6) 3-loweralkylamino-1-adamantanamino,
(7) 1-loweralkylamino-3-adamantanamino,
(8) amino,
(9) NR₁₃R₁₄ wherein R₁₃ and R₁₄ are each independently selected from the group consisting of hydrogen, loweralkyl, substituted loweralkyl, cycloalkyl, substituted cycloalkyl, aminoloweralkyl wherein the amino portion of the aminoloweralkyl group is further substituted with unsubstituted or substituted alkyl, alkenyl, cycloalkyl, cycloalkenyl, arylaryl, alkoxy, aryloxy, substituted alkoxy, and substituted aryloxy
or
R₁₃ and R₁₄ together with the atom to which they are attached form a 3-10 membered heterocycloalkyl ring, which is optionally substituted with one or more substituents independently selected from the group consisting of
   (a) halogen,
   (b) hydroxy,
   (c) C₁-C₃-alkoxy,
   (d) C₁-C₃-alkoxy-C₁-C₃-alkoxy,
   (e) oxo,
   (f) C₁-C₁₂-alkyl,
   (g) substituted loweralkyl,
   (h) halo-C₁-C₁₂-alkyl,
   (i) amino,
   (j) alkylamino,
   (k) dialkylamino,
   and
   (l) C₁-C₃-alkoxy-C₁-C₁₂-alkyl;

R₄ is selected from the group consisting of
(1) CH₂NH—CHR₁₅—(CH₂)ₘ—NHSO₂R_{B}, wherein m is 1 to 6 and R₁₅ is H or loweralkyl,
(2) CH₂NH—CHR₁₅—(CH₂)ₚ—CONHSO₂R_{B}, wherein p is 0 to 6 and R₁₅ is H or loweralkyl,
(3) CH₂NH—CHR₁₅—(CH₂)ₚ—COOH, wherein p is 0 to 6 and R₁₅ is H or loweralkyl,
(4) CH₂NR_{F}—CHR₁₅—(CH₂)_{q}—NR_{G}SO₂R_{B}, wherein q is 2 to 4 and R₁₅ is H or loweralkyl, R_{F} and R_{G} are independently hydrogen, lower alkyl or taken together represents a —CH₂—,
(5) H,
(6) CH₂NHCH₂PO₃H₂,
(7) aminoloweralkyl wherein the amino portion of the aminoloweralkyl group is further substituted with unsubstituted or substituted alkyl, alkenyl, cycloalkyl, cycloalkenyl, arylaryl, alkoxy, aryloxy, substituted alkoxy, and substituted aryloxy;

R_{B} is selected from the group consisting of
a) aryl,
b) C₁-C₁₂-alkyl,
c) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) amino,
   (f) C₁-C₁₂-alkylamino,
   (g) C₁-C₁₂-dialkylamino,
   (h) alkenyl,
   (i) alkynyl,
   (j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) heteroaryl,
j) heterocycloalkyl,
k) aryl substituted with one or more substituents selected from the group consisting of
   (a) halogen,
   (b) hydroxy,
   (c) C₁-C₁₂-alkoxy,
   (d) C₁-C₆-alkoxy-C₁-C₆-alkoxy,
   (e) amino,
   (f) amino-C₁-C₆-alkoxy,
   (g) C₁-C₁₂-alkylamino,
   (h) C₁-C₁₂-alkylamino-C₁-C₆-alkoxy,
   (i) C₁-C₁₂-dialkylamino,
   (j) C₁-C₁₂-dialkylamino-C₁-C₆-alkoxy,
   (k) alkenyl,
   (l) alkynyl,
   (m) C₁-C₁₂-thioalkoxy,
   (n) C₁-C₁₂-alkyl,
l) heteroaryl substituted with one or more substituents selected from the group consisting of
   (a) halogen,
   (b) hydroxy,
   (c) C₁-C₁₂-alkoxy,
   (d) C₁-C₆-alkoxy-C₁-C₆-alkoxy,
   (e) amino,
   (f) amino-C₁-C₆-alkoxy,
   (g) C₁-C₁₂-alkylamino,
   (h) C₁-C₁₂-alkylamino-C₁-C₆-alkoxy,
   (i) C₁-C₁₂-dialkylamino,
   (j) C₁-C₁₂-dialkylamino-C₁-C₆-alkoxy,
   (k) alkenyl,
   (l) alkynyl,
   (m) C₁-C₁₂-thioalkoxy,
   (n) C₁-C₁₂-alkyl;

R_{C} is each selected from the group consisting of
a) hydrogen,
b) C₁-C₁₂-alkyl,
c) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) amino,
   (f) C₁-C₁₂-alkylamino,
   (g) C₁-C₁₂-dialkylamino,
   (h) alkenyl,
   (i) alkynyl,
   (j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
k) C(═O) R₇ wherein R₇ is previously defined,
l) C(═O) CHR₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl,
   or
   R₈ and R₁₀ or R₉ and R₁₀ taken together with the atom to which they are attached form a 3-10 membered heterocycloalkyl ring which is optionally substituted with one or more substituents independently selected from the group consisting of
   (a) halogen,
   (b) hydroxyl,
   (c) C₁-C₃-alkoxy,
   (d) C₁-C₃-alkoxy-C₁-C₃-alkoxy,
   (e) oxo,
   (f) C₁-C₃-alkyl,
   (g) halo-C₁-C₃-alkyl,
   (h) C₁-C₃-alkoxy-C₁-C₃-alkyl;

R_{D} and R_{E} are each independently selected from the group consisting of
a) hydrogen,
b) C₁-C₁₂-alkyl,
c) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) amino,
   (f) C₁-C₁₂-alkylamino,
   (g) C₁-C₁₂-dialkylamino,
   (h) alkenyl,
   (i) alkynyl,
   (j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
   or
   R_{D} and R_{E} taken together with the atom to which they are attached form a 3-10 membered heterocycloalkyl ring which optionally contains one to two hetero functionalities selected from the group consisting of —O—, —N—, —NH, —N(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)ₙ— wherein n is 1 or 2 and the 3-10 membered heterocycloalkyl ring is optionally substituted with one or more substituents independently selected from the group consisting of
   (a) halogen,
   (b) hydroxyl,
   (c) C₁-C₃-alkoxy,
   (d) C₁-C₃-alkoxy-C₁-C₃-alkoxy,
   (e) oxo,
   (f) C₁-C₃-alkyl,
   (g) halo-C₁-C₃-alkyl,
   (h) C₁-C₃-alkoxy-C₁-C₃-alkyl,
and
k) C(═O) R₇ wherein R₇ is previously defined,
l) C(═O) CH R₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl,
   or
   R₈ and R₁₀ or R₉ and R₁₀ taken together with the atom to which they are attached form a 3-10 membered heterocycloalkyl ring which is optionally substituted with one or more substituents independently selected from the group consisting of
   (a) halogen,
   (b) hydroxyl,
   (c) C₁-C₃-alkoxy,
   (d) C₁-C₃-alkoxy-C₁-C₃-alkoxy,
   (e) oxo,
   (f) C₁-C₃-alkyl,
   (g) halo-C₁-C₃-alkyl,
   (h) C₁-C₃-alkoxy-C₁-C₃-alkyl,
m) C(═O) CH R₈NR₉R₇ wherein R₇, R₈ and R₉ are as previously defined;
or a pharmaceutically acceptable salt, ester, solvate, alkylated quaternary ammonium salt, stereoisomer, tautomer or prodrug thereof.

In a further embodiment, the compound has the structure of Formula I

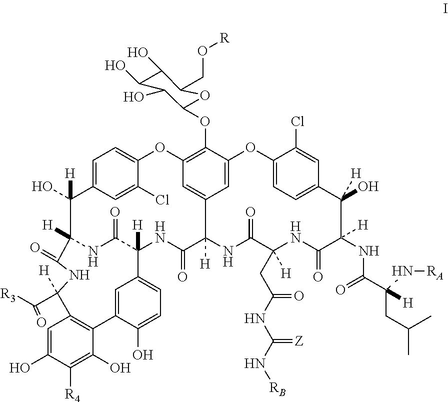

or a pharmaceutically acceptable, salt, ester, solvate, alkylated quaternary ammonium salt, stereoisomer, tautomer or prodrug thereof, wherein R, etc. have the meanings as defined herein.

In a further embodiment, the compound has the structure of Formula II

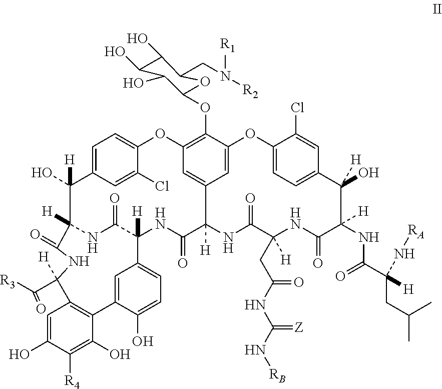

or a pharmaceutically acceptable salt, ester, solvate, alkylated quaternary ammonium salt, stereoisomer, tautomer or prodrug thereof, wherein R, etc. have the meanings as defined herein.

In a further embodiment, the compound has the structure of Formula III

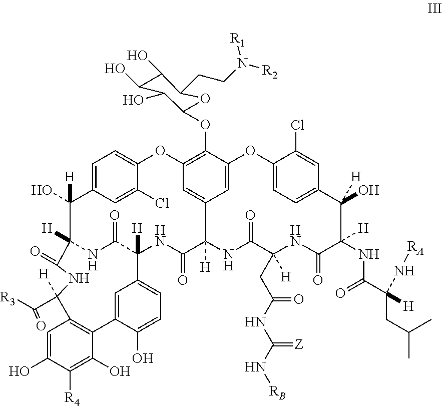

or a pharmaceutically acceptable salt, ester, solvate, alkylated quaternary ammonium salt, stereoisomer, tautomer or prodrug thereof, wherein R, etc. have the meanings as defined herein.

In a further embodiment, the compound has the structure of Formula IV

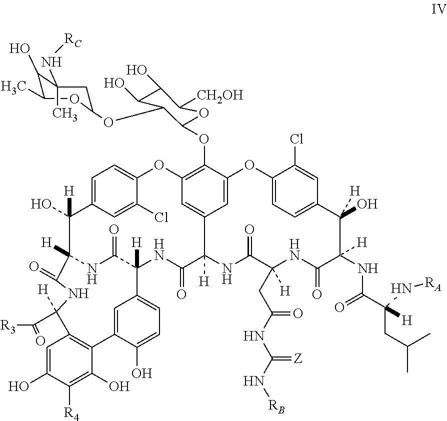

or a pharmaceutically acceptable salt, ester, solvate, alkylated quaternary ammonium salt, stereoisomer, tautomer or prodrug thereof, wherein R, etc. have the meanings as defined herein.

In a further embodiment, the compound has the structure of Formula V

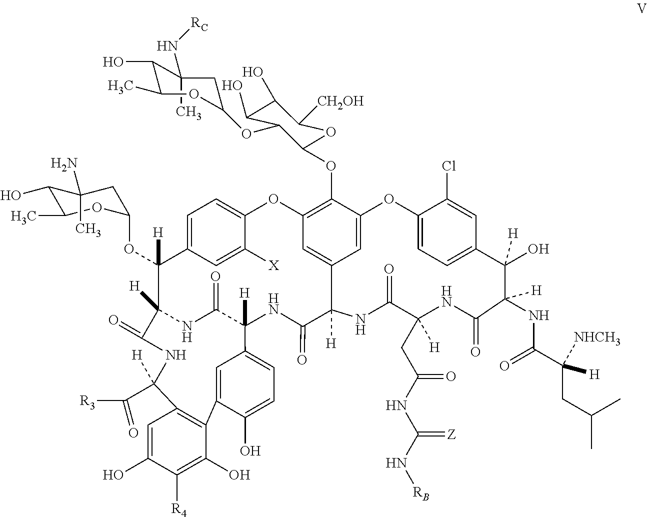

or a pharmaceutically acceptable salt, ester, solvate, alkylated quaternary ammonium salt, stereoisomer, tautomer or prodrug thereof, wherein R, etc. have the meanings as defined herein.

In a further embodiment, the compound has the structure of Formula VI

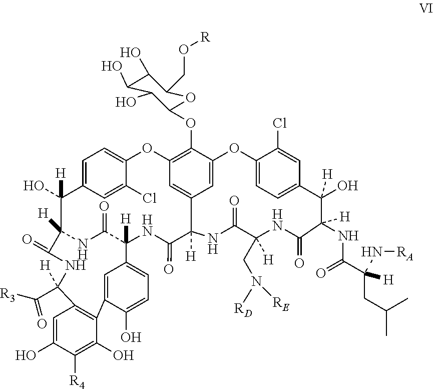

or a pharmaceutically acceptable salt, ester, solvate, alkylated quaternary ammonium salt, stereoisomer, tautomer or prodrug thereof, wherein R, etc. have the meanings as defined herein.

In a further embodiment, the compound has the structure of Formula VII

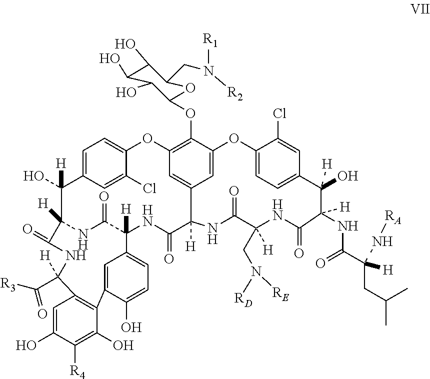

or a pharmaceutically acceptable salt, ester, solvate, alkylated quaternary ammonium salt, stereoisomer, tautomer or prodrug thereof, wherein R, etc. have the meanings as defined herein.

In a further embodiment, the compound has the structure of Formula VIII

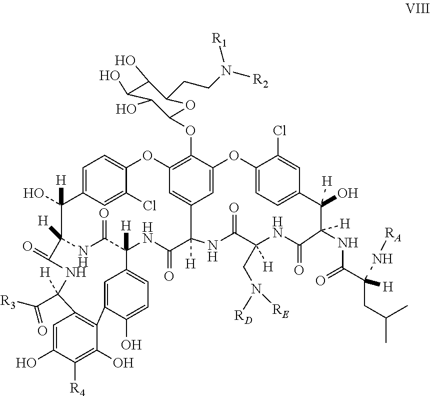

or a pharmaceutically acceptable salt, ester, solvate, alkylated quaternary ammonium salt, stereoisomer, tautomer or prodrug thereof, wherein R, etc. have the meanings as defined herein.

In a further embodiment, the compound has the structure of Formula IX

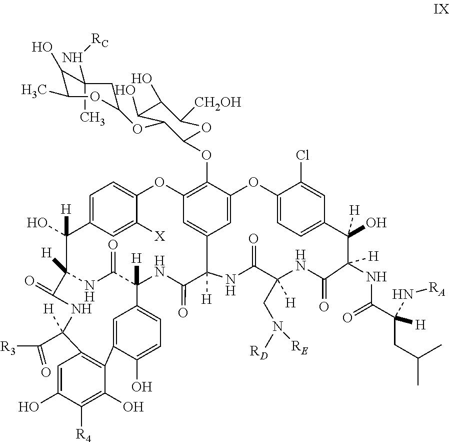

or a pharmaceutically acceptable salt, ester, solvate, alkylated quaternary ammonium salt, stereoisomer, tautomer or prodrug thereof, wherein R, etc. have the meanings as defined herein.

In a further embodiment, the compound has the structure of Formula X

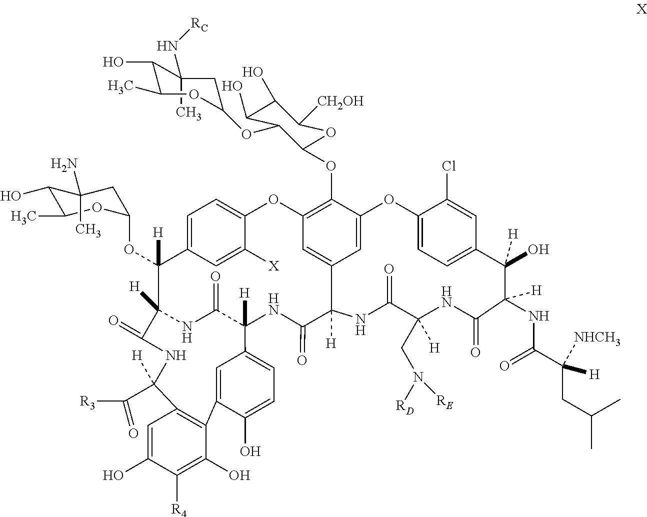

or a pharmaceutically acceptable salt, ester, solvate, alkylated quaternary ammonium salt, stereoisomer, tautomer or prodrug thereof, wherein R, etc. have the meanings as defined herein.

In a further embodiment, the compound has the structure of Formula XI

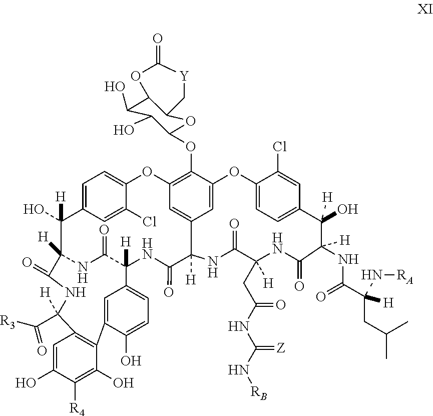

or a pharmaceutically acceptable salt, ester, solvate, alkylated quaternary ammonium salt, stereoisomer, tautomer or prodrug thereof, wherein R, etc. have the meanings as defined herein.

In a further embodiment, the compound has the structure of Formula XII

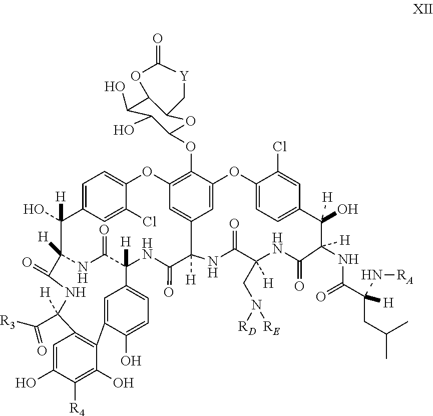

or a pharmaceutically acceptable salt, ester, solvate, alkylated quaternary ammonium salt, stereoisomer, tautomer or prodrug thereof, wherein R, etc. have the meanings as defined herein.

In a further embodiment of any of the above structures, R_{A} is methyl and R₄ is hydrogen. In embodiment, R_{A} is hydrogen and R₄ is hydrogen. In another embodiment, X is hydrogen and R₄ is hydrogen. In a further embodiment, X is chlorine and R₄ is hydrogen. In yet a further embodiment, R_{A} is methyl and R₄ is CH₂NHCH₂PO₃H₂. In another embodiment, R_{A} is hydrogen and R₄ is CH₂NHCH₂PO₃H₂. In one embodiment, R_{A} is hydrogen and R₄ is CH₂NH—CHR₁₅—(CH₂)ₙ—NHSO₂R_{B}, wherein m is 1 to 6 and R₁₅ is H or loweralkyl. In another embodiment, R_{A} is hydrogen and R₄ is CH₂NR_{F}—CHR₁₅—(CH₂)_{q}—NR_{G}SO₂R_{B}, wherein q is 2 to 4, R₁₅, R_{F}, and R_{G} is H or loweralkyl, R_{F} and R_{G} together represents —CH₂—. In yet another embodiment, R_{A} is hydrogen and R₄ is CH₂NH—CHR₁₅—(CH₂)ₚ—CONHSO₂R_{B}, wherein p is 0 to 6 and R₁₅ is H or loweralkyl. In a further embodiment, R_{A} is hydrogen and R₄ is CH₂NH—CHR₁₅—(CH₂)ₚ—COOH, wherein p is 0 to 6 and R₁₅ is H or loweralkyl. In yet a further embodiment, R_{A} is methyl and R₄ is CH₂NH—CHR₁₅—(CH₂)ₘ—NHSO₂R_{B}, wherein m is 1 to 6 and R₁₅ is H or loweralkyl. In one embodiment, R_{A} is methyl and R₄ is CH₂NH—CHR₁₅—(CH₂)ₚ—CONHSO₂R_{B}, wherein p is 0 to 6 and R₁₅ is H or loweralkyl. In another embodiment, R_{A} is methyl and R₄ is CH₂NH—CHR₁₅—(CH₂)ₚ—COOH, wherein p is 0 to 6 and R₁₅ is H or loweralkyl.

In a further embodiment of any of the aforementioned embodiments, R₃ is selected from the group consisting of
(1) OH,
(2) 1-adamantanamino,
(3) 2-adamantanamino,
(4) 3-amino-1-adamantanamino,
(5) 1-amino-3-adamantanamino,
(6) 3-loweralkylamino-1-adamantanamino,
(7) 1-loweralkylamino-3-adamantanamino,
(8) amino
(9) NR₁₃R₁₄ wherein R₁₃ and R₁₄ are each independently selected from the group consisting of hydrogen, loweralkyl, substituted loweralkyl, cycloalkyl, substituted cycloalkyl, aminoloweralkyl wherein the amino portion of the aminoloweralkyl group is further substituted with unsubstituted or substituted alkyl, alkenyl, cycloalkyl, cycloalkenyl, arylaryl, alkoxy, aryloxy, substituted alkoxy, and substituted aryloxy
or
R₁₃ and R₁₄ together with the atom to which they are attached form a 3-10 membered heterocycloalkyl ring, which is optionally substituted with one or more substituents independently selected from the group consisting of
   (a) halogen,
   (b) hydroxy,
   (c) C₁-C₃-alkoxy,
   (d) C₁-C₃-alkoxy-C₁-C₃-alkoxy,
   (e) oxo,
   (f) C₁-C₁₂-alkyl,
   (g) substituted loweralkyl,
   (h) halo-C₁-C₁₂-alkyl,
   (i) amino,
   (j) alkylamino,
   (k) dialkylamino,
   and
   (l) C₁-C₃-alkoxy-C₁-C₁₂-alkyl.

In a further embodiment, R₃ is OH. In another embodiment, R₃ is 2-adamantanamino. In yet another embodiment, R₃ is dimethylamino. In one embodiment, R₃ is dimethylaminoethylamino. In another embodiment, R₃ is N-methylpiperazino.

In a further embodiment of any of the aforementioned embodiments, R₁ and R₂ are each independently selected from the group consisting of
a) hydrogen,
b) C₁-C₁₂-alkyl,
c) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) amino,
   (f) C₁-C₁₂-alkylamino,
   (g) C₁-C₁₂-dialkylamino,
   (h) alkenyl,
   (i) alkynyl,
   (j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
   or
   R₁ and R₂ taken together with the atom to which they are attached form a substituted heteroaryl or 3-10 membered heterocycloalkyl ring which optionally contains one to two hetero functionalities selected from the group consisting of —O—, —N—, —NH, —N(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)ₙ— wherein n is 1 or 2 and the 3-10 membered heterocycloalkyl ring is optionally substituted with one or more substituents independently selected from the group consisting of
   (a) halogen,
   (b) hydroxyl,
   (c) C₁-C₃-alkoxy,
   (d) C₁-C₃-alkoxy-C₁-C₃-alkoxy,
   (e) oxo,
   (f) C₁-C₃-alkyl,
   (g) halo-C₁-C₃-alkyl,
   (h) C₁-C₃-alkoxy-C₁-C₃-alkyl,
and
k) C(═O) R₇,
l) C(═O) CH R₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl, or
   R₈ and R₁₀ or R₉ and R₁₀ taken together with the atom to which they are attached form a 3-10 membered heterocycloalkyl ring which is optionally substituted with one or more substituents independently selected from the group consisting of
   (a) halogen,
   (b) hydroxyl,
   (c) C₁-C₃-alkoxy,
   (d) C₁-C₃-alkoxy-C₁-C₃-alkoxy,
   (e) oxo,
   (f) C₁-C₃-alkyl,
   (g) halo-C₁-C₃-alkyl,
   (h) C₁-C₃-alkoxy-C₁-C₃-alkyl.

In a further embodiment of any of the aforementioned embodiments, R₁ and R₂ are hydrogen. In another embodiment, R₁ is C₁-C₁₂-alkyl and R₂ is hydrogen. In yet another embodiment, R_{I} is C₁-C₁₂-alkyl substituted with aryl or substituted aryl and R₂ is hydrogen. In a further embodiment, R₁ is C(═O)C₁-C₁₂-alkyl and R₂ is hydrogen. In yet a further embodiment, R₁ is C(═O)CH₂ NH C₁-C₁₂-alkyl and R₂ is hydrogen. In one embodiment, R₁ is C₁-C₁₂-alkyl substituted C₁-C₁₂-alkoxy and R₂ is hydrogen. In another embodiment, R₁ is C₁-C₁₂-alkyl substituted C₁-C₁₂-thioalkoxy and R₂ is hydrogen. In yet another embodiment, R₁ is C₁-C₁₂-alkyl substituted C₁-C₁₂-alkylamino and R₂ is hydrogen.

In a further embodiment of any of the aforementioned embodiments, R is selected from the group consisting of
(1) hydrogen,
(2) cycloalkyl,
(3) cycloalkenyl,
(4) C₁-C₁₂-alkyl,
(5) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) —COOR₅ wherein R₅ is hydrogen or loweralkyl,
   (f) —C(O)NR₅R₆ wherein R₅ is as previously defined and R₆ is hydrogen or loweralkyl,
   (g) amino,
   (h) —NR₅R₆ wherein R₅ and R₆ are as previously defined,
      or
      R₅ and R₆ are taken together with the atom to which they are attached from a 3-10 membered heterocycloalkyl ring which is optionally substituted with one or more substituents independently selected from the group consisting of
      (i) halogen,
      (ii) hydroxy,
      (iii) C₁-C₃-alkoxy,
      (iv) C₁-C₃-alkoxy-C₁-C₃-alkoxy,
      (v) oxo,
      (vi)
      (vii) halo-C₁-C₁₂-alkyl,
      and
      (viii) C₁-C₃-alkoxy-C₁-C₁₂-alkyl,
   (i) aryl,
   (j) substituted aryl,
   (k) heteroaryl,
   (l) substituted heteroaryl,
   (m) mercapto,
   (n) C₁-C₁₂-thioalkoxy,
(6) C(═O)O R₁₁, wherein R₁₁ is hydrogen, loweralkyl, substituted loweralkyl, aryl, substituted aryl, heteroaryl or substituted heteroaryl,
(7) C(═O)NR₁₁ R₁₂, wherein R₁₁ is as previously defined and R₁₂ is hydrogen, loweralkyl, substituted loweralkyl, aryl, substituted aryl, heteroaryl or substituted heteroaryl,
   or
   R₁₁ and R₁₂ together with the atom to which they are attached form a 3-10 membered heterocycloalkyl ring, which is optionally substituted with one or more substituents independently selected from the group consisting of
      (a) halogen,
      (b) hydroxy,
      (c) C₁-C₃-alkoxy,
      (d) C₁-C₃-alkoxy-C₁-C₃-alkoxy,
      (e) oxo,
      (f) C₁-C₁₂-alkyl,
      (g) substituted loweralkyl,
      (h) halo-C₁-C₁₂-alkyl,
      (i) amino,
      (j) alkylamino,
      (k) dialkylamino,
      and
      (l) C₁-C₃-alkoxy-C₁-C₁₂-alkyl,
      or
      R and its connected oxygen atom taken together is halogen.

In a further embodiment of any of the aforementioned embodiments, R is hydrogen. In another embodiment, R is C₁-C₁₂-alkyl. In one embodiment, R is C₁-C₁₂-alkyl substituted with aryl or substituted aryl. In a further embodiment, R is C(═O)NHC₁-C₁₋₂-alkyl. In yet a further embodiment, R₁ is C(═O)NHC₁-C₁₂-alkyl substituted with aryl or substituted aryl. In one embodiment, R is C(═O)OC₁-C₁₂-alkyl. In another embodiment, R₁ is C(═O)NHC₁-C₁₂-alkyl substituted with heteroaryl or substituted heteroaryl.

In a further embodiment of any of the aforementioned embodiments, R_{B} is selected from the group consisting of
a) aryl,
b) C₁-C₁₂-alkyl,
c) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) amino,
   (f) C₁-C₁₂-alkylamino,
   (g) C₁-C₁₂-dialkylamino,
   (h) alkenyl,
   (i) alkynyl,
   (j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) heteroaryl,
j) heterocycloalkyl,
k) substituted aryl,
l) substituted heteroaryl.

In a further embodiment of any of the aforementioned embodiments, R_{B} is C₁-C₁₂-alkyl. In another embodiment, R_{B} is C₁-C₁₂-alkyl substituted with aryl or substituted aryl. In yet another embodiment, R_{B} is C₁-C₁₂-alkyl substituted with heteroaryl or substituted heteroaryl.

In a further embodiment of any of the aforementioned embodiments, R_{C} is each selected from the group consisting of
a) hydrogen,
b) C₁-C₁₂-alkyl,
c) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) amino,
   (f) C₁-C₁₂-alkylamino,
   (g) C₁-C₁₂-dialkylamino,
   (h) alkenyl,
   (i) alkynyl,
   (j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
k) C(═O) R₇ wherein R₇ is previously defined,
l) C(═O) CH R₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl,
   or
   R₈ and R₁₀ or R₉ and R₁₀ taken together with the atom to which they are attached form a 3-10 membered heterocycloalkyl ring which is optionally substituted with one or more substituents independently selected from the group consisting of
   (a) halogen,
   (b) hydroxyl,
   (c) C₁-C₃-alkoxy,
   (d) C₁-C₃-alkoxy-C₁-C₃-alkoxy,
   (e) oxo,
   (f) C₁-C₃-alkyl,
   (g) halo-C₁-C₃-alkyl,
   (h) C₁-C₃-alkoxy-C₁-C₃-alkyl.

In a further embodiment of any of the aforementioned embodiments, R_{C} is hydrogen. In another embodiment, R_{C} is C₁-C₁₂-alkyl. In yet another embodiment, R_{C} is C₁-C₁₂-alkyl substituted with aryl or substituted aryl. In a further embodiment, R_{C} is C₁-C₁₂-alkyl substituted with heteroaryl or substituted heteroaryl. In one embodiment, R_{C} is C(═O)C₁-C₁₂-alkyl. In another embodiment, R_{C} is C(═O)CH₂ NH C₁-C₂-alkyl. In yet another embodiment, R_{C} is C₁-C₁₂-alkyl substituted C₁-C₁₂-alkoxy. In a further embodiment, R_{C} is C₁-C₁₂-alkyl substituted C₁-C₁₂-thioalkoxy. In yet a further embodiment, R_{C} is C₁-C₁₂-alkyl substituted C₁-C₁₂-alkylamino.

In a further embodiment of any of the aforementioned embodiments, R_{D} and R_{E} are each independently selected from the group consisting of
a) hydrogen,
b) C₁-C₁₂-alkyl,
c) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) amino,
   (f) C₁-C₁₂-alkylamino,
   (g) C₁-C₁₂-dialkylamino,
   (h) alkenyl,
   (i) alkynyl,
   (j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
   or
   R_{D} and R_{E} taken together with the atom to which they are attached form a 3-10 membered heterocycloalkyl ring which optionally contains one to two hetero functionalities selected from the group consisting of —O—, —N—, —NH, —N(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)ₙ— wherein n is 1 or 2 and the 3-10 membered heterocycloalkyl ring is optionally substituted with one or more substituents independently selected from the group consisting of
   (a) halogen,
   (b) hydroxyl,
   (c) C₁-C₃-alkoxy,
   (d) C₁-C₃-alkoxy-C₁-C₃-alkoxy,
   (e) oxo,
   (f) C₁-C₃-alkyl,
   (g) halo-C₁-C₃-alkyl,
   (h) C₁-C₃-alkoxy-C₁-C₃-alkyl,
and
k) C(═O) R₇ wherein R₇ is previously defined,
l) C(═O) CH R₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl,
   or
   R₈ and R₁₀ or R₉ and R₁₀ taken together with the atom to which they are attached form a 3-10 membered heterocycloalkyl ring which is optionally substituted with one or more substituents independently selected from the group consisting of
   (a) halogen,
   (b) hydroxyl,
   (c) C₁-C₃-alkoxy,
   (d) C₁-C₃-alkoxy-C₁-C₃-alkoxy,
   (e) oxo,
   (f) C₁-C₃-alkyl,
   (g) halo-C₁-C₃-alkyl,
   (h) C₁-C₃-alkoxy-C₁-C₃-alkyl,
m) C(═O) CH R₈NR₉R₇ wherein R₇, R₈ and R₉ are as previously defined.

In a further embodiment of any of the aforementioned embodiments, R_{D} and R_{E} are hydrogen. In another embodiment, R_{D} is C₁-C₁₂-alkyl and R_{E} is hydrogen. In yet another embodiment, R_{D} is C₁-C₁₂-alkyl substituted with aryl or substituted aryl and R_{E} is hydrogen. In one embodiment, R_{D} is C(═O)C₁-C₁₂-alkyl and R_{E} is hydrogen. In a further embodiment, R_{D} is C(═O)CH₂ NH C₁-C₂-alkyl and R_{E} is hydrogen. In yet a further embodiment, R_{D} is C₁-C₁₂-alkyl substituted C₁-C₁₂-alkoxy and R_{E} is hydrogen. In another embodiment, R_{D} is C₁-C₁₂-alkyl substituted C₁-C₁₂-thioalkoxy and R_{E} is hydrogen. In one embodiment, R_{D} is C₁-C₁₂-alkyl substituted C₁-C₁₂-alkylamino and R_{E} is hydrogen.

In a further embodiment of any of the above structures, Y is oxygen and R₄ is hydrogen. In another embodiment, Z is oxygen and R₄ is hydrogen. In yet another embodiment, Y is NH and R₄ is hydrogen. In a further embodiment, Z is sulfur and R₄ is hydrogen. In yet a further embodiment, Z is oxygen and R₄ is CH₂NHCH₂PO₃H₂. In one embodiment, Y is oxygen and R₄ is CH₂NHCH₂PO₃H₂. In another embodiment, Y is NH and R₄ is CH₂NHCH₂PO₃H₂.

In a further embodiment of any of the aforementioned embodiments, R₁ is hydrogen and R₂ is COCHR₈NHR₁₅ wherein R₁₅ is substituted arylalkyl and R₈ is as previously defined.

In another aspect are compounds selected from Compound (23), Compound (24), Compound (25), Compound (26), Compound (27), Compound (28), Compound (29), Compound (30), Compound (31), Compound (32), Compound (33), Compound (34), Compound (44), Compound (45), Compound (46), Compound (48), Compound (49), Compound (50), Compound (51), Compound (57), Compound (58), Compound (59), Compound (60), Compound (73), Compound (74), Compound (75), Compound (76), Compound (77), Compound (78), Compound (79), Compound (80), Compound (81), Compound (84), Compound (85), Compound (86), Compound (87), Compound (88), Compound (89), Compound (90), Compound (91), Compound (92), Compound (93), Compound (94), Compound (95), Compound (96), Compound (97), Compound (98), Compound (99), Compound (100), Compound (101), Compound (102), Compound (103), Compound (104), Compound (105), Compound (106), Compound (107), Compound (108), Compound (124), Compound (125), Compound (126), Compound (127), Compound (128), Compound (129), Compound (130), Compound (131), Compound (132), Compound (133), Compound (134), Compound (135), Compound (136), Compound (137), Compound (138), Compound (140), Compound (141), Compound (142), Compound (143), Compound (145), Compound (151), Compound (152), Compound (153), Compound (154), Compound (155), Compound (156), Compound (157), Compound (158), Compound (159), Compound (160), Compound (161), Compound (163), Compound (164), Compound (165), Compound (166), Compound (167), Compound (168), Compound (169), Compound (170), Compound (171), Compound (172), and Compound (173),

In another aspect are pharmaceutical compositions comprising a therapeutically effective amount of any of the aforementioned compounds, together with a pharmaceutically acceptable carrier.

In another aspect are methods of treating a mammal in need of such treatment comprising administering to the mammal an antibacterial effective amount of any of the aforementioned compounds together with a pharmaceutically acceptable carrier. In one embodiment, the mammal has a bacterial infection that is resistant to another antibiotic, including: vancomycin, desmethylvancomycin, eremomycin, teicoplanin (complex of five compounds), dalbavancin, oritavancin, telavancin, and A82846B (LY264826) having compounds having structures A, B, C, D, E, F, G and H; or combinations of such antibiotics.

In another aspect, described herein is the use of a compound described herein in the manufacture of a medicament for the treatment of a bacterial-related disease or condition. In one embodiment, the bacterial-related disease or condition arises from a bacteria that is resistant to another antibiotic, including: vancomycin, desmethylvancomycin, eremomycin, teicoplanin (complex of five compounds), dalbavancin, oritavancin, telavancin, and A82846B (LY264826) having compounds having structures A, B, C, D, E, F, G and H; or combinations of such antibiotics.

In another aspect, described herein are articles of manufacture, comprising packaging material, a compound of any of Formula I, Formula II, Formula III, Formula IV, Formula V, Formula VI, Formula VII, Formula VIII, Formula IX, Formula X, Formula XI or Formula XII, which is effective for treatment, prevention or amelioration of one or more symptoms of a bacterial-mediated disease or condition, within the packaging material, and a label that indicates that the compound or composition, or pharmaceutically acceptable salt, pharmaceutically acceptable N-oxide, pharmaceutically acceptable acyl glucuroide metabolite, pharmaceutically acceptable prodrug, or pharmaceutically acceptable solvate thereof, is used for treatment, prevention or amelioration of one or more symptoms of a bacterial-mediated disease or condition, are provided.

In another aspect are methods of making a compound of Formulas I-V and XI, comprising:
modifying a compound from the group consisting of Formulas i, ii, iii, iv and v,

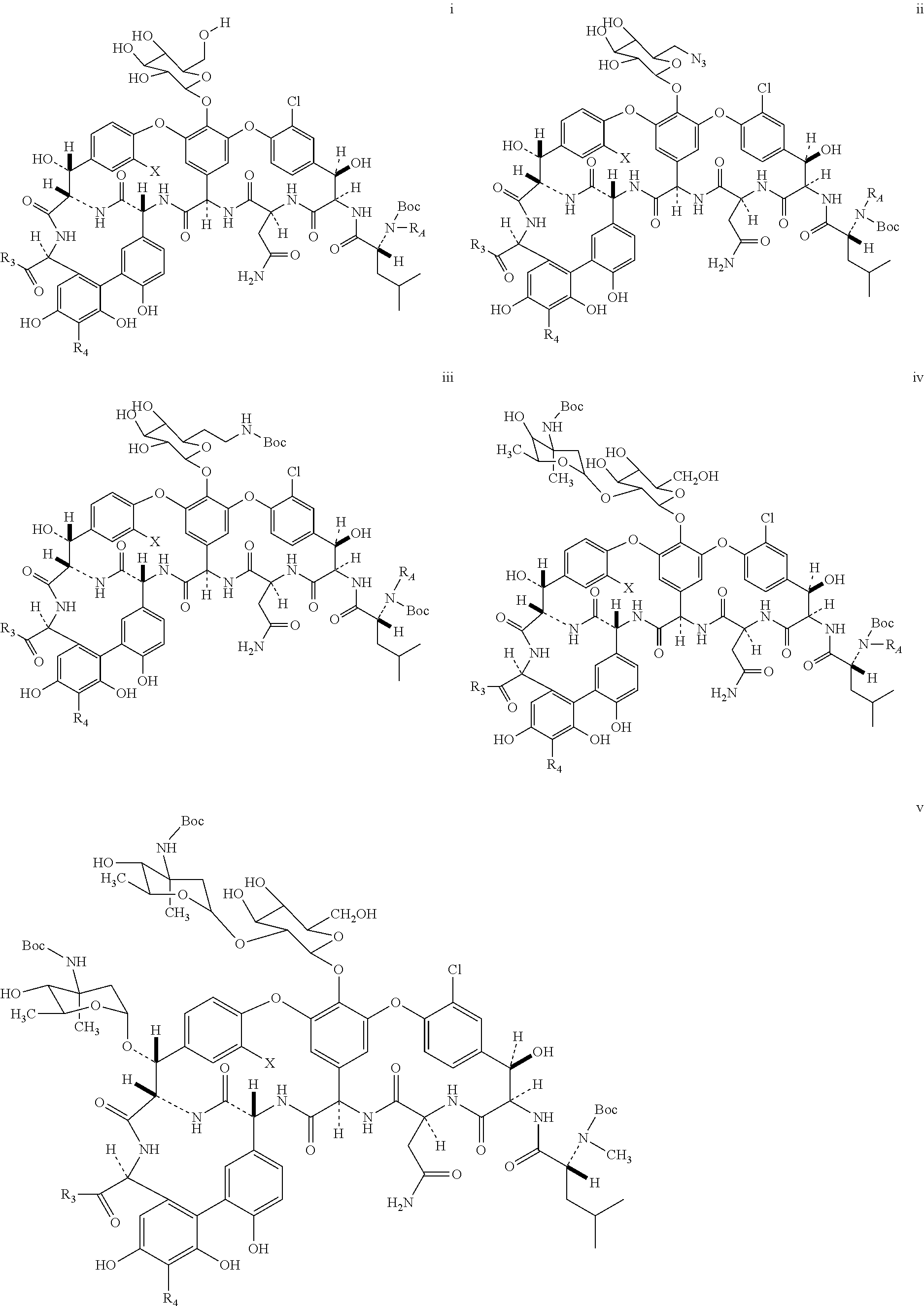

wherein R_{A} is hydrogen or methyl, X is chlorine or hydrogen, R₃ is alkoxy, 2-adamantanamino, or loweralkylamino as defined herein, or R₄ is hydrogen or properly protected CH₂NHCH₂PO₃H₂, or Boc-aminoloweralkyl as defined herein, by a technique selected from the group consisting of,
   (a) acylation of the primary amide group of the 3^{rd} amino acid asparagine with an R_{B}-isocyanate or R_{B}-thioisocyanate in the presence of a base such as dimethylaminopyridine and the like,
   (b) removal of the Boc protecting group with mild acid such as trifluoroacetic acid,
   (c) if the R₃ is alkoxy, removal of the alkoxy group by mild base or acid hydrolysis to give the carboxylic acid derivative,
   (d) reduction of the azide function to an amine,
   (e) alkylation of the primary alcohol of the mono-sugar or the amino substituent on the amino-substituted sugar moiety of the 4^{th} amino acid of the compound with an alkyl halide having structure R₁-J where J is a halogen or R_{C}-J where J is a halogen
   (f) acylation of the primary alcohol of the mono-sugar or the amino substituent on the amino-substituted sugar moiety of the 4^{th} amino acid of the compound with an acyl group having the structure, C(═O) R₇,
   (g) acylation of the primary alcohol of the mono-sugar or the amino substituent on the amino-substituted sugar moiety of the 4^{th} amino acid of the compound with an acyl group having the structure, C(═O) CHR₈NR₉R₁₀,
   (h) reaction of the amino substituent on the amino-substituted sugar moiety of the 4^{th} amino acid of the compound with an aldehyde or ketone followed by reductive amination of the resulting imine,
   (i) conversion of the acid moiety on the macrocyclic ring of the compound with substituted amide as defined by R₃,
   (j) phosgene reaction on primary alcohol or primary amine of the mono-sugar moiety of the 4^{th} amino acid of the compound with the adjacent hydroxyl group,
   (k) dipolar cycloaddition of the azide with alkyne to form 1,2,3-trizole,
   (l) a combination of (a) and (b),
   (m) a combination of (a), (b) and (c),
   (n) a combination of (a), (c), (i) and (b),
   (o) a combination of (a), (e), and (b),
   (p) a combination of (a), (f) and (b),
   (q) a combination of (a), (g) and (b),
   (r) a combination of (a), (h) and (b),
   (s) a combination of (a), (d) and (b),
   (t) a combination of (a), (d), (c) and (b),
   (u) a combination of (a), (c), (i), (d) and (b),
   (v) a combination of (a), (c), (d) and (b),
   (w) a combination of (a), (c), (i), (d), (e) and (b),
   (x) a combination of (a), (c), (i), (d), (f) and (b),
   (y) a combination of (a), (c), (i), (d), (g) and (b),
   (z) a combination of (a), (c), (i), (d), (h) and (b),
   (aa) a combination of (a), (c), (d), (e) and (b),
   (bb) a combination of (a), (c), (d), (f) and (b),
   (cc) a combination of (a), (c), (d), (g) and (b),
   (dd) a combination of (a), (c), (d), (h) and (b),
   (ee) a combination of (a), (j), and (b),
   (ff) a combination of (a), (j), (c), (i) and (b),
   (gg) a combination of (a), (d), (j), and (b),
   (hh) a combination of (a), (d), (j), (c), (i) and (b),
   (ii) a combination of (a), (k), and (b),
   (jj) a combination of (a), (k), (c), (i) and (b),
to form a compound having a formula selected from the group consisting of:

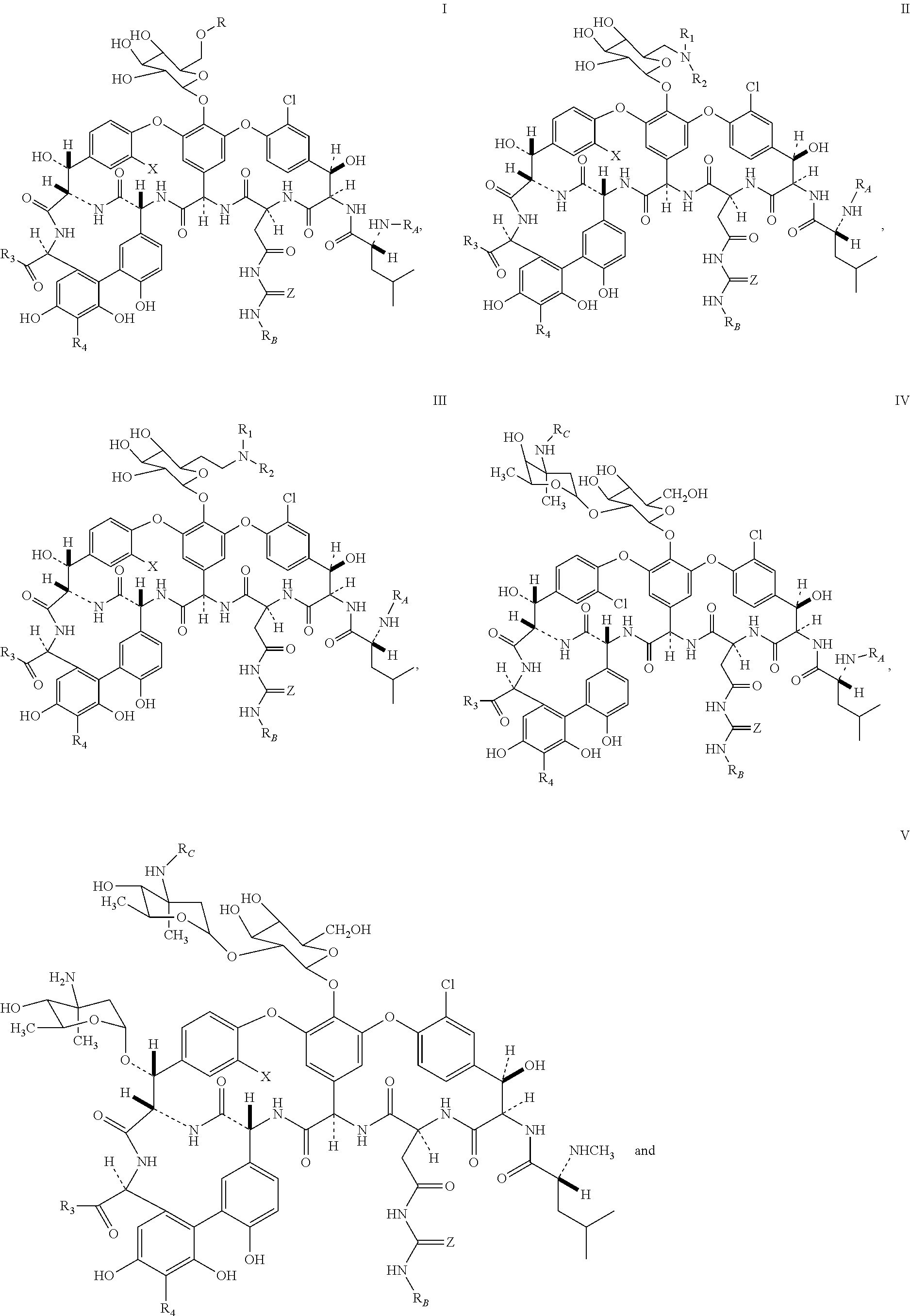

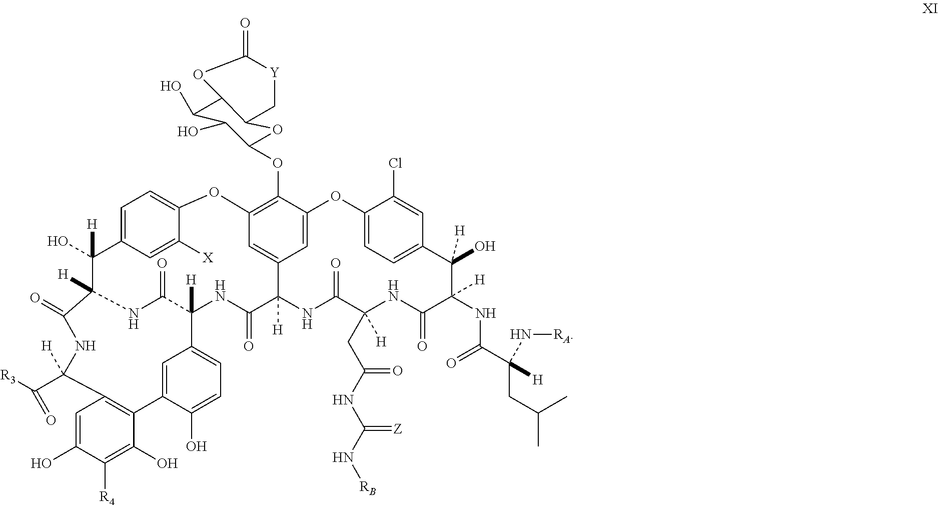

wherein R, R₁, R₂, R₃, R₄, R_{A}, R_{B}, R_{C}, X, Y, and Z are as defined herein.

In another aspect are methods of making a compound of Formulas VI-X and XII, comprising:
modifying a compound from the group consisting of Formulas vi, vii, viii, ix and x,

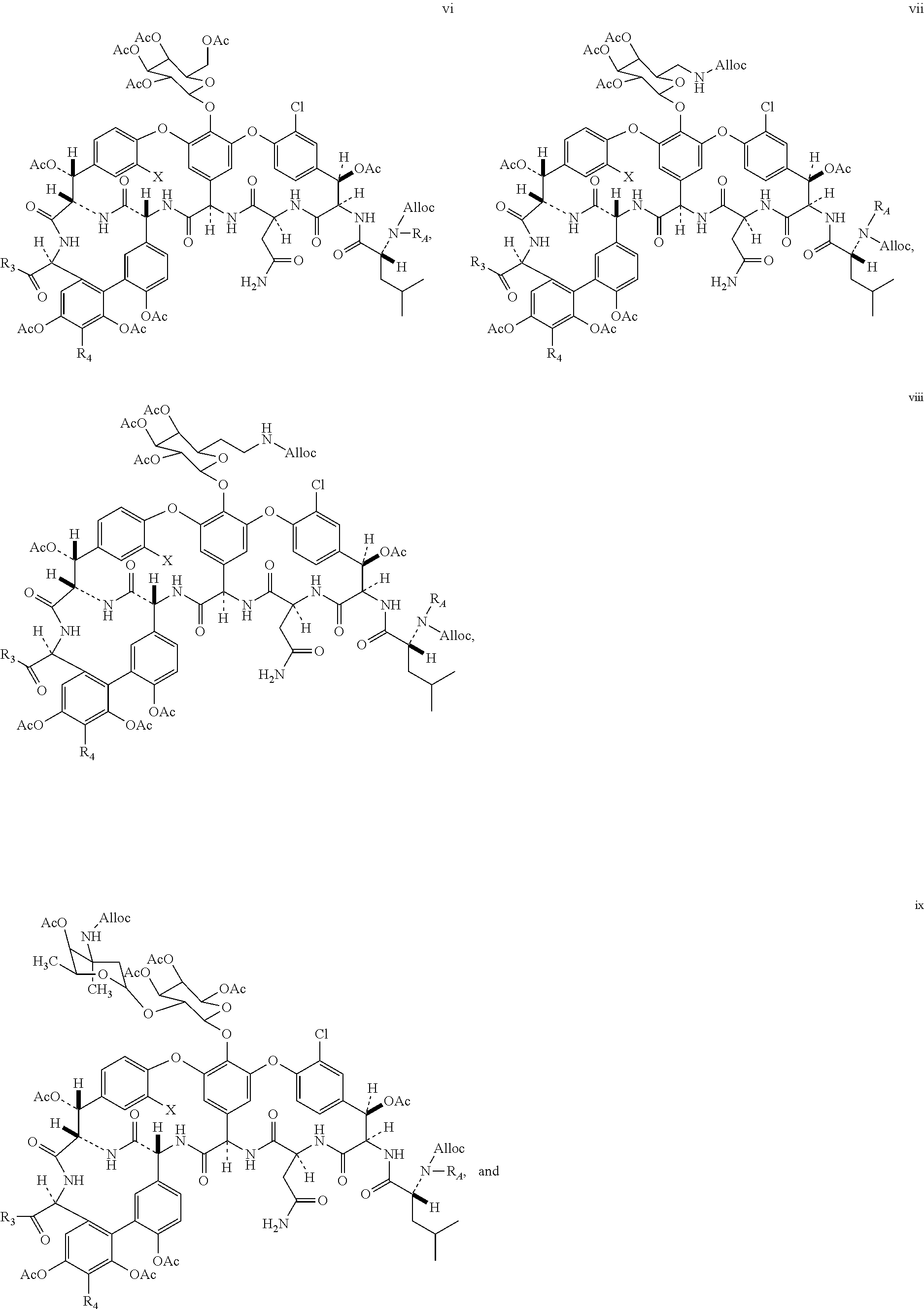

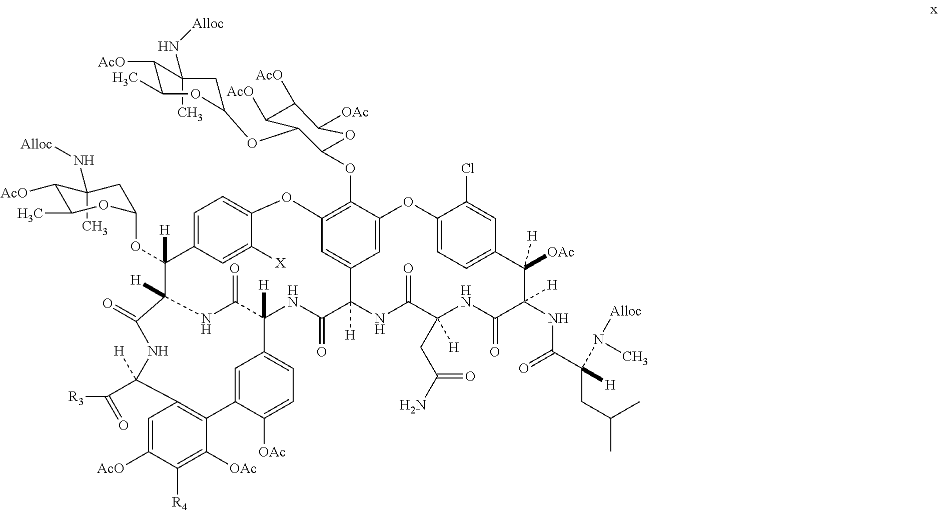

wherein R_{A} is hydrogen or methyl, X is chlorine or hydrogen, R₃ is alkoxy, 2-adamantanamino, or loweralkylamino as defined herein, or R₄ is hydrogen or properly protected CH₂NHCH₂PO₃H₂, or Boc-aminoloweralkyl as defined herein, by a technique selected from the group consisting of,
   (a) Hofmann degradation of the primary amide group of the 3^{rd} amino acid asparagine with phenyliodine-bis-trifluoroacetate to give the primary amine,
   (b) alkylation of the primary amine with an alkyl halide having structure R_{D}-J where J is a halogen or R_{E}-J where J is a halogen,
   (c) acylation of the primary amine with an acyl group having the structure, C(═O) R₇,
   (d) acylation of the primary amine with an acyl group having the structure, C(═O) CHR₈NR₉R₁₀,
   (e) removal of the N-Alloc protecting group with the use of Pd(OAc)₂, PPh₃, and (nBu)₃SnH,
   (f) hydrolysis of all acetate groups to give the alcohol,
   (g) if the R₃ is alkoxy, removal of the alkoxy group by mild base or acid hydrolysis to give the carboxylic acid derivative,
   (h) alkylation of the primary alcohol of the mono-sugar or the amino substituent on the amino-substituted sugar moiety of the 4^{th} amino acid of the compound with an alkyl halide having structure R-J where J is a halogen, R₁-J where J is a halogen or R_{C}-J where J is a halogen
   (i) acylation of the primary alcohol of the mono-sugar or the amino substituent on the amino-substituted sugar moiety of the 4^{th} amino acid of the compound with an acyl group having the structure, C(═O) R₇,
   (j) acylation of the primary alcohol of the mono-sugar or the amino substituent on the amino-substituted sugar moiety of the 4^{th} amino acid of the compound with an acyl group having the structure, C(═O) CHR₈NR₉R₁₀,
   (k) reaction of the amino substituent on the amino-substituted sugar moiety of the 4^{th} amino acid of the compound with an aldehyde or ketone followed by reductive amination of the resulting imine,
   (l) conversion of the acid moiety on the macrocyclic ring of the compound with substituted amide as defined by R₃,
   (m) phosgene reaction on primary alcohol or primary amine of the mono-sugar moiety of the 4^{th} amino acid of the compound with the adjacent hydroxyl group,
   (n) a combination of (a), (e) and (f),
   (o) a combination of (a), (b), (e) and (f),
   (p) a combination of (a), (c), (e) and (f),
   (q) a combination of (a), (d), (e) and (f),
   (r) a combination of (a), (c), (e), (f) and (g),
   (s) a combination of (a), (c), (e), (f), (g) and (l),
   (t) a combination of (a), (d), (e), (f) and (g),
   (u) a combination of (a), (d), (e), (f), (g) and (l),
   (v) a combination of (a), (c), (e), (h) and (f),
   (w) a combination of (a), (d), (e), (h), and (f),
   (x) a combination of (a), (c), (e), (h), (f) and (g),
   (y) a combination of (a), (d), (e), (h), (f) and (g),
   (z) a combination of (a), (c), (e), (h), (f), (g) and (l),
   (aa) a combination of (a), (d), (e), (h), (f), (g) and (l),
   (bb) a combination of (a), (c), (e), (i) and (f),
   (cc) a combination of (a), (d), (e), (i), and (f),
   (dd) a combination of (a), (c), (e), (i), (f) and (g),
   (ee) a combination of (a), (d), (e), (i), (f) and (g),
   (ff) a combination of (a), (c), (e), (i), (f), (g) and (l),
   (gg) a combination of (a), (d), (e), (i), (f), (g) and (l),
   (hh) a combination of (a), (c), (e), (j) and (f),
   (ii) a combination of (a), (d), (e), (j), and (f),
   (jj) a combination of (a), (c), (e), (j), (f) and (g),
   (kk) a combination of (a), (d), (e), (j), (f) and (g),
   (ll) a combination of (a), (c), (e), (j), (f), (g) and (l),
   (mm) a combination of (a), (d), (e), (j), (f), (g) and (l),
   (nn) a combination of (a), (c), (e), (k) and (f),
   (oo) a combination of (a), (d), (e), (k), and (f),
   (pp) a combination of (a), (c), (e), (k), (f) and (g),
   (qq) a combination of (a), (d), (e), (k), (f) and (g),
   (rr) a combination of (a), (c), (e), (k), (f), (g) and (l),
   (ss) a combination of (a), (d), (e), (k), (f), (g) and (l),
to form a compound having a formula selected from the group consisting of:

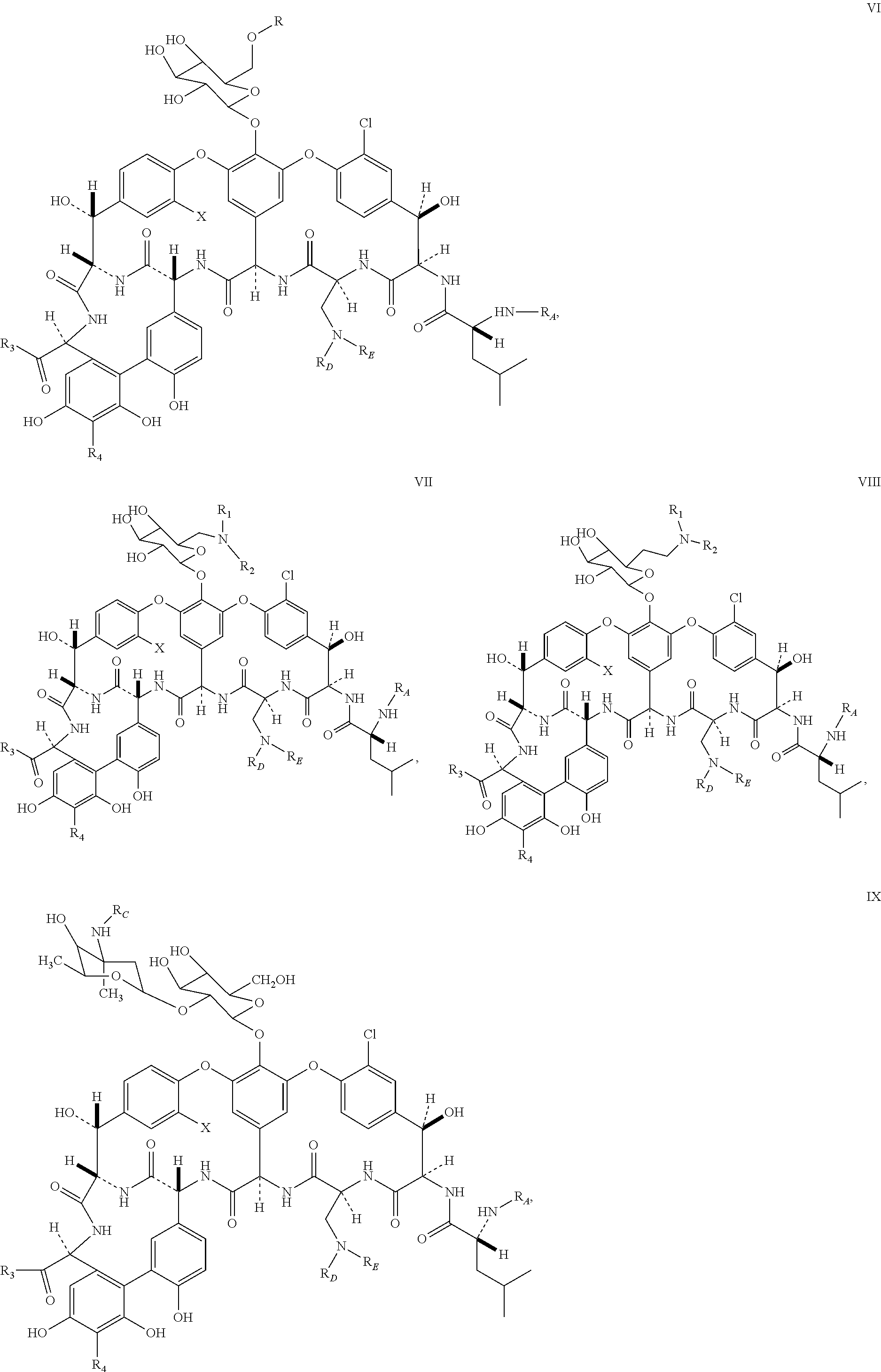

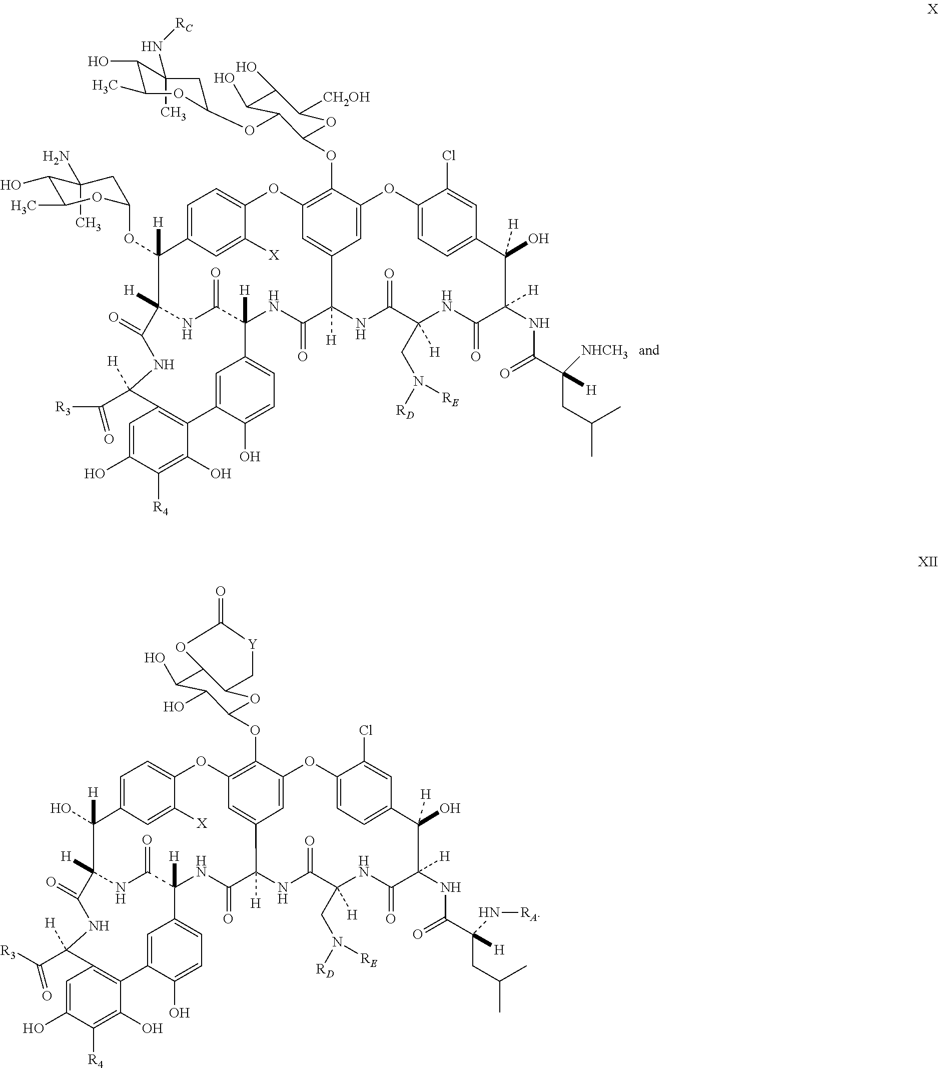

wherein R, R₁, R₂, R₃, R₄, R_{A}, R_{C}, R_{D}, R_{E}, X, Y and Z are as defined herein.

### DETAILED DESCRIPTION

The materials and associated techniques and apparatuses described herein will now be described with reference to several embodiments. Important properties and characteristics of the described embodiments are illustrated in the structures in the text. While the compositions, compounds and methods described herein are described in conjunction with these embodiments, it should be understood that the compositions, compounds and methods described herein are not to be limited to these embodiments. On the contrary, the compositions, compounds and methods described herein cover alternatives, modifications, and equivalents as are included within the spirit and scope of the appended claims. In the following description, numerous specific details are set forth in order to provide a thorough understanding of the compositions, compounds and methods described herein. The compositions, compounds and methods described herein are optionally practiced without some or all of these specific details. Well known process operations have not been described in detail in order not to unnecessarily obscure the compositions, compounds and methods described herein.

There is a continuing need to identify new derivative compounds which possess improved antibacterial activity, which have less potential for developing resistance, which possess improved effectiveness bacterial infections that resist treatment with currently available antibiotics, or which possess unexpected selectivity against target microorganisms.

Therefore, described herein are semi-synthetic glycopeptides that have antibacterial activity. The semi-synthetic glycopeptides described herein are based on hydrolysis of the disaccharide moiety of the amino acid-4 of the parent glycopeptide to monosaccharide; conversion of the monosaccharide to the amino-sugar; acylation of the amino substituent on the amino-substituted sugar moiety on these scaffolds with certain acyl groups; and conversion of the acid moiety on the macrocyclic ring of these scaffolds to certain substituted amides. Key reaction is the treatment of properly protected intermediate compound with isocyanate or carrying a Hofmann degradation of the primary amide of the 3^{rd} amino acid asparagines with phenyl-bis-trifluoroacetate to give the primary amine. Also provided are methods for synthesis of the compounds, pharmaceutical compositions containing the compounds, and methods of use of the compounds for the treatment and/or prophylaxis of diseases, especially bacterial infections.

### Compounds

Described herein are compounds having a structure selected from the group consisting of Formulas I, II, III, IV, V, VI, VII, VIII, IX, X, XI, and XII:

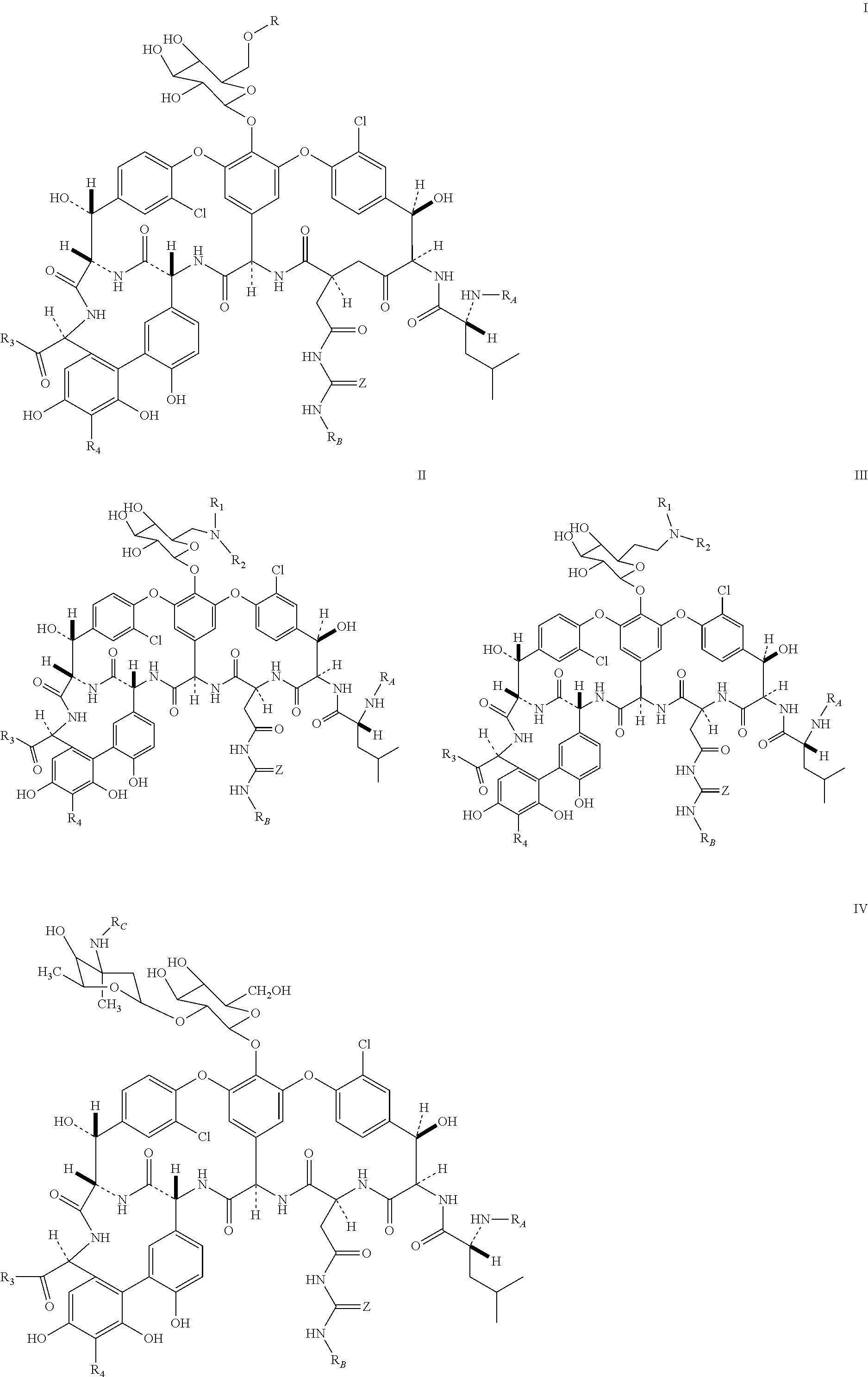

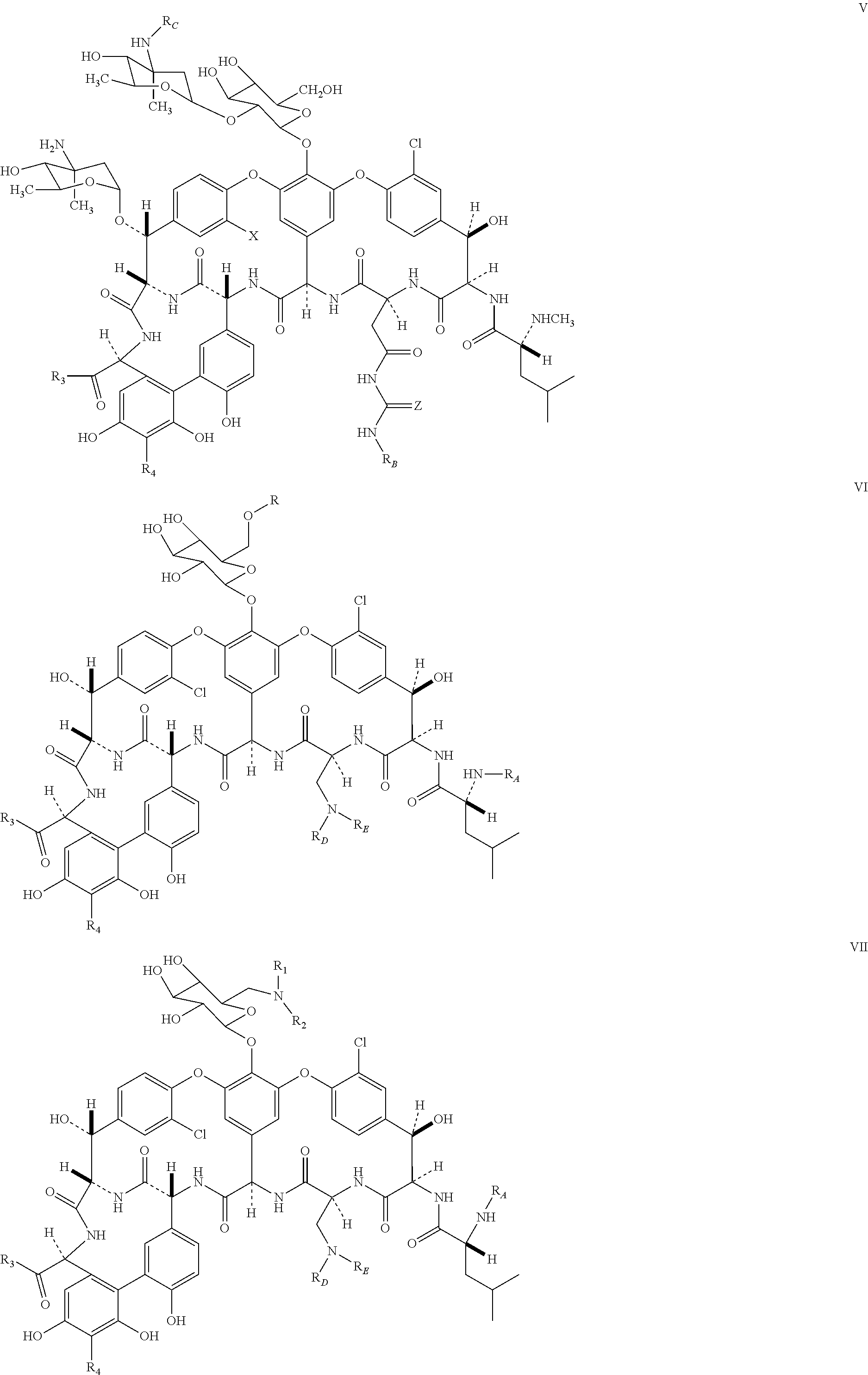

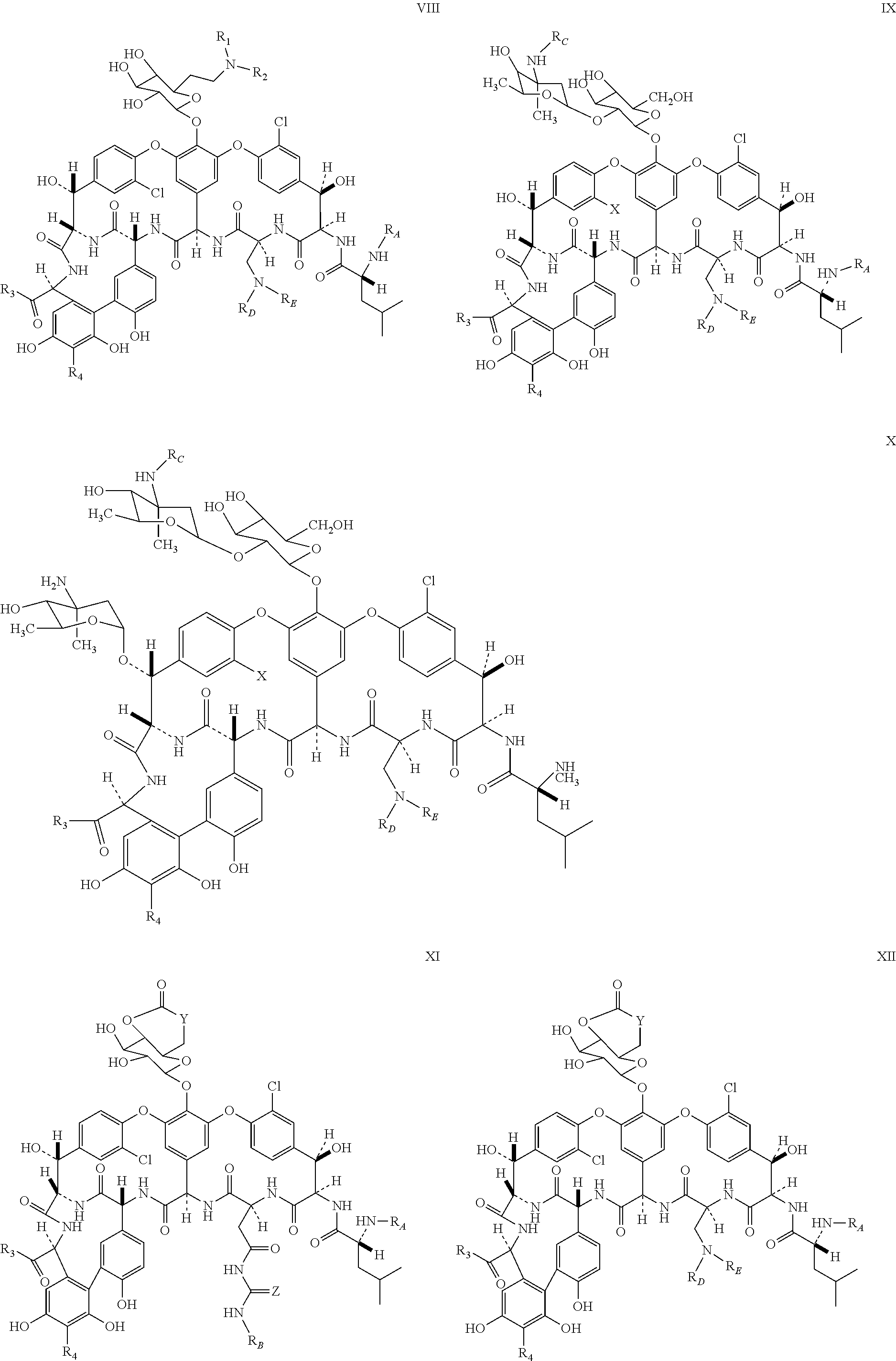

wherein,
R_{A} is selected from the group consisting of
a) hydrogen,
b) methyl,
c) C₂-C₁₂-alkyl;
R₁ and R₂ are each independently selected from the group consisting of
a) hydrogen,
b) C₁-C₁₂-alkyl,
c) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) amino,
   (f) C₁-C₁₂-alkylamino,
   (g) C₁-C₁₂-dialkylamino,
   (h) alkenyl,
   (i) alkynyl,
   (j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
   or
   R₁ and R₂ taken together with the atom to which they are attached form a substituted heteroaryl or 3-10 membered heterocycloalkyl ring which optionally contains one to two hetero functionalities selected from the group consisting of —O—, —N—, —NH, —N(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)ₙ— wherein n is 1 or 2 and the 3-10 membered heterocycloalkyl ring is optionally substituted with one or more substituents independently selected from the group consisting of
   (a) halogen,
   (b) hydroxyl,
   (c) C₁-C₃-alkoxy,
   (d) C₁-C₃-alkoxy-C₁-C₃-alkoxy,
   (e) oxo,
   (f) C₁-C₃-alkyl,
   (g) halo-C₁-C₃-alkyl,
   (h) C₁-C₃-alkoxy-C₁-C₃-alkyl,
and
k) C(═O) R₇,
l) C(═O) CH R₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl,
   or
   R₈ and R₁₀ or R₉ and R₁₀ taken together with the atom to which they are attached form a 3-10 membered heterocycloalkyl ring which is optionally substituted with one or more substituents independently selected from the group consisting of
   (a) halogen,
   (b) hydroxyl,
   (c) C₁-C₃-alkoxy,
   (d) C₁-C₃-alkoxy-C₁-C₃-alkoxy,
   (e) oxo,
   (f) C₁-C₃-alkyl,
   (g) halo-C₁-C₃-alkyl,
   (h) C₁-C₃-alkoxy-C₁-C₃-alkyl;
R₇ is selected from the group consisting of
a) hydrogen,
b) C₁-C₁₂-alkyl,
c) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) amino,
   (f) C₁-C₁₂-alkylamino,
   (g) C₁-C₁₂-dialkylamino,
   (h) alkenyl,
   (i) alkynyl,
   (j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
k) C₁-C₁₂-alkylamino;

X is selected from the group consisting of
(1) hydrogen,
(2) chlorine;

Y is selected from the group consisting of
(1) oxygen,
(2) NR₁, wherein R₁ is as previously defined;

Z is selected from the group consisting of
(1) oxygen,
(2) sulfur;

R is selected from the group consisting of
(1) hydrogen,
(2) cycloalkyl,
(3) cycloalkenyl,
(4) C₁-C₁₂-alkyl,
(5) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) —COOR₅ wherein R₅ is hydrogen or loweralkyl,
   (O—C(O)NR₅R₆ wherein R₅ is as previously defined and R₆ is hydrogen or loweralkyl,
   (g) amino,
   (h) —NR₅R₆ wherein R₅ and R₆ are as previously defined,
      or
      R₅ and R₆ are taken together with the atom to which they are attached form a 3-10 membered heterocycloalkyl ring which is optionally substituted with one or more substituents independently selected from the group consisting of
      (i) halogen,
      (ii) hydroxy,
      (iii) C₁-C₃-alkoxy,
      (iv) C₁-C₃-alkoxy-C₁-C₃-alkoxy,
      (v) oxo,
      (vi) C₁-C₁₂-alkyl,
      (vii) halo-C₁-C₁₂-alkyl,
      and
      (viii) C₁-C₃-alkoxy-C₁-C₁₂-alkyl,
   (i) aryl,
   (j) substituted aryl,
   (k) heteroaryl,
   (l) substituted heteroaryl,
   (m) mercapto,
   (n) C₁-C₁₂-thioalkoxy,
(6) C(═O)O R₁₁, wherein R₁₁ is hydrogen, loweralkyl, substituted loweralkyl, aryl, substituted aryl, heteroaryl or substituted heteroaryl,
(7) C(═O)NR₁₁ R₁₂, wherein R₁₁ is as previously defined and R₁₂ is hydrogen, loweralkyl, substituted loweralkyl, aryl, substituted aryl, heteroaryl or substituted heteroaryl,
   or
   R₁₁ and R₁₂ together with the atom to which they are attached form a 3-10 membered heterocycloalkyl ring, which is optionally substituted with one or more substituents independently selected from the group consisting of
      (a) halogen,
      (b) hydroxy,
      (c) C₁-C₃-alkoxy,
      (d) C₁-C₃-alkoxy-C₁-C₃-alkoxy,
      (e) oxo,
      (f) C₁-C₁₂-alkyl,
      (g) substituted loweralkyl,
      (h) halo-C₁-C₁₂-alkyl,
      (i) amino,
      (j) alkylamino,
      (k) dialkylamino,
      and
      (l) C₁-C₃-alkoxy-C₁-C₁₂-alkyl,
   or
   R and its connected oxygen atom taken together is halogen;

R₃ is selected from the group consisting of
(1) OH,
(2) 1-adamantanamino,
(3) 2-adamantanamino,
(4) 3-amino-1-adamantanamino,
(5) 1-amino-3-adamantanamino,
(6) 3-loweralkylamino-1-adamantanamino,
(7) 1-loweralkylamino-3-adamantanamino,
(8) amino,
(9) NR₁₃R₁₄ wherein R₁₃ and R₁₄ are each independently selected from the group consisting of hydrogen, loweralkyl, substituted loweralkyl, cycloalkyl, substituted cycloalkyl, aminoloweralkyl wherein the amino portion of the aminoloweralkyl group is further substituted with unsubstituted or substituted alkyl, alkenyl, cycloalkyl, cycloalkenyl, arylaryl, alkoxy, aryloxy, substituted alkoxy, and substituted aryloxy
or
R₁₃ and R₁₄ together with the atom to which they are attached form a 3-10 membered heterocycloalkyl ring, which is optionally substituted with one or more substituents independently selected from the group consisting of
   (a) halogen,
   (b) hydroxy,
   (c) C₁-C₃-alkoxy,
   (d) C₁-C₃-alkoxy-C₁-C₃-alkoxy,
   (e) oxo,
   (f) C₁-C₁₂-alkyl,
   (g) substituted loweralkyl,
   (h) halo-C₁-C₁₂-alkyl,
   (i) amino,
   (j) alkylamino,
   (k) dialkylamino,
   and
   (l) C₁-C₃-alkoxy-C₁-C₁₂-alkyl;

R₄ is selected from the group consisting of
(1) CH₂NH—CHR₁₅—(CH₂)ₘ—NHSO₂R_{B}, wherein m is 1 to 6 and R₁₅ is H or loweralkyl,
(2) CH₂NH—CHR₁₅—(CH₂)ₚ—CONHSO₂R_{B}, wherein p is 0 to 6 and R₁₅ is H or loweralkyl,
(3) CH₂NH—CHR₁₅—(CH₂)ₚ—COOH, wherein p is 0 to 6 and R₁₅ is H or loweralkyl,
(4) CH₂NR_{F}—CHR₁₅—(CH₂)_{q}—NR_{G}SO₂R_{B}, wherein q is 2 to 4 and R₁₅ is H or loweralkyl, R_{F} and R_{G} are independently hydrogen, lower alkyl or taken together represents a —CH₂—,
(5) H,
(6) CH₂NHCH₂PO₃H₂,
(7) aminoloweralkyl wherein the amino portion of the aminoloweralkyl group is further substituted with unsubstituted or substituted alkyl, alkenyl, cycloalkyl, cycloalkenyl, arylaryl, alkoxy, aryloxy, substituted alkoxy, and substituted aryloxy;

R_{B} is selected from the group consisting of
a) aryl,
b) C₁-C₁₂-alkyl,
c) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) amino,
   (f) C₁-C₁₂-alkylamino,
   (g) C₁-C₁₂-dialkylamino,
   (h) alkenyl,
   (i) alkynyl,
   (j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) heteroaryl,
j) heterocycloalkyl,
k) aryl substituted with one or more substituents selected from the group consisting of
   (a) halogen,
   (b) hydroxy,
   (c) C₁-C₁₂-alkoxy,
   (d) C₁-C₆-alkoxy-C₁-C₆-alkoxy,
   (e) amino,
   (f) amino-C₁-C₆-alkoxy,
   (g) C₁-C₁₂-alkylamino,
   (h) C₁-C₁₂-alkylamino-C₁-C₆-alkoxy,
   (i) C₁-C₁₂-dialkylamino,
   (j) C₁-C₁₂-dialkylamino-C₁-C₆-alkoxy,
   (k) alkenyl,
   (l) alkynyl,
   (m) C₁-C₁₂-thioalkoxy,
   (n) C₁-C₁₂-alkyl,
l) heteroaryl substituted with one or more substituents selected from the group consisting of
   (a) halogen,
   (b) hydroxy,
   (c) C₁-C₁₂-alkoxy,
   (d) C₁-C₆-alkoxy-C₁-C₆-alkoxy,
   (e) amino,
   (f) amino-C₁-C₆-alkoxy,
   (g) C₁-C₁₂-alkylamino,
   (h) C₁-C₁₂-alkylamino-C₁-C₆-alkoxy,
   (i) C₁-C₁₂-dialkylamino,
   (j) C₁-C₁₂-dialkylamino-C₁-C₆-alkoxy,
   (k) alkenyl,
   (l) alkynyl,
   (m) C₁-C₁₂-thioalkoxy,
   (n) C₁-C₁₂-alkyl;

R_{C} is each selected from the group consisting of
a) hydrogen,
b) C₁-C₁₂-alkyl,
c) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) amino,
   (f) C₁-C₁₂-alkylamino,
   (g) C₁-C₁₂-dialkylamino,
   (h) alkenyl,
   (i) alkynyl,
   (j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
k) C(═O) R₇ wherein R₇ is previously defined,
l) C(═O) CHR₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl,
   or
   R₈ and R₁₀ or R₉ and R₁₀ taken together with the atom to which they are attached form a 3-10 membered heterocycloalkyl ring which is optionally substituted with one or more substituents independently selected from the group consisting of
   (a) halogen,
   (b) hydroxyl,
   (c) C₁-C₃-alkoxy,
   (d) C₁-C₃-alkoxy-C₁-C₃-alkoxy,
   (e) oxo,
   (f) C₁-C₃-alkyl,
   (g) halo-C₁-C₃-alkyl,
   (h) C₁-C₃-alkoxy-C₁-C₃-alkyl;

R_{D} and R_{E} are each independently selected from the group consisting of
a) hydrogen,
b) C₁-C₁₂-alkyl,
c) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) amino,
   (f) C₁-C₁₂-alkylamino,
   (g) C₁-C₁₂-dialkylamino,
   (h) alkenyl,
   (i) alkynyl,
   (j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
   or
   R_{D} and R_{E} taken together with the atom to which they are attached form a 3-10 membered heterocycloalkyl ring which optionally contains one to two hetero functionalities selected from the group consisting of —O—, —N—, —NH, —N(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)ₙ— wherein n is 1 or 2 and the 3-10 membered heterocycloalkyl ring is optionally substituted with one or more substituents independently selected from the group consisting of
   (a) halogen,
   (b) hydroxyl,
   (c) C₁-C₃-alkoxy,
   (d) C₁-C₃-alkoxy-C₁-C₃-alkoxy,
   (e) oxo,
   (f) C₁-C₃-alkyl,
   (g) halo-C₁-C₃-alkyl,
   (h) C₁-C₃-alkoxy-C₁-C₃-alkyl,
and
k) C(═O) R₇ wherein R₇ is previously defined,
l) C(═O) CH R₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl,
   or
   R₈ and R₁₀ or R₉ and R₁₀ taken together with the atom to which they are attached form a 3-10 membered heterocycloalkyl ring which is optionally substituted with one or more substituents independently selected from the group consisting of
   (a) halogen,
   (b) hydroxyl,
   (c) C₁-C₃-alkoxy,
   (d) C₁-C₃-alkoxy-C₁-C₃-alkoxy,
   (e) oxo,
   (f) C₁-C₃-alkyl,
   (g) halo-C₁-C₃-alkyl,
   (h) C₁-C₃-alkoxy-C₁-C₃-alkyl,
m) C(═O) CH R₈NR₉R₇ wherein R₇, R₈ and R₉ are as previously defined;
or a pharmaceutically acceptable salt, ester, solvate, alkylated quaternary ammonium salt, stereoisomer, tautomer or prodrug thereof.

Also provided herein are pharmaceutical compositions which comprise a therapeutically effective amount of a compound as defined above in combination with a pharmaceutically acceptable carrier or diluent.

According to the methods of treatment provided herein, bacterial infections are treated or prevented in a patient such as a human or lower mammal by administering to the patient a therapeutically effective amount of a compound provided herein, in such amounts and for such time as is necessary to achieve the desired result.

In a further aspect are provided processes and intermediates for the preparation of semi-synthetic glycopeptides of Formulas I, II, III, IV, V, VI, VII, VIII, IX, X, XI, and XII above.

In another embodiment are provided compounds of Formulas II, III, VII, and VIII wherein R₁ is hydrogen and R₂ are selected from the group consisting of hydrogen, unsubstituted or substituted alkyl, alkenyl, cycloalkyl, cycloalkenyl, heterocycloalkyl, aryl, arylalkyl, alkylaryl, and heteroaryl, and said aryl, alkylaryl, arylalkyl or heteroaryl group optionally containing one or more optionally substituted aryl, heteroaryl, or condensed rings, C(═O) R₇. C(═) CHR₈NR₉R₁₀ or R₁ and R₂ together with the atom to which they are attached form a substituted heteroaryl or cycloheterocyclic ring which optionally contains additional heteroatom selected from the group consisting of optionally substituted O, N, and S. In specific embodiments, R₂ is hydrogen or methyl substituted with an unsubstituted or substituted biphenyl, for example biphenyl or chloro-biphenyl.

In another embodiment are provided compounds of Formulas I-X and XII wherein R₇ is selected from the group consisting of
a) hydrogen,
b) C₁-C₁₂-alkyl,
c) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) amino,
   (f) C₁-C₁₂-alkylamino,
   (g) C₁-C₁₂-dialkylamino,
   (h) alkenyl,
   (i) alkynyl,
   (j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
k) C₃-C₁₂-alkylamino.

In another embodiment are provided compounds of Formulas I and VI wherein R is selected from the group consisting of
(1) hydrogen,
(2) cycloalkyl,
(3) cycloalkenyl,
(4) C₁-C₁₂-alkyl,
(5) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) —COOR₅ wherein R₅ is hydrogen or loweralkyl,
   (f) —C(O)NR₅R₆ wherein R₅ is as previously defined and R₆ is hydrogen or loweralkyl,
   (g) amino,
   (h) —NR₅R₆ wherein R₅ and R₆ are as previously defined,
      or
         R₅ and R₆ are taken together with the atom to which they are attached form a 3-10 membered heterocycloalkyl ring which is optionally substituted with one or more substituents independently selected from the group consisting of halogen,
         (i) halogen,
         (ii) hydroxy,
         (iii) C₁-C₃-alkoxy,
         (iv) C₁-C₃-alkoxy-C₁-C₃-alkoxy,
         (v) oxo,
         (vi) C₁-C₁₂-alkyl,
         (vii) halo-C₁-C₁₂-alkyl,
         and
         (viii) C₁-C₃-alkoxy-C₁-C₁₂-alkyl,
   (i) aryl,
   (j) substituted aryl,
   (k) heteroaryl,
   (l) substituted heteroaryl,
   (m) mercapto,
   (n) C₁-C₁₂-thioalkoxy,
(6) C(═O)O R₁₁, wherein R₁₁ is hydrogen, loweralkyl, substituted loweralkyl, aryl, substituted aryl, heteroaryl or substituted heteroaryl,
(7) C(═O)N R₁₁ R₁₂, wherein R₁₁ is as previously defined and R₁₂ is hydrogen, loweralkyl, substituted loweralkyl, aryl, substituted aryl, heteroaryl or substituted heteroaryl,
   or
   R₁₁ and R₁₂ together with the atom to which they are attached form a 3-10 membered heterocycloalkyl ring, which is optionally substituted with one or more substituents independently selected from the group consisting of
      (a) halogen,
      (b) hydroxy,
      (c) C₁-C₃-alkoxy,
      (d) C₁-C₃-alkoxy-C₁-C₃-alkoxy,
      (e) oxo,
      (f) C₁-C₁₂-alkyl,
      (g) substituted loweralkyl,
      (h) halo-C₁-C₁₂-alkyl,
      (i) amino,
      (j) alkylamino,
      (k) dialkylamino,
      and
      (l) C₁-C₃-alkoxy-C₁-C₁₂-alkyl,
or
R and its connected oxygen atom taken together is halogen.

In another embodiment are provided compounds of Formulas I-XII wherein X is chlorine and R₄ is hydrogen.

In another embodiment are provided compounds of Formulas I-XII wherein X is hydrogen and R₄ is hydrogen.

In another embodiment are provided compounds of Formulas XI and XII wherein Y is oxygen and R₄ is hydrogen.

In another embodiment are provided compounds of Formulas XI and XII wherein Y is NH and R₄ is hydrogen.

In another embodiment are provided compounds of Formulas I-V and XI wherein Z is oxygen and R₄ is hydrogen.

In another embodiment are provided compounds of Formulas I-V and XI wherein Z is sulfur and R₄ is hydrogen.

In another embodiment are provided compounds of Formulas I-IV, VI-IX and XI-XII wherein R_{A} is methyl and R₄ is hydrogen.

In another embodiment are provided compounds of Formulas I-IV, VI-IX and XI-XII wherein R_{A} is hydrogen and R₄ is hydrogen.

In another embodiment are provided compounds of Formulas I-IV, VI-IX and XI-XII wherein R_{A} is methyl or hydrogen and R₃ is selected from the group consisting of
(1) OH,
(2) 1-adamantanamino,
(3) 2-adamantanamino,
(4) 3-amino-1-adamantanamino,
(5) 1-amino-3-adamantanamino,
(6) 3-loweralkylamino-1-adamantanamino,
(7) 1-loweralkylamino-3-adamantanamino,
(8) amino
(9) NR₁₃R₁₄ wherein R₁₃ and R₁₄ are each independently selected from the group consisting of hydrogen, loweralkyl, substituted loweralkyl, cycloalkyl, substituted cycloalkyl, aminoloweralkyl wherein the amino portion of the aminoloweralkyl group is further substituted with unsubstituted or substituted alkyl, alkenyl, cycloalkyl, cycloalkenyl, arylaryl, alkoxy, aryloxy, substituted alkoxy, and substituted aryloxy
   or
   R₁₃ and R₁₄ together with the atom to which they are attached form a 3-10 membered heterocycloalkyl ring, which is optionally substituted with one or more substituents independently selected from the group consisting of
      (a) halogen,
      (b) hydroxy,
      (c) C₁-C₃-alkoxy,
      (d) C₁-C₃-alkoxy-C₁-C₃-alkoxy,
      (e) oxo,
      (f) C₁-C₁₂-alkyl,
      (g) substituted loweralkyl,
      (h) halo-C₁-C₁₂-alkyl,
      (i) amino,
      (j) alkylamino,
      (k) dialkylamino,
   and
      (l) C₁-C₃-alkoxy-C₁-C₁₂-alkyl.

In another embodiment are provided compounds of Formulas I-IV, VI-IX and XI-XII wherein R_{A} is methyl or hydrogen and R₄ is selected from the group consisting of
(1) (CH₂NH—CHR₁₅—(CH₂)ₘ—NHSO₂R_{B}, wherein m is 1 to 6 and R₁₅ is H or loweralkyl,
(2) CH₂NH—CHR₁₅—(CH₂)ₚ—CONHSO₂R_{B}, wherein p is 0 to 6 and R₁₅ is H or loweralkyl,
(3) CH₂NH—CHR₁₅—(CH₂)ₚ—COOH, wherein p is 0 to 6 and R₁₅ is H or loweralkyl,
(4) CH₂NR_{F}—CHR₁₅—(CH₂)_{q}—NR_{G}SO₂R_{B}, wherein q is 2 to 4 and R₁₅ is H or loweralkyl, R_{F} and R_{G} are independently hydrogen, lower alkyl or taken together represents a —CH₂—,
(5) H,
(6) CH₂NHCH₂PO₃H₂,
(7) aminoloweralkyl wherein the amino portion of the aminoloweralkyl group is further substituted with unsubstituted or substituted alkyl, alkenyl, cycloalkyl, cycloalkenyl, arylaryl, alkoxy, aryloxy, substituted alkoxy, and substituted aryloxy.

In another embodiment are provided intermediate compounds of Formulas i, ii, iii, iv, v, vi, vii, viii, ix, and x wherein R_{A} is hydrogen or methyl, X is chlorine or hydrogen, and R₄ is hydrogen, CH₂NHCH₂PO₃H₂, or aminoloweralkyl, R₃ is alkoxy or amino for the synthesis of antibacterial agents of Formulas I-XII.

### DEFINITIONS

Unless otherwise noted, terminology used herein should be given its normal meaning as understood by one of skill in the art.

The term “alkyl” as used herein refers to saturated, straight- or branched-chain hydrocarbon radicals derived from a hydrocarbon moiety containing between one and twenty carbon atoms by removal of a single hydrogen atom.

The term substituted alkyl as used herein refers to alkyl substituted by one, two or three groups consisting of halogen, alkoxy, amino, alkylamino, dialkylamino, hydroxy, aryl, heteroaryl, alkenyl or alkynyl group.

The term “alkenyl” as used herein refers to unsaturated, straight- or branched-chain hydrocarbon radicals derived from a hydrocarbon moiety containing between two and twenty carbon atoms by removal of a single hydrogen atom.

The term “cycloalkyl” as used herein refers to a monovalent group derived from a monocyclic or bicyclic saturated carbocyclic ring compound containing between three and twenty carbon atoms by removal of a single hydrogen atom.

The term substituted cycloalkyl as used herein refers to cycloalkyl substituted by one, two or three groups consisting of halogen, alkoxy, amino, alkylamino, dialkylamino, hydroxy, aryl, heteroaryl, alkenyl or alkynyl groups.

The term “cycloalkenyl” as used herein refers to a monovalent group derived from a monocyclic or bicyclic unsaturated carbocyclic ring compound containing between three and twenty carbon atoms by removal of a single hydrogen atom.

The terms “C₁-C₃-alkyl”, “C₁-C₆-alkyl”, and “C₁-C₁₂-alkyl” as used herein refer to saturated, straight- or branched-chain hydrocarbon radicals derived from a hydrocarbon moiety containing between one and three, one and six, and one and twelve carbon atoms, respectively, by removal of a single hydrogen atom. Examples of C₁-C₃-alkyl radicals include methyl, ethyl, propyl and isopropyl. Examples of C₁-C₆-alkyl radicals include, but not limited to, methyl, ethyl, propyl, isopropyl, n-butyl, tert-butyl, neopentyl and n-hexyl. Examples of C₁-C₁₂-alkyl radicals include, but not limited to, methyl, ethyl, propyl, isopropyl, n-butyl, tert-butyl, neopentyl, n-hexyl. N-heptyl, n-octyl, n-nonyl, n-decyl, n-undecyl and n-docecyl.

The term loweralkyl as used herein refers to C₁-C₁₂-alkyl as defined above.

The term substituted loweralkyl as used herein refers to C₁-C₁₂-alkyl substituted by one, two or three groups consisting of halogen, alkoxy, amino, alkylamino, dialkylamino, hydroxy, aryl, heteroaryl, alkenyl or alkynyl groups.

The term “C₃-C₁₂-cycloalkyl” denoted a monovalent group derived from a monocyclic or bicyclic saturated carbocyclic ring compound by removal of a single hydrogen atom. Examples include cyclopropyl, cyclobutyl, cyclopentyl, cyclohexyl, bicyclo[2.2.1]heptyl, and bicyclo[2.2.2]octyl.

The terms “C₁-C₃-alkoxy”, “C₁-C₆-alkoxy” as used herein refers to the C₁-C₃-alkyl group and C₁-C₆-alkyl group, as previously defined, attached to the parent molecular moiety through an oxygen atom. Examples of C₁-C₆-alkoxy radicals include, but not limited to, methoxy, ethoxy, propoxy, isopropoxy, n-butoxy, tert-butoxy, neopentoxy and n-hexoxy.

The term “loweralkylamino” as used herein refers to C₁-C₁₂-alkyl groups, as previously defined, attached to the parent molecular moiety through a nitrogen atom. Examples of loweralkylamino include, but are not limited to methylamino, dimethylamino, ethylamino, diethylamino, propylamino and decylamino.

The term “oxo” denotes a group wherein two hydrogen atoms on a single carbon atom in an alkyl group as defined above are replaced with a single oxygen atom (i.e. a carbonyl group).

The term “aryl” as used herein refers to a mono- or bicyclic carbocyclic ring system having one or two aromatic rings including, but not limited to, phenyl, naphthyl, tetrahydronaphthyl, indanyl, indenyl and the like and is optionally un-substituted or substituted (including bicyclic aryl groups) with one, two or three substituents independently selected from loweralkyl, substituted loweralkyl, haloalkyl, C₁-C₁₂-alkoxy, thioalkoxy, C₁-C₁₂-thioalkoxy, aryloxy, amino, alkylamino, dialkylamino, acylamino, cyano, hydroxy, halogen, mercapto, nitro, carboxaldehyde, carboxy, alkoxycarbonyl and carboxamide. In addition, substituted aryl groups include tetrafluorophenyl and pentafluorophenyl.

The term “substituted aryl” as used herein refers to a mono- or bicyclic carbocyclic ring system having one or two aromatic rings including, but not limited to, phenyl, naphthyl, tetrahydronaphthyl, indanyl, indenyl and the like substituted (including bicyclic aryl groups) with one, two or three substituents independently selected from loweralkyl, substituted loweralkyl, haloalkyl, C₁-C₁₂-alkoxy, thioalkoxy, C₁-C₁₂-thioalkoxy, alkoxyalkylalkoxy, aryloxy, amino, aminoalkyl, aminoalkylalkoxy, alkylamino, alkylaminoalkyl, alkylaminoalkylalkoxy, dialkylamino, dialkylaminoalkyl, dialkylaminoalkylalkoxy, acylamino, cyano, hydroxy, halogen, mercapto, nitro, carboxaldehyde, carboxy, alkoxycarbonyl, aryl, heteroaryl, heterocyclaryl and carboxamide. In addition, substituted aryl groups include tetrafluorophenyl and pentafluorophenyl.

The term “arylalkyl” as used herein refers to an aryl group as defined above attached to the parent molecular moiety through an alkyl group wherein the alkyl group is of one to twelve carbon atoms.

The term “substituted arylalkyl” as used herein refers to a substituted aryl group as defined above attached to the parent molecular moiety through an alkyl group wherein the alkyl group is of one to twelve carbon atoms.

The term “alkylaryl” as used herein refers to an alkyl group as defined above attached to the parent molecular moiety through an aryl group.

The term “halo” and “halogen” as used herein refer to an atom selected from fluorine, chlorine, bromine and iodine.

The term “alkylamino” refers to a group having the structure —NHR′ wherein R′ is alkyl, as previously defined. Examples of alkylamino include methylamino, ethylamino, iso-propylamino, and the like.

The term “dialkylamino” refers to a group having the structure —NHR′R″ wherein R′ and R″ are independently selected from alkyl, as previously defined. Additionally, R′ and R″ taken together optionally be —(CH₂)ₖ— where k is an integer of from 2 to 6. Examples of dialkylamino include dimethylamino, diethylamino, methylpropylamino, piperidino, and the like.

The term “haloalkyl” denotes an alkyl group, as defined above, having one, two or three halogen atoms attached thereto and is exemplified by such group as chloromethyl, bromoethyl, trifluoromethyl, and the like.

The term “alkoxycarbonyl” represents as ester group; i.e. an alkoxy group, attached to the parent molecular moiety through a carbonyl group such as methoxycarbonyl, ethoxycarbonyl, and the like.

The term “thioalkoxy” refers to an alkyl group previously defined attached to the parent molecular moiety through a sulfur atom.

The term “carboxaldehyde” as used herein refers to a group of formula —CHO.

The term “carboxy” as used herein refers to a group of formula —CO₂H.

The term “carboxamide” as used herein refers to a group of formula —CONHR′R″ wherein R′ and R″ are independently selected from hydrogen, alkyl, substituted loweralkyl, or R′ and R″ taken together optionally be —(CH₂)ₖ— where k is an integer of from 2 to 6.

The term “heteroaryl”, as used herein, refers to a cyclic or bicyclic aromatic radical having from five to ten ring atoms in each ring of which at least one atom of the cyclic or bicyclic ring is selected from optionally substituted S, O, and N; zero, one or two ring atoms are additional heteroatoms independently selected from optionally substituted S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, naphthyridinyl; and the like.

The term “substituted heteroaryl” as used herein refers to a cyclic or bicyclic aromatic radical having from five to ten ring atoms in each ring of which at least one atom of the cyclic or bicyclic ring is selected from optionally substituted S, O, and N; zero, one or two ring atoms are additional heteroatoms independently selected from optionally substituted S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, naphthyridinyl; and the like substituted with one, two or three substituents independently selected from loweralkyl, substituted loweralkyl, haloalkyl, C₁-C₁₂-alkoxy, thioalkoxy, C₁-C₁₂-thioalkoxy, alkoxyalkylalkoxy, aryloxy, amino, aminoalkyl, aminoalkylalkoxy, alkylamino, alkylaminoalkyl, alkylaminoalkylalkoxy, dialkylamino, dialkylaminoalkyl, dialkylaminoalkylalkoxy, acylamino, cyano, hydroxy, halogen, mercapto, nitro, carboxaldehyde, carboxy, alkoxycarbonyl, aryl, heteroaryl, heterocyclaryl and carboxamide.

The term “heterocycloalkyl” as used herein, refers to a non-aromatic partially unsaturated or fully saturated 3- to 10-membered ring system, which includes single rings of 3 to 8 atoms in size and bi- or tri-cyclic ring systems which includes aromatic six-membered aryl or heteroaryl rings fused to a non-aromatic ring. These heterocycloalkyl rings include those having from one to three heteroatoms independently selected from oxygen, sulfur and nitrogen, in which the nitrogen and sulfur heteroatoms optionally be oxidized and the nitrogen heteroatom optionally be quaternized. Representative heterocycloalkyl rings include, but not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “heteroarylalkyl” as used herein, refers to a heteroaryl group as defined above attached to the parent molecular moiety through an alkylene group wherein the alkylene group is of one to four carbon atoms.

“Protecting group” refers to an easily removable group which is known in the art to protect a functional group, for example, a hydroxyl, ketone or amine, against undesirable reaction during synthetic procedures and to be selectively removable. Examples of such protecting groups are known, cf, for example, T. H. Greene and P. G. M. Wuts, Protective Groups in Organic Synthesis, 2nd edition, John Wiley & Sons, New York (1991). Examples of hydroxy-protecting groups include, but not limited to, methylthiomethyl, tert-dimethylsilyl, tert-butyldiphenylsilyl, ethers such as methoxymethyl, and esters including acetyl, benzoyl, and the like. Examples of ketone protecting groups include, but not limited to, ketals, oximes, O-substituted oximes for example O-benzyl oxime, O-phenylthiomethyl oxime, 1-isopropoxycyclohexyl oxime, and the like. Examples of amine protecting groups include, but are not limited to, tert-butoxycarbonyl (Boc) and carbobenzyloxy (Cbz).

A term “protected-hydroxy” refers to a hydroxy group protected with a hydroxy protecting group, as defined above.

The term amino acid refers to amino acids having D or L stereochemistry, and also refers to synthetic, non-natural amino acids having side chains other than those found in the 20 common amino acids. Non-natural amino acids are commercially available or are optionally prepared according to U.S. Pat. No. 5,488,131 and references therein. Amino acids are optionally further substituted to contain modifications to their amino, carboxy, or side-chain groups. These modifications include the numerous protecting group commonly used in peptide synthesis (T. H. Greene and P. G. M. Wuts, Protective Groups in Organic Synthesis, 2nd edition, John Wiley & Sons, New York, 1991).

The term “substituted heteroaryl” as used herein, refers to a heteroaryl group as defined herein substituted by independent replacement of one, two or three of the hydrogen atoms thereon with Cl, Br, F, I, OH, CN, C₁-C₁₂-alkyl, alkoxy, C₁-C₁₂-alkoxy substituted with aryl, haloalkyl, thioalkyl, amino, alkylamino, dialkylamino, mercapto, nitro, carboxaldehyde, carboxy, alkoxycarbonyl and carboxamide. In addition, any one substituent is optionally an aryl, heteroaryl, or heterocycloalkyl group.

The term “substituted heterocycloalkyl” as used herein, refers to a heterocycloalkyl group as defined herein substituted by independent replacement of one, two or three of the hydrogen atoms thereon with Cl, Br, F, I, OH, CN, C₁-C₁₂-alkyl, C₁-C₁₂-alkoxy, C₁-C₁₂-alkoxy substituted with aryl, haloalkyl, thioalkyl, amino, alkylamino, dialkylamino, mercapto, nitro, carboxaldehyde, carboxy, alkoxycarbonyl and carboxamide. In addition, any one substituent is optionally aryl, heteroaryl, or heterocycloalkyl group.

The term “stereoisomer” as used herein, refers to either of two forms of a compound having the same molecular formula and having their constituent atoms attached in the same order, but having different arrangement if their atoms in space about an asymmetric center. If asymmetric centers exist in the described compounds, except where otherwise noted, the compounds described herein include the various stereoisomers and mixtures thereof. Accordingly, except where otherwise noted, it is intended that a mixture of stereo-orientations or an individual isomer of assigned or unassigned orientation is present.

The term “tautomer” as used herein refers to either of the two forms of a chemical compound that exhibits tautomerism, which is the ability of certain chemical compounds to exist as a mixture of two interconvertible isomers in equilibrium via proton transfer. The keto and enol forms of carbonyl compounds are examples of tautomers. They are interconvertible in the presence of traces of acids and bases via a resonance stabilized anion, the enolate ion.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. For example, S. M. Berge, et al. describes pharmaceutically acceptable salts in detail in J. Pharmaceutical Sciences, 66: 1-19 (1977), incorporated herein by reference for this purpose. The salts are prepared in situ during the final isolation and purification of the compounds described herein, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

The term “pharmaceutically acceptable ester” refers to esters which hydrolyze in vivo and include those that break down in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Representative examples of particular esters include, but are not limited to, formates, acetates, propionates, butyrates, acrylates and ethylsuccinates.

The term “solvate” as used herein refers to a compound formed by salvation, the combination of solvent molecules with molecules or ions of solute composed of a compound described herein. The term “pharmaceutically acceptable solvate” refers to those solvates which are, within the scope of sound medical judgment, suitable for use in contact with the tissues of humans and lover animals without undue toxicity, irritation, allergic response and the like, and are commensurate with a reasonable benefit/risk ratio.

The term “alkylated quaternary ammonium salt” as used herein refers to a compound formed by alkylation of the nitrogen atom of the primary, secondary or tertiary amine of the molecule with alkyl halide to form alkyl quaternary ammonium salt.

The term “pharmaceutically acceptable prodrugs” refers to those prodrugs of the compounds described herein which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds described herein. The term “prodrug” refers to compounds that are transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference for this purpose.

### Synthetic Methods

Synthesis of the compounds described herein is broadly summarized as follows. The compounds described herein are made, for example, by chemical modifications of the Compound A, Compound B, Compound H and Compound C scaffolds. In particular, the semi-synthetic glycopeptides described herein are made by chemical modification of Compound A, Compound B, Compound H and Compound C or of the monosaccharide of glycopeptides made by subjecting the parent glycopeptide in acidic medium to hydrolyze the disaccharide moiety of the amino acid-4 of the parent glycopeptide to give the monosaccharide; protection of the amino function by t-butoxycarbonyl group, carbobenzyloxy group or allyloxycarbonyl group; conversion of the acid moiety on the macrocyclic ring of these scaffolds to certain substituted amides and treatment of the compound with isocyante. Alternatively, if amino function on the monosaccharide is required, conversion of the monosaccharide to the amino-sugar derivative; acylation of the amino substituent on the amino-substituted sugar moiety on these scaffolds with certain acyl groups; protection of the amino function by t-butoxycarbonyl group, carbobenzyloxy group or allyloxycarbonyl group; conversion of the acid moiety on the macrocyclic ring of these scaffolds to certain substituted amides and treatment of the compound with isocyante. The compounds described herein are made, for example, by coupling the amino-sugar moiety of functionalized or unfunctionalized glycopeptides from the above scaffolds with the appropriate acyl and/or amino groups under amide formation conditions and conversion of the acid moiety on the macrocyclic ring of the resulting glycopeptide derivative to certain substituted amides; or a combination of an alkylation modification of the substituent on the amino-substituted sugar moiety on this scaffold with certain alkyl groups or acylation modification of the amino substituent on the amino-substituted sugar moiety on this scaffold with certain acyl groups, α-amino acid or β-amino acids or derivatives thereof, and conversion of the acid moiety on the macrocyclic ring of this scaffold to certain substituted amides. In another series, the compounds described herein are made, for example, by chemical modifications of the Compound A, Compound B, Compound H and Compound C scaffolds. In particular, the semi-synthetic glycopeptides described herein are made by chemical modification of Compound A, Compound B, Compound H and Compound C or of the monosaccharide of the about glycopeptides made by subjecting the parent glycopeptide in acidic medium to hydrolyze the disaccharide moiety of the amino acid-4 of the parent glycopeptide to give the monosaccharide; protection of the amino function by t-butoxycarbonyl group, carbobenzyloxy group or allyloxycarbonyl group; conversion of the acid moiety on the macrocyclic ring of these scaffolds to certain substituted amides and Hofmann degradation of the primary amide of the 3rd amino acid asparagine with phenyl-bis-trifluoroacetate to the primary amine. In some embodiments, synthesis of compounds also involves the use of protecting or blocking groups in order to maximize yields, minimize unwanted side products, or improve purification.

In particular, the semi-synthetic glycopeptides of the compounds described herein are made, for example, by modifying Compound A, Compound B, Compound H and Compound C scaffolds. The glycopeptide starting material is optionally unsubstituted or substituted at the 7^{th} amino acid at the 4′ position of the phenyl ring with CH₂NHCH₂PO₃H₂, or aminoloweralkyl as defined herein.

Selective hydrolysis of Compound A, Compound B, Compound H or Compound C in which the 7^{th} amino acid at the 4′ position of the phenyl ring substituted with hydrogen, CH₂NHCH₂PO₃H₂, or aminoloweralkyl as defined herein with acid gives the monosaccharide intermediate.

In general, compound of Formulas I-V and XI, described herein are made by modifying a compound from the group consisting of Formulas i, ii, iii, iv and v,

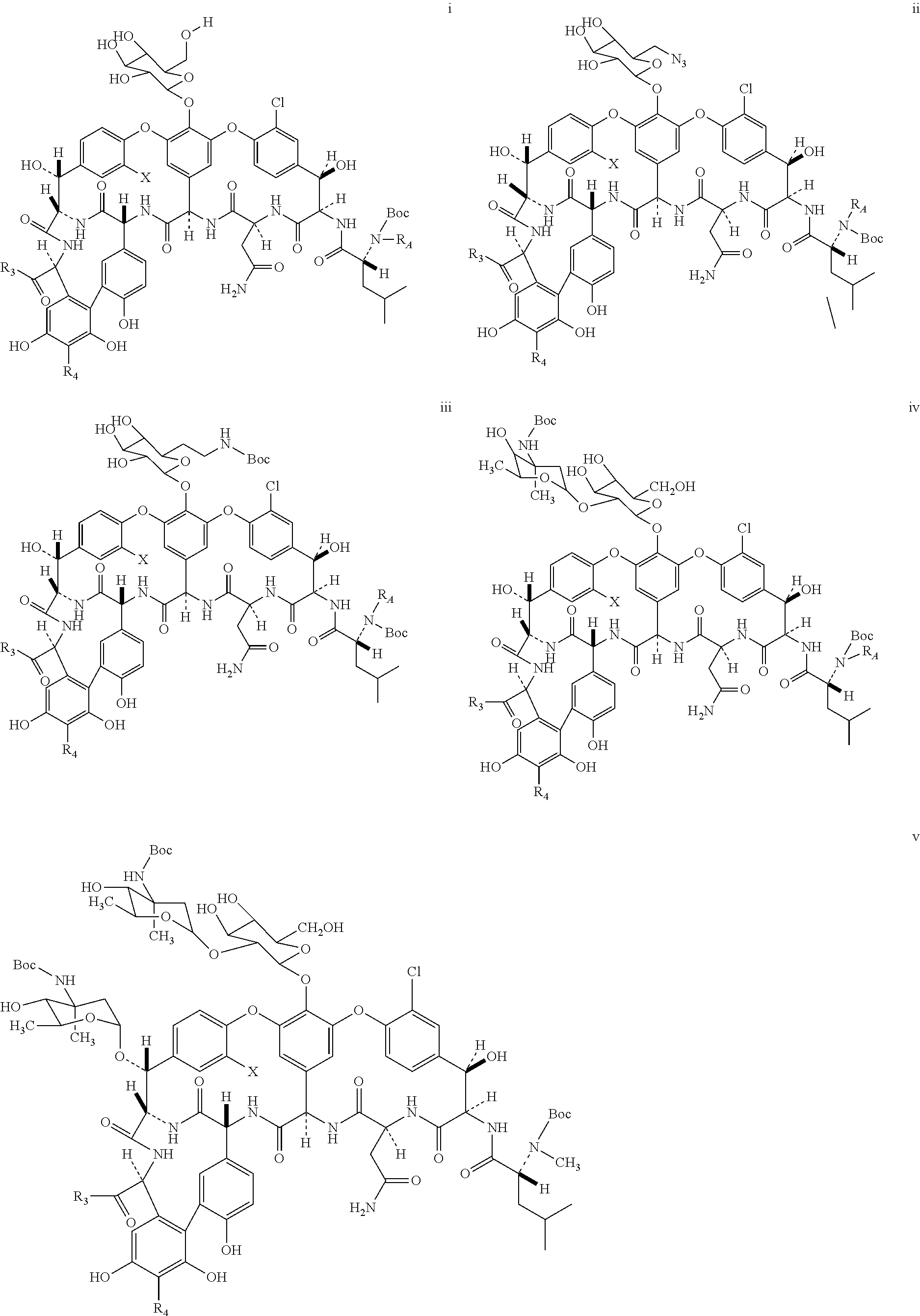

wherein R_{A} is hydrogen or methyl, X is chlorine or hydrogen, R₃ is alkoxy, 2-adamantanamino, or loweralkylamino as defined herein, or R₄ is hydrogen or properly protected CH₂NHCH₂PO₃H₂, or Boc-aminoloweralkyl as defined herein, by a technique selected from the group consisting of,
   (a) acylation of the primary amide group of the 3^{rd} amino acid asparagine with an R_{B}-isocyanate or R_{B}-thioisocyanate in the presence of a base such as dimethylaminopyridine and the like,
   (b) removal of the Boc protecting group with mild acid such as trifluoroacetic acid,
   (c) if the R₃ is alkoxy, removal of the alkoxy group by mild base or acid hydrolysis to give the carboxylic acid derivative,
   (d) reduction of the azide function to an amine,
   (e) alkylation of the primary alcohol of the mono-sugar or the amino substituent on the amino-substituted sugar moiety of the 4^{th} amino acid of the compound with an alkyl halide having structure R₁-J where J is a halogen or R_{C}-J where J is a halogen
   (f) acylation of the primary alcohol of the mono-sugar or the amino substituent on the amino-substituted sugar moiety of the 4^{th} amino acid of the compound with an acyl group having the structure, C(═O) R₇,
   (g) acylation of the primary alcohol of the mono-sugar or the amino substituent on the amino-substituted sugar moiety of the 4^{th} amino acid of the compound with an acyl group having the structure, C(═O) CHR₈NR₉R₁₀,
   (h) reaction of the amino substituent on the amino-substituted sugar moiety of the 4^{th} amino acid of the compound with an aldehyde or ketone followed by reductive amination of the resulting imine,
   (i) conversion of the acid moiety on the macrocyclic ring of the compound with substituted amide as defined by R₃,
   (j) phosgene reaction on primary alcohol or primary amine of the mono-sugar moiety of the 4^{th} amino acid of the compound with the adjacent hydroxyl group,
   (k) dipolar cycloaddition of the azide with alkyne to form 1,2,3-trizole,
   (l) a combination of (a) and (b),
   (m) a combination of (a), (b) and (c),
   (n) a combination of (a), (c), (i) and (b),
   (o) a combination of (a), (e), and (b),
   (p) a combination of (a), (f) and (b),
   (q) a combination of (a), (g) and (b),
   (r) a combination of (a), (h) and (b),
   (s) a combination of (a), (d) and (b),
   (t) a combination of (a), (d), (c) and (b),
   (u) a combination of (a), (c), (i), (d) and (b),
   (v) a combination of (a), (c), (d) and (b),
   (w) a combination of (a), (c), (i), (d), (e) and (b),
   (x) a combination of (a), (c), (i), (d), (f) and (b),
   (y) a combination of (a), (c), (i), (d), (g) and (b),
   (z) a combination of (a), (c), (i), (d), (h) and (b),
   (aa) a combination of (a), (c), (d), (e) and (b),
   (bb) a combination of (a), (c), (d), (f) and (b),
   (cc) a combination of (a), (c), (d), (g) and (b),
   (dd) a combination of (a), (c), (d), (h) and (b),
   (ee) a combination of (a), (j), and (b),
   (ff) a combination of (a), (j), (c), (i) and (b),
   (gg) a combination of (a), (d), (j), and (b),
   (hh) a combination of (a), (d), (j), (c), (i) and (b),
   (ii) a combination of (a), (k), and (b),
   (jj) a combination of (a), (k), (c), (i) and (b),
to form a compound having a formula selected from the group consisting of

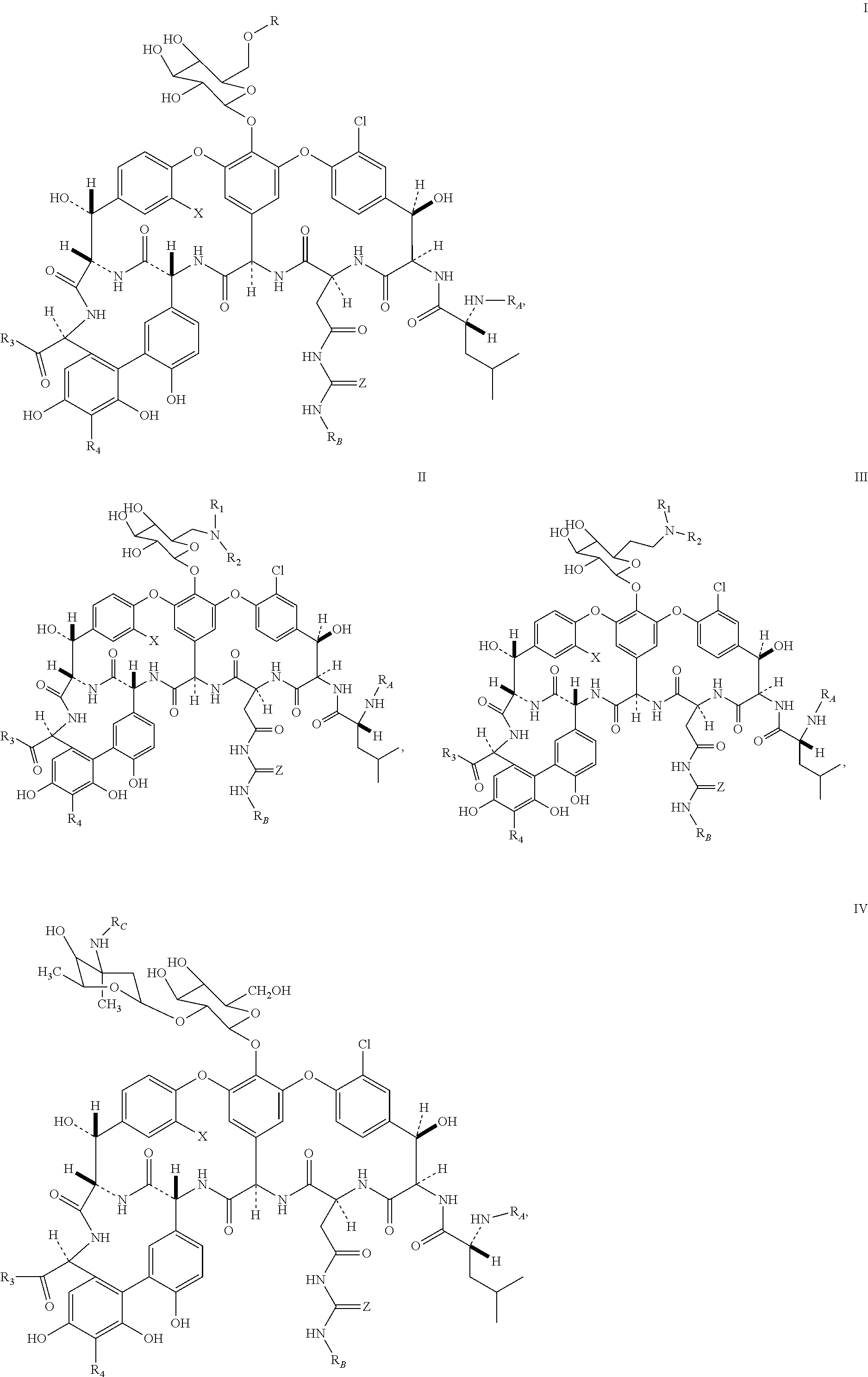

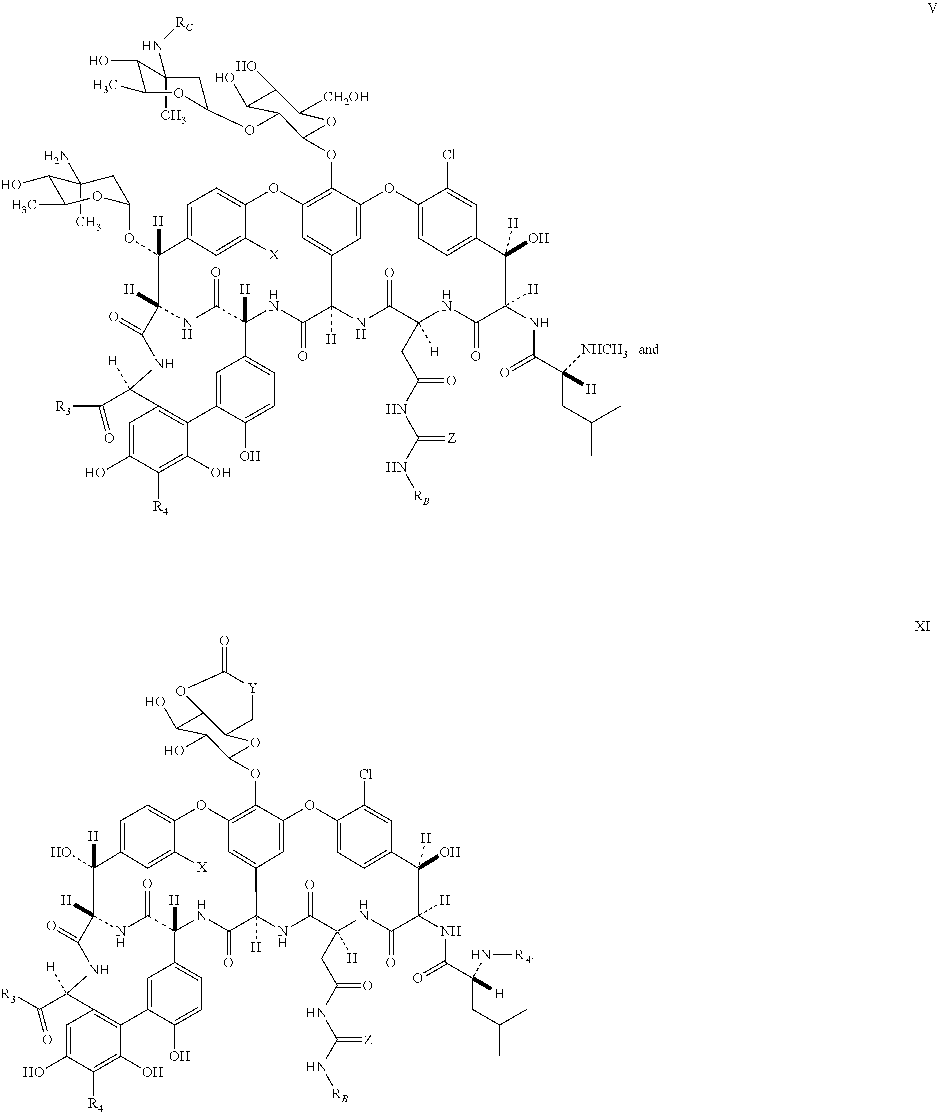

wherein R, R₁, R₂, R₃, R₄, R_{A}, R_{B}, R_{C}, X, Y, and Z are as defined herein.

In general the compounds a compound of Formulas VI-X and XII, described herein are made by modifying a compound from the group consisting of Formulas vi, vii, viii, ix and x,

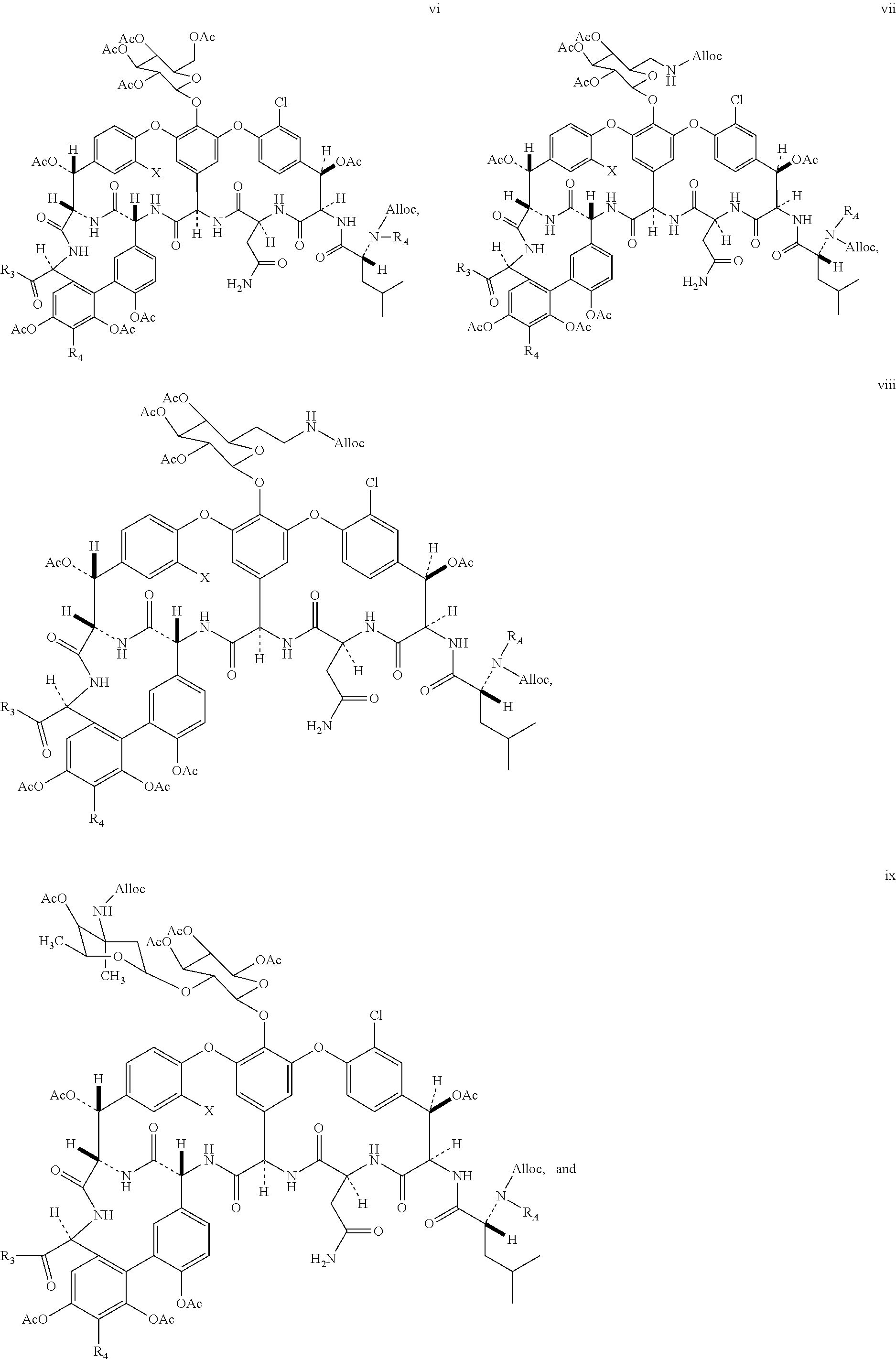

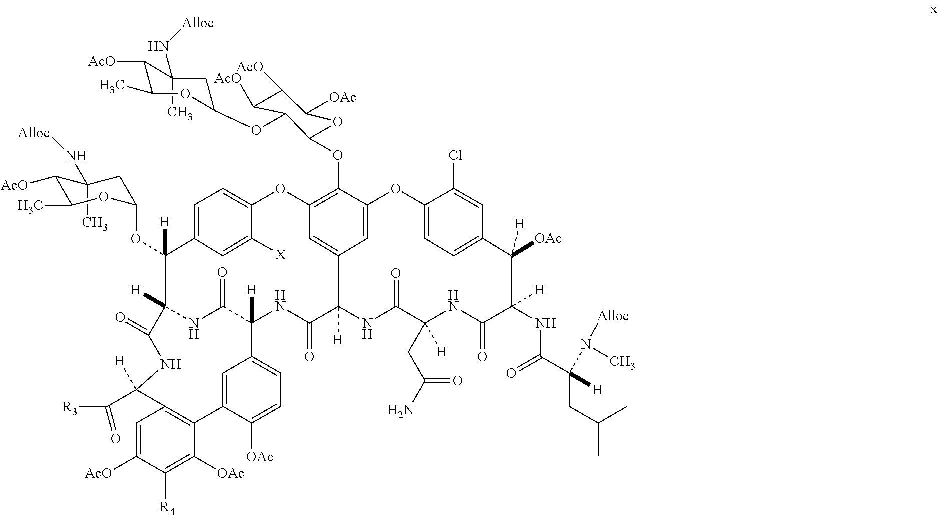

wherein R_{A} is hydrogen or methyl, X is chlorine or hydrogen, R₃ is alkoxy, 2-adamantanamino, or loweralkylamino as defined herein, or R₄ is hydrogen or properly protected CH₂NHCH₂PO₃H₂, or Boc-aminoloweralkyl as defined herein, by a technique selected from the group consisting of,
   (a) Hofmann degradation of the primary amide group of the 3^{rd} amino acid asparagine with phenyliodine-bis-trifluoroacetate to give the primary amine,
   (b) alkylation of the primary amine with an alkyl halide having structure R_{D}-J where J is a halogen or R_{E}-J where J is a halogen,
   (c) acylation of the primary amine with an acyl group having the structure, C(═O) R₇,
   (d) acylation of the primary amine with an acyl group having the structure, C(═O) CHR₈NR₉R₁₀,
   (e) removal of the N-Alloc protecting group with the use of Pd(OAc)₂, PPh₃, and (nBu)₃SnH,
   (f) hydrolysis of all acetate groups to give the alcohol,
   (g) if the R₃ is alkoxy, removal of the alkoxy group by mild base or acid hydrolysis to give the carboxylic acid derivative,
   (h) alkylation of the primary alcohol of the mono-sugar or the amino substituent on the amino-substituted sugar moiety of the 4^{th} amino acid of the compound with an alkyl halide having structure R-J where J is a halogen, R₁-J where J is a halogen or R_{C}-J where J is a halogen
   (i) acylation of the primary alcohol of the mono-sugar or the amino substituent on the amino-substituted sugar moiety of the 4^{th} amino acid of the compound with an acyl group having the structure, C(═O) R₇,
   (j) acylation of the primary alcohol of the mono-sugar or the amino substituent on the amino-substituted sugar moiety of the 4^{th} amino acid of the compound with an acyl group having the structure, C(═O) CHR₈NR₉R₁₀,
   (k) reaction of the amino substituent on the amino-substituted sugar moiety of the 4^{th} amino acid of the compound with an aldehyde or ketone followed by reductive amination of the resulting imine,
   (l) conversion of the acid moiety on the macrocyclic ring of the compound with substituted amide as defined by R₃,
   (m) phosgene reaction on primary alcohol or primary amine of the mono-sugar moiety of the 4^{th} amino acid of the compound with the adjacent hydroxyl group,
   (n) a combination of (a), (e) and (f),
   (o) a combination of (a), (b), (e) and (f),
   (p) a combination of (a), (c), (e) and (f),
   (q) a combination of (a), (d), (e) and (f),
   (r) a combination of (a), (c), (e), (f) and (g),
   (s) a combination of (a), (c), (e), (f), (g) and (l),
   (t) a combination of (a), (d), (e), (f) and (g),
   (u) a combination of (a), (d), (e), (f), (g) and (l),
   (v) a combination of (a), (c), (e), (h) and (f),
   (w) a combination of (a), (d), (e), (h), and (f),
   (x) a combination of (a), (c), (e), (h), (f) and (g),
   (y) a combination of (a), (d), (e), (h), (f) and (g),
   (z) a combination of (a), (c), (e), (h), (f), (g) and (l),
   (aa) a combination of (a), (d), (e), (h), (f), (g) and (l),
   (bb) a combination of (a), (c), (e), (i) and (f),
   (cc) a combination of (a), (d), (e), (i), and (f),
   (dd) a combination of (a), (c), (e), (i), (f) and (g),
   (ee) a combination of (a), (d), (e), (i), (f) and (g),
   (ff) a combination of (a), (c), (e), (i), (f), (g) and (l),
   (gg) a combination of (a), (d), (e), (i), (f), (g) and (l),
   (hh) a combination of (a), (c), (e), (j) and (f),
   (ii) a combination of (a), (d), (e), (j), and (f),
   (jj) a combination of (a), (c), (e), (j), (f) and (g),
   (kk) a combination of (a), (d), (e), (j), (f) and (g),
   (ll) a combination of (a), (c), (e), (j), (f), (g) and (l),
   (mm) a combination of (a), (d), (e), (j), (f), (g) and (l),
   (nn) a combination of (a), (c), (e), (k) and (f),
   (oo) a combination of (a), (d), (e), (k), and (f),
   (pp) a combination of (a), (c), (e), (k), (f) and (g),
   (qq) a combination of (a), (d), (e), (k), (f) and (g),
   (rr) a combination of (a), (c), (e), (k), (f), (g) and (l),
   (ss) a combination of (a), (d), (e), (k), (f), (g) and (l),
to form a compound having a formula selected from the group consisting of:

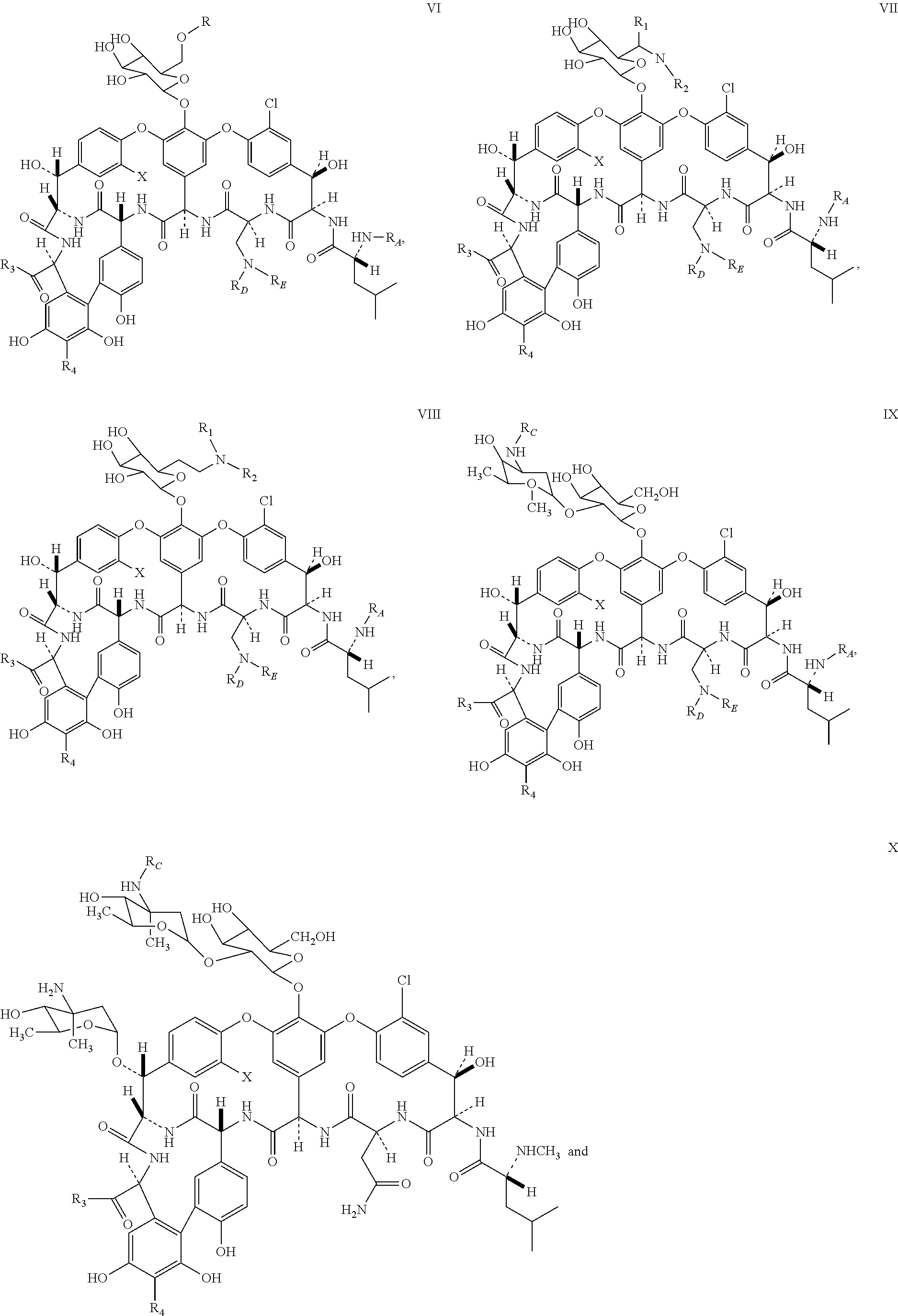

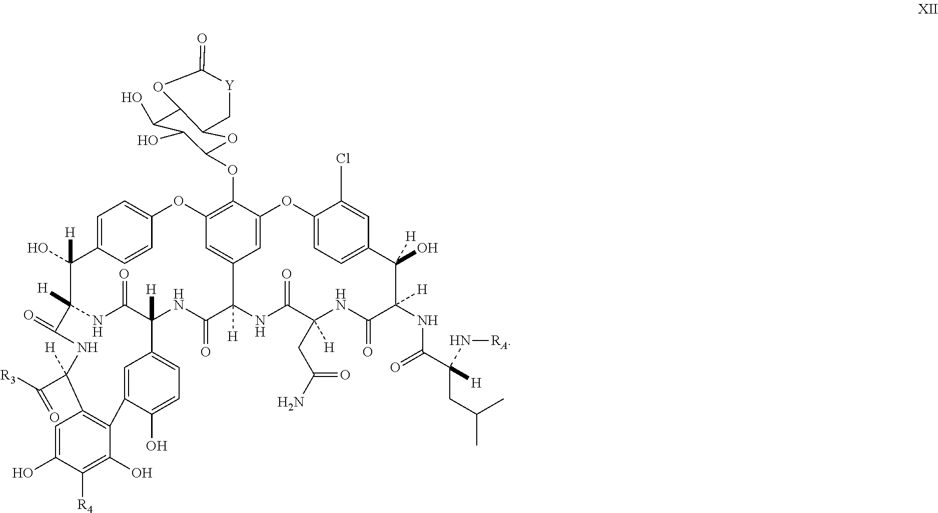

wherein R, R₁, R₂, R₃, R₄, R_{A}, R_{C}, R_{D}, R_{E}, X, Y and Z are as defined herein.

In particular, the semi-synthetic glycopeptides described herein are made, for example, by modifying Compound A, Compound B, Compound H or Compound C scaffolds. These natural glycopeptide starting material is optionally unsubstituted or substituted at R₄ with CH₂NHCH₂PO₃H₂, or aminoloweralkyl as defined herein.

Substitutions at R₄ are introduced, for example, via Mannich reaction wherein the glycopeptide is treated with an amine and formaldehyde under basic conditions (for example, as described in The Journal of Antibiotics, Vol. 50, No. 6, p. 509-513).

### Pharmaceutical Compositions

Pharmaceutical compositions described herein comprise a therapeutically effective amount of a compound described herein formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. Some examples of materials which serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants are also present in the composition, according to the judgment of the formulator. The pharmaceutical compositions described herein are administered to humans and other animals orally, rectally, parenterally, intracisternally, intravaginally, intraperitoneally, topically (as by powders, ointments, or drops), bucally, or as an oral or nasal spray, or a liquid aerosol or dry powder formulation for inhalation.

Liquid dosage forms for oral administration include pharmaceutically acceptable emulsions, microemulsions, solutions, suspensions, syrups and elixirs. In addition to the active compounds, the liquid dosage forms optionally contain inert diluents such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions optionally also include adjuvants such as wetting agents, emulsifying and suspending agents, sweetening, flavoring, and perfuming agents.

Injectable preparations, for example, sterile injectable aqueous or oleaginous suspensions are formulated using suitable dispersing or wetting agents and suspending agents. The sterile injectable preparation are optionally a sterile injectable solution, suspension or emulsion in a nontoxic parenterally acceptable diluent or solvent, for example, as a solution in 1,3-butanediol. Among the acceptable vehicles and solvents that are optionally employed are water, Ringer's solution, U.S.P. and isotonic sodium chloride solution. In addition, sterile, fixed oils are optionally employed as a solvent or suspending medium. For this purpose any bland fixed oil is optionally employed including synthetic mono- or diglycerides. In addition, fatty acids such as oleic acid are used in the preparation of injectables.

The injectable formulations are sterilized, for example, by filtration through a bacterial-retaining filter, or by incorporating sterilizing agents in the form of sterile solid compositions which is dissolved or dispersed in sterile water or other sterile injectable medium prior to use.

In order to prolong the effect of a drug, it is often desirable to slow the absorption of the drug from subcutaneous or intramuscular injection. This is accomplished, for example, by the use of a liquid suspension of crystalline or amorphous material with poor water solubility. The rate of absorption of the drug then depends upon its rate of dissolution which, in turn, depends upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release is optionally controlled. Examples of other biodegradable polymers include poly(orthoesters) and poly(anhydrides). Depot injectable formulations are also prepared, for example, by entrapping the drug in liposomes or microemulsions which are compatible with body tissues.

Compositions for rectal or vaginal administration are preferably suppositories which are optionally prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form optionally comprises buffering agents.

Solid compositions of a similar type are optionally employed as fillers in soft and hard-filled gelatin capsules using such excipients as lactose or milk sugar as well as high molecular weight polyethylene glycols and the like.

The solid dosage forms of tablets, dragees, capsules, pills, and granules are prepared, for example, with coatings and shells such as enteric coatings and other documented coatings. They optionally contain opacifying agents and also are of a composition that they release the active ingredient(s) only, or preferentially, in a certain part of the intestinal tract, optionally, in a delayed manner. Examples of embedding compositions which are used include polymeric substances and waxes.

Solid compositions of a similar type are optionally employed as fillers in soft and hard-filled gelatin capsules using such excipients as lactose or milk sugar as well as high molecular weight polyethylene glycols and the like.

The active compounds are optionally in micro-encapsulated form with one or more excipients as noted above. The solid dosage forms of tablets, dragees, capsules, pills, and granules are optionally prepared with coatings and shells such as enteric coatings, release controlling coatings and other documented coatings. In such solid dosage forms the active compound is admixed, for example, with at least one inert diluent such as sucrose, lactose or starch. Such dosage forms optionally comprise additional substances other than inert diluents, e.g., tableting lubricants and other tableting aids such a magnesium stearate and microcrystalline cellulose. In the case of capsules, tablets and pills, the dosage forms optionally comprise buffering agents. They optionally contain opacifying agents and are of a composition that they release the active ingredient(s) only, or preferentially, in a certain part of the intestinal tract, optionally, in a delayed manner. Examples of embedding compositions which are used include polymeric substances and waxes.

Dosage forms for topical or transdermal administration of a compound described herein include ointments, pastes, creams, lotions, gels, powders, solutions, sprays, inhalants or patches. The active component is admixed under sterile conditions with a pharmaceutically acceptable carrier and any needed preservatives or buffers as required. Ophthalmic formulations, ear drops, and the like are also contemplated.

The ointments, pastes, creams and gels optionally contain, in addition to an active compound described herein, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Compositions described herein are optionally formulated for delivery as a liquid aerosol or inhalable dry powder. Liquid aerosol formulations are nebulized, for example, predominantly into particle sizes that are delivered to the terminal and respiratory bronchioles where bacteria reside in patients with bronchial infections, such as chronic bronchitis and pneumonia. Pathogenic bacteria are commonly present throughout airways down to bronchi, bronchioli and lung parenchema, particularly in terminal and respiratory bronchioles. During exacerbation of infection, bacteria can also be present in alveoli. Liquid aerosol and inhalable dry powder formulations are preferably delivered throughout the endobronchial tree to the terminal bronchioles and eventually to the parenchymal tissue.

Aerosolized formulations described herein are delivered, for example, using an aerosol forming device, such as a jet, vibrating porous plate or ultrasonic nebulizer, preferably selected to allow the formation of a aerosol particles having with a mass medium average diameter predominantly between 1 to 5μ. Further, the formulation preferably has balanced osmolarity ionic strength and chloride concentration, and the smallest aerosolizable volume able to deliver effective dose of the compounds described herein to the site of the infection. Additionally, the aerosolized formulation preferably does not impair negatively the functionality of the airways and does not cause undesirable side effects.

Aerosolization devices suitable for administration of aerosol formulations described herein include, for example, jet, vibrating porous plate, ultrasonic nebulizers and energized dry powder inhalers, that are able to nebulize the formulation into aerosol particle size predominantly in the size range from 1-5μ. Predominantly in this application means that at least 70% but preferably more than 90% of all generated aerosol particles are within 1-5μ range. A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An ultrasonic nebulizer works by a piezoelectric crystal that shears a liquid into small aerosol droplets. A variety of suitable devices are available, including, for example, AeroNeb™ and AeroDose™ vibrating porous plate nebulizers (AeroGen, Inc., Sunnyvale, Calif.), Sidestream® nebulizers (Medic-Aid Ltd., West Sussex, England), Pari LC® and Pari LC Star® jet nebulizers (Pari Respiratory Equipment, Inc., Richmond, Va.), and Aerosonic™ (DeVilbiss Medizinische Produkte (Deutschland) GmbH, Heiden, Germany) and UltraAire® (Omron Healthcare, Inc., Vernon Hills, Ill.) ultrasonic nebulizers.

Compounds described herein are formulated, for example, for use as topical powders and sprays that contain, in addition to the compounds described herein, excipients such as lactose, talc, silicic acid, aluminum hydroxide, calcium silicates and polyamide powder, or mixtures of these substances. Sprays optionally contain customary propellants such as chlorofluorohydrocarbons.

Transdermal patches have the added advantage of providing controlled delivery of a compound to the body. Such dosage forms made, for example, by dissolving or dispensing the compound in the proper medium. Absorption enhancers are optionally used to increase the flux of the compound across the skin. The rate is controlled, for example, by either providing a rate controlling membrane or by dispersing the compound in a polymer matrix or gel.

According to the methods of treatment described herein, bacterial infections are treated or prevented in a patient such as a human or lower mammal by administering to the patient a therapeutically effective amount of a compound described herein, in such amounts and for such time as is necessary to achieve the desired result. By a “therapeutically effective amount” of a compound described herein is meant a sufficient amount of the compound to treat bacterial infections, at a reasonable benefit/risk ratio applicable to any medical treatment. The total daily usage of the compounds and compositions described herein will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors known in the medical arts.

The total daily dose of the compounds described herein administered to a human or other mammal in single or in divided doses is in amounts, for example, from 0.01 to 50 mg/kg body weight or more usually from 0.1 to 25 mg/kg body weight. Single dose compositions contain, for example, such amounts or submultiples thereof to make up the daily dose. In general, treatment regimens described herein comprise administration to a patient in need of such treatment from about 10 mg to about 2000 mg of the compound(s) described herein per day in single or multiple doses.

### Abbreviations

Abbreviations which may have been used in the descriptions of the schemes and the examples that follow are: AcOH for acetic acid; AIBN for azobisisobutyronitrile; nBu for normal butyl; (Boc)2O for di-tert-butyl dicarbonate, Bu₃SnH for tributyltin hydride; CDI for carbonyldiimidazole; DBU for 1,8-diazabicyclo[5.4.0]undec-7-ene; DCC for dicyclohexyl carbodiimide; DCM for dichloromethane; DEAD for diethylazodicarboxylate; DMF for dimethylformamide; DIEA or DIPEA for N,N-diisopropylethylamine; DMP for 2,2-dimethoxypropane; DMSO for dimethylsulfoxide (or methylsulfoxide); DPPA for diphenylphosphoryl azide; Et₃N for triethylamine; EtOAc for ethyl acetate; Et₂O for diethyl ether; EtOH for ethanol; HOAc for acetic acid; HOSu for N-hydroxysuccinimide; LiHMDS or LiN(TMS)₂ for lithium bis(trimethylsilyl)amide; MCPBA for meta-chloroperbenzoic acid; MeOH for methanol; MsCl for methanesulfonyl chloride; NaHMDS or NaN(TMS)₂ for sodium bis(trimethylsilyl)amide; NMO for N-methylmorpholine N-oxide; SOCl₂ for thionyl chloride; PPTS for pyridium p-toluene sulfonate; Pd(OAc)₂ for palladium (II) acetate; PPh₃ for triphenylphosphine; Py for pyridine; TFA for trifluoroacetic acid; TEA for triethylamine; THF for tetrahydrofuran; TMSCl for trimethylsilyl chloride; TMSCF₃ for trimethyl(trifluoromethyl)-silane; TPP for triphenylphosphine; TPAP for tetra-n-propylammonium perruthenate; DMAP for 4-dimethylamino pyridine; TsOH for p-toluene sulfonic acid; MsOH for methanesulfonic acid; OMs for mesylate, OTs for tosylate; OTf for triflate; Boc for tert-butoxycarbonyl; Fmoc for N-fluorenylmethoxycarbonyl; Su for succinimide; Ph for phenyl; HBPyU for O-benzotriazol-1-yl-N,N,N′,N′,-bis(tetramethylene)uronium hexafluorophosphate; PyBOP for benzotriazol-1-yloxytripyrrolidinophosphonium hexafluorophosphate; HATU for N,N,N′,N′-tetramethyl-O-(7-azabenzotriazol-1-yl)uranium hexafluorophosphate.

### Methicillin-Resistant Staphylococcus aureus

Staphylococcus aureus (S. aureus), a spherical bacterium, is the most common cause of staph infections. S. aureus has been known to cause a range of illnesses from minor skin infections, such as pimples, impetigo, boils, cellulitis folliculitis, furuncles, carbuncles, scalded skin syndrome, abscesses, to life-threatening diseases such as pneumonia, meningitis, osteomyelitis endocarditis, toxic shock syndrome, and septicemia. Further, S. aureus is one of the most common causes of nosocomial infections, often causing postsurgical wound infections.

Methicillin was introduced in the late 1950s to treat infections caused by penicillin-resistant S. aureus. It has been reported previously that S. aureus isolates had acquired resistance to methicillin (methicillin-resistant S. aureus, MRSA). The methicillin resistance gene (mecA) encodes a methicillin-resistant penicillin-binding protein that is not present in susceptible strains. mecA is carried on a mobile genetic element, the staphylococcal cassette chromosome mec (SCCmec), of which four forms have been described that differ in size and genetic composition. The methicillin-resistant penicillin-binding protein allows for resistance to β-lactam antibiotics and obviates their clinical use during MRSA infections.

In one aspect is a method for treating a subject having a resistant bacterium comprising administering to the subject a compound of Formula (I)-(XII) or a pharmaceutically acceptable salt, ester, solvate, alkylated quaternary ammonium salt, stereoisomer, tautomer or prodrug thereof. In one embodiment, the bacterium is a Gram-positive bacteria. In another embodiment, the Gram-positive bacterium is S. aureus. In further embodiment, the S. aureus is resistant or refractory to a beta-lactam antibiotic. In yet a further embodiment, the beta-lactam antibiotic belongs to the class of penicillins. In a further embodiment, the beta-lactam antibiotic is methicillin. In yet another embodiment, the subject has a methicillin-resistant S. aureus bacteria. In one embodiment the beta-lactam antibiotic is flucloxacillin. In another embodiment is a method for treating a subject having a dicloxacillin-resistant bacteria comprising administering to the subject a compound of Formula (I)-(XII) or a pharmaceutically acceptable salt, ester, solvate, alkylated quaternary ammonium salt, stereoisomer, tautomer or prodrug thereof wherein the subject is refractory to dicloxacillin. Also disclosed herein is a method for treating a subject having a methicillin-resistant bacteria comprising administering a compound of Formula (I)-(XII) or a pharmaceutically acceptable salt, ester, solvate, alkylated quaternary ammonium salt, stereoisomer, tautomer or prodrug thereof wherein the subject has been determined to have a methicillin-resistant bacteria. In one embodiment the subject is screened for methicillin-resistant bacteria. In another embodiment, the subject screening is performed through a nasal culture. In a further embodiment the methicillin-resistant bacteria is detected by swabbing the nostril(s) of the subject and isolating the bacteria. In another embodiment, Real-time PCR and/or Quantitative PCR is employed to determine whether the subject has a methicillin-resistant bacteria.

In one embodiment is a method for treating a subject having a first-generation cephalosporin-resistant bacteria comprising administering a compound of Formula (I)-(XII) or a pharmaceutically acceptable salt, ester, solvate, alkylated quaternary ammonium salt, stereoisomer, tautomer or prodrug thereof wherein the subject is refractory to a first-generation cephalosporin. In one embodiment, the bacteria is resistant to a first-generation cephalosporin. In a further embodiment, the bacteria is resistant to cefacetrile. In another embodiment, the bacteria is resistant to cefadroxil. In yet another embodiment, the bacteria is resistant to cefalexin. In one embodiment, the bacteria is resistant to cefaloglycin. In another embodiment, the bacteria is resistant to cefalonium. In another embodiment, the bacteria is resistant to cefaloridine. In yet another embodiment, the bacteria is resistant to cefalotin. In a further embodiment, the bacteria is resistant to cefapirin. In yet a further embodiment, the bacteria is resistant to cefatrizine. In one embodiment, the bacteria is resistant to cefazaflur. In another embodiment, the bacteria is resistant to cefazedone. In yet another embodiment, the bacteria is resistant to cefazolin. In a further embodiment, the bacteria is resistant to cefradine. In yet a further embodiment, the bacteria is resistant to cefroxadine. In one embodiment, the bacteria is resistant to ceftezole.

In one embodiment is a method for treating a subject having a second-generation cephalosporin-resistant bacteria comprising administering a compound of Formula (I)-(XII) or a pharmaceutically acceptable salt, ester, solvate, alkylated quaternary ammonium salt, stereoisomer, tautomer or prodrug thereof wherein the subject is refractory to a second-generation cephalosporin. In another embodiment, the bacteria is resistant to a second-generation cephalosporin. In a further embodiment, the bacteria is resistant to cefaclor. In another embodiment, the bacteria is resistant to cefonicid. In yet another embodiment, the bacteria is resistant to cefprozil. In one embodiment, the bacteria is resistant to cefuroxime. In another embodiment, the bacteria is resistant to cefuzonam. In another embodiment, the bacteria is resistant to cefmetazole. In yet another embodiment, the bacteria is resistant to cefotetan. In a further embodiment, the bacteria is resistant to cefoxitin.

In one embodiment is a method for treating a subject having a third-generation cephalosporin-resistant bacteria comprising administering a compound of Formula (I)-(XII) or a pharmaceutically acceptable salt, ester, solvate, alkylated quaternary ammonium salt, stereoisomer, tautomer or prodrug thereof wherein the subject is refractory to a third-generation cephalosporin. In another embodiment, the bacteria is resistant to a third-generation cephalosporin. In a further embodiment, the bacteria is resistant to cefcapene. In another embodiment, the bacteria is resistant to cefdaloxime. In yet another embodiment, the bacteria is resistant to cefdinir. In one embodiment, the bacteria is resistant to cefditoren. In another embodiment, the bacteria is resistant to cefixime. In another embodiment, the bacteria is resistant to cefmenoxime. In yet another embodiment, the bacteria is resistant to cefodizime. In a further embodiment, the bacteria is resistant to cefotaxime. In yet a further embodiment, the bacteria is resistant to cefpimizole. In one embodiment, the bacteria is resistant to cefpodoxime. In another embodiment, the bacteria is resistant to cefteram. In yet another embodiment, the bacteria is resistant to ceftibuten. In a further embodiment, the bacteria is resistant to ceftiofur. In yet a further embodiment, the bacteria is resistant to ceftiolene. In one embodiment, the bacteria is resistant to ceftizoxime. In another embodiment, the bacteria is resistant to ceftriaxone. In yet another embodiment, the bacteria is resistant to cefoperazone. In yet a further embodiment, the bacteria is resistant to ceftazidime.

In one embodiment is a method for treating a subject having a fourth-generation cephalosporin-resistant bacteria comprising administering a compound of Formula (I)-(XII) or a pharmaceutically acceptable salt, ester, solvate, alkylated quaternary ammonium salt, stereoisomer, tautomer or prodrug thereof wherein the subject is refractory to a fourth-generation cephalosporin. In another embodiment, the bacteria is resistant to a fourth-generation cephalosporin. In a further embodiment, the bacteria is resistant to cefclidine. In another embodiment, the bacteria is resistant to cefepime. In yet another embodiment, the bacteria is resistant to cefluprenam. In one embodiment, the bacteria is resistant to cefoselis. In another embodiment, the bacteria is resistant to cefozopran. In another embodiment, the bacteria is resistant to cefpirome. In yet another embodiment, the bacteria is refractory to cefquinome.

In one embodiment is a method for treating a subject having a carbapenem-resistant bacteria comprising administering a compound of Formula (I)-(XII) or a pharmaceutically acceptable salt, ester, solvate, alkylated quaternary ammonium salt, stereoisomer, tautomer or prodrug thereof wherein the subject is refractory to a carbapenem. In another embodiment, the bacteria is resistant to a carbapenem. In a further embodiment, the bacteria is resistant to imipenem. In another embodiment, the bacteria is resistant to meropenem. In yet another embodiment, the bacteria is resistant to ertapenem. In one embodiment, the bacteria is resistant to faropenem. In another embodiment, the bacteria is resistant to doripenem. In another embodiment, the bacteria is resistant to panipenem. In yet another embodiment, the bacteria is resistant to biapenem.

### Vancomycin-Intermediate and Vancomycin-Resistant Staphylococcus aureus

Vancomycin-intermediate Staphylococcus aureus and vancomycin-resistant staphylococcus aureus are specific types of antimicrobial-resistant Staph bacteria that are refractory to vancomycin treatment. S. aureus isolates for which vancomycin MICs are 4-8 μg/mL are classified as vancomycin-intermediate and isolates for which vancomycin MICs are ≧16 μg/mL are classified as vancomycin-resistant (Clinical and Laboratory Standards Institute/NCCLS. Performance Standards for Antimicrobial Susceptibility Testing. Sixteenth informational supplement. M100-S16. Wayne, Pa.: CLSI, 2006).

As used herein, the term “minimum inhibitory concentration” (MIC) refers to the lowest concentration of an antibiotic that is needed to inhibit growth of a bacterial isolate in vitro. A common method for determining the MIC of an antibiotic is to prepare several tubes containing serial dilutions of the antibiotic, that are then inoculated with the bacterial isolate of interest. The MIC of an antibiotic is determined from the tube with the lowest concentration that shows no turbidity (no growth).

In one aspect is a method of treating a subject having a bacterial infection comprising administering to the subject a compound of Formula (I)-(XII) or a pharmaceutically acceptable salt, ester, solvate, alkylated quaternary ammonium salt, stereoisomer, tautomer or prodrug thereof wherein the bacterial infection comprises a vancomycin-intermediate Staphylococcus aureus bacterium. In one embodiment, the vancomycin-intermediate Staphylococcus aureus bacterium has a MIC of between about 4 to about 8 μg/mL. In another embodiment, the vancomycin-intermediate Staphylococcus aureus bacterium has a MIC of about 4 μg/mL. In yet another embodiment, the vancomycin-intermediate Staphylococcus aureus bacterium has a MIC of about 5 μg/mL. In a further embodiment, the vancomycin-intermediate Staphylococcus aureus bacterium has a MIC of about 6 μg/mL. In yet a further embodiment, the vancomycin-intermediate Staphylococcus aureus bacterium has a MIC of about 7 μg/mL. In one embodiment, the vancomycin-intermediate Staphylococcus aureus bacterium has a MIC of about 8 μg/mL.

In another aspect is a method of treating a subject having a bacterial infection comprising administering to the subject a compound of Formula (I)-(XII) or a pharmaceutically acceptable salt, ester, solvate, alkylated quaternary ammonium salt, stereoisomer, tautomer or prodrug thereof wherein the bacterial infection comprises a vancomycin-resistant Staphylococcus aureus bacterium. In one embodiment, the vancomycin-resistant Staphylococcus aureus bacterium has a MIC of between about 16 μg/mL. In another embodiment, the vancomycin-resistant Staphylococcus aureus bacterium has a MIC of about ≧16 μg/mL. In yet another embodiment, the vancomycin-resistant Staphylococcus aureus bacterium has a MIC of about 20 μg/mL. In a further embodiment, the vancomycin-resistant Staphylococcus aureus bacterium has a MIC of about 25 μg/mL.

In one embodiment, conditions treated by the compounds described herein include, but are not limited to, endocarditis, osteomyelitis, neningitis, skin and skin structure infections, genitourinary tract infections, abscesses, and necrotizing infections. In another embodiment, the compounds disclosed herein are used to treat conditions, such as, but not limited to, diabetic foot infections, decubitus ulcers, burn infections, animal or human bite wound infections, synergistic-necrotizing gangrene, necrotizing fascilitis, intra-abdominal infection associated with breeching of the intestinal barrier, pelvic infection associated with breeching of the intestinal barrier, aspiration pneumonia, and post-operative wound infections. In another embodiment, the conditions listed herein are caused by, contain, or result in the presence of VISA and/or VISA.

### Vancomycin-Resistant Enterococci

Enterococci are bacteria that are normally present in the human intestines and in the female genital tract and are often found in the environment. These bacteria sometimes cause infections. In some cases, enterococci have become resistant to vancomycin (also known as vancomycin-resistant enterococci or VRE.) Common forms of resistance to vancomycin occur in enterococcal strains that involve the acquisition of a set of genes endoding proteins that direct peptidoglycan precursors to incorporate D-Ala-D-Lac instead of D-Ala-D-Ala. The six different types of vancomycin resistance shown by enterococcus are: Van-A, Van-B, Van-C, Van-D, Van-E and Van-F. In some cases, Van-A VRE is resistant to both vancomycin and teicoplanin, while in other cases, Van-B VRE is resistant to vancomycin but sensitive to teicoplanin; in further cases Van-C is partly resistant to vancomycin, and sensitive to teicoplanin.

In one aspect, is a method of treating a subject having a vancomycin-resistant enterococci comprising administering to the subject a compound of Formula (I)-(XII) or a pharmaceutically acceptable salt, ester, solvate, alkylated quaternary ammonium salt, stereoisomer, tautomer or prodrug thereof wherein the enterococci has developed resistance to vancomycin. In one embodiment, the subject has been previously treated with vancomycin for a sustained period of time. In another embodiment, the subject has been hospitalized. In yet another embodiment, the subject has a weakened immune system such as patients in Intensive Care Units or in cancer or transplant wards. In a further embodiment, the subject has undergone surgical procedures such as, for example, abdominal or chest surgery. In yet a further embodiment, the subject has been colonized with VRE. In one embodiment, the subject has a medical device such that an infection has developed. In another embodiment, the medical device is a urinary catheter or central intravenous (IV) catheter.

In another embodiment, is a method of treating a subject having a vancomycin-resistant enterococci comprising administering to the subject a compound of Formula (I)-(XII) or a pharmaceutically acceptable salt, ester, solvate, alkylated quaternary ammonium salt, stereoisomer, tautomer or prodrug thereof wherein the enterococcus has Van-A resistance.

In another embodiment, is a method of treating a subject having a vancomycin-resistant enterococci comprising administering to the subject a compound of Formula (I)-(XII) or a pharmaceutically acceptable salt, ester, solvate, alkylated quaternary ammonium salt, stereoisomer, tautomer or prodrug thereof wherein the enterococcus has Van-B resistance.

In another embodiment, is a method of treating a subject having a vancomycin-resistant enterococci comprising administering to the subject a compound of Formula (I)-(XII) or a pharmaceutically acceptable salt, ester, solvate, alkylated quaternary ammonium salt, stereoisomer, tautomer or prodrug thereof wherein the enterococcus has Van-C resistance.

### EXAMPLES

The following examples provide details concerning the synthesis, properties and activities and applications of semi-synthetic glycopeptides described herein. It should be understood the following is representative only.

### Example 1

### Synthesis of Compound (1)

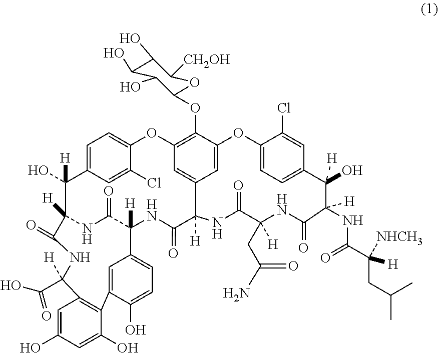

Vancomycin (30 g) was added slowly to a mixture solution (300 ml, TFA: H₂O=9:1) at 10° C. Then the reaction mixture was stirred at 10° C. for 2 hrs (with reaction progress checked by HPLC). The reaction mixture was quenched with 1500 ml cold diethyl ether, the precipitate was filtered and washed by ether several times, then dried under vacuum. The crude product was purified by reverse phase column (MeCN:H₂O=10%˜20%) to afford Compound (1) as a white solid. (yield=45%).

### Example 2

### Synthesis of Compound (2)

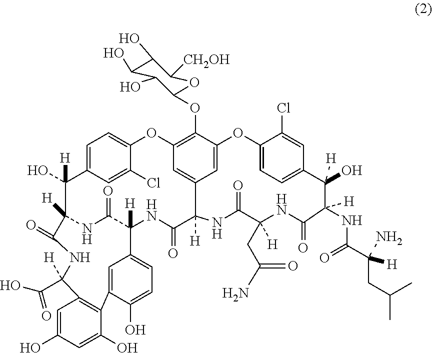

Using a procedure similar to the preparation of Compound (1), and replacing vancomycin with desmethylvancomycin, Compound (2) is made.

### Example 3

### Synthesis of Compound (3)

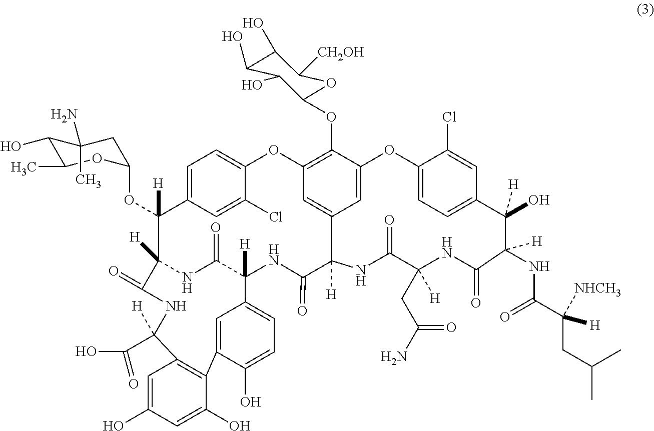

Using a procedure similar to the preparation of Compound (1), and replacing vancomycin with LY264826, Compound (3) is made.

### Example 4

### Synthesis of Compound (4)

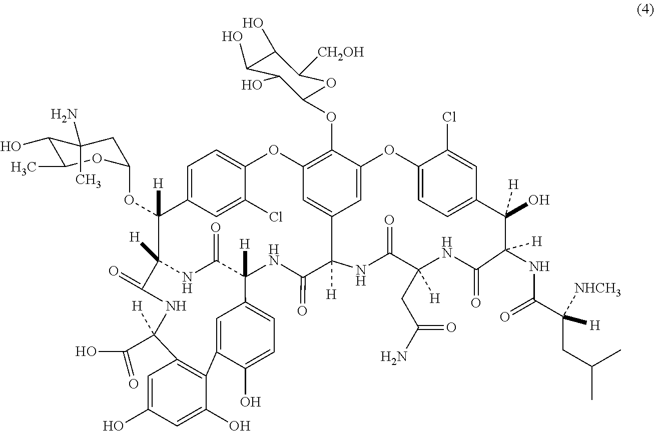

Using a procedure similar to the preparation of Compound (1), and replacing vancomycin with eremomycin, Compound (4) is made.

### Example 5

### Synthesis of Compound (5)

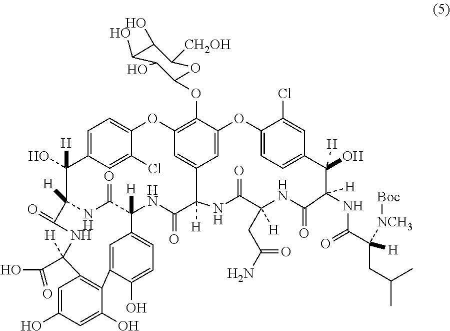

Compound (1) (5.0 g, 3.72 mmol) was dissolved in THF/H₂O (35 ml/35 ml). TEA (0.77 ml, 5.58 mmol) was then added. The reaction mixture was cooled down to 15° C. and then (Boc)₂O (0.89 g, 4.08 mmol) was added slowly. After the addition, the reaction mixture was allowed to be stirred at 15° C. for 7 hrs. It was concentrated and the crude was purified by reverse phase column (MeCN:H₂O=1:5-3:10). 3 g of Compound (5) was obtained as a white solid (yield=60%).

### Example 6

### Synthesis of Compound (6)

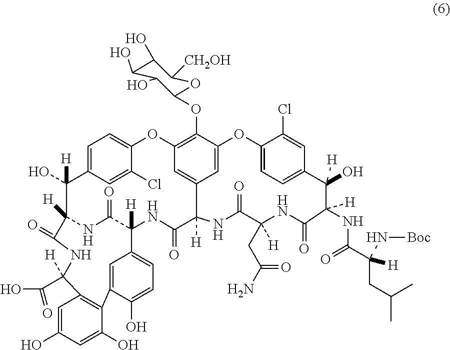

Using a procedure similar to the preparation of Compound in, and replacing Compound (1) with Compound (2), Compound (6) is made.

### Example 7

### Synthesis of Compound (7)

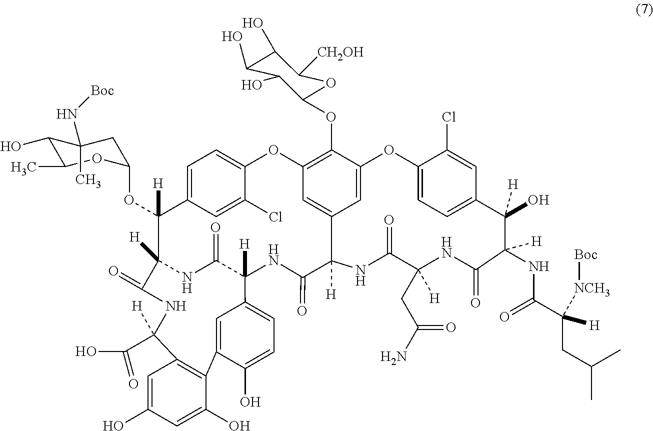

Using a procedure similar to the preparation of Compound (5), and replacing Compound (1) with Compound (3), Compound (7) is made.

### Example 8

### Synthesis of Compound (8)

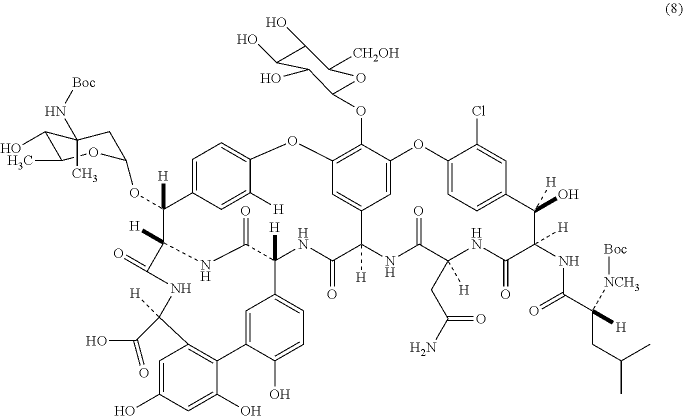

Using a procedure similar to the preparation of Compound (5), and replacing Compound (1) with Compound (4) Compound (8) is made.

### Example 9

### Synthesis of Compound (9)

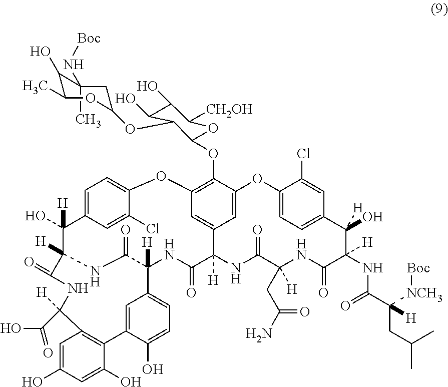

Using a procedure similar to the preparation of Compound (5), and replacing Compound (1) with vancomycin, Compound (9) is made.

### Example 10

### Synthesis of Compound (10)

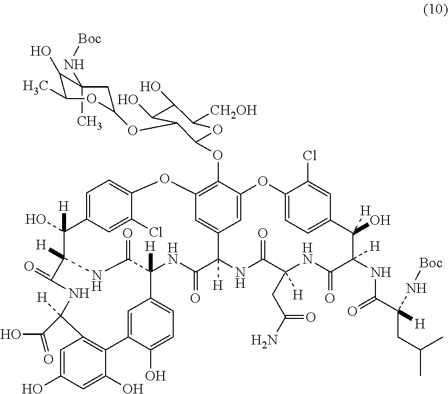

Using a procedure similar to the preparation of Compound (5), and replacing Compound (1) with desmethylvancomycin Compound (10) is made.

### Example 11

### Synthesis of Compound (11)

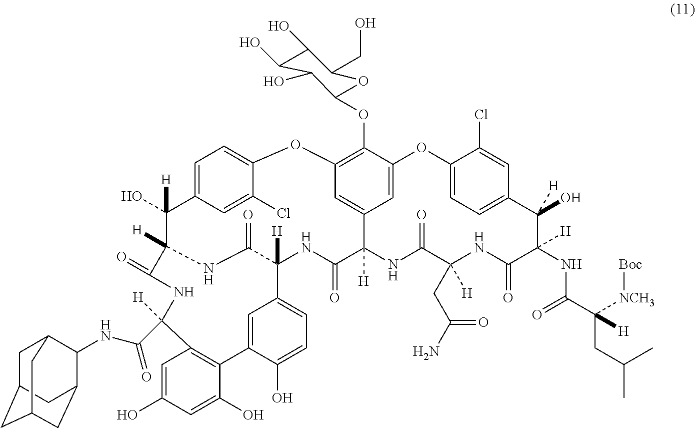

Compound (5) (1 g, 0.712 mmol) and 2-adamantylamine hydrochloride (0.4 g, 2.1 mmol) were dissolved in anhydrous DMSO (12 ml). DIEA was added the solution to adjust the pH of reaction mixture to 8. HATU (0.3 g, 0.789 mmol) was then added in the presence of DIEA. Stirring was continued for about 1 hr, checking the progress of the reaction to completion by TLC. The resulting mixture was then added to 120 ml of water and filtered. The cake was washed for two times with water and dried in vacuum. Purification by running a normal phase silica column (MeOH: CH₂Cl₂=1:7-1:3) gave the Compound (11) as white solid (850 mg, yield=77%).

### Example 12

### Synthesis of Compound (12)

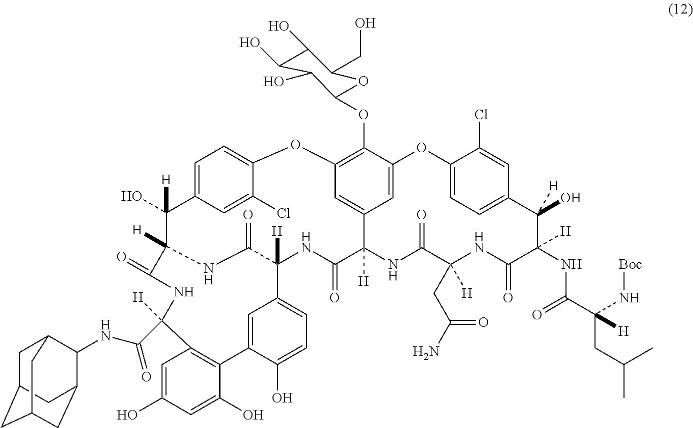

Using a procedure similar to the preparation of Compound (11), and replacing Compound (1) with Compound (1), Compound (12) is made.

### Example 13

### Synthesis of Compound (13)

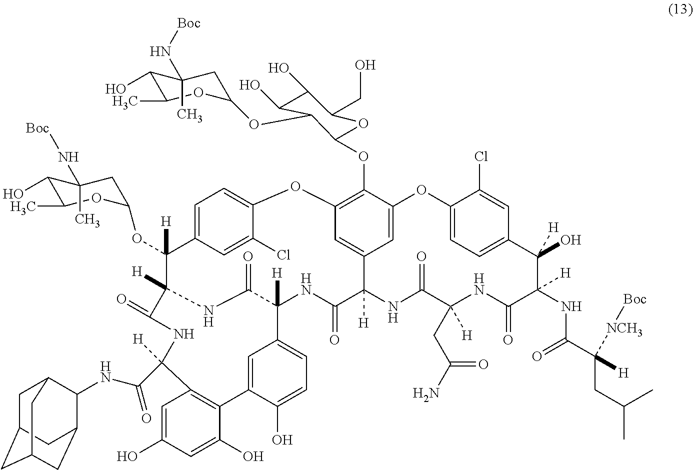

Using a procedure similar to the preparation of Compound (11), and replacing Compound (1) with Compound (7), Compound (13) is made.

### Example 14

### Synthesis of Compound (14)

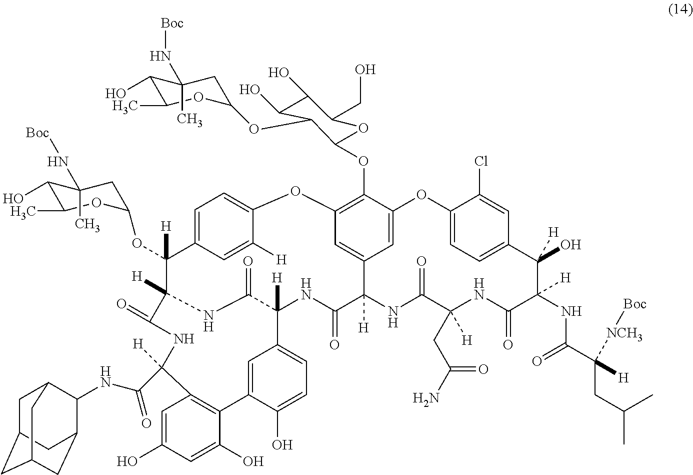

Using a procedure similar to the preparation of Compound (11), and replacing Compound (5) with Compound (8) Compound (14) is made.

### Example 15

### Synthesis of Compound (15)

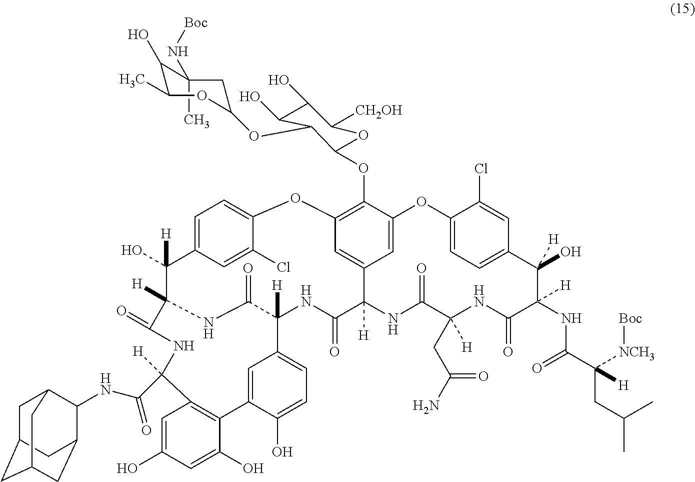

Using a procedure similar to the preparation of Compound (11), and replacing Compound (5) with Compound (9), Compound (15) is made.

### Example 16

### Synthesis of Compound (16)

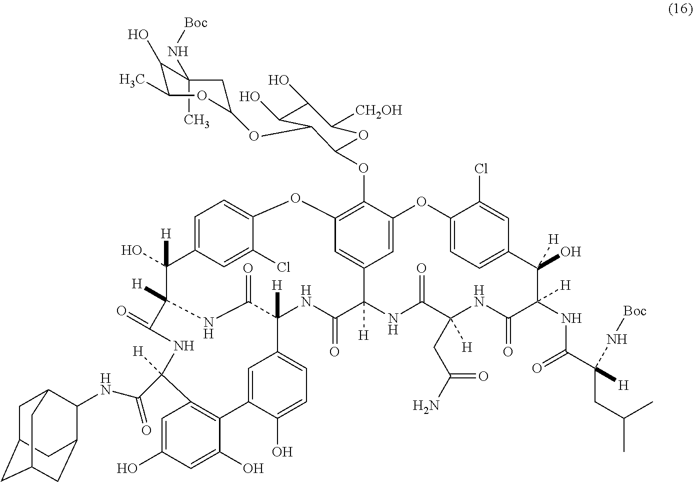

Using a procedure similar to the preparation of Compound (11), and replacing Compound (5) with Compound (10), Compound (16) is made.

### Example 17

### Synthesis of Compound (17)

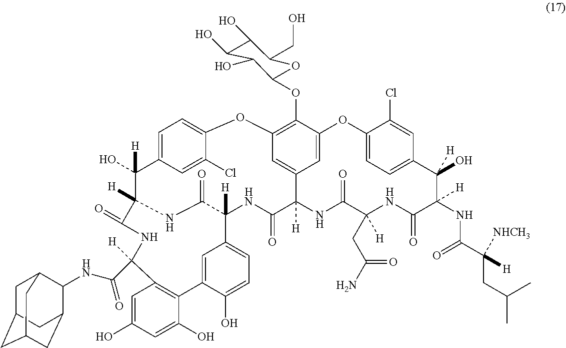

To a suspension of Compound (11) (380 mg) in CH₂Cl₂ (4 ml) at 0, was added TFA (0.5 ml) dropwise. The reaction mixture was stirred at 0° C. for 1 hour and then at room temperature for another hour. The reaction was follow by HPLC until the analysis showed no starting material present. Ether (30 ml) was added and the forming solid was collected and washed with ether twice. The collected white solid was dried and purified by preparative HPLC to yield Compound (17) as TFA salt.

### Example 18

### Synthesis of Compound (18)

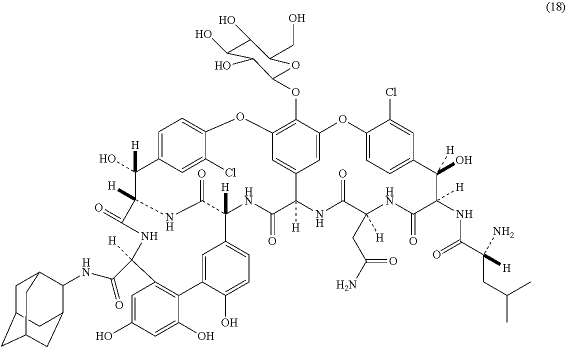

Using a procedure similar to the preparation of Compound (17), and replacing Compound (11) with Compound (12), Compound (18) as TFA salt is made.

### Example 19

### Synthesis of Compound (19)

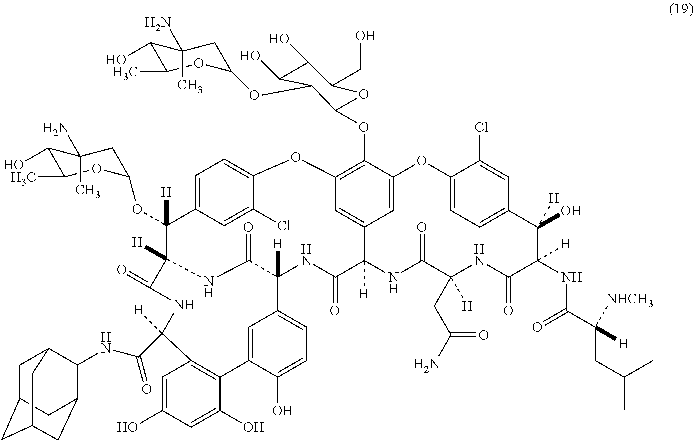

Using a procedure similar to the preparation of Compound (17), and replacing Compound (11) with Compound (13), Compound (19) as TFA salt is made.

### Example 20

### Synthesis of Compound (20)

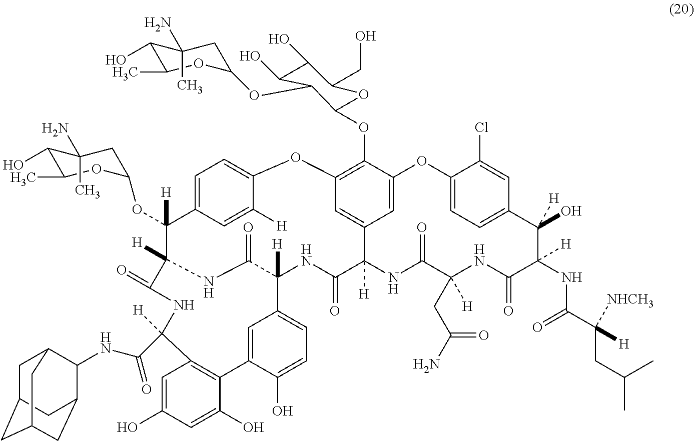

Using a procedure similar to the preparation of Compound (17), and replacing Compound (11) with Compound (14), Compound (20) as TFA salt is made.

### Example 21

### Synthesis of Compound (21)

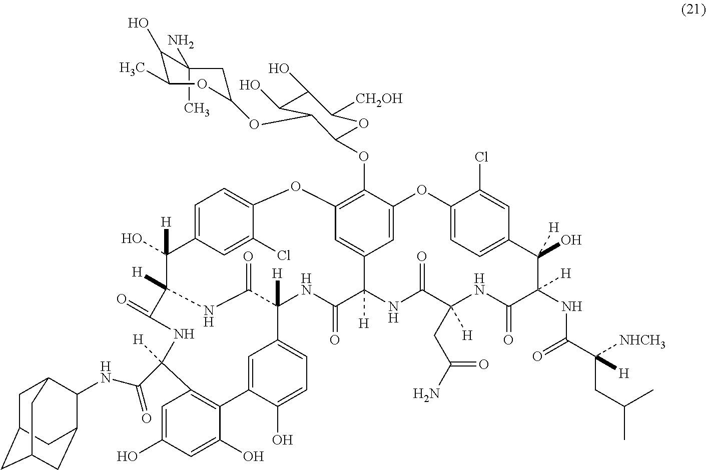

Using a procedure similar to the preparation of Compound (17), and replacing Compound (11) with Compound (15), Compound (21) as TFA salt is made.

### Example 22

### Synthesis of Compound (22)

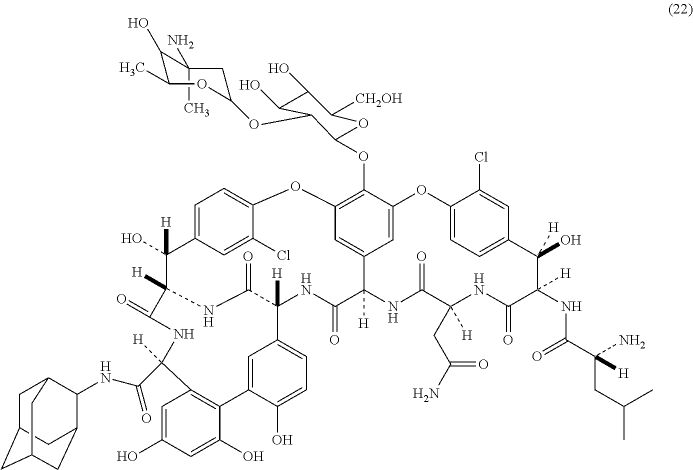

Using a procedure similar to the preparation of Compound (17), and replacing Compound (11) with Compound (16), Compound (22) as TFA salt is made.

### Example 23

### Synthesis of Compound (23)

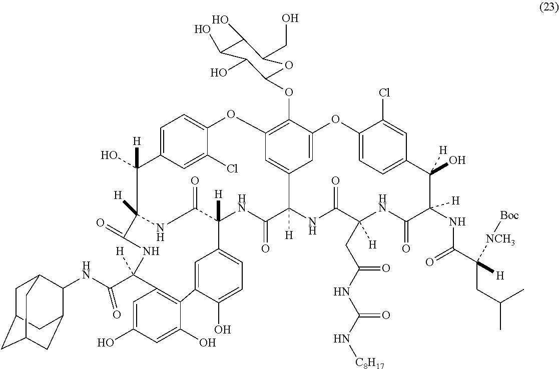

To Compound (11) (1.0 g, 0.65 mmol) and DMAP (0.25 g, 2.0 mmol) in dry DMF (15 ml) at room temperature, was added slowly C₈H₁₇NCO (0.20 g, 1.30 mmol). After stirring at room temperature for 15 hours, the reaction mixture was precipitated in ether and the solid was washed with water and collected to yield Compound (23) (1.0 g, 91% yield) as a white solid. Modification of conditions for the preparation of Compound (23) was conducted as follows. Compound (11) (100 mg) was azeotroped with toluene three times. This was dissolved in 1 ml dry DMF. DBU (3.0 equivalent) in 1 ml dry DMF was added under argon atmosphere in an ice bath followed by the addition of isocyanate C₈H₁₇NCO (2.0 equivalent) in 1 ml DMF. The mixture was stirred at room temperature overnight. The reaction was checked for completion by HPLC-MS. The reaction was quenched by adding water, and then filtered. The cake was washed three times with water. The crude compound was purified by preparative HPLC to afford Compound (23).

### Example 24

### Synthesis of Compound (24)

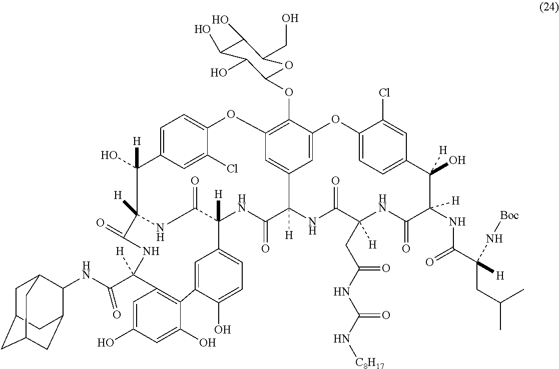

Using a procedure similar to the preparation of Compound (23), and replacing Compound (11) with Compound (12), Compound (24) is made.

### Example 25

### Synthesis of Compound (25)

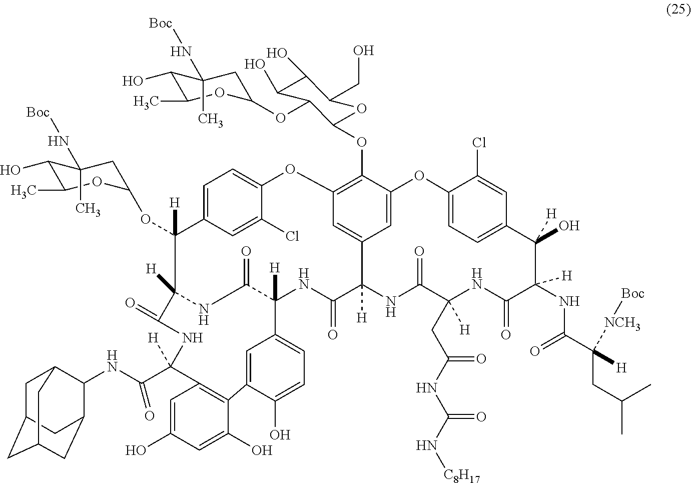

Using a procedure similar to the preparation of Compound (23), and replacing Compound (11) with Compound (13), Compound (25) is made.

### Example 26

### Synthesis of Compound (26)

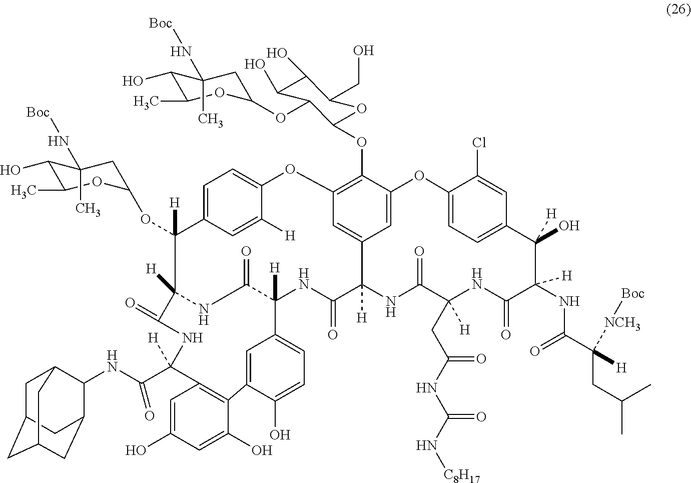

Using a procedure similar to the preparation of Compound (23), and replacing Compound (11) with Compound (14), Compound (26) is made.

### Example 27

### Synthesis of Compound (27)

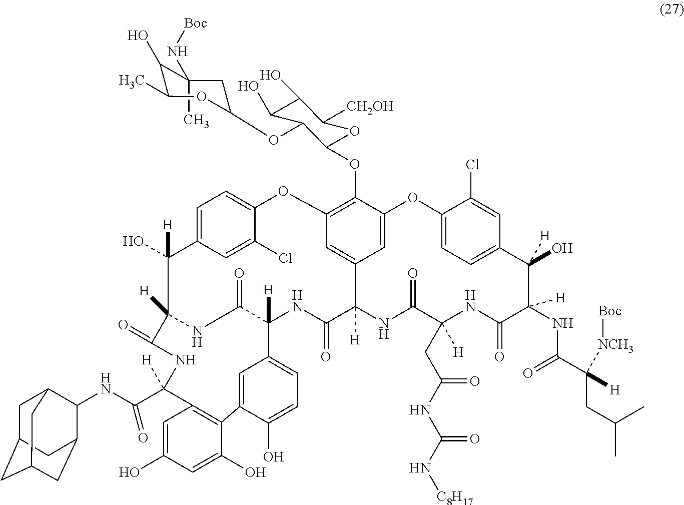

Using a procedure similar to the preparation of Compound (23), and replacing Compound (11) with Compound (15), Compound (27) is made.

### Example 28

### Synthesis of Compound (28)

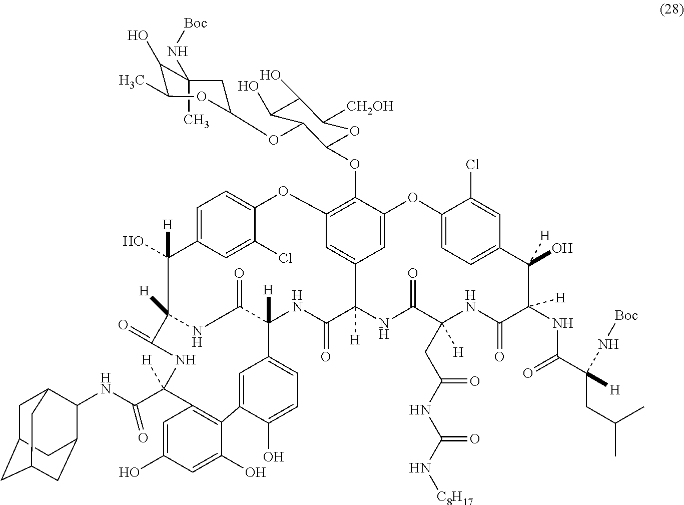

Using a procedure similar to the preparation of Compound (23), and replacing Compound (11) with Compound (16), Compound (28) is made.

### Example 29

### Synthesis of Compound (29)

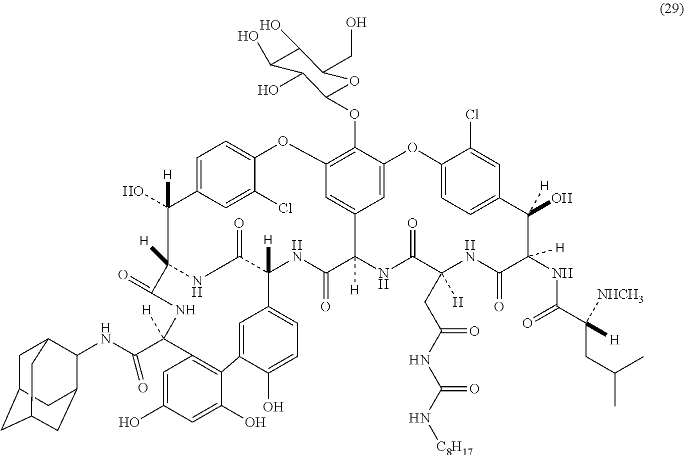

To a suspension of Compound (23) (1.0 g, 0.58 mmol) in CH₂Cl₂ (16 ml) at 0° C., was added TFA (4 ml) dropwise. The reaction mixture was stirred at 0° C. for 1 hour. Ether (80 ml) was added and the forming solid was collected and washed with ether 3 times. The collected white solid was dried and purified by preparative HPLC to yield Compound (29) as TFA salt (150 mg, 15%) as a white solid. Preparation HPLC conditions: Eluent:65/35 of MeCN/H₂O (with 0.1% TFA); Flow rate: 10 ml/min; Column size: 250*22 mm; Retention time: approximately 10 min.

### Example 30

### Synthesis of Compound (30)

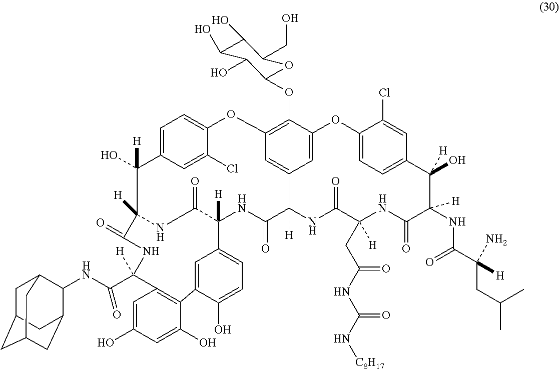

Using a procedure similar to the preparation of Compound (29), and replacing Compound (23) with Compound (24), Compound (30) as TFA salt is made.

### Example 31

### Synthesis of Compound (31)

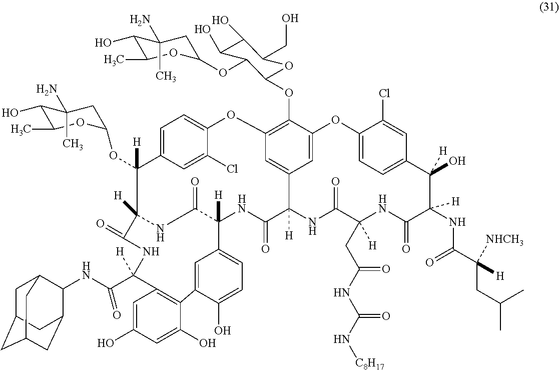

Using a procedure similar to the preparation of Compound (29), and replacing Compound (23) with Compound (25), Compound (31) as TFA salt is made.

### Example 32

### Synthesis of Compound (32)

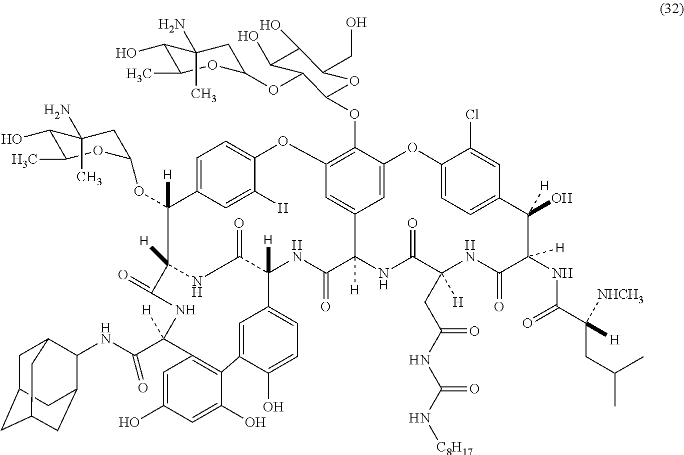

Using a procedure similar to the preparation of Compound (29), and replacing Compound (23) with Compound (26), Compound (32) as TFA salt is made.

### Example 33

### Synthesis of Compound (33)

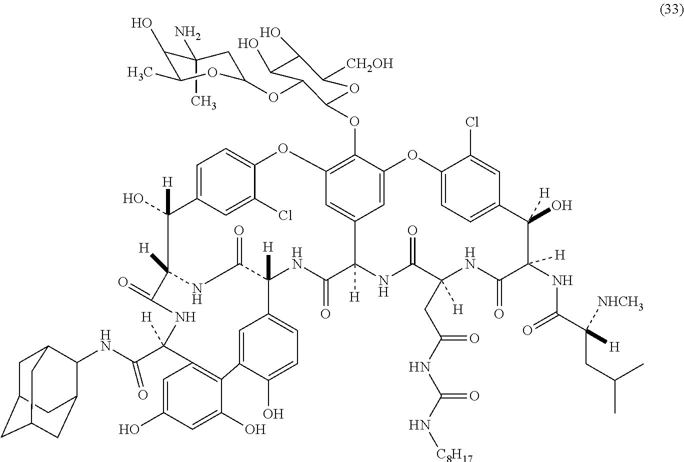

Using a procedure similar to the preparation of Compound (29), and replacing Compound (23) with Compound (27), Compound (33) as TFA salt is made.

### Example 34

### Synthesis of Compound (34)

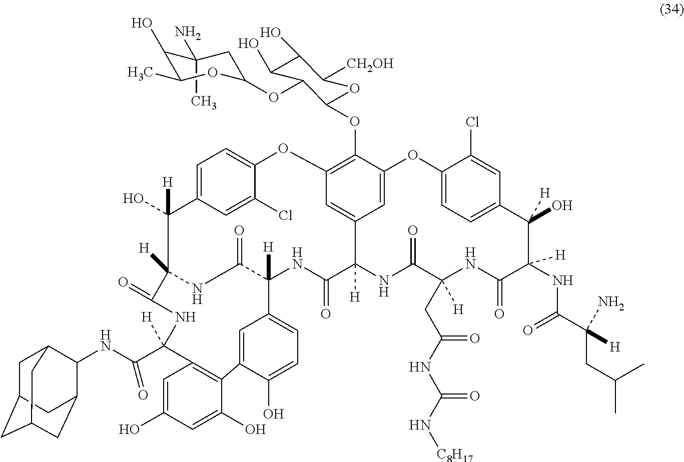

Using a procedure similar to the preparation of Compound (29), and replacing Compound (23) with Compound (28), Compound (34) as TFA salt is made.

### Example 35

### Synthesis of Compound (35)

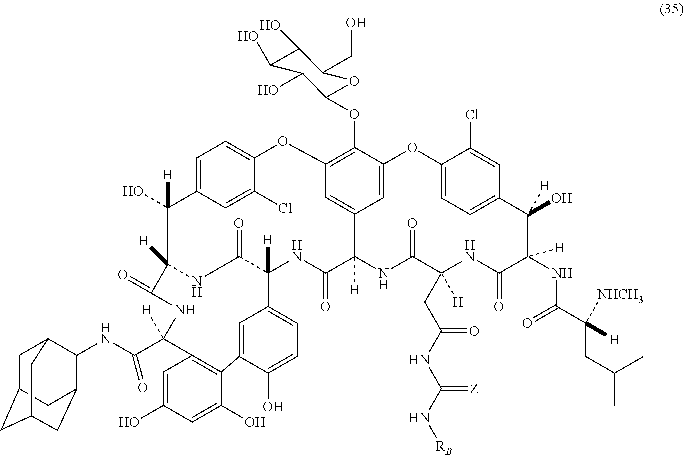

Using a procedure similar to the preparation of Compound (23), and reacting Compound (11) with the appropriate isocyanate or thioisocyanate (R_{B}—NCO or R_{B}—NCS), and treating the resulting product with TFA following the procedure as outlined in Example 29 to yield Compound (35) as a TFA salt where Z is O or S and R_{B} is loweralkyl, substituted loweralkyl, phenyl, pyridyl, substituted aryl or substituted heteroaryl is made.

### Example 36

### Synthesis of Compound (36)

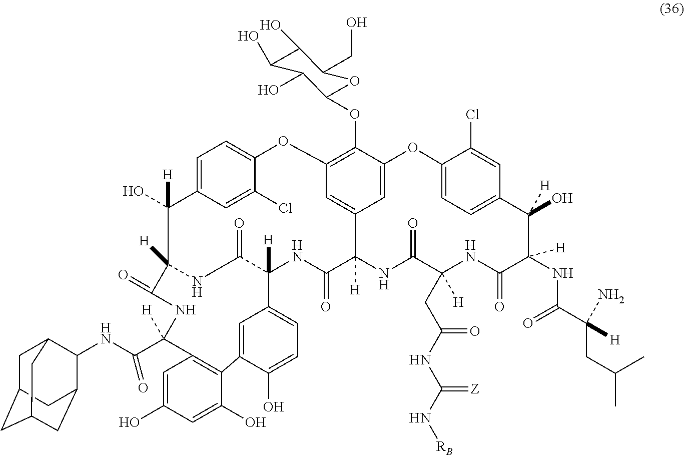

Using a procedure similar to the preparation of Compound (23), and reacting Compound (12) with the appropriate isocyanate or thioisocyanate (R_{B}—NCO or R_{B}—NCS), and treating the resulting product with TFA following the procedure as outlined in Example 29 to yield Compound (36) as a TFA salt where Z is O or S and R_{B} is loweralkyl, substituted loweralkyl, phenyl, pyridyl, substituted aryl or substituted heteroaryl is made.

### Example 37

### Synthesis of Compound (37)

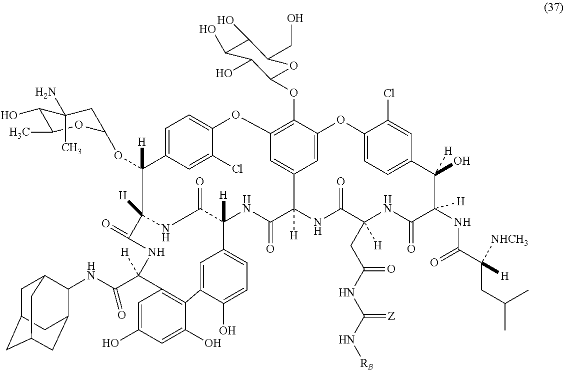

Using a procedure similar to the preparation of Compound (23), and reacting Compound (13) with the appropriate isocyanate or thioisocyanate (R_{B}—NCO or R_{B}—NCS), and treating the resulting product with TFA following the procedure as outlined in Example 29 to yield Compound (37) as a TFA salt where Z is O or S and R_{B} is loweralkyl, substituted loweralkyl, phenyl, pyridyl, substituted aryl or substituted heteroaryl is made.

### Example 38

### Synthesis of Compound (38)

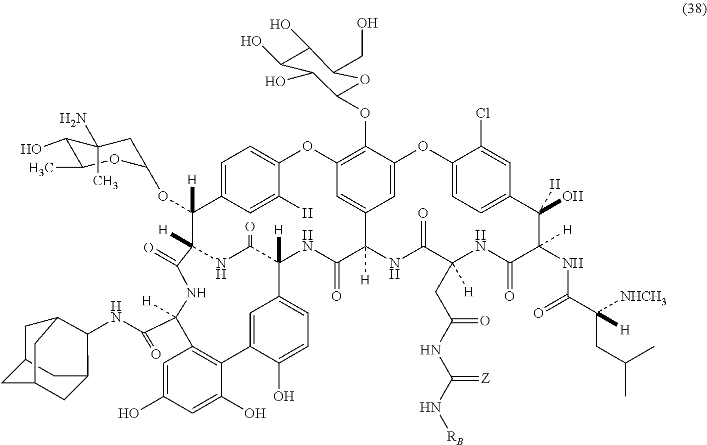

Using a procedure similar to the preparation of Compound (23), and reacting Compound (14) with the appropriate isocyanate or thioisocyanate (R_{B}—NCO or R_{B}—NCS), and treating the resulting product with TFA following the procedure as outlined in Example 29 to yield Compound (38) as a TFA salt where Z is O or S and R_{B} is loweralkyl, substituted loweralkyl, phenyl, pyridyl, substituted aryl or substituted heteroaryl is made.

### Example 39

### Synthesis of Compound (39)

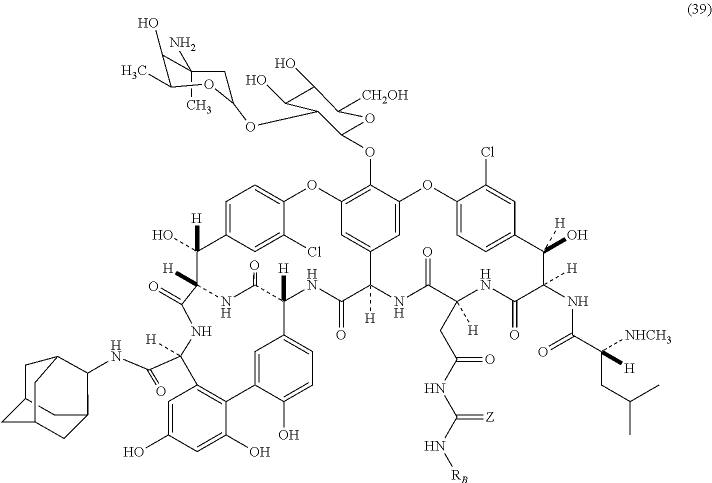

Using a procedure similar to the preparation of Compound (23), and reacting Compound (15) with the appropriate isocyanate or thioisocyanate (R_{B}—NCO or R_{B}—NCS), and treating the resulting product with TFA following the procedure as outlined in Example 29 to yield Compound (39) as a TFA salt where Z is O or S and R_{B} is loweralkyl, substituted loweralkyl, phenyl, pyridyl, substituted aryl or substituted heteroaryl is made.

### Example 40

### Synthesis of Compound (40)

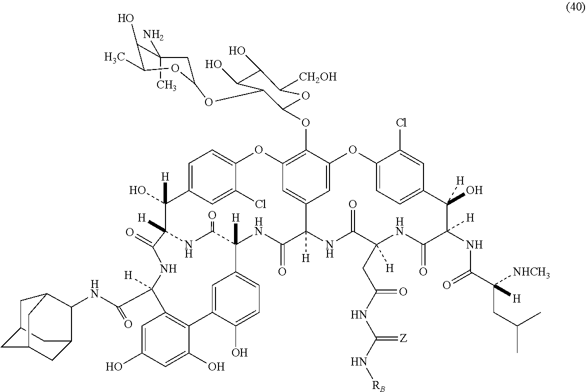

Using a procedure similar to the preparation of Compound (23), and reacting Compound (16) with the appropriate isocyanate or thioisocyanate (R_{B}—NCO or R_{B}—NCS), and treating the resulting product with TFA following the procedure as outlined in Example 29 to yield Compound (40) as a TFA salt where Z is O or S and R_{B} is loweralkyl, substituted loweralkyl, phenyl, pyridyl, substituted aryl or substituted heteroaryl is made.

### Example 41

### Synthesis of Compound (41)

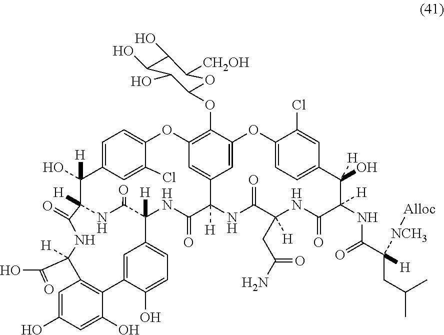

To a solution of Compound (1) (7.30 g, 5.59 mmol) dissolved into H₂O (28 mL) and THF (28 mL) was added Alloc-OSu (2.07 g, 11.18 mmol, 2 eq.) at room temperature. To the above mixture, DIPEA (1.4 mL) was added dropwise at room temperature (approx. 5 min). After stirring at room temperature for 1.5 hour, the reaction mixture was then monitored by analytical HPLC until the reaction was complete. The volatile solvents were removed under reduced pressure, and the residual material was re-dissolved into MeOH (10 mL). This clear solution was poured slowly into ethyl ether (200 mL) with stirring. A mass of white precipitate formed rapidly. 7.18 g of white solid Compound (41) was collected by filtration under vacuum.

### Example 42

### Synthesis of Compound (42)

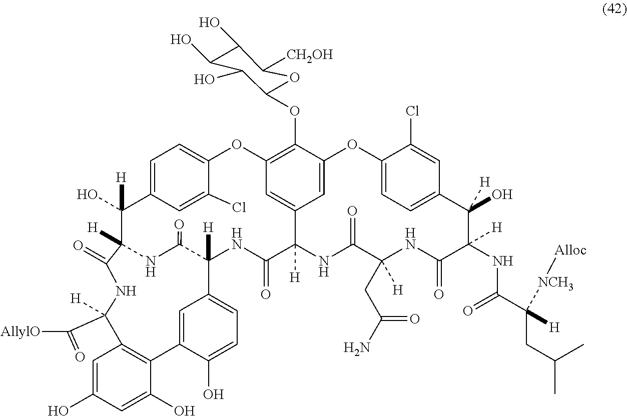

To a solution of Compound (41) (7.18 g, 5.16 mmol) in DMF (50 mL) was added NaHCO₃ (5.20 g, 61.9 mmol, 10 eq.) at room temperature. To the stirring suspension was added dropwise allyl bromide (6.25 g, 51.6 mmol, 12 eq.) at room temperature (approx. 10 min). The reaction mixture was stirring at room temperature and followed by HPLC analysis until completion (approx. 24 hours). The un-dissolved inorganic solid was removed by filtration. The clear filtrate was poured slowly into ethyl ether (200 mL) to yield a syrup-like residue. The upper solvents were removed by decantation. The residual syrup was dissolved into MeOH (20 mL) and was poured into ethyl ether again. The formed solid was collected by filtration under vacuum. This operation was repeated twice again. Finally, 6.79 g of Compound (42) was obtained as a white solid.

### Example 43

### Synthesis of Compound (43)

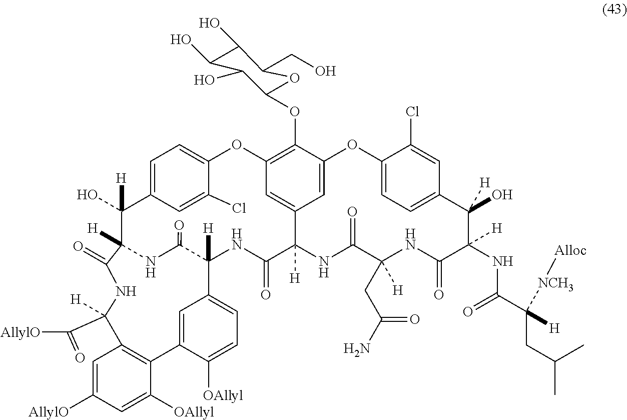

To a solution of Compound (42) (1.43 g, 1.0 mmol) in DMF (5 mL) was added Cs₂CO₃ (1.14 g, 3.5 mmol) with stirring rapidly at room temperature. To the stirring suspension was added dropwise allyl bromide (375 mg, 3.1 mmol) at room temperature within 30 min. After stirred at room temperature overnight, the undissolved solid was removed by filtration. The clear filtration was poured slowly into ethyl ether to form a mass of white solid. After standing for 30 min, the upper clear solvent was removes by decantation. The residual solid was re-dissolved into MeOH (20 mL) and was poured into ethyl ether again. The formed solid was collected by filtration under vacuum. This operation was repeated once again. 1.09 g of crude Compound (43) was collected by filtration as a white solid. Further purification conducted by preparative HPLC gave the pure Compound (43). Separation column: ALL TIMA C18, 22 mm I.D.×250 mm, 5 μm; Mobile phase: CH₃CN/H₂O=50/50; Pump flow rate: 10 ml/min.

### Example 44

### Synthesis of Compound (44)

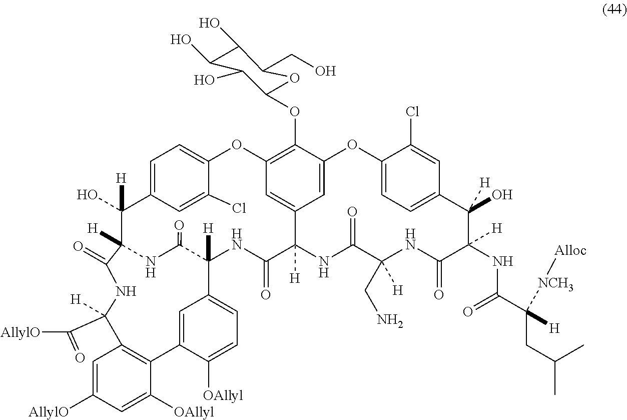

To a solution of Compound (43) (3 g, 1.93 mmol) dissolved into deionized H₂O (20 mL) and CH₃CN (20 mL) was added phenyliodine-bis-trifluoroacetate (1.78 g, 2.5 eq.) at 0° C. The reaction was warmed to room temperature naturally and stirred overnight. All solvents were removed under vacuum. The residual solid was washed with ether (3×30 mL). The further purification was conducted by a silica gel flash column chromatography (silica gel: 300-400 mesh; eluent: CH₂Cl₂/MeOH=80/20→40/60). Compound (44) (1.2 g) was obtained as a yellowish solid.

### Example 45

### Synthesis of Compound (45)

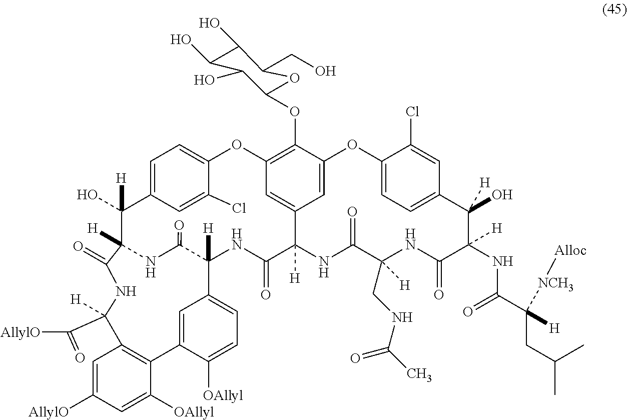

To a mixture of Compound (44) (152 mg, 0.10 mmol) and pyridine (24 mg, 0.30 mmol) in dry DMF (0.5 ml) at room temperature under N₂, was added slowly a solution of acetyl chloride (8 mg, 0.10 mmol) in dry DMF (0.5 ml). After stirring at room temperature for 1 hour, HPLC showed a new product formed with retention time of approximately 14 min. The reaction mixture was precipitated in ether and the forming solid was washed with ether and collected to yield Compound (45) (110 mg, 71%) as a white solid.

### Example 46

### Synthesis of Compound (46)

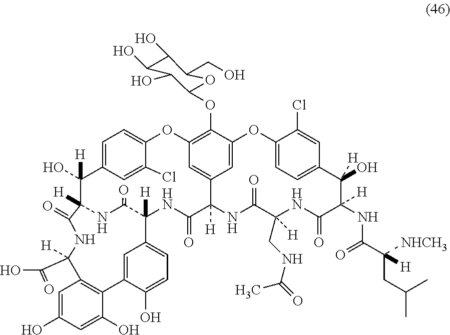

To a mixture of Compound (45) (110 mg), Pd(OAc)₂ (22 mg, 0.10 mmol) and PPh₃ (105 mg, 0.40 mmol) in DMF/AcOH (1 ml/1 ml) at room temperature, was added Bu₃SnH (2.91 g, 10.0 mmol) in one shot. The reaction mixture was stirred at room temperature for 10 min. Ether was added and the forming solid was collected and washed with ether a few times until a white color was achieved. The collected white solid was dried and purified by preparative HPLC to yield Compound (46) as a TFA salt (7 mg, 7%). Preparative HPLC conditions: Eluent: 50/50 of MeCN/H₂O (with 0.1% TFA); Flow rate: 10 ml/min; Column size: 250*22 mm; Retention time: around 14.5 min.

### Example 47

### Synthesis of Compound (47)

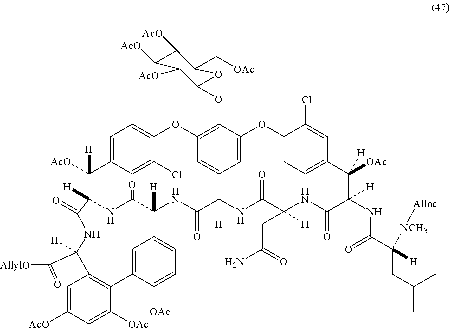

To a solution of Compound (42) in CH₂Cl₂ (5 g, 3.5 mmol) was added dropwise pyridine (20 mL) and acetic anhydride (25 mL) successively at room temperature with stirring rapidly. A catalytically amount of DMAP (500 mg) was added. The reaction was stirred at room temperature for 3 days. The reaction mixture was washed with brine (3×30 mL), dried on MgSO₄, and condensed under reduced pressure to yield 5.0 g of crude Compound (47) as a yellowish solid. Further purification was conducted by a silica gel flash column chromatography (silica gel: 300-400 mesh; eluent: CH₂Cl₂/MeOH=100/0→30/70) to yield 3.0 g of Compound (47) as an off-white solid.

### Example 48

### Synthesis of Compound (48)

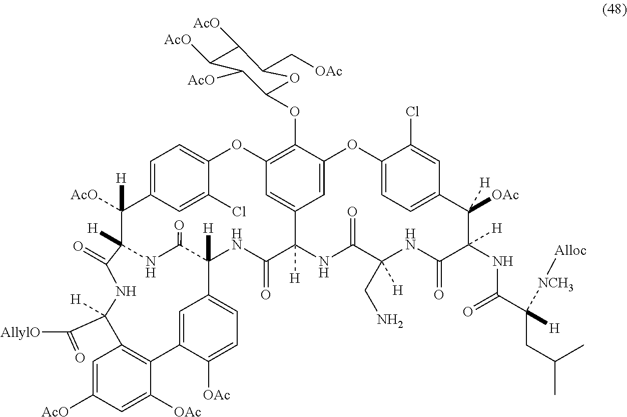

To a solution of Compound (47) (3 g, 1.66 mmol) dissolved into deionized H₂O (25 mL) and CH₃CN (25 mL) was added phenyliodine-bis-trifluoroacetate (1.78 g, 2.5 eq.) at 0° C. The reaction was warmed to room temperature naturally and stirred overnight. The volatile solvents were removed under vacuum. The residue was re-dissolved into CH₂Cl₂ (50 mL). The organic phase was washed with brine (3×30 mL), dried over anhydrous Na₂SO₄ and condensed to give the crude Compound (48). Further purification conducted by a silica gel flash chromatography (silica gel: 300-400 mesh; eluent: CH₂Cl₂/MeOH=100/0→40/60) to afford 2.6 g of Compound (48) as an off-white solid.

### Example 49

### Synthesis of Compound (49)

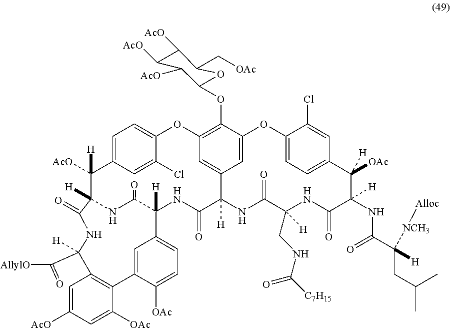

To a stirring solution of Compound (48) (900 mg, 0.51 mmol) in DMF (10 mL) was added octanoic acid (73 mg, 1 eq.), HATU (385 mg, 1 eq.), and DIPEA (1 mL) successively. After stirring for 50 min, the reaction mixture was poured into CH₂Cl₂ (50 mL). The newly formed solution was washed with brine (3×30 mL), dried over anhydrous MgSO₄ and condensed under reduced pressure to yield the crude Compound (49). Purification was conducted by preparative HPLC. Separation column: ALL TIMA C18, 22 mm I.D.×250 mm, 5 μm; Mobile phase: CH₃CN/H₂O=88/12; Pump flow rate: 10 mL/min. The crude was also purified by normal silica gel flash column chromatography (silica gel: 300-400 mesh; eluent: Hexanes/EtOAc=50/50/to 0/100).

### Example 50

### Synthesis of Compound (50)

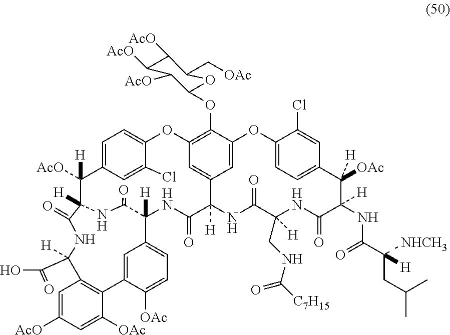

Using a similar procedure as the preparation of Compound (46), and replacing Compound (45) with Compound (49), Compound (50) was prepared.

### Example 51

### Synthesis of Compound (51)

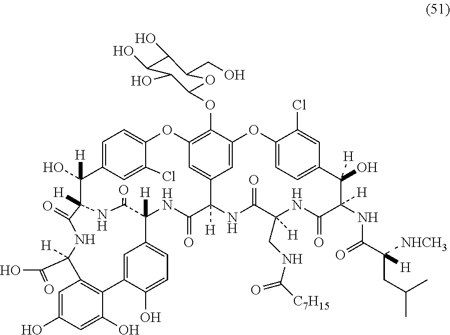

To 0.10 mmol of Compound (50) in 5 mL of methanol/water mixture (3:1) in a round bottom flask is added 1.1 mmol of potassium carbonate. The mixture is stirred at room temperature for 20 hours yielding Compound (51).

### Example 52

### Synthesis of Compound (52)

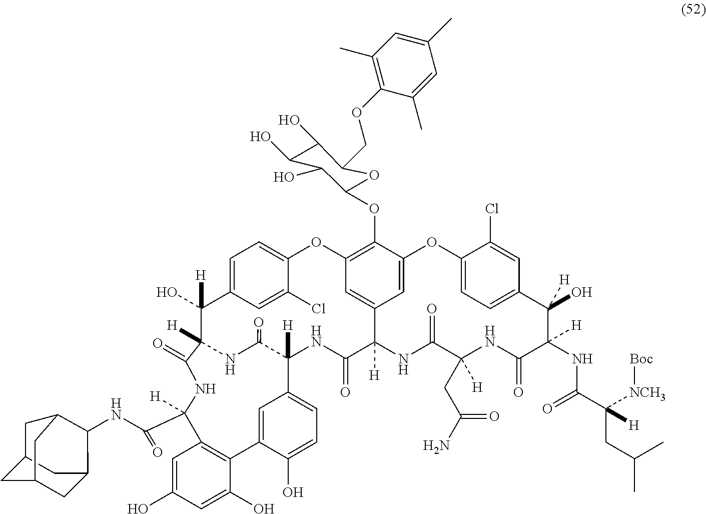

Compound (11) (1 g, 0.649 mmol) was azeotroped with toluene 3 times and then dissolved in anhydrous pyridine. Mesitylenesulfonyl chloride (426 mg, 1.95 mmol) in 1 ml of anhydrous pyridine was added to the solution dropwise at 0° C., and the mixture was kept stirring for 2 hour. The reaction mixture was poured into water and filtered. The solid was purified by flashing normal phase column (MeOH/DCM=1/10˜1/5) to give Compound (52) as a white solid (500 mg, yield=50%). LC-MS (ESI): 1620 (M⁺+1-Boc),

### Example 53

### Synthesis of Compound (53)

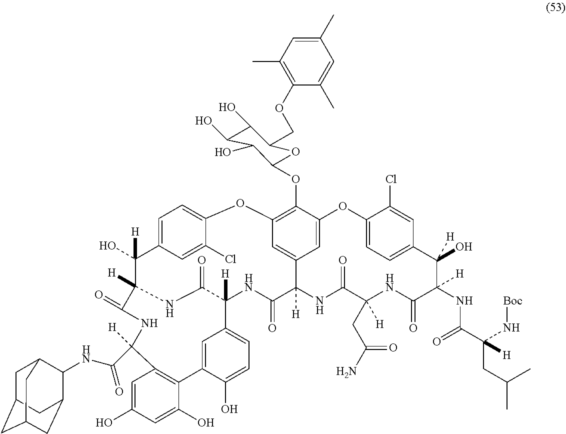

Using a procedure similar to the preparation of Compound (52), and replacing Compound an with Compound (11) Compound (53) is prepared.

### Example 54

### Synthesis of Compound (54)

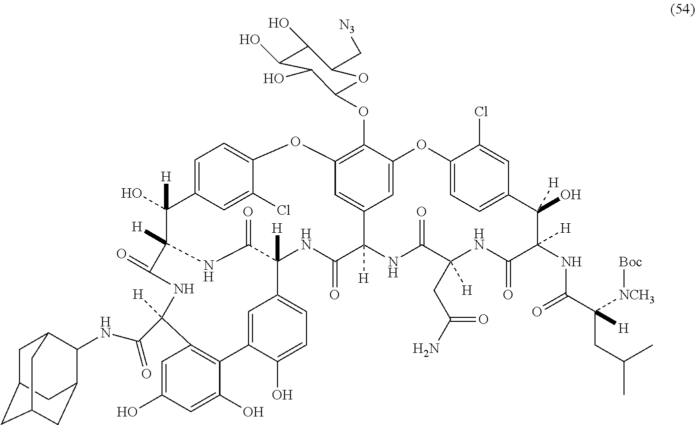

A solution of Compound (52) (1 g, 0.581 mmol) and sodium azide (377 mg, 5.81 mmol, 10 eq.) in anhydrous DMF was heated to 70° C. overnight. The reaction mixture was cooled and added to water. The solid was filtered, washed with water, and purified by flashing normal phase column (MeOH/DCM=1/12˜1/9) to give Compound (54) as a pale yellow solid (500 mg, yield=50%). LC-MS (ESI): 1463 (M⁺+1-Boc).

### Example 55

### Synthesis of Compound (55)

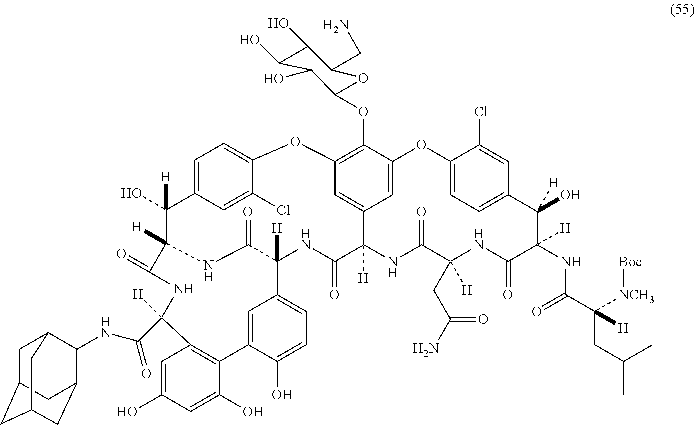

To a solution of Compound (54) (1 g, 0.639 mmol) in 5 ml THF containing a few drops of water was added n-Bu₃P (905 mg, 4.47 mmol). The mixture was heated to reflux overnight, then cooled to room temperature, and poured into water. The solid was filtered, washed with water, and purified by flashing reverse phase column (MeCN/H₂O=1/9˜4/3) to afford Compound (55) as a pale yellow solid (100 mg, yield=10%). LC-MS (ESI): 1537 (M⁺+1).

### Example 56

### Synthesis of Compound (56)

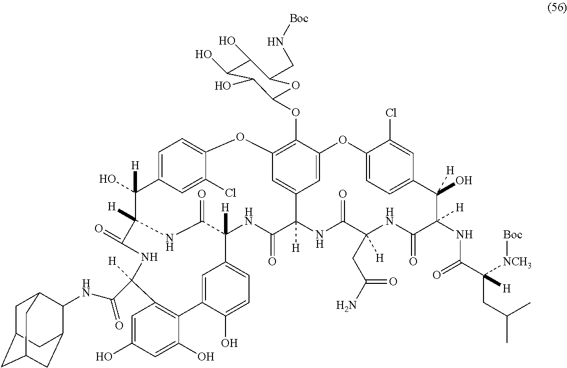

To a solution of Compound (55) (380 mg) in 2 ml of THF containing 10 drops of water was added di-tert butyl dicarbonate (1.05 eq) and TEA (2.0 eq). The mixture was stirred at room temperature for 5 hours. The reaction was checked for completion by HPLC-MS. The solvent was evaporated to afford Compound (56) upon purification by prep-HPLC.

### Example 57

### Synthesis of Compound (57)

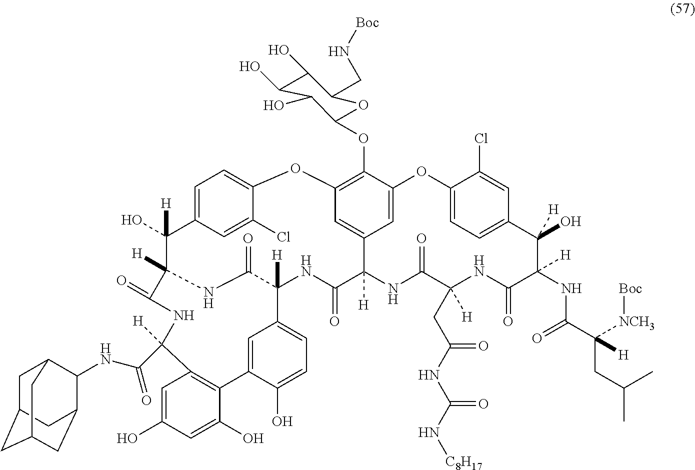

Using Compound (56) (100 mg) was azeotroped with toluene for three times. It was the dissolved in 1 ml dry DMF. DBU (3.0 equivalent) in 1 ml dry DMF was added under argon atmosphere in an ice bath followed by the addition of isocyanate C₈H₁₇NCO (2.0 equivalent) in 1 ml DMF. The mixture was stirred at room temperature overnight. The reaction was checked for completion by HPLC-MS. The reaction was quenched by adding water, and then filled. The cake was washed three times with water. The crude compound was purified by preparative HPLC to afford Compound (57).

### Example 58

### Synthesis of Compound (58)

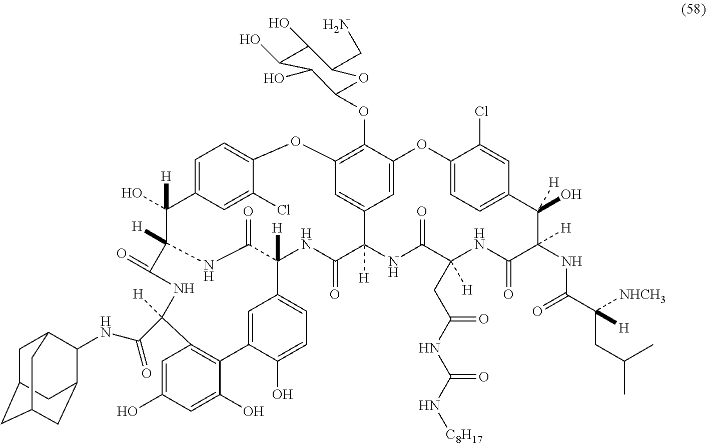

Compound (57) in 2 ml of TEA/DCM (1/1) was stirred for 1 hour in an ice-bath. The reaction was checked for completion by HPLC-MS. The solvent was removed under reduced pressure at 0° C. The residue was washed with ether and filtered to give Compound (58) as a TFA salt.

### Example 59

### Synthesis of Compound (59)

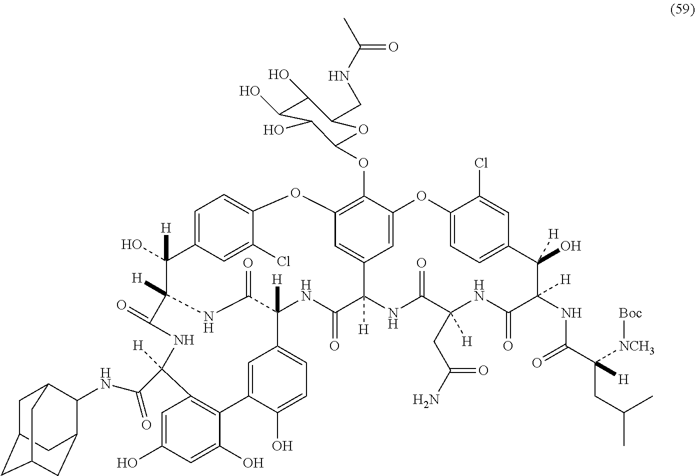

Using a procedure similar to the preparation of Compound (45), and replacing Compound (44) with Compound (55), Compound (59) is made.

### Example 60

### Synthesis of Compound (60)

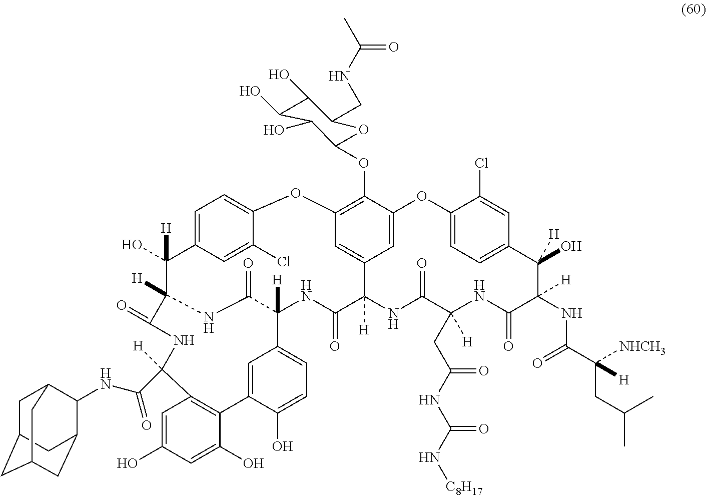

Using a procedure similar to the preparation of Compound (29), and replacing Compound (23) with Compound (59), Compound (60) is prepared.

### Example 61

### Synthesis of Various Carboxamide Glycopeptides Derivatives (61-66)

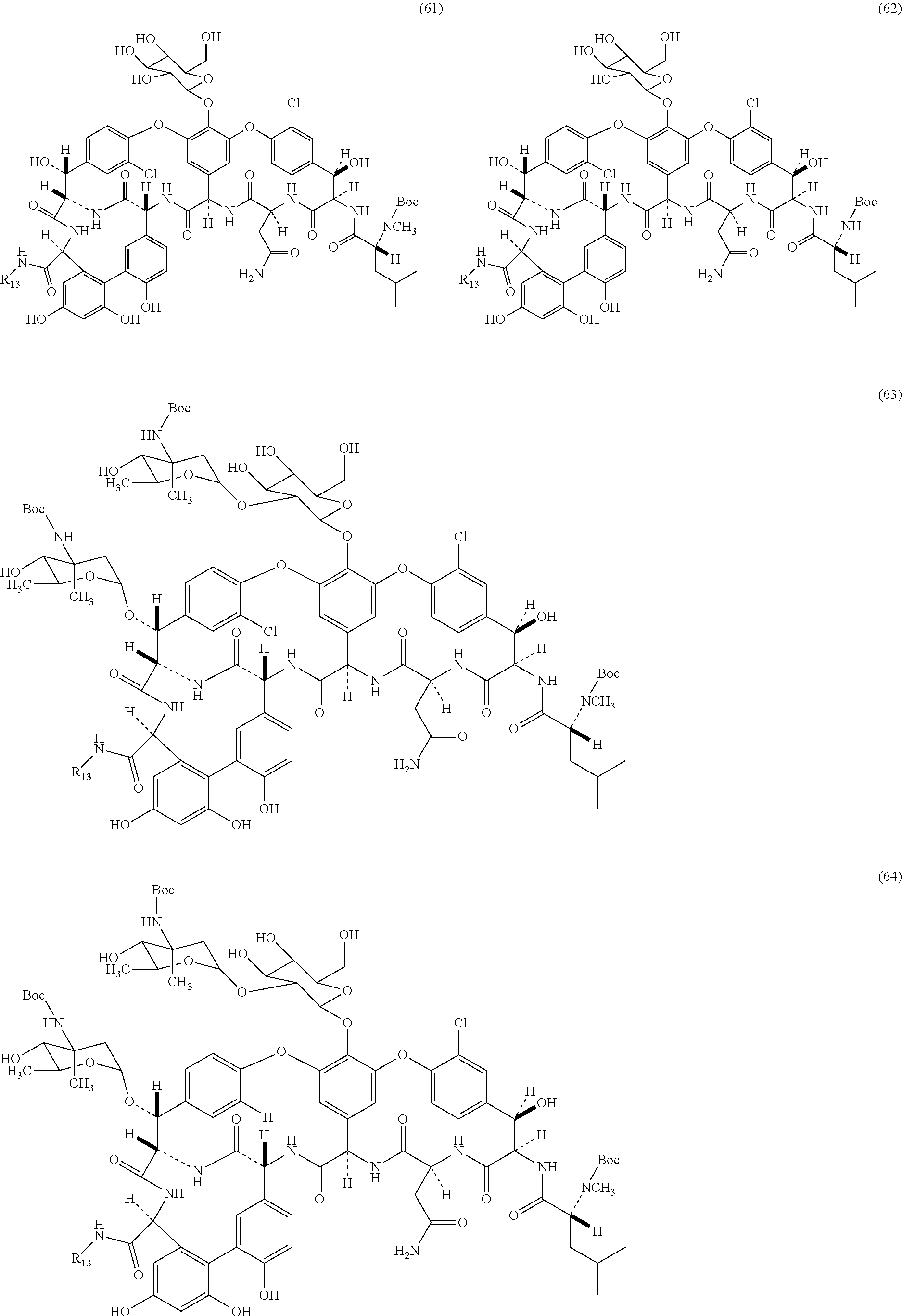

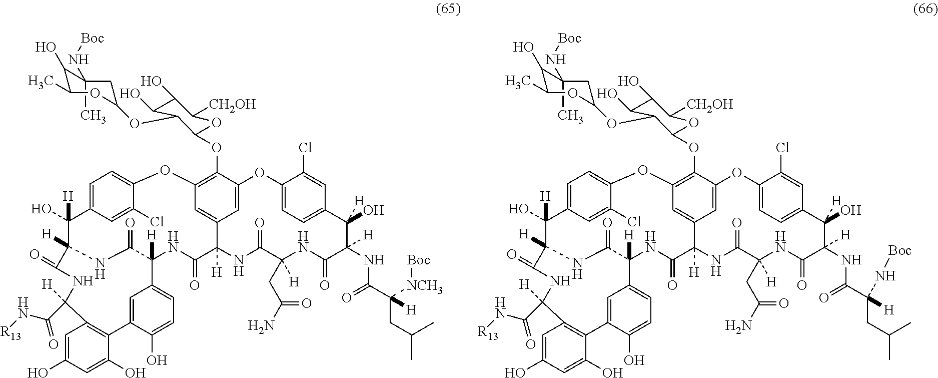

Using a similar to the preparation of Compound (11-16), and replacing 2-adamantylamine hydrochloride with R₁₃—NH₂ hydrochloride and reacting it with Compound (5-10), Compound (61-66) wherein R₁₃ is as defined, is prepared.

### Example 61

### Synthesis of Various Carboxamide Glycopeptides Derivatives (67-72)

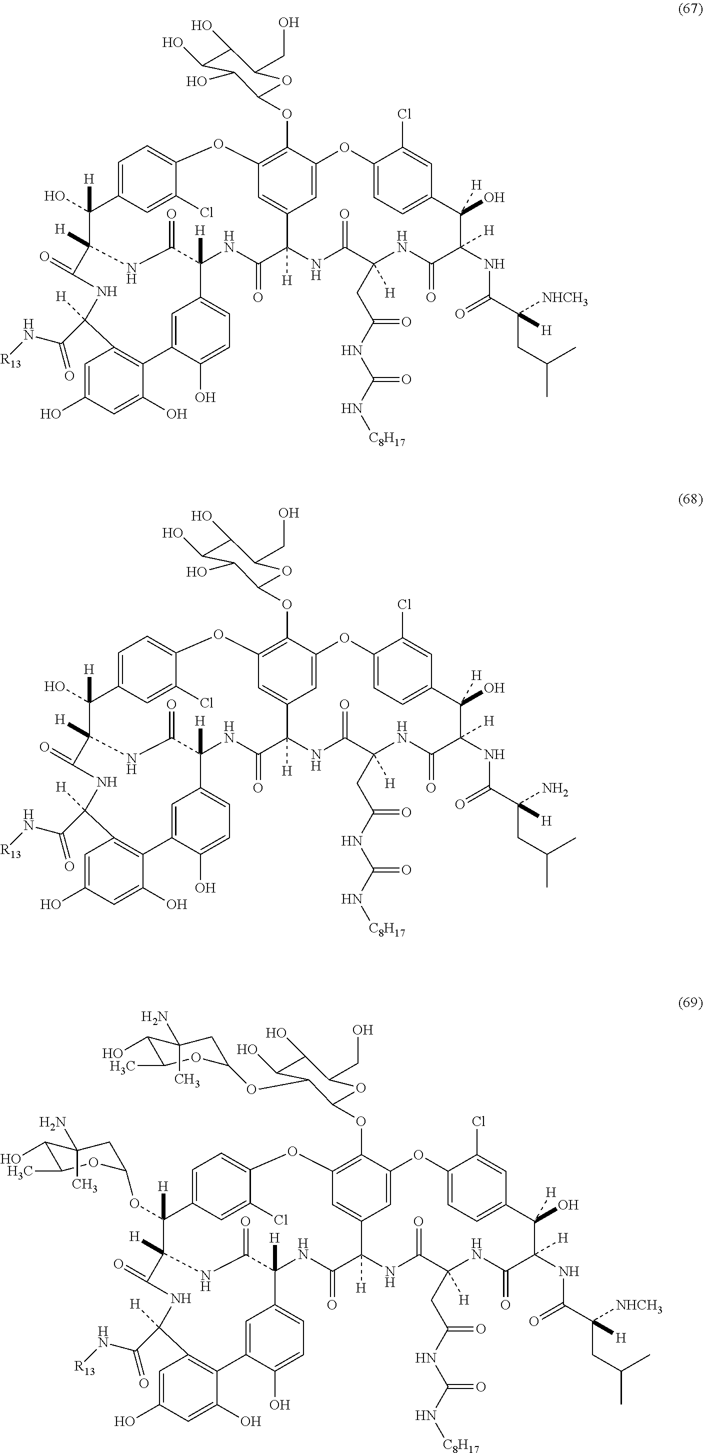

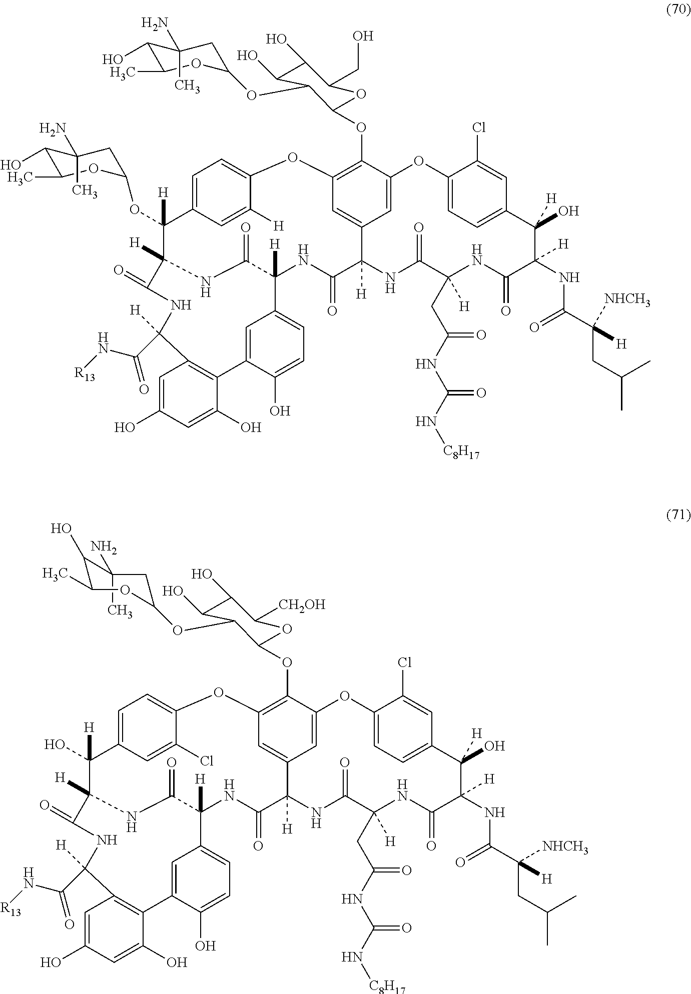

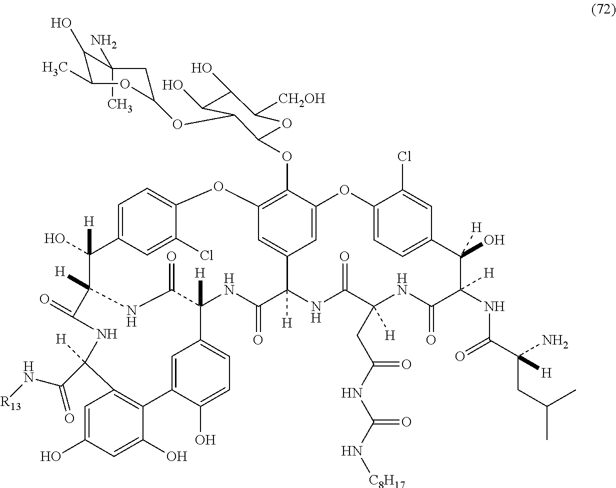

Following the synthetic methodology as Example 23 followed with the removal of the protecting group with a procedure similar to Example 30, Compound (67-72), wherein R₁₃ is as defined, is prepared from Compound (61-66).

### Example 62

### Synthesis of Compound (73&74)

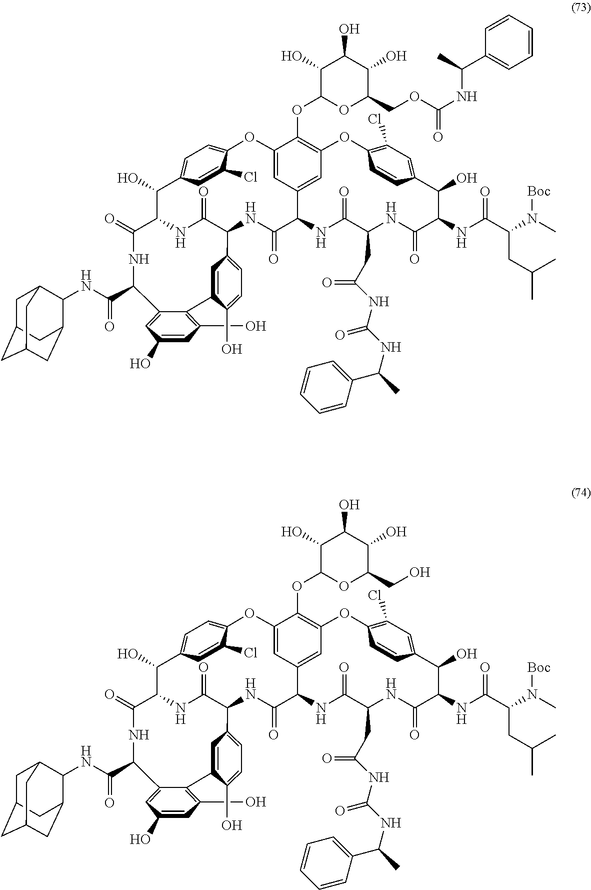

Using a procedure similar to the preparation of Compound (23), and replacing C₈H₁₇NCO with (1-isocyanatoethyl)benzene, Compound (73) and also Compound (74) were made.

### Example 63

### Synthesis of Compound (75)

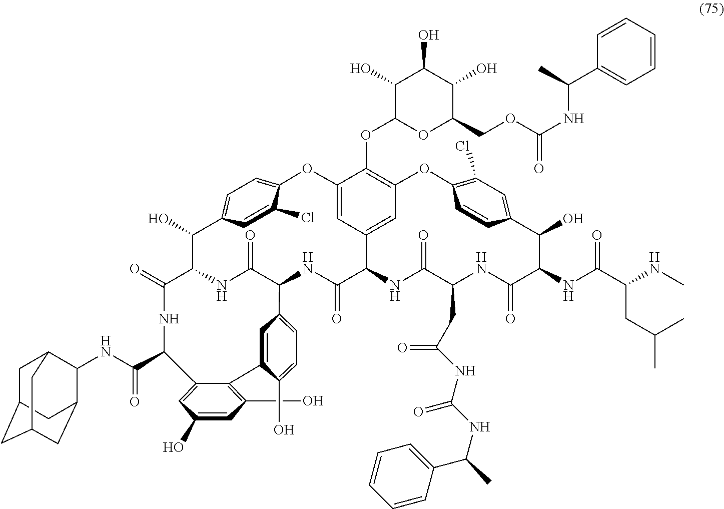

Using a procedure similar to the preparation of Compound (29), and replacing Compound (23) with Compound (73), Compound (75) was prepared as a TFA salt.

### Example 64

### Synthesis of Compound (76)

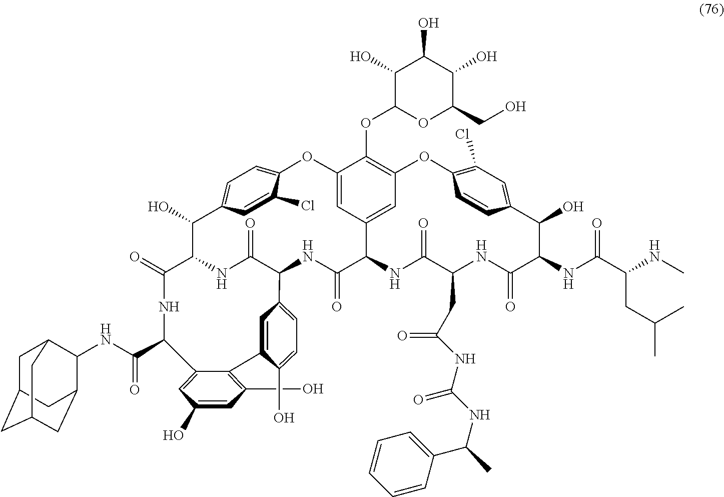

Using a procedure similar to the preparation of Compound (29), and replacing Compound (23) with Compound (74), Compound (76) was prepared as a TFA salt.

### Example 65

### Synthesis of Compound (77)

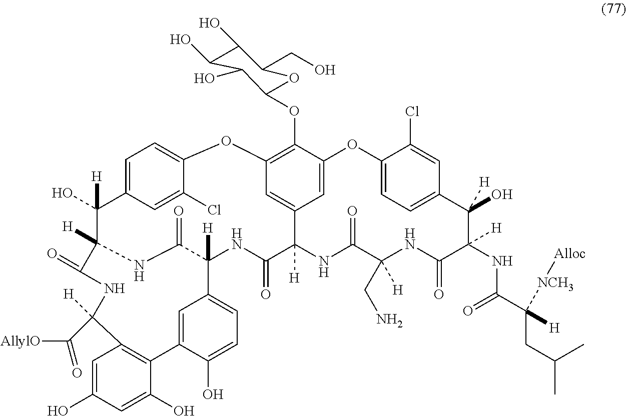

To 0.10 mmol of Compound (48) in 10 mL of allyl alcohol in a round bottom flask is added 1.1 mmol of potassium carbonate. The mixture is stirred at room temperature for 20 hours yielding Compound (77).

### Example 66

### Synthesis of Compound (78)

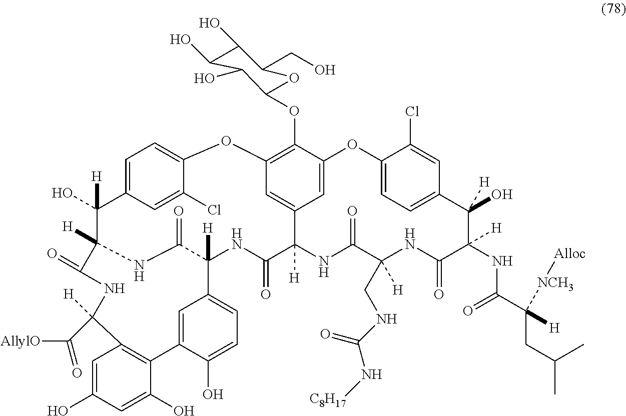

Using a procedure similar to the preparation of Compound (23) and replacing Compound (11) with Compound (77), Compound (78) is made.

### Example 67

### Synthesis of Compound (79)

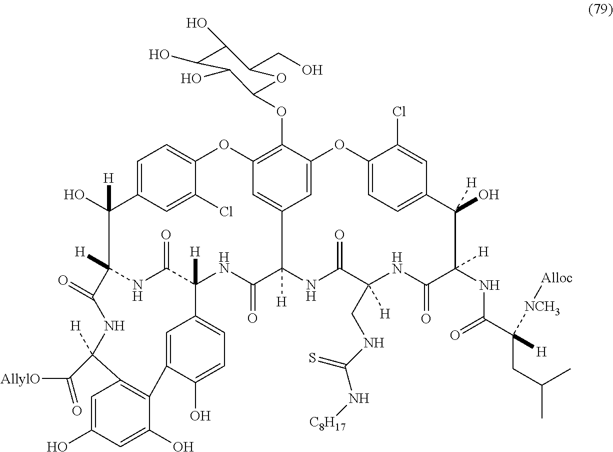

Using a procedure similar to the preparation of Compound (23) and replacing Compound (11) with Compound (77) and C₈H₁₇NCO with C₈H₁₇NCS, Compound (79) is made.

### Example 68

### Synthesis of Compound (80)

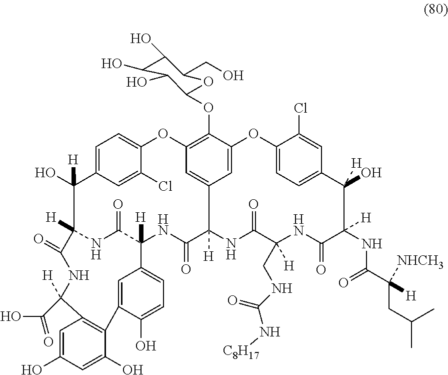

Using a procedure similar to the preparation of Compound (46) and replacing Compound (45) with Compound (78), Compound (80) is prepared.

### Example 69

### Synthesis of Compound (81)

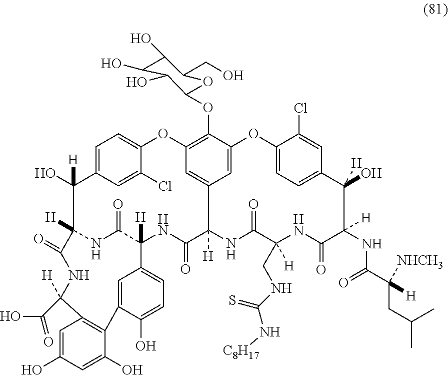

Using a procedure similar to the preparation of Compound (46) and replacing Compound (45) with Compound (79), Compound (81) is made.

### Example 70

### Synthesis of Compound (82)

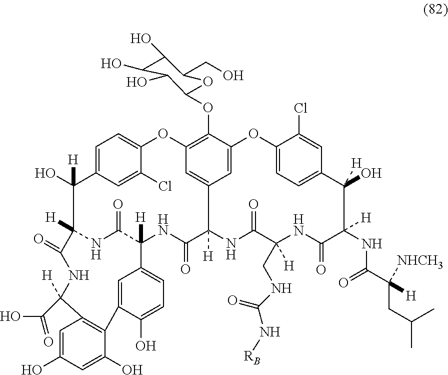

Using a procedure similar to the preparation of Compound (23) and replacing Compound (11) with Compound (77) and C₈H₁₇NCO with R_{B}NCO, and subjecting the resulted product with deprotection methodology as in Example 46, Compound (82) is prepared.

### Example 71

### Synthesis of Compound (83 &84)

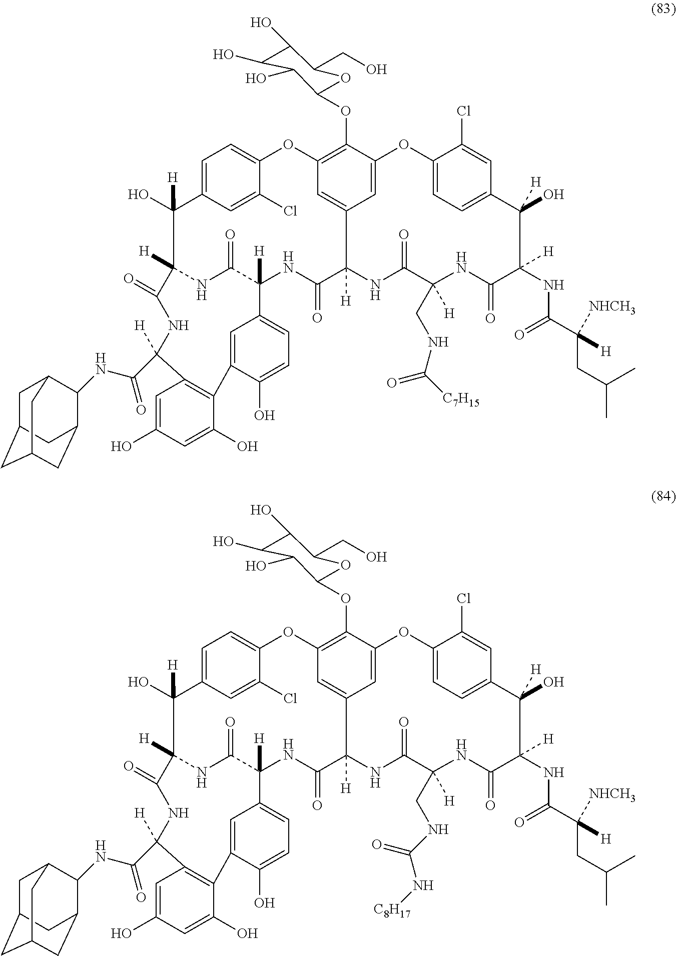

Using a procedure similar to the preparation of Compound (11) as in Example 11, replacing Compound (5) with Compound (51 or 80), Compound (83) and Compound (84) is prepared.

### Example 72

### Synthesis of Compound (85)

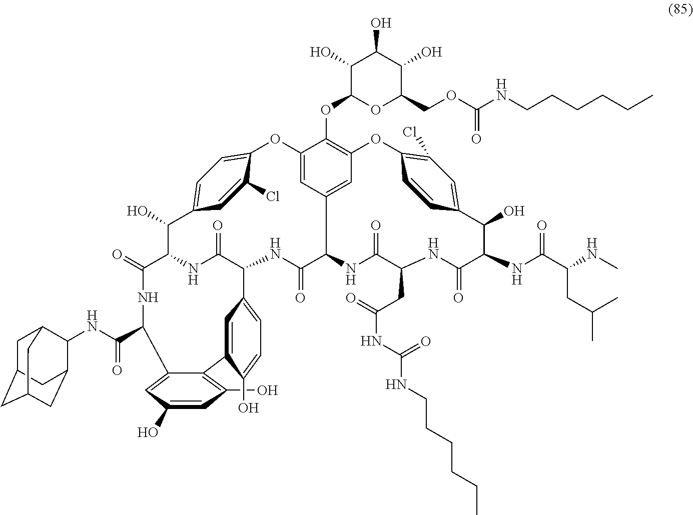

Using a procedure similar to the preparation of Compound (23) (example 23), replacing C₈H₁₇NCO with reagent C₆H₁₃NCO, nitrogen protected Boc-85 was produced. Subsequent de-protection of Boc-85 by treatment with TFA with a procedure similar to the preparation of Compound (29) (example 29), Compound (85) was prepared as a TFA salt.

### Example 73

### Synthesis of Compound (86)

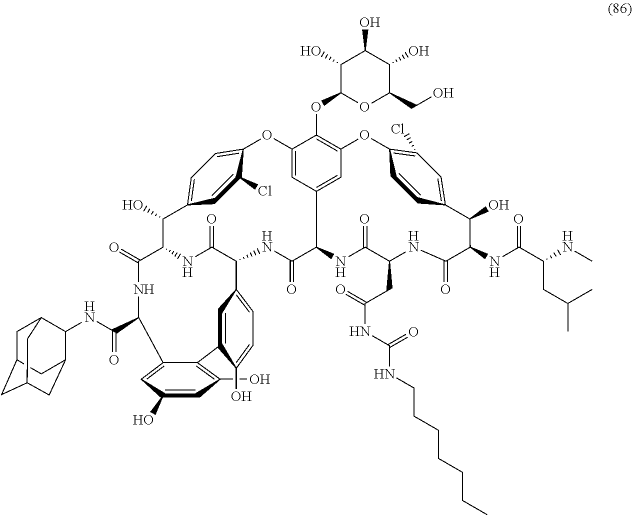

Using a procedure similar to the preparation of Compound (85) (example 72), and replacing reagent C₆H₁₃NCO with reagent C₇H₁₅NCO, Compound (86) was prepared as a TFA salt.

### Example 74

### Synthesis of Compounds (87), (88), (89), (90) and (91)

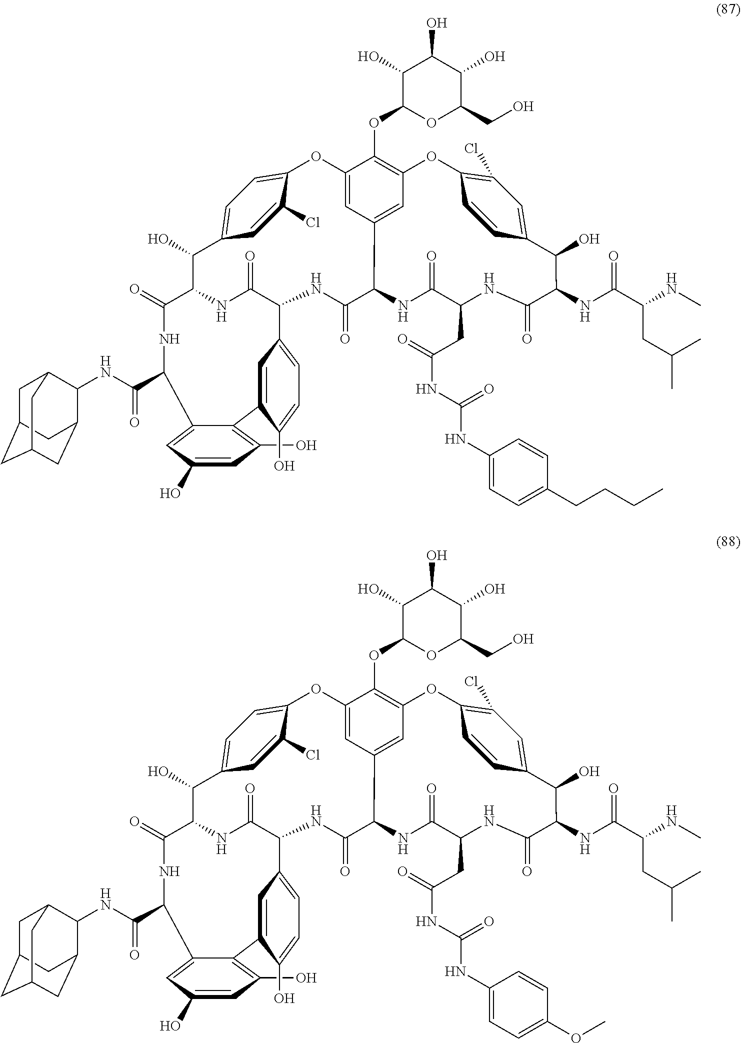

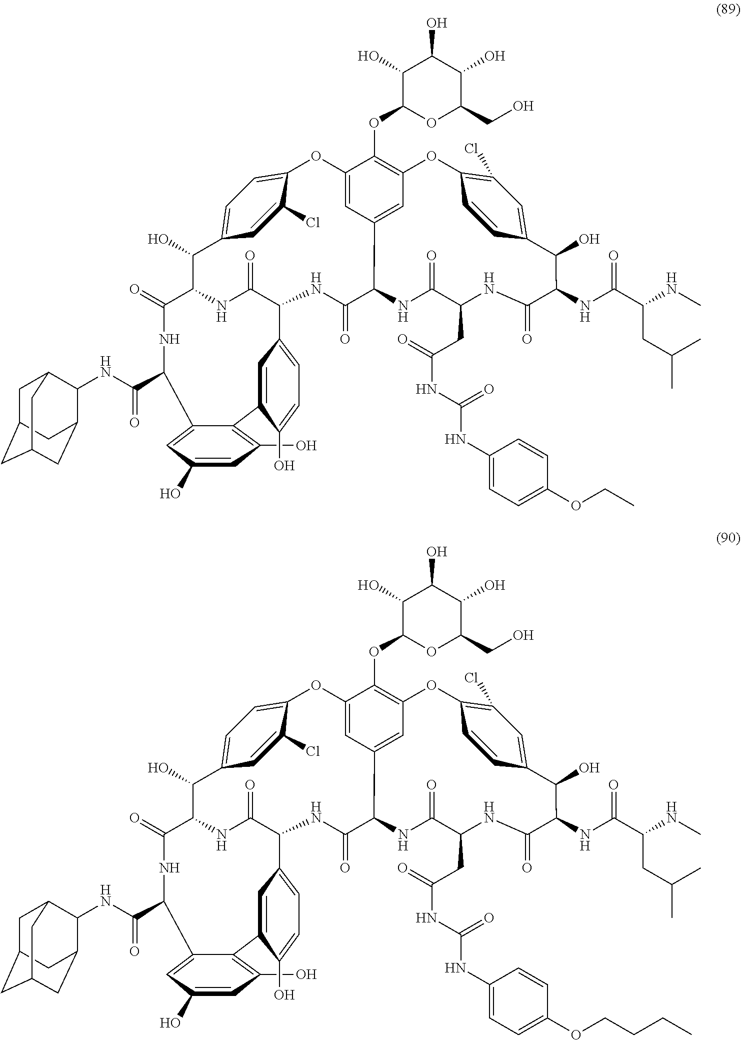

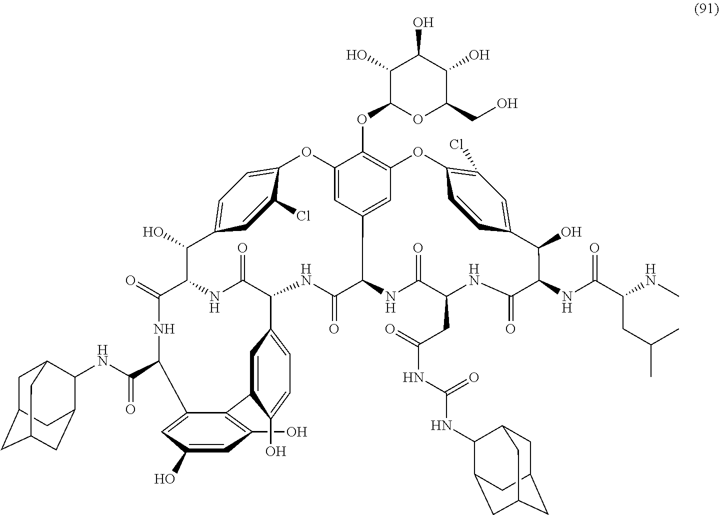

Using a procedure similar to the preparation of Compound (85) (example 72), and replacing reagent C₆H₁₃NCO with reagents 1-butyl-4-isocyanatobenzene, 1-methoxy-4-isocyanatobenzene, 1-ethoxy-4-isocyanatobenzene, 1-butoxy-4-isocyanatobenzene and 2-adamantyl isocyanate, Compounds (87), (88), (89), (90) and (91), respectively, were prepared as a TFA salt. LC-MS (M⁺+1): Compound (87): 1613.5; Compound (88): 1587.5; Compound (89): 1601.5; Compound (90): 1629.5; Compound (81): 1615.6.

### Example 75

### Synthesis of Compounds (92), (93), (94), (95), (96) and (97)

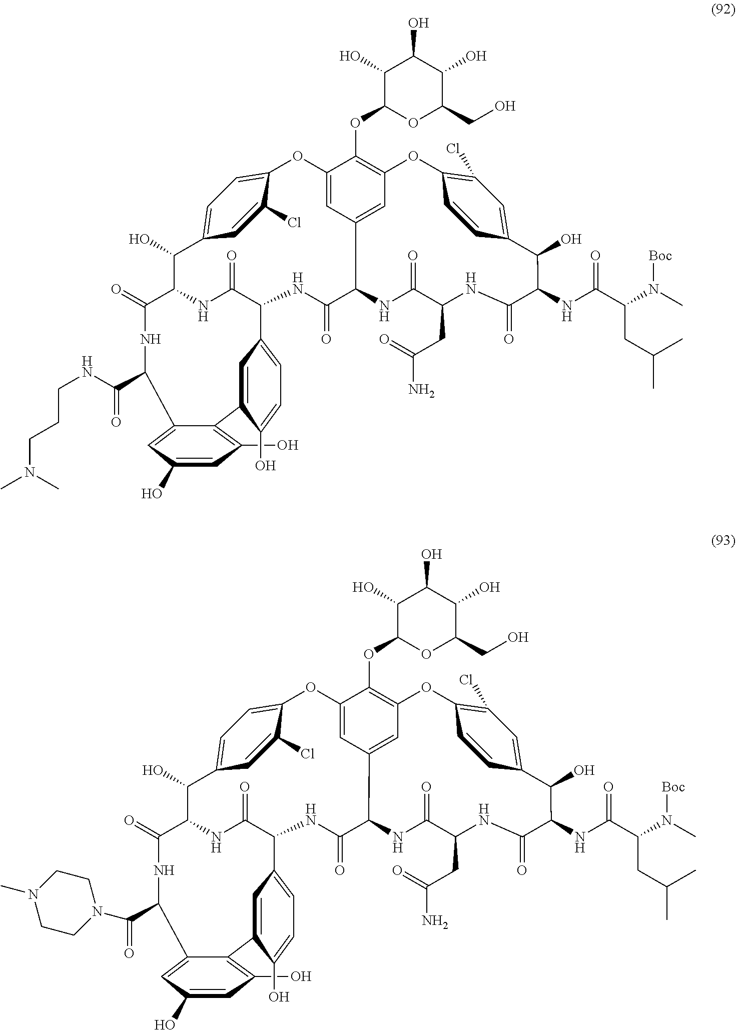

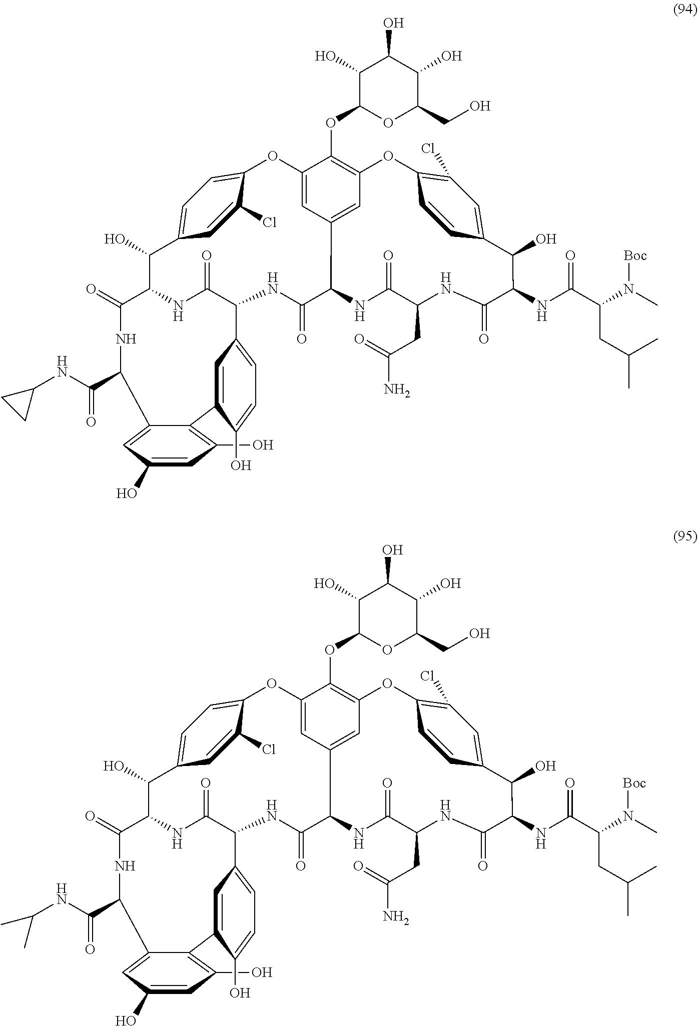

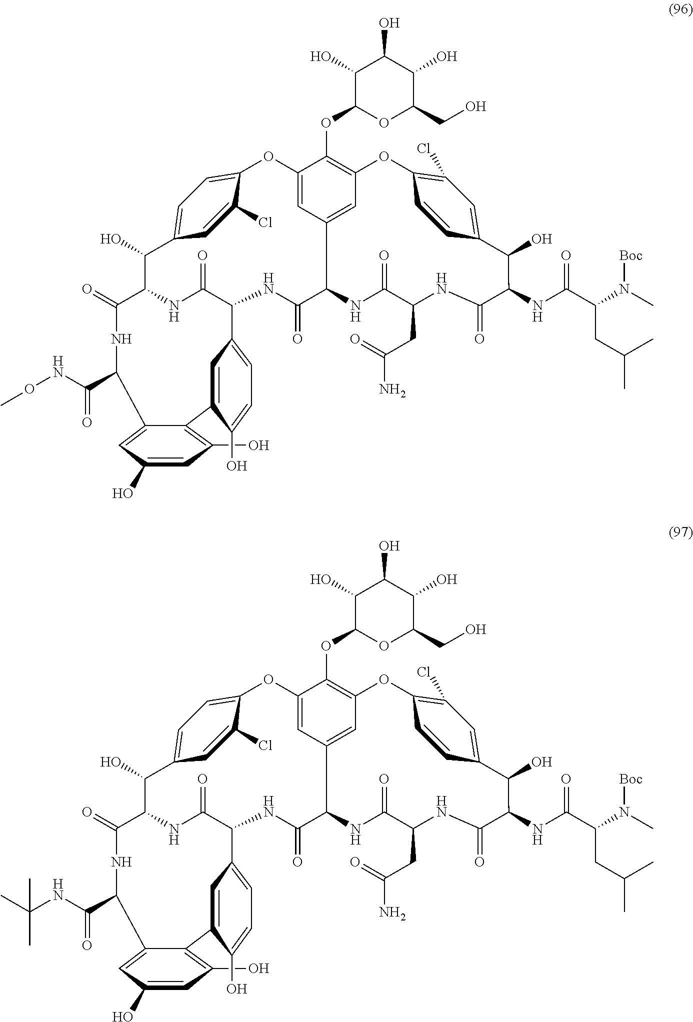

Using a procedure similar to the preparation of Compound (11) (example 11), and replacing reagent 2-adamantylamine with N¹, N¹-dimethylpropane-1,3-diamine, 1-methylpiperazine, cyclopropanamine, propan-2-amine, O-methylhydroxylamine and 2-methylpropan-2-amine, Compounds (92), (93), (94), (95), (96) and (97), respectively, were prepared.

### Example 76

### Synthesis of Compounds (98) and (99)

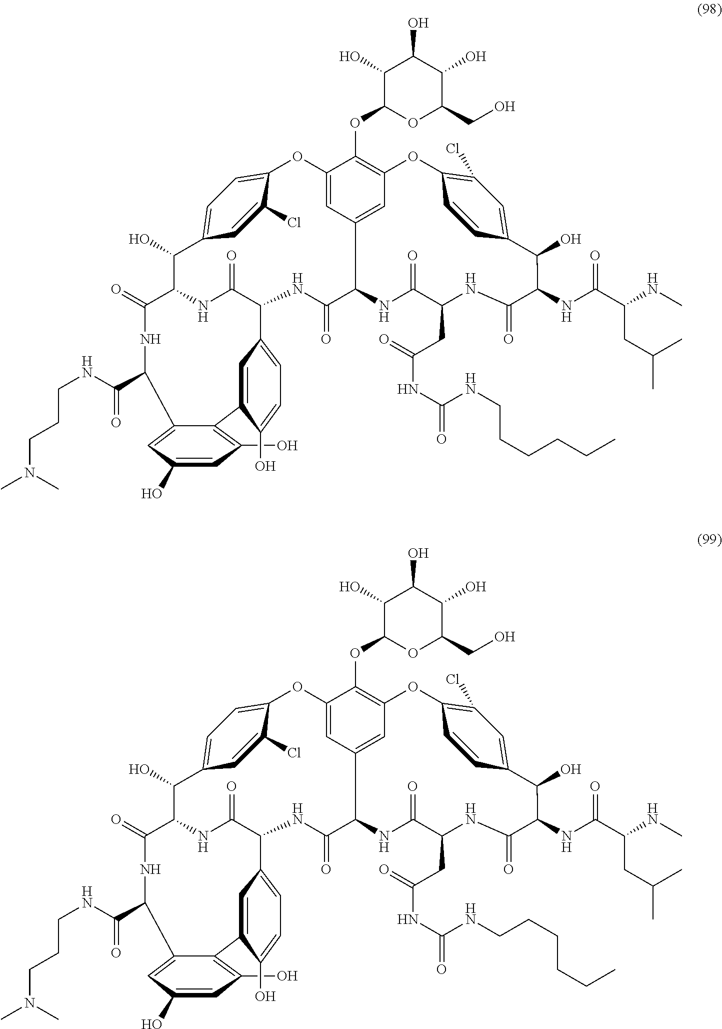

Using a procedure similar to the preparation of Compound (23) (example 23), replacing Compound (11) with Compound (92), and substituting the isocyanate C₈H₁₇NCO with various isocyanate, nitrogen protected acylureas were prepared. Subsequent de-protection of acylamides by treatment with TFA with a procedure similar to the preparation of Compound (29) (example 29), Compounds (98) and (99) were prepared as a TFA salt. LC-MS (M⁺+1): Compound (98): 1544.6; Compound (99): 1516.5.

### Example 77

### Synthesis of Compounds (100) and (101)

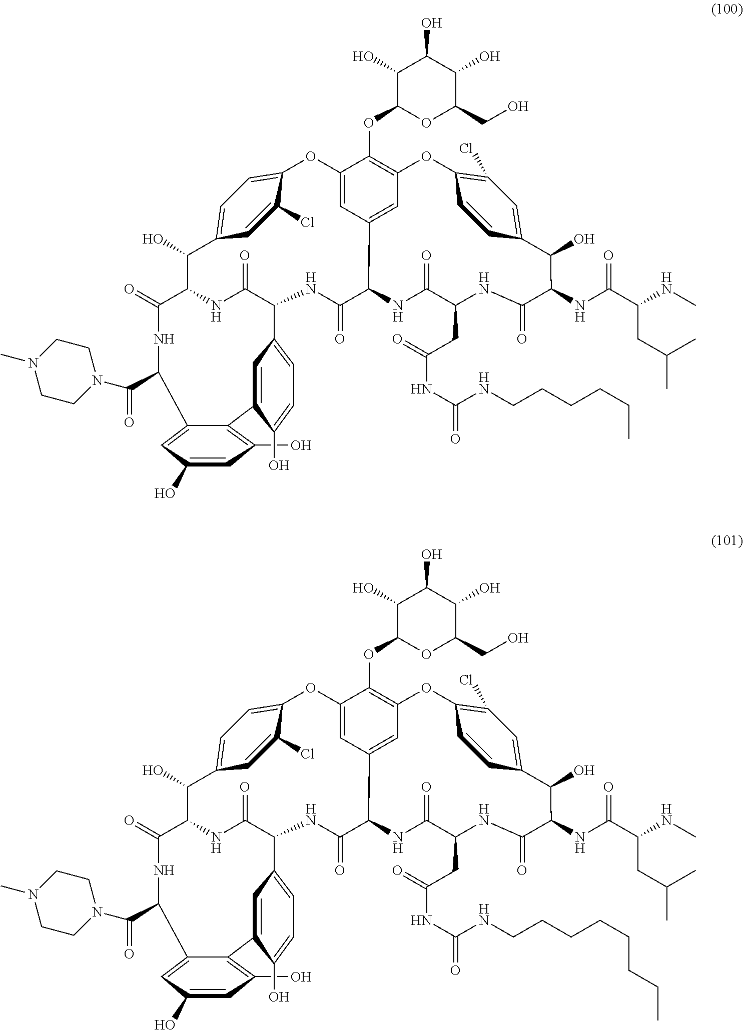

Using a procedure similar to the preparation of Compound (23) (example 23), replacing Compound (11) with Compound (93), and substituting the isocyanate C₈H₁₇NCO with various isocyanates, nitrogen protected acylureas were prepared. Subsequent de-protection of acylamides by treatment with TFA with a procedure similar to the preparation of Compound (29) (example 29), Compounds (100) and (101) were prepared as a TFA salt. LC-MS (M⁺+1): Compound (100): 1514.5; Compound (101): 1542.5.

### Example 78

### Synthesis of Compounds (102) and (103)

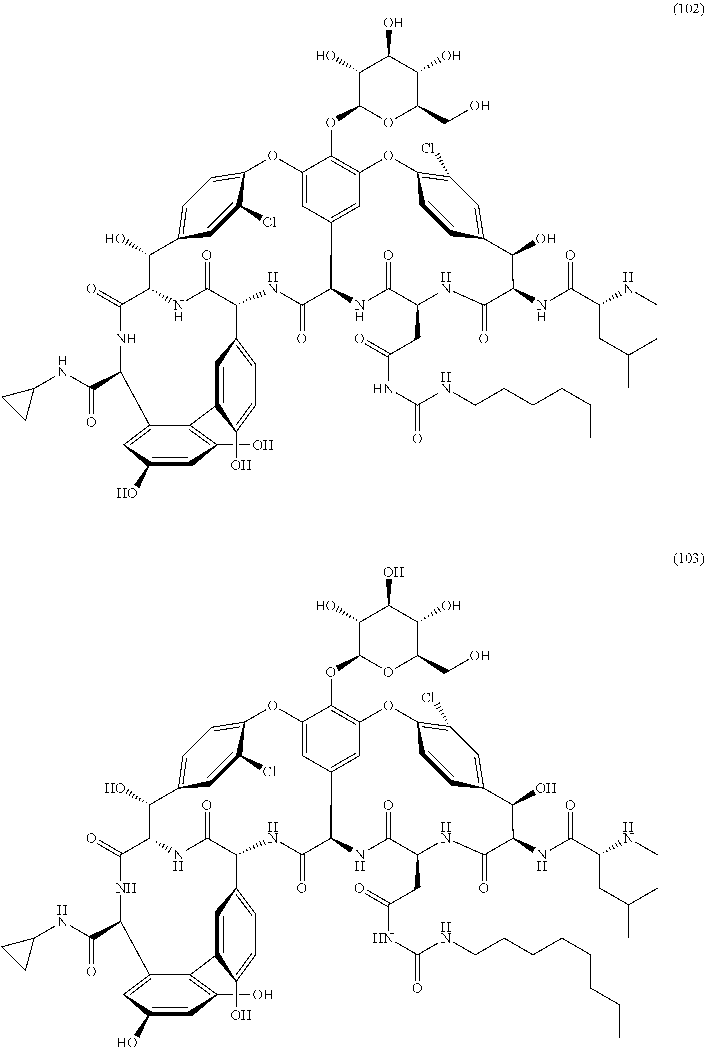

Using a procedure similar to the preparation of Compound (23) (example 23), replacing Compound (11) with Compound (94), and substituting the isocyanate C₈H₁₇NCO with various isocyanates, nitrogen protected acylureas were prepared. Subsequent de-protection of acylamides by treatment with TFA with a procedure similar to the preparation of Compound (29) (example 29), Compounds (102) and (103) were prepared as a TFA salt. LC-MS (M⁺+1): Compound (102): 1471.5; Compound (103): 1499.5.

### Example 79

### Synthesis of Compound (104)

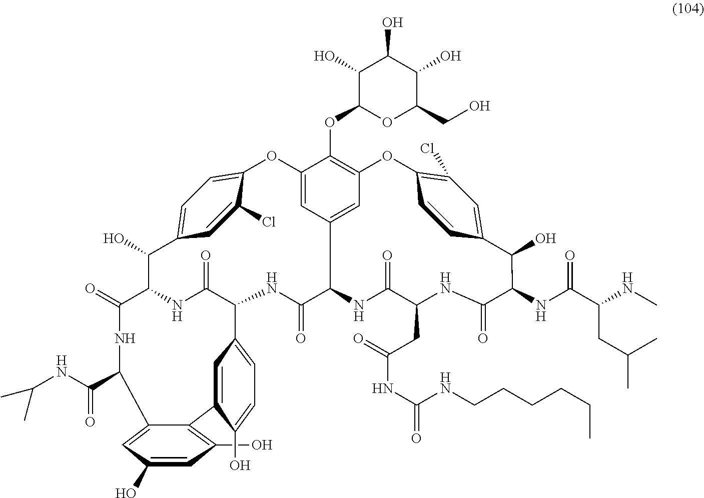

Using a procedure similar to the preparation of Compound (23) (example 23), replacing Compound (11) with Compound (95), and substituting the isocyanate C₈H₁₇NCO with C₆H₁₃NCO, nitrogen protected acylureas were prepared. Subsequent de-protection of acylamides by treatment with TFA with a procedure similar to the preparation of Compound (29) (example 29), Compound (104) was prepared as a TFA salt. LC-MS (M⁺+1): 1473.5.

### Example 80

### Synthesis of Compounds (105) and (106)

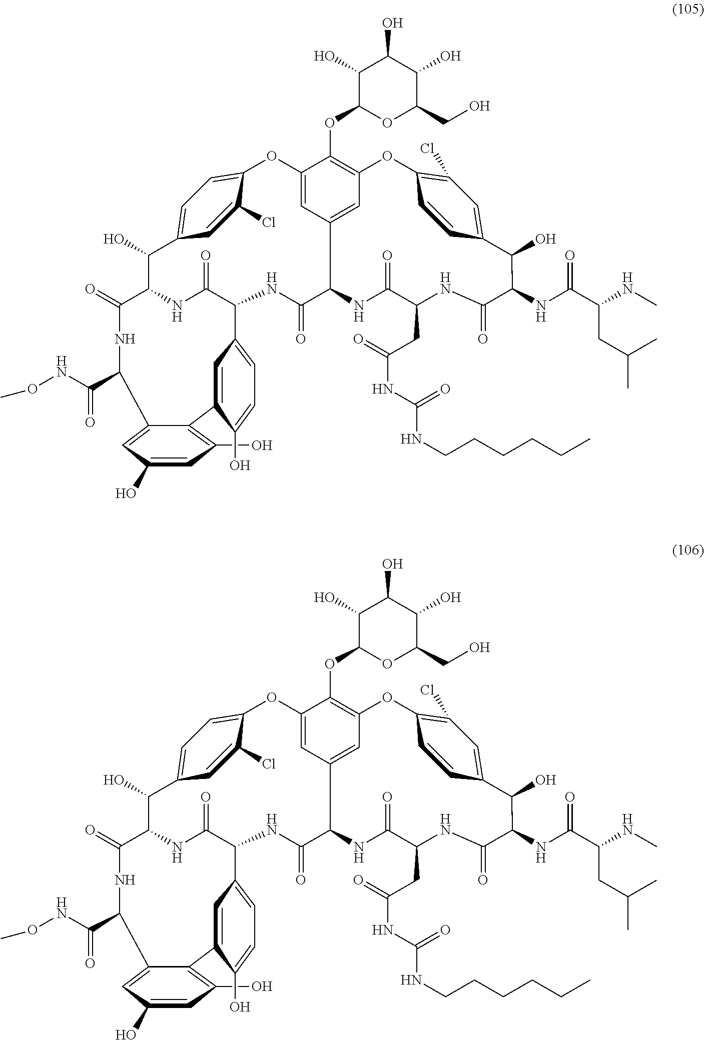

Using a procedure similar to the preparation of Compound (23) (example 23), replacing Compound (11) with Compound (96), and substituting the isocyanate C₈H₁₇NCO with various isocyanates, nitrogen protected acylureas were prepared. Subsequent de-protection of acylamides by treatment with TFA with a procedure similar to the preparation of Compound (29) (example 29), Compounds (105) and (106) were prepared as a TFA salt. LC-MS (M⁺+1): Compound (105): 1461.5; Compound (106): 1489.5.

### Example 81

### Synthesis of Compounds (107) and (108)

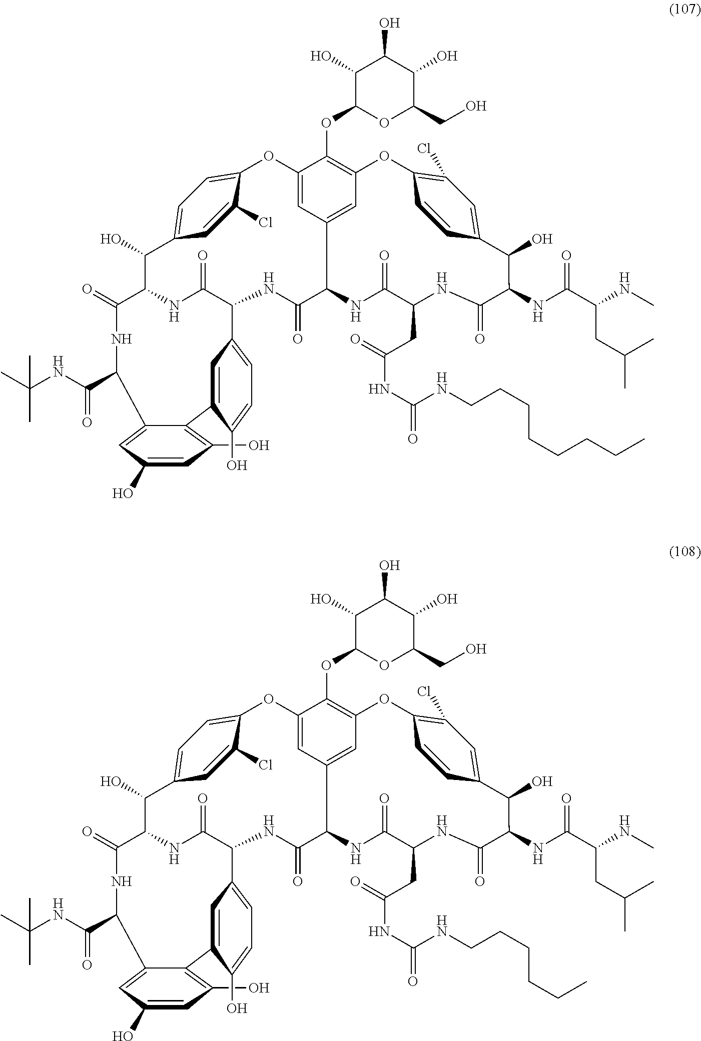

Using a procedure similar to the preparation of Compound (23) (example 23), replacing Compound (11) with Compound (97), and substituting the isocyanate C₈H₁₇NCO with various isocyanates, nitrogen protected acylureas were prepared. Subsequent de-protection of acylamides by treatment with TFA with a procedure similar to the preparation of Compound (29) (example 29), Compounds (107) and (108) were prepared as a TFA salt. LC-MS (M⁺+1): Compound (107): 1515.5; Compound (108): 1478.5.

### Example 82

### Synthesis of tert-butyl 2-(4-isocyanatophenoxy)ethyl(methyl)carbamate

To a mixture 2-(methylamino) ethanol (5.0 g, 66.5 mmol) in 15 ml of ethyl acetate was added a solution of (Boc)₂O (14.5 g, 66.5 mmol) in 5 ml of ethyl acetate dropwise with cooling in an ice bath. The resulting mixture was stirred at room temperature for 2 hours, and the solvent was removed by evaporation under reduced pressure. The residue was dissolved in ethyl acetate, washed with water, dried over Na₂SO₄ and filtered. After removing the solvent, the crude tert-butyl 2-hydroxyethyl(methyl)carbamate was used without further purification for the next reaction (10.5 g, 90%) A solution of diisopropyl azodicarboxylate (5.22 g, 25.9 mmol) in 5 ml of THF was added dropwise to a solution of 4-nitryl phenol (3.0 g, 21.56 mmol), tert-butyl 2-hydroxyethyl(methyl)carbamate (4.53 g, 25.9 mmol) and triphenylphosphine (6.78 g, 25.9 mmol) in 60 ml of THF with ice-bath cooling under nitrogen atmosphere. The resulting mixture was stirred at room temperature overnight. The solvent was removed under reduced pressure by evaporation. The residue was mixed with ether and filtered. The filtrate was concentrated and purified by flashing silica gel column (Petroleum ether/Ethyl acetate=10/1˜8/1) to afford the intermediate tert-butyl methyl(2-(4-nitrophenoxy)ethyl)carbamate (2.48 g, 39%). To a solution of this intermediate tert-butyl methyl(2-(4-nitrophenoxy)ethyl)carbamate (2.48 g, 8.4 mmol) in methanol was added Pd/C under hydrogen atmosphere. The mixture was heated to 50° C. for 1 hour, and then cooled down to room temperature and filtered. The filtrate was concentrated to give the crude tert-butyl 2-(4-aminophenoxy)ethyl(methyl)carbamate which was used without further purification for the next reaction (2.10 g, 95%). To a solution of triphosgene (206 mg, 0.695 mmol) in DCM was added tert-butyl 2-(4-aminophenoxy)ethyl(methyl)carbamate (500 mg, 1.88 mmol) with ice-bath cooling followed by dropwise addition of TEA (380 mg, 3.76 mmol). After that, the mixture was stirred at room temperature for 2 hours. The solvent was removed under reduced pressure without heating. The residue mixed with ether and filtered. The filtrate was concentrated to give tert-butyl 2-(4-isocyanatophenoxy)ethyl(methyl)carbamate (500 mg).

### Example 83

### Synthesis of tert-butyl 2-(4-isocyanatophenoxy)ethyl(ethyl)carbamate

Using a procedure similar to the preparation of tert-butyl 2-(4-isocyanatophenoxy)ethyl(methyl)carbamate (example 82), replacing 2-(methylamino)ethanol with 2-(ethylamino)ethanol, the isocyanate, tert-butyl 2-(4-isocyanatophenoxy)ethyl(ethyl)carbamate were made.

### Example 84

### Synthesis of Compounds (109), (110), (111), (112), (113), (114), (115), (116), (117), (118), (119), (120), (121), (122) and (123)

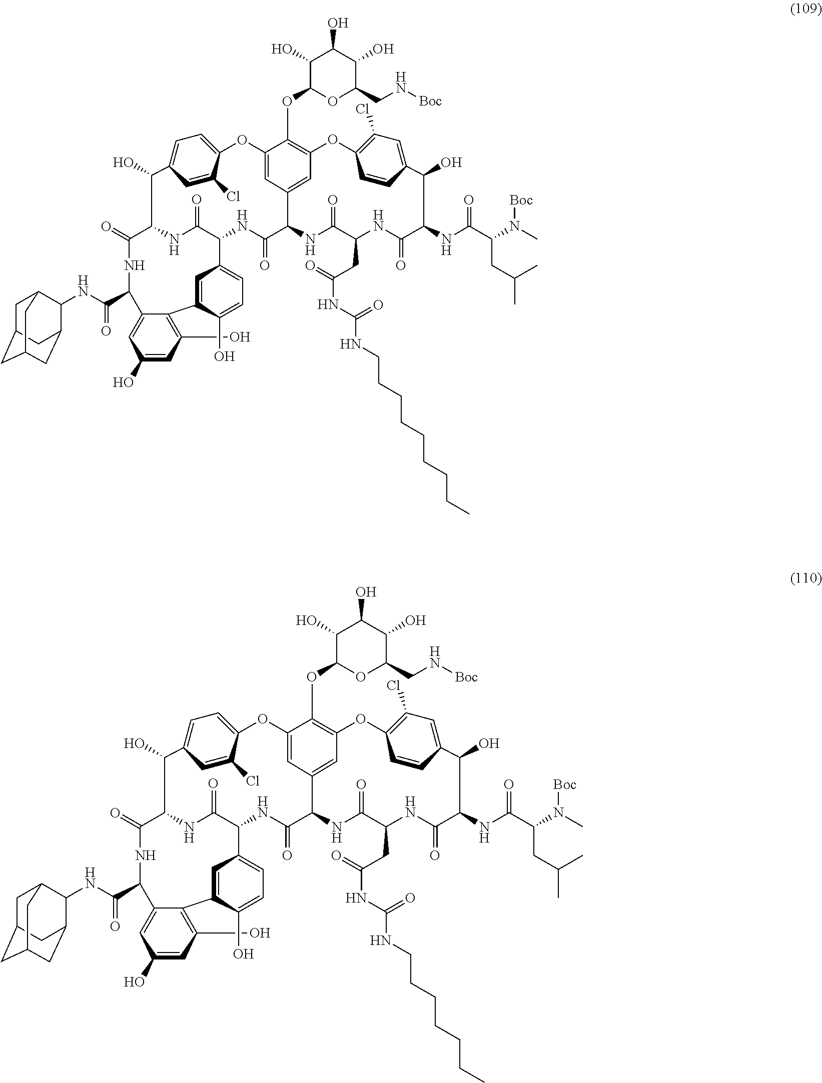

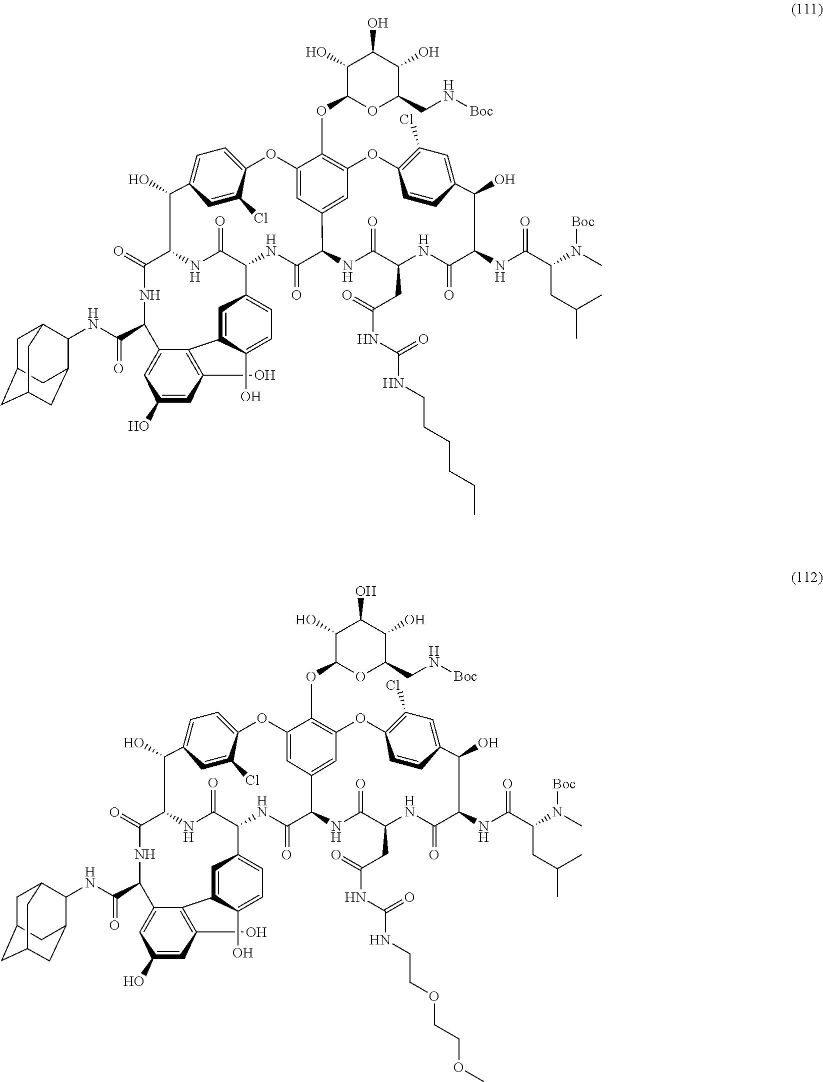

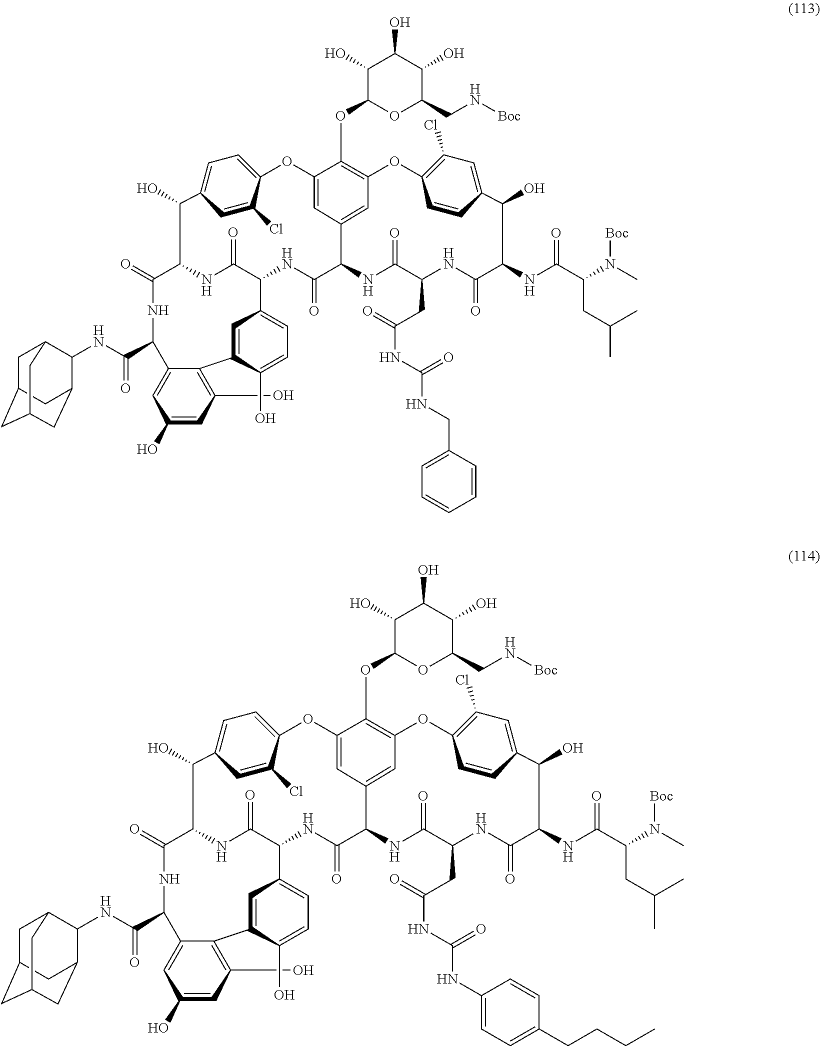

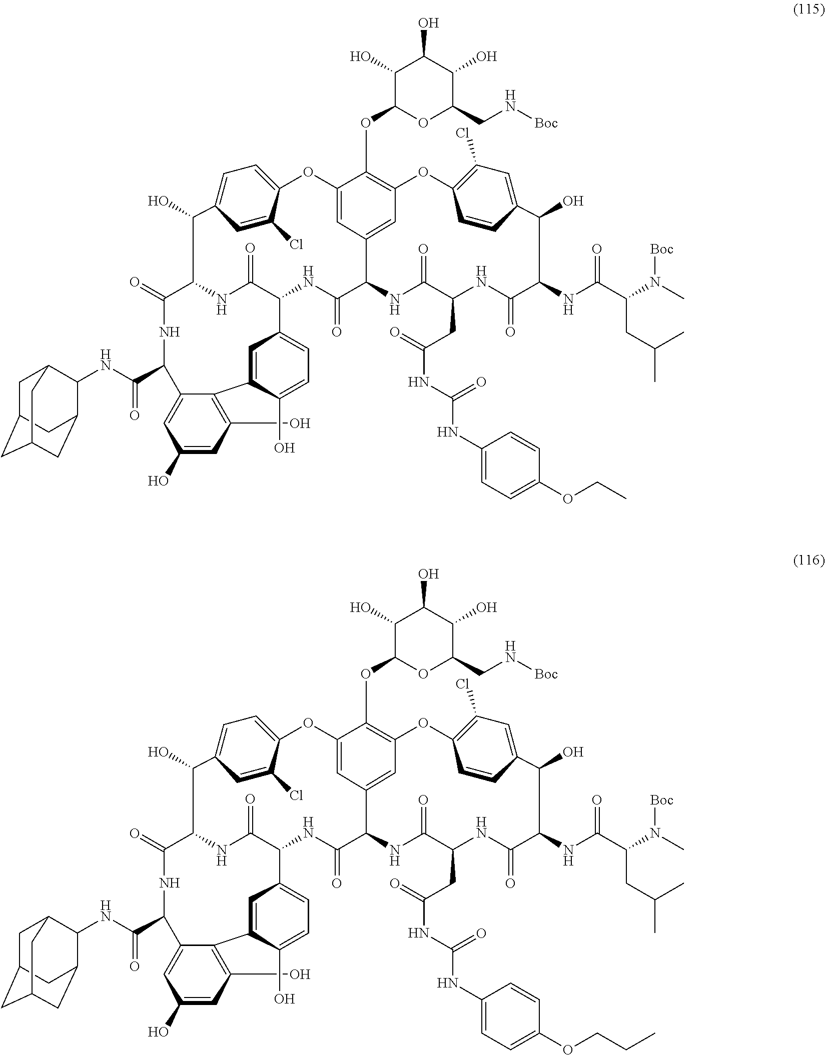

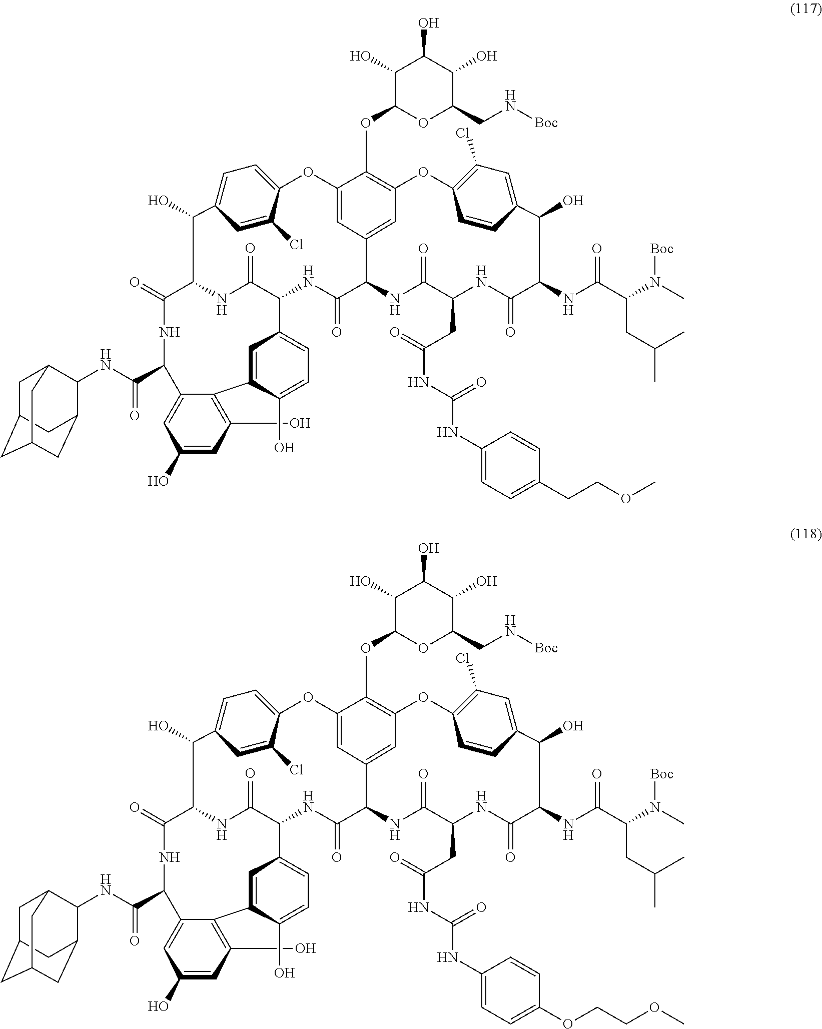

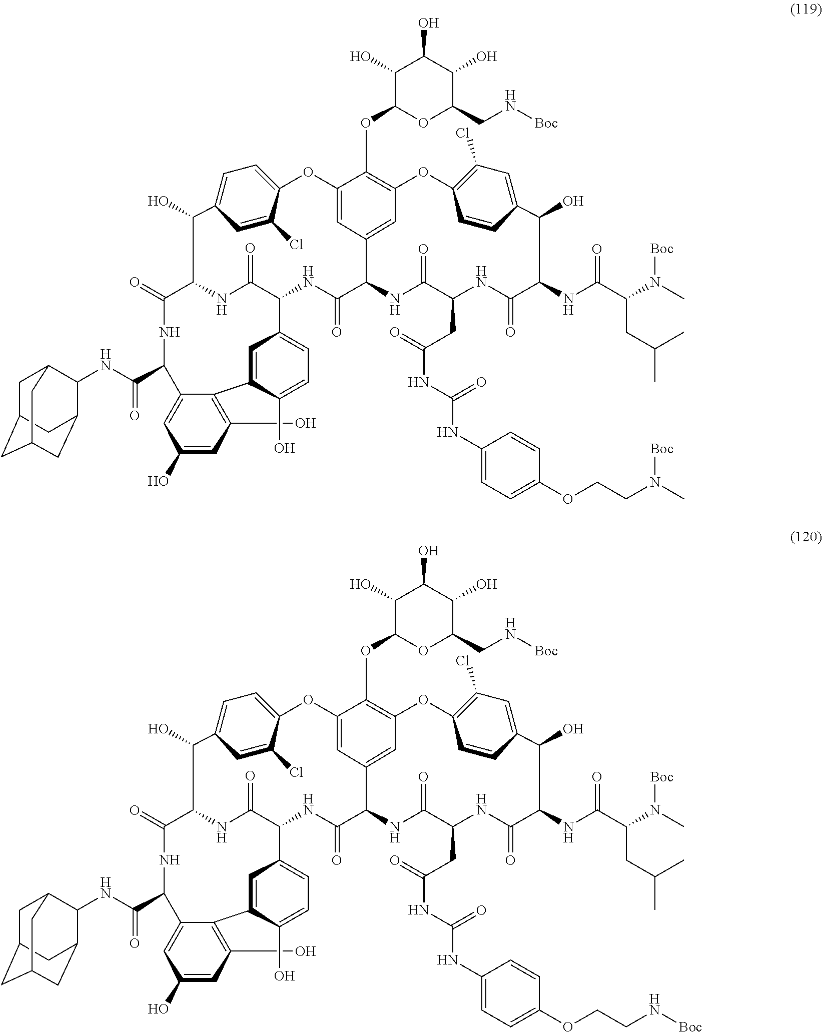

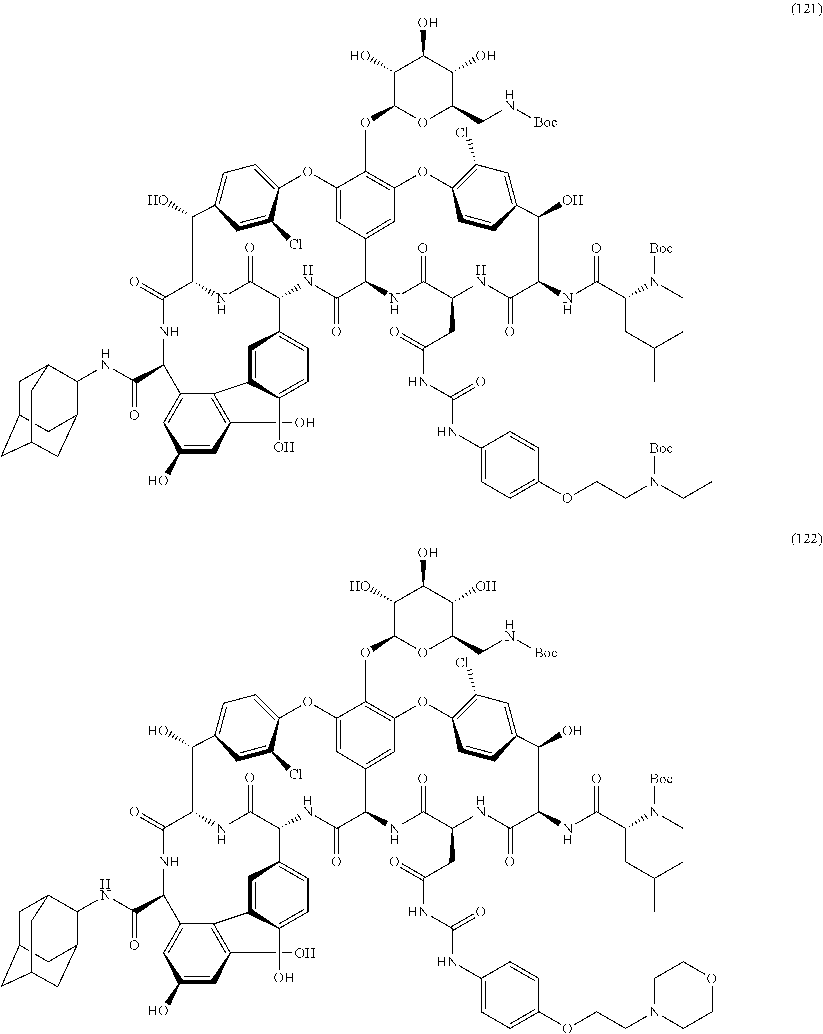

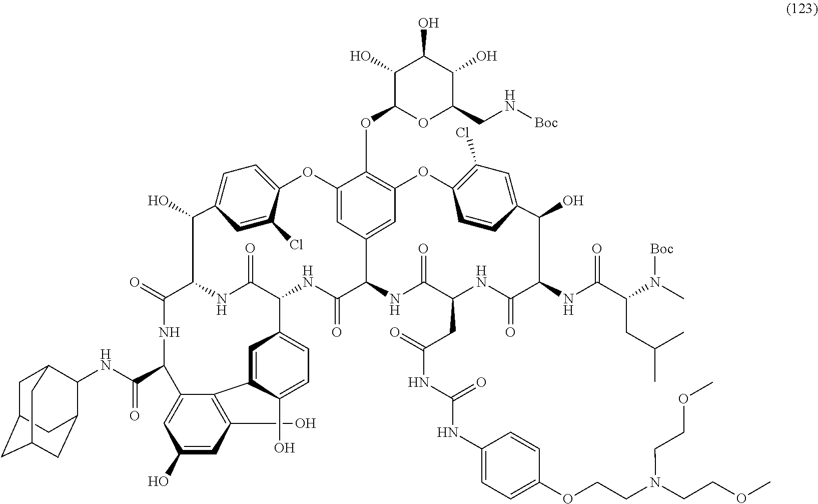

Using a procedure similar to the preparation of Compound (57) as in example 57 and replacing the isocyanate C₈H₁₇ NCO with an appropriate isocyanate, Compounds (109), (110), (111), (112), (113), (114), (115), (116), (117), (118), (119), (120), (121), (122) and (123) were made.

### Example 85

### Synthesis of tert-butyl 2-(4-isocyanatophenoxy)ethyl(propyl)carbamate

Using a procedure similar to the preparation of tert-butyl 2-(4-isocyanatophenoxy)ethyl(methyl)carbamate (example 82), replacing 2-(methylamino)ethanol with 2-(propylamino)ethanol, the isocyanate, tert-butyl 2-(4-isocyanatophenoxy)ethyl(propyl)carbamate is made.

### Example 86

### Synthesis of Compounds (124), (125), (126), (127), (128), (129), (130), (131), (132), (133), (134), (135), (136), (137) and (138)

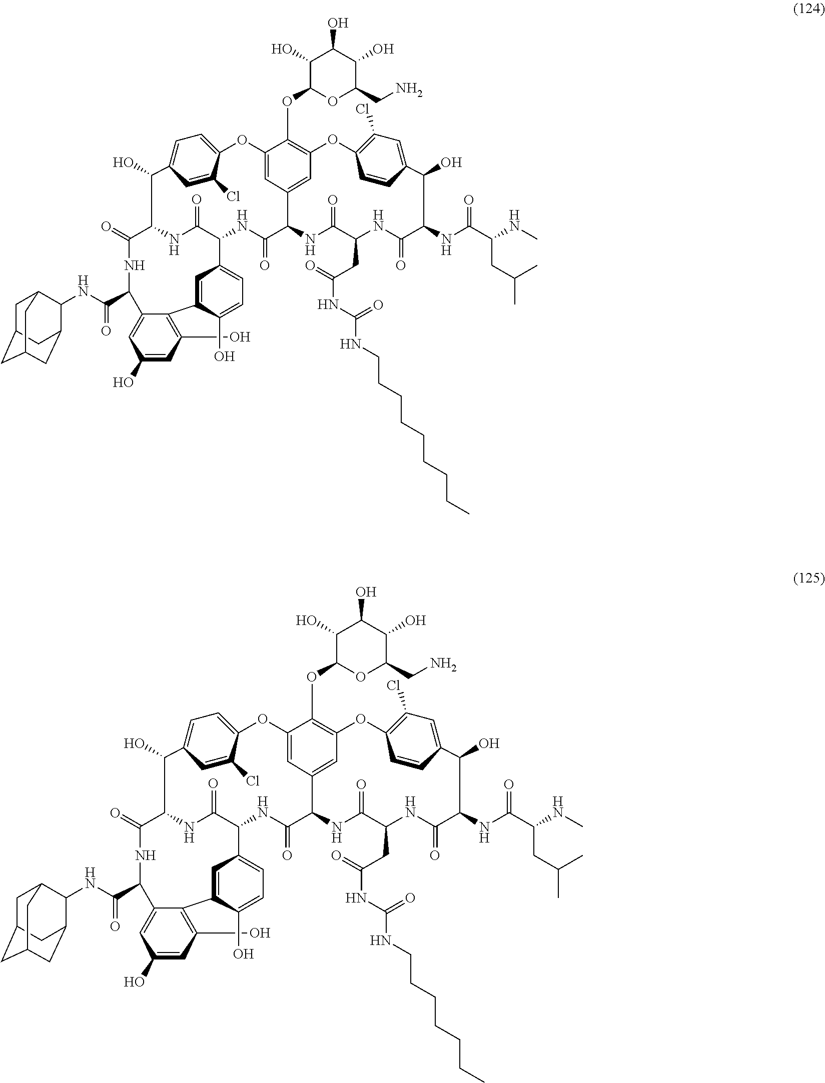

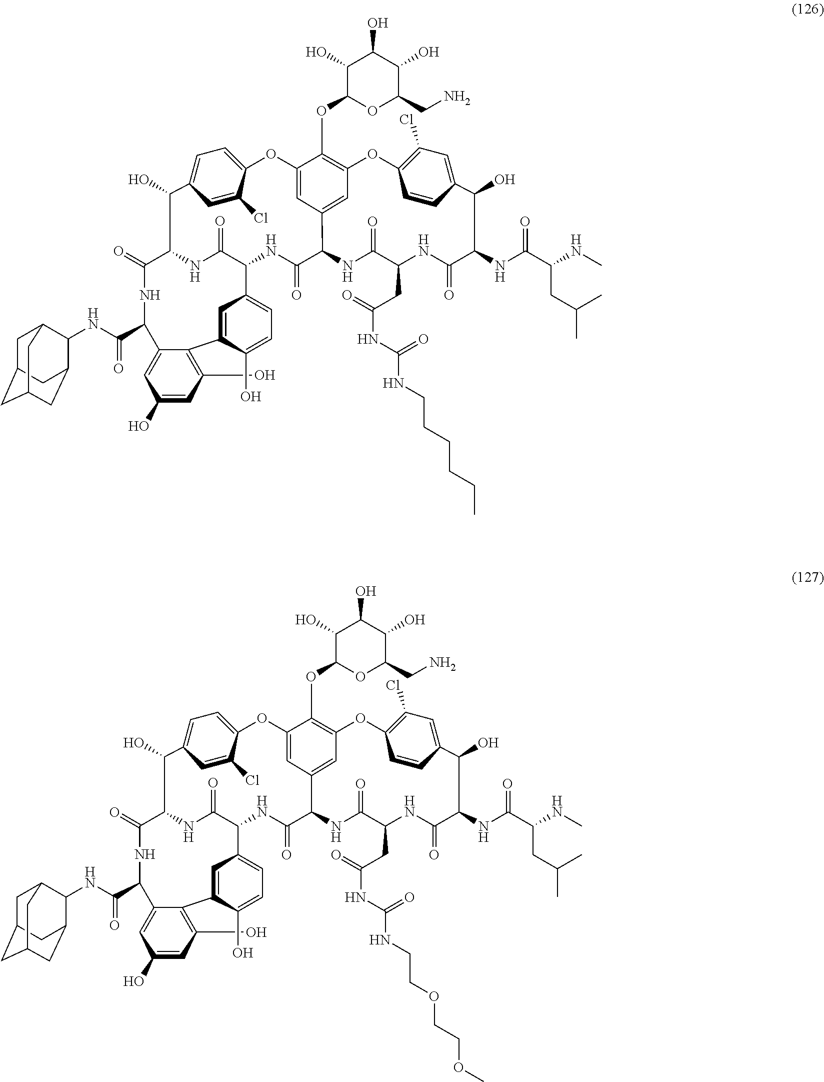

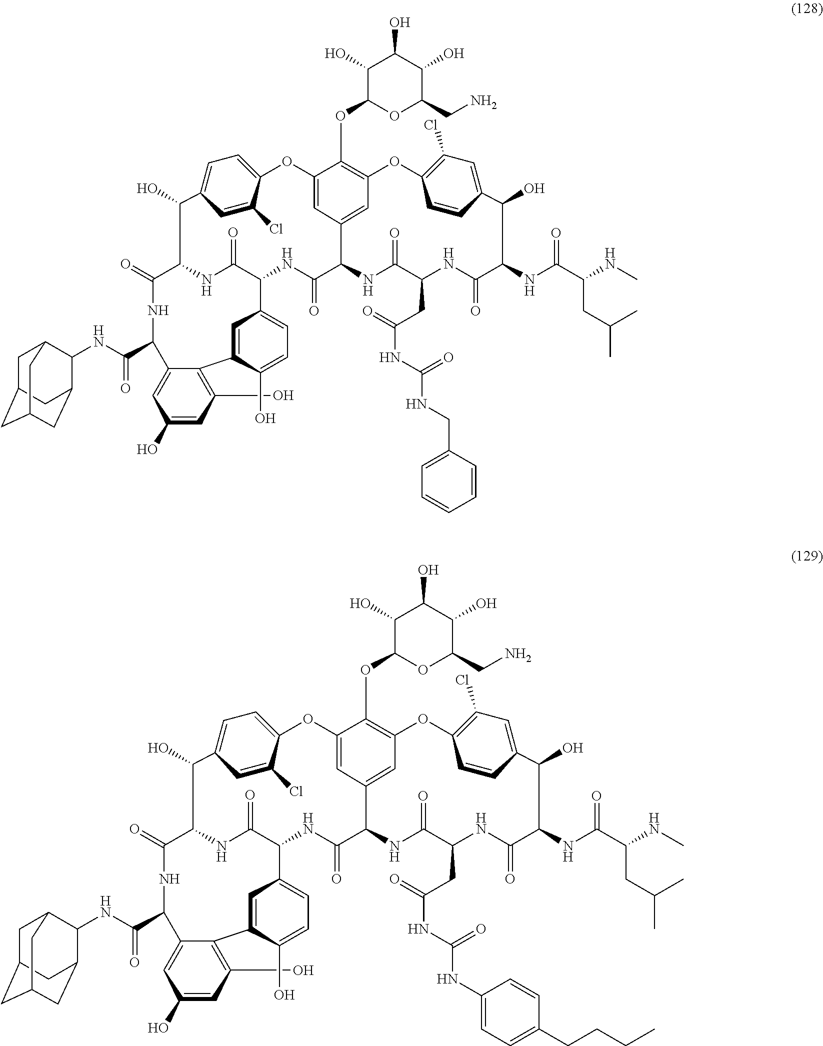

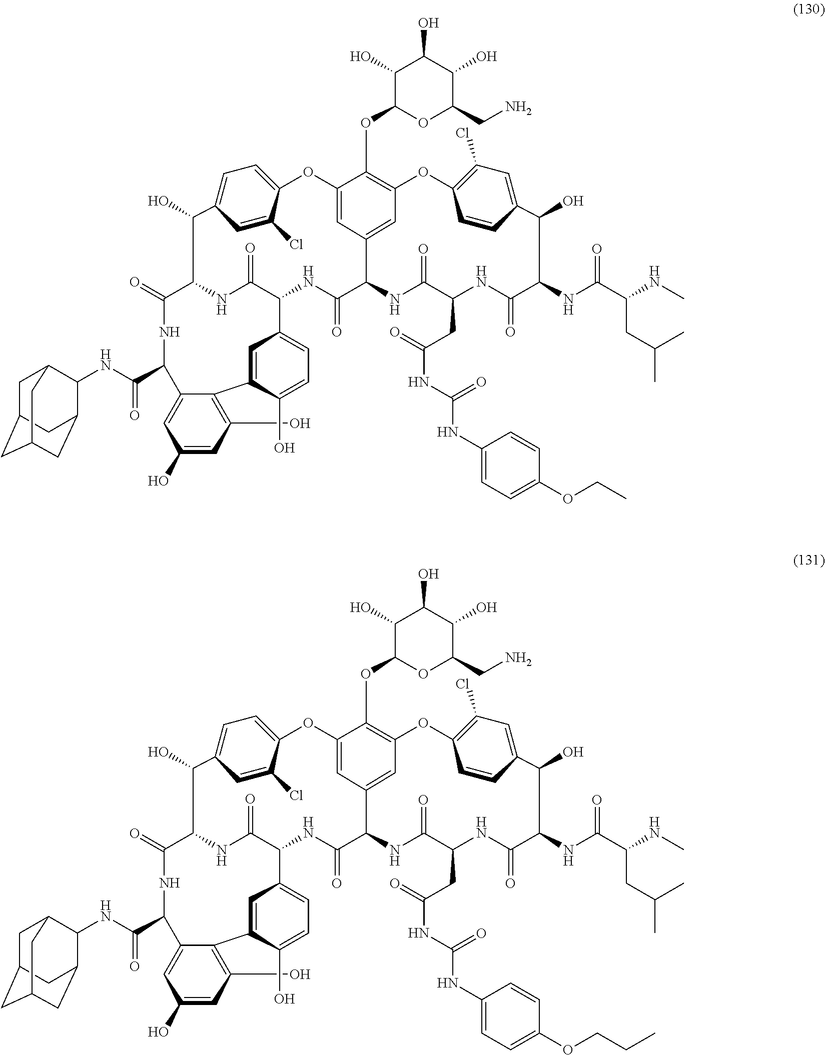

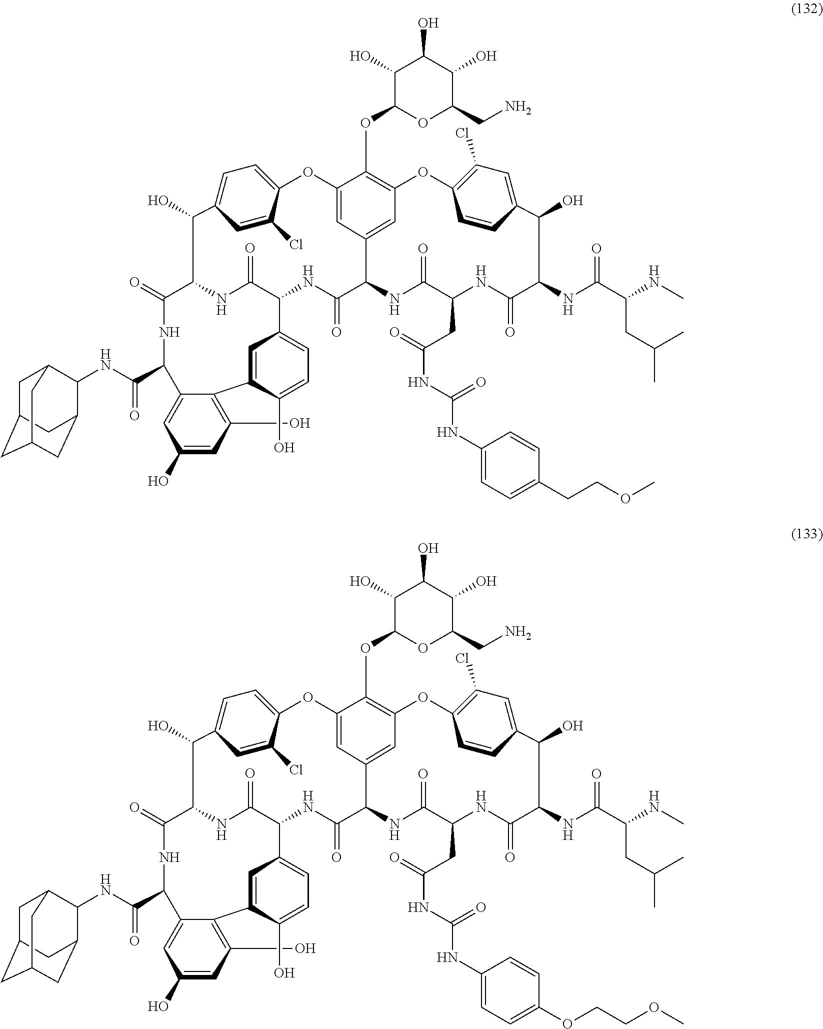

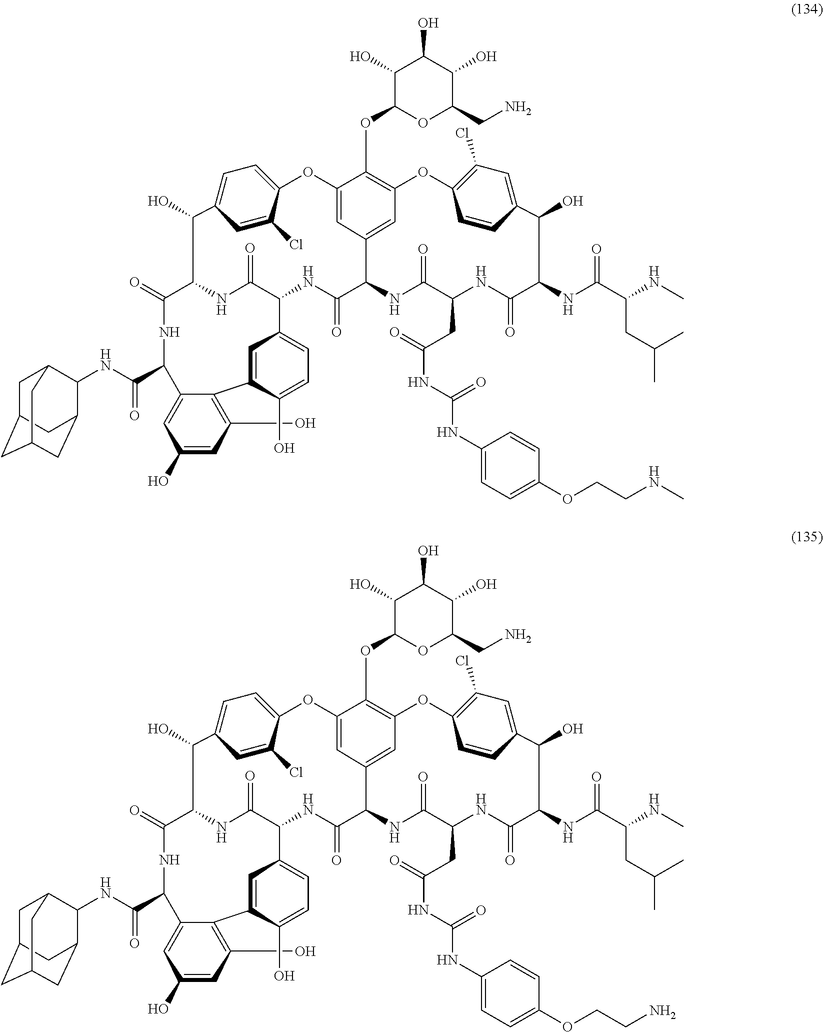

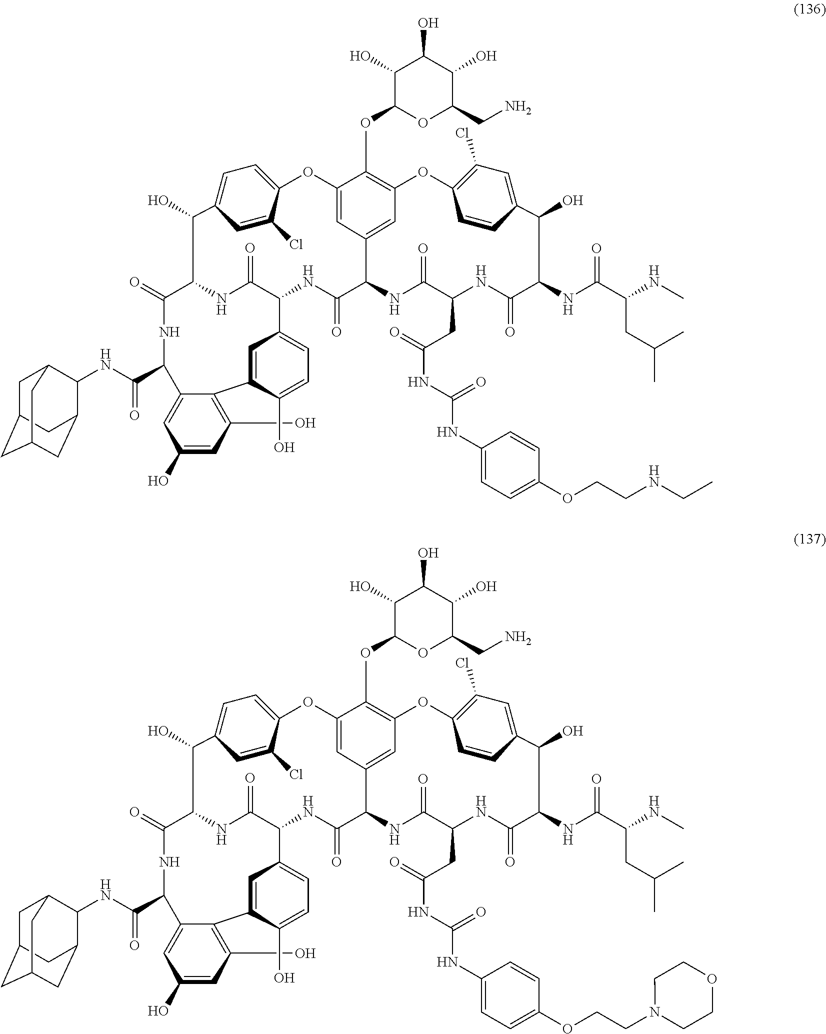

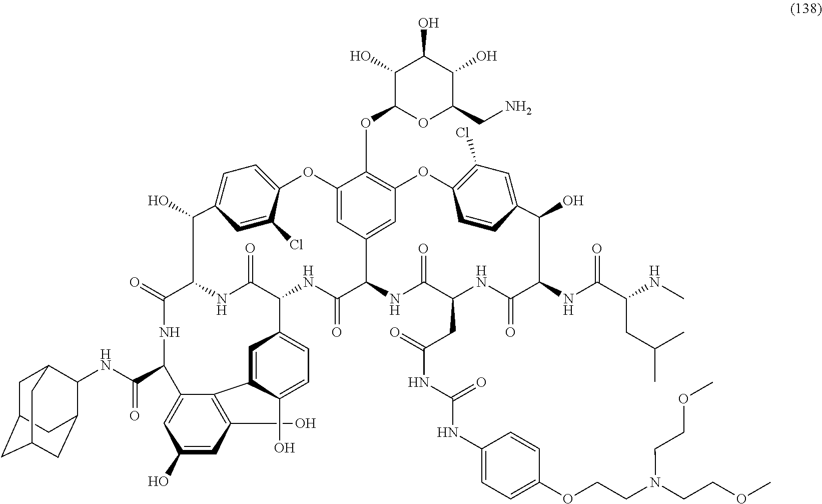

Using a procedure similar to the preparation of Compound (58) as in example 58 and replacing Compound (57) with Compounds (109), (110), (111), (112), (113), (114), (115), (116), (117), (118), (119), (120), (121), (122) and (123), the acylurea derivatives Compounds (124), (125), (126), (127), (128), (129), (130), (131), (132), (133), (134), (135), (136), (137) and (138) were made as TFA salts.

### Example 87

Alternate Synthesis of Compound (21)

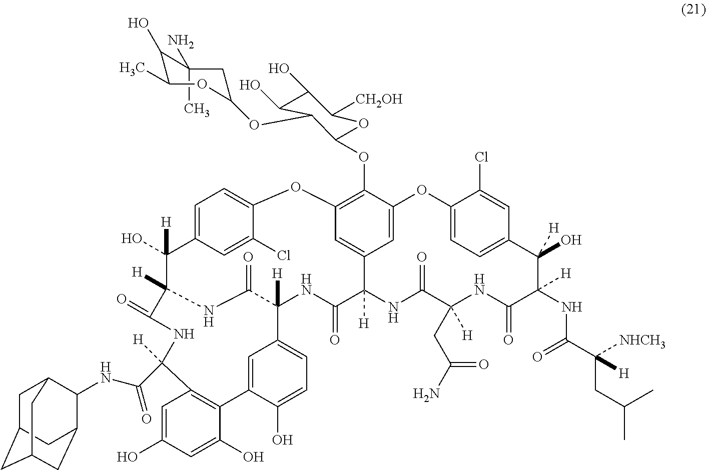

To a solution of vancomycin hydrochloride (100.0 g) in DMSO (800 mL) was added 2-adamantylamine hydrochloride (20.0 g), DIPEA (35.0 g) and HATU (28.1 g) with stirring at ambient temperature. The reaction mixture was stirred overnight. Analytical HPLC showed the reaction completed. DMSO was removed under vacuum. The residue was subjected to purification by reverse phase silica gel column chromatography (C18 silica gel, CH₃CN—H₂O:5%-30%). The collected fraction was condensed to give Compound (21) (45 g) as a white powder.

### Example 88

### Synthesis of Compound (139)

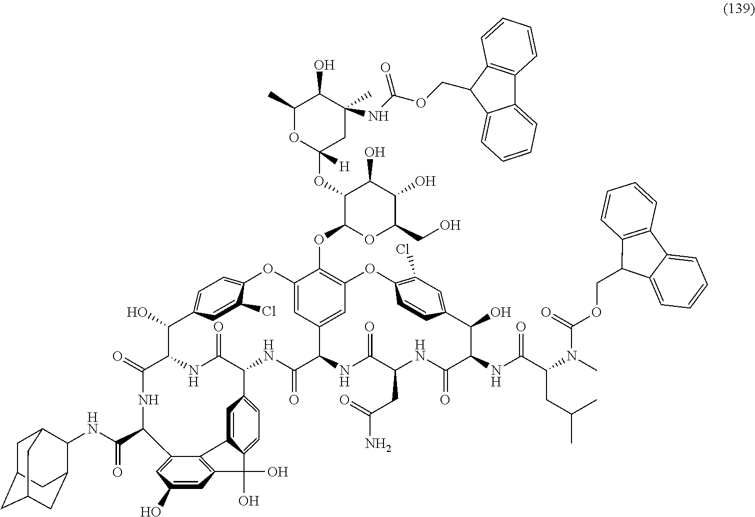

To a solution of Compound (21) (35.0 g) in 1,4-dioxane (50 mL) and water (50 mL) was added Fmoc-OSu (9-fluorenylmethyloxycarbonyl-O-succinimide) (11.0 g) with stirring at room temperature. After the reaction mixture was stirred at ambient temperature for 2 hr, the solvent was removed under reduced pressure. The resulting solid was collected by filtration under vacuum and was purified by silica gel column chromatography (silica gel, MeOH—CH2C12: 10%-20%) to give Compound (139), (20 g) as a white solid.

### Example 89

### Synthesis of Compound (140)

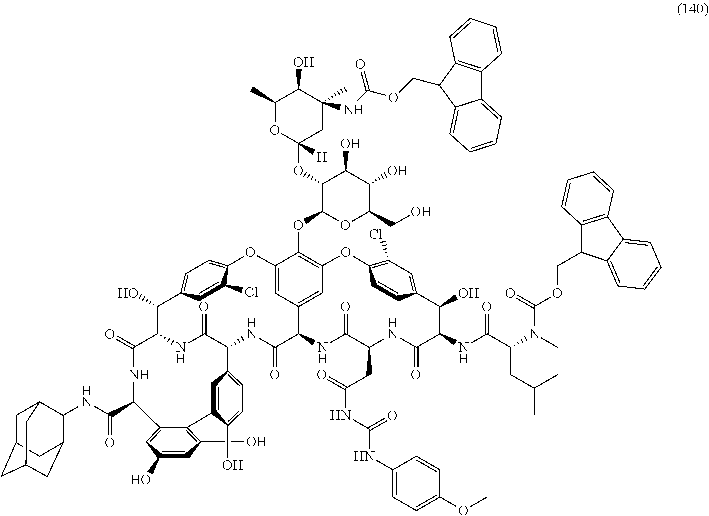

Using a procedure similar to the preparation of Compound (57) as in Example 57 and replacing Compound (56) with Compound (139), and isocyanate C₈H₁₇NCO with 1-isocyanato-4-methoxybenzene, Compound (140) was made.

### Example 90

Alternate Synthesis of Compound (141)

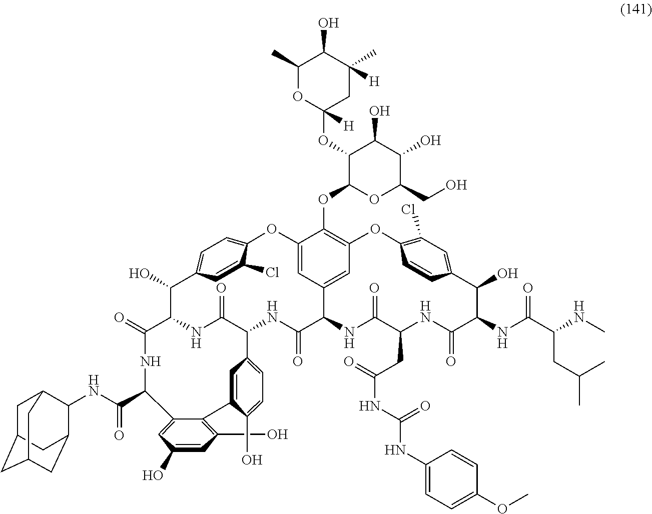

Compound (140) obtained from Example 89 was dissolved into DMF (9 mL) and then diethylamine (3 eq.) was added at ambient temperature. After stirring at room temperature for 2 hr, the reaction mixture was poured into ether. The formed solid was applied on preparative HPLC to give Compound (141).

### Example 91

### Synthesis of Compound (142) & (143)

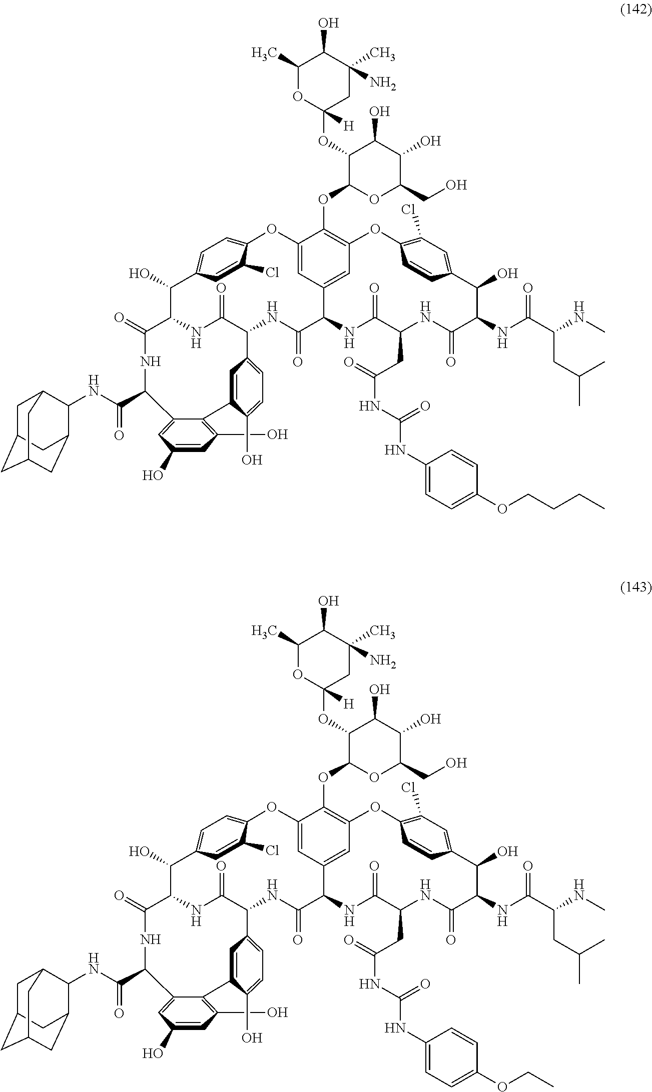

Using a procedure provided in Examples 89 and 90 in the preparation of Compound (141) and replacing 1-isocyanato-4-methoxybenzene with 1-isocyanato-4-butoxybenzene or 1-isocyanato-4-ethoxybenzene, Compound (142) and Compound (143) were prepared, respectively.

### Example 92

### Synthesis of Compound (144)

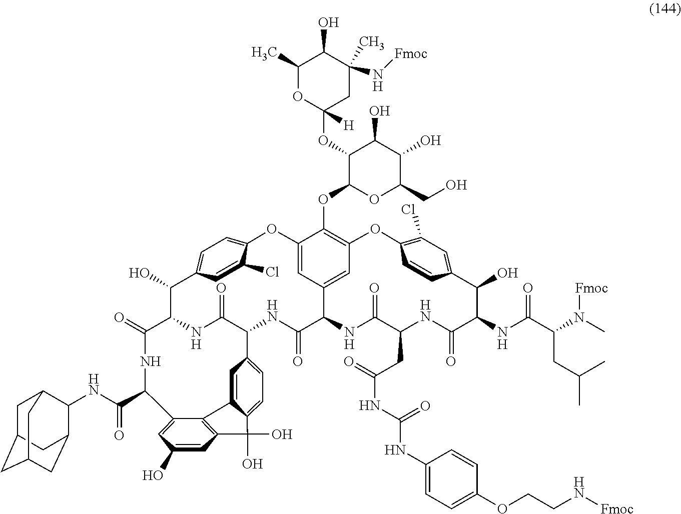

Using a procedure similar to the preparation of Compound (140) as in Example 89 and replacing 1-isocyanato-4-methoxybenzene with 1-isocyanato-4-(2-(9-fluorenylmethyloxycarbonylamino)ethoxy), Compound (144) is prepared.

### Example 93

### Synthesis of Compound (145)

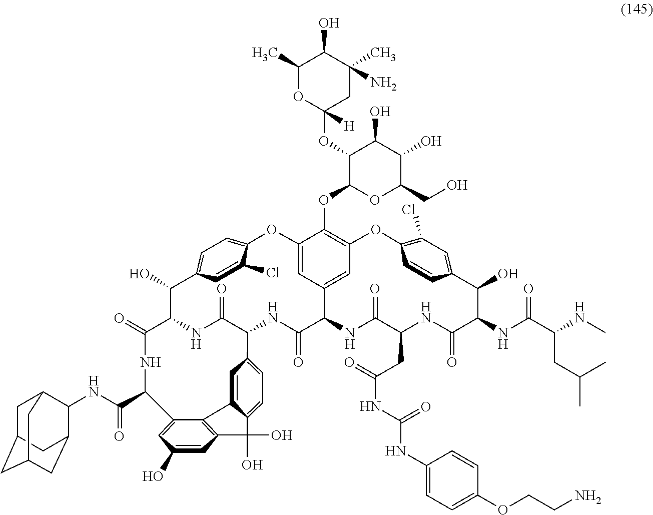

Using a procedure similar to the preparation of Compound (141) as in Example 90 and replacing Compound (140) with Compound (144), Compound (145) is made.

### Example 94

### Synthesis of Compounds (146), (147), (148), (149) and (150)

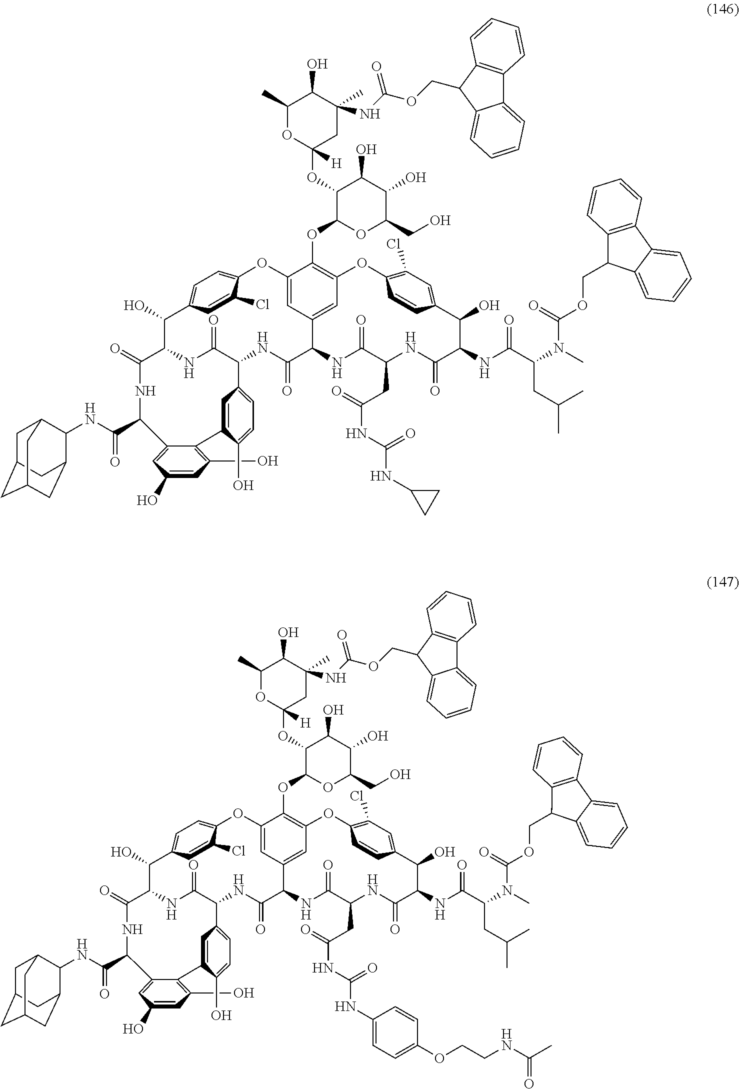

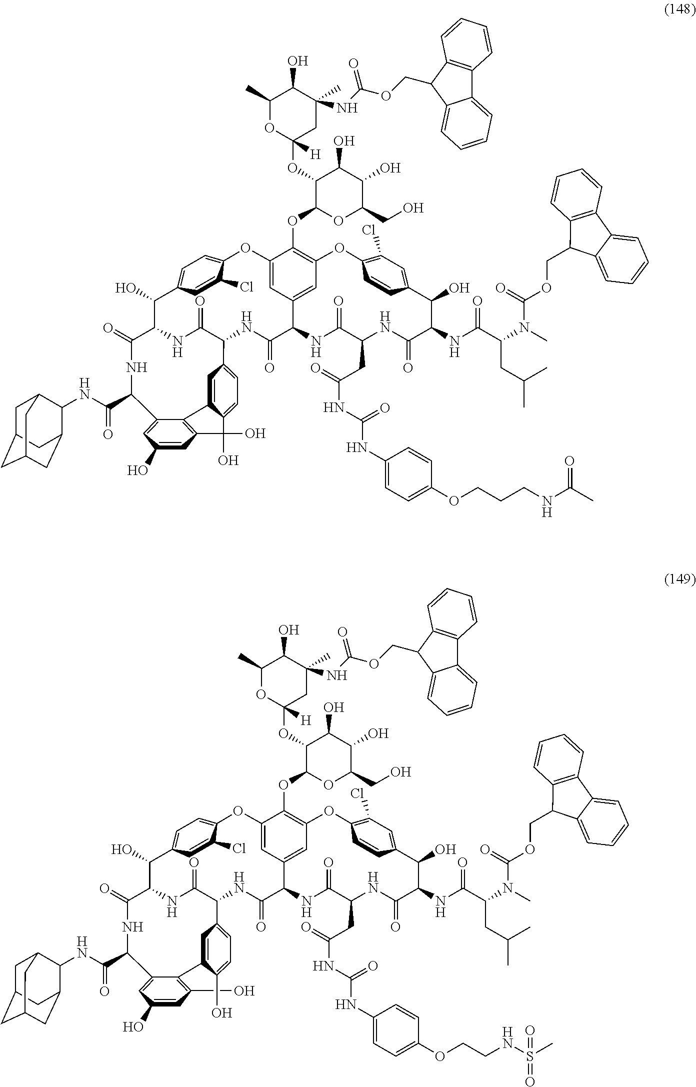

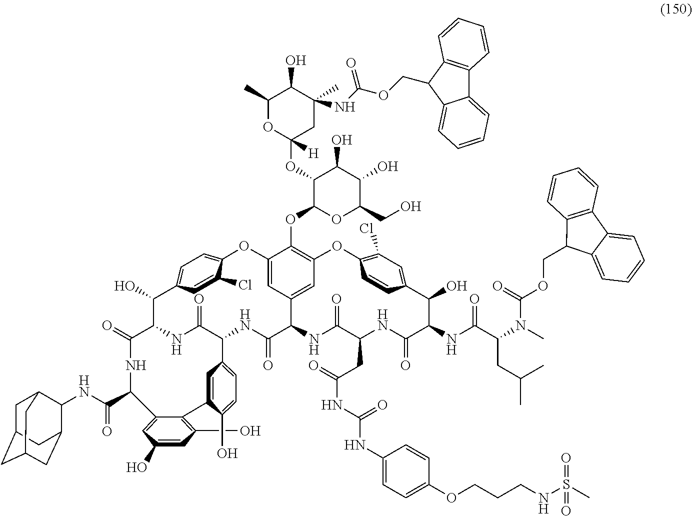

Using a procedure similar to the preparation of Compound (140) as in Example 89 and replacing 1-isocyanato-4-methoxybenzene with other appropriate isocyanates, Compounds (146), (147), (148), (148) and (150) are prepared.

### Example 95

### Synthesis of Compounds (151), (152), (153), (154) and (155)

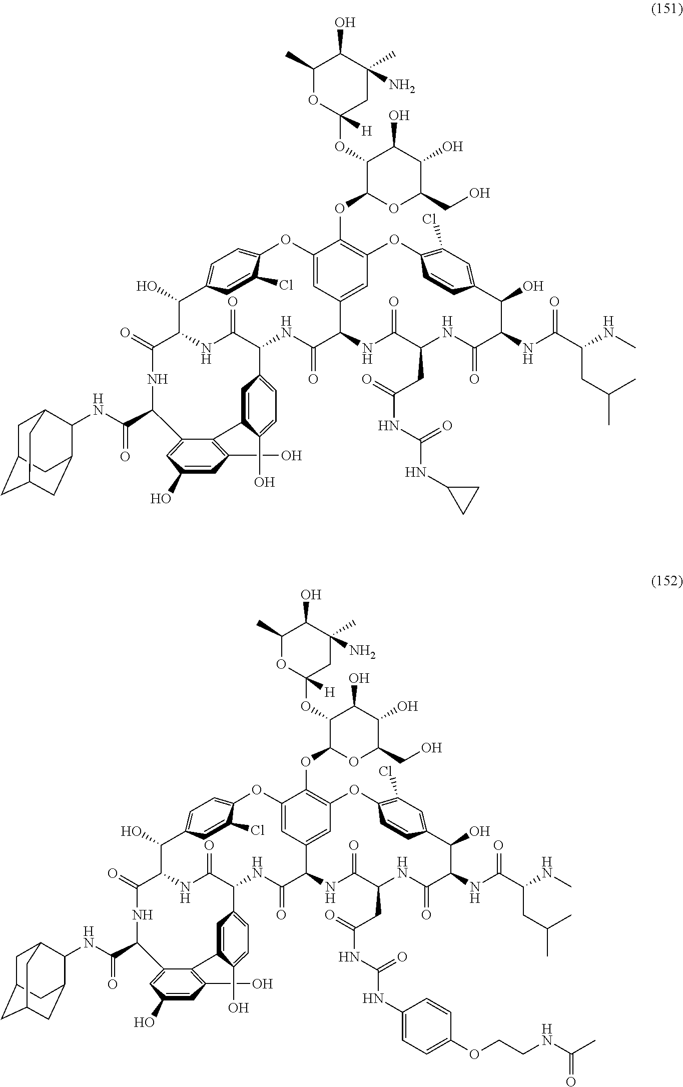

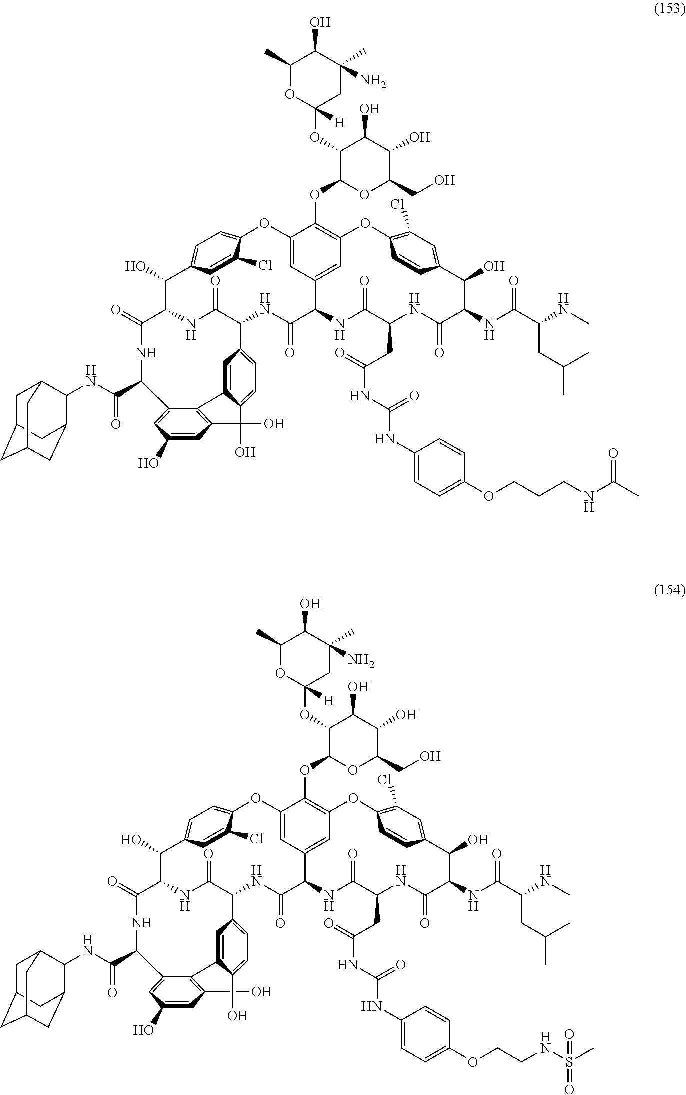

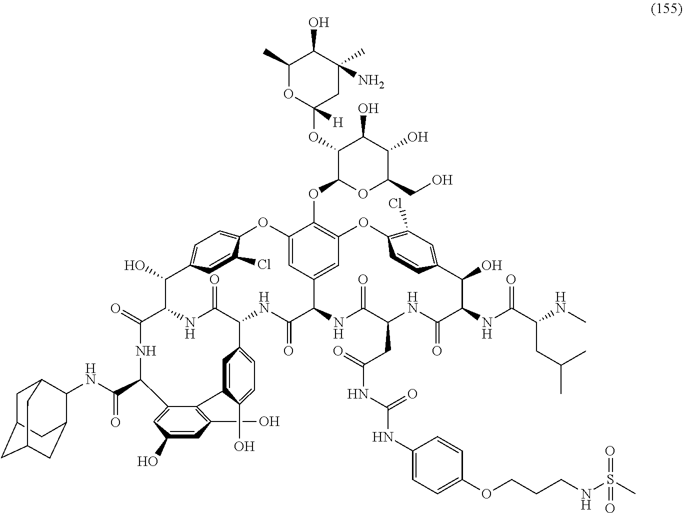

Using a procedure similar to the preparation of Compound (141) as in Example 90 and replacing Compound (140) with Compounds (146), (147), (148), (149), and (150), Compounds (151), (152), (153), (154), and (155) are prepared, respectively.

### Example 96

### Synthesis of Compounds (156), (157), (158), (158), (160) and (161)

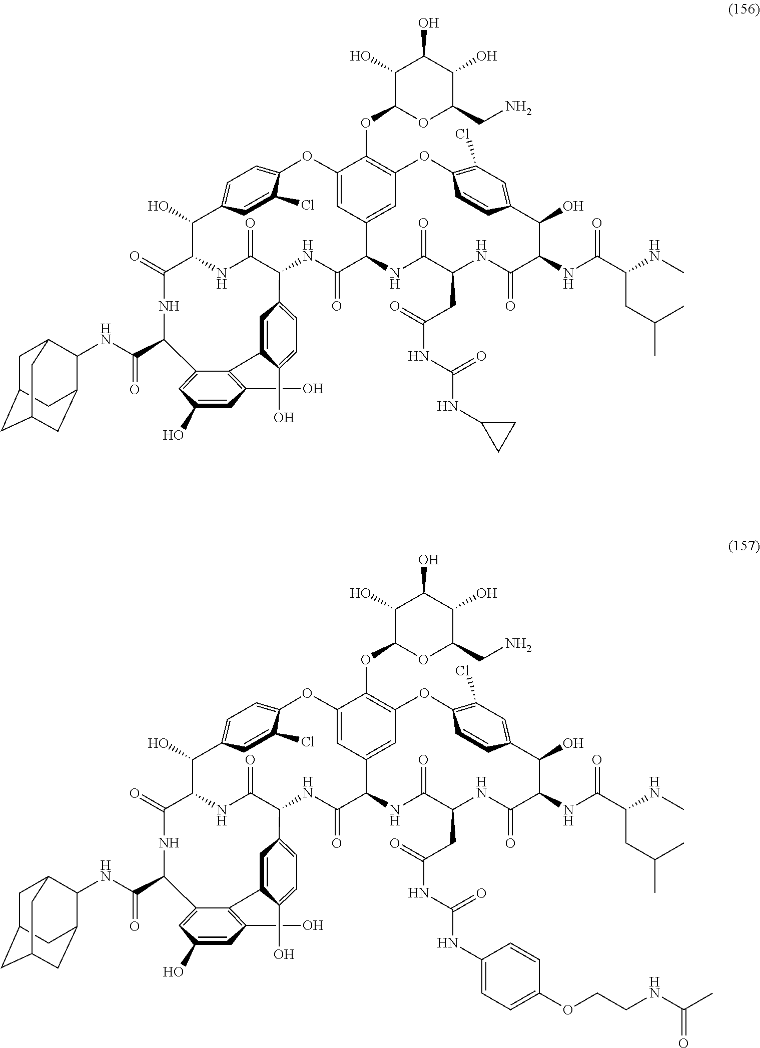

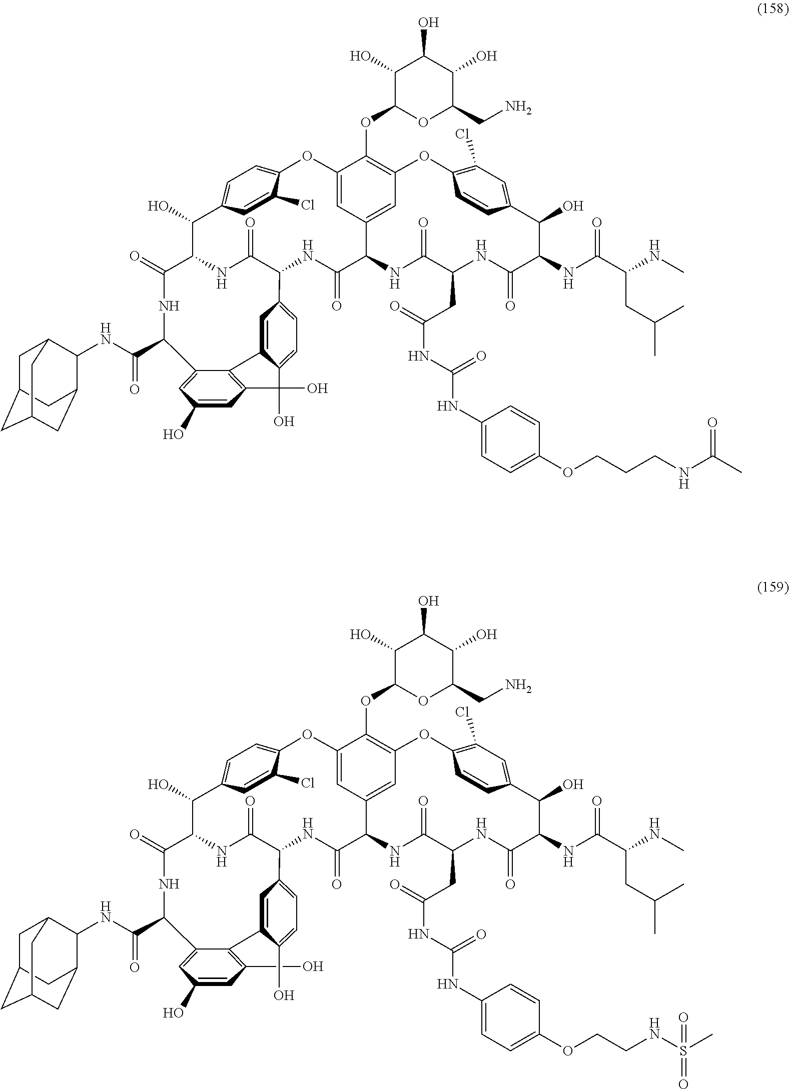

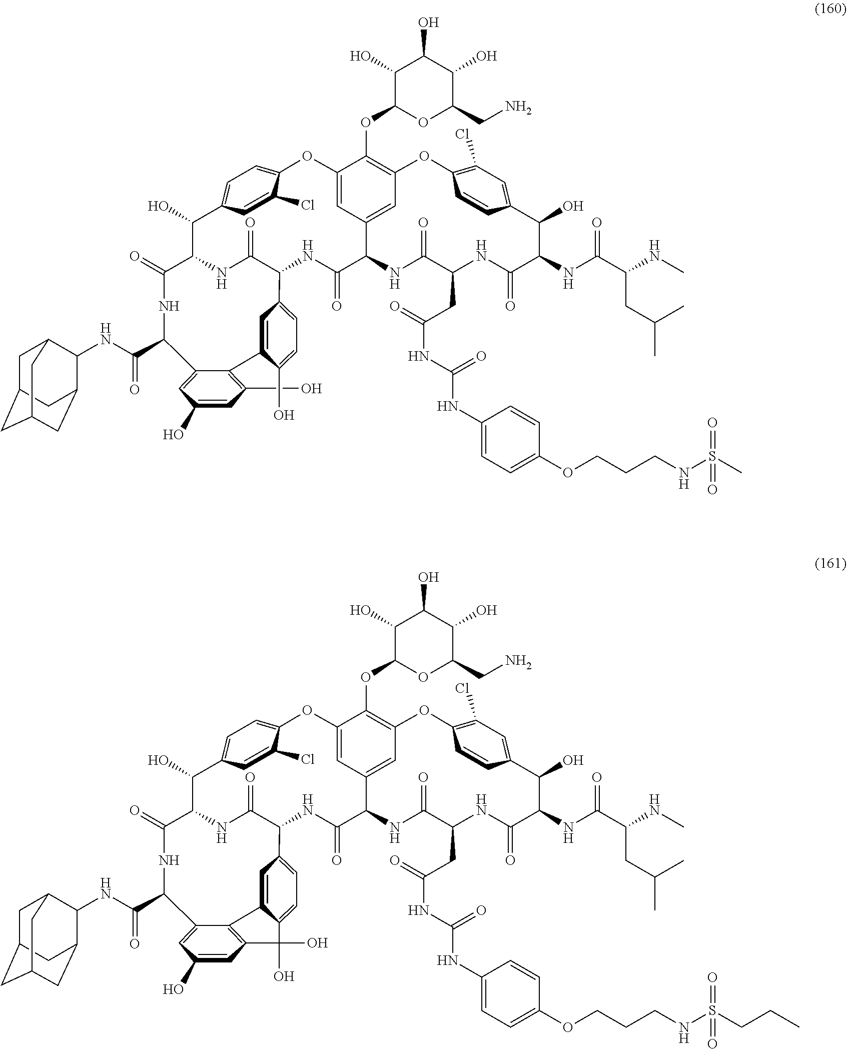

Using a procedure provided in Examples 84 and 86 in the preparation of various acylurea derivatives such as Compounds (124), and using appropriate isocyanates, acylurea Compounds (156), (157), (158), (158), (160), and (161) are prepared.

### Example 97

### Synthesis of Compound (162)

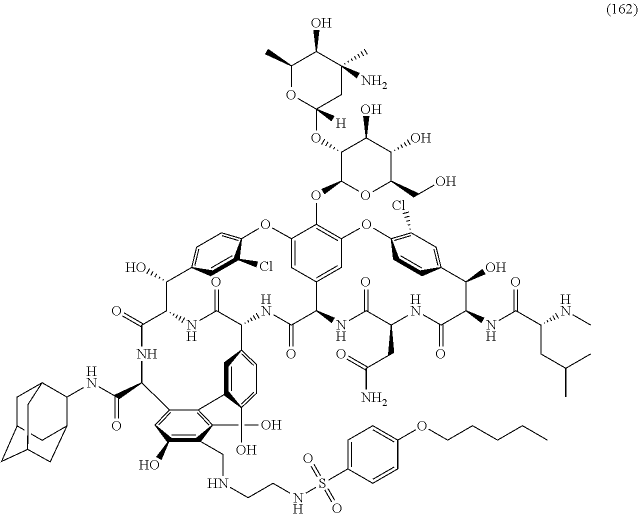

To a solution of mixture of N-(2-aminoethyl)-4-(pentyloxy)benzenesulfonamide (151 mg, 0.53 mmol) and Compound (139) (1 g, 0.53 mmol) in acetonitrile (30 mL) and water (30 mL) was added 37% aqueous formaldehyde (1.2 g, 14.8 mmol) and acetic acid (640 mg, 10.7 mmol) at room temperature. The reaction mixture was stirred for an additional 20 hr at room temperature. The volatile solvents were removed under reduced pressure. The formed solid was collected by filtration and washed with EtOAc. The crude product was dissolved into DMF (5 mL). After diethylamine (22 mg) was added, the reaction mixture was stirred at room temperature for 40 minutes and then was poured into ether (20 mL). The formed solid was applied on preparative HPLC to give Compound (162) as a white powder.

### Example 98

### Synthesis of Compound (163)

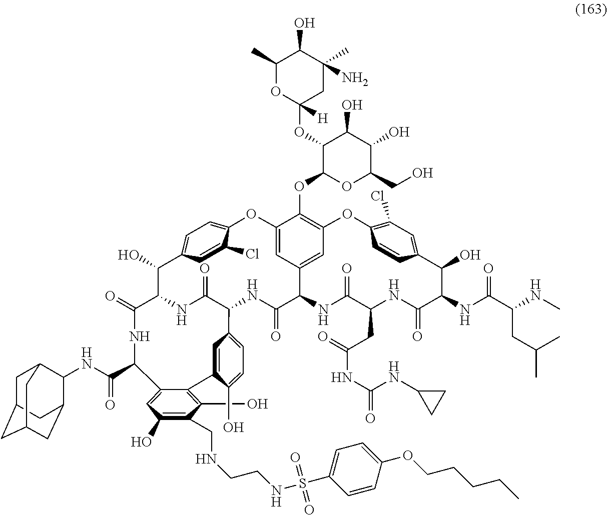

Using a procedure similar to the preparation of Compound (162) as in Example 97 and replacing Compound (139) with Compound (146), Compound (163) is made.

### Example 99

### Synthesis of Compounds (164), (165), (166), (167), (168) and (169)

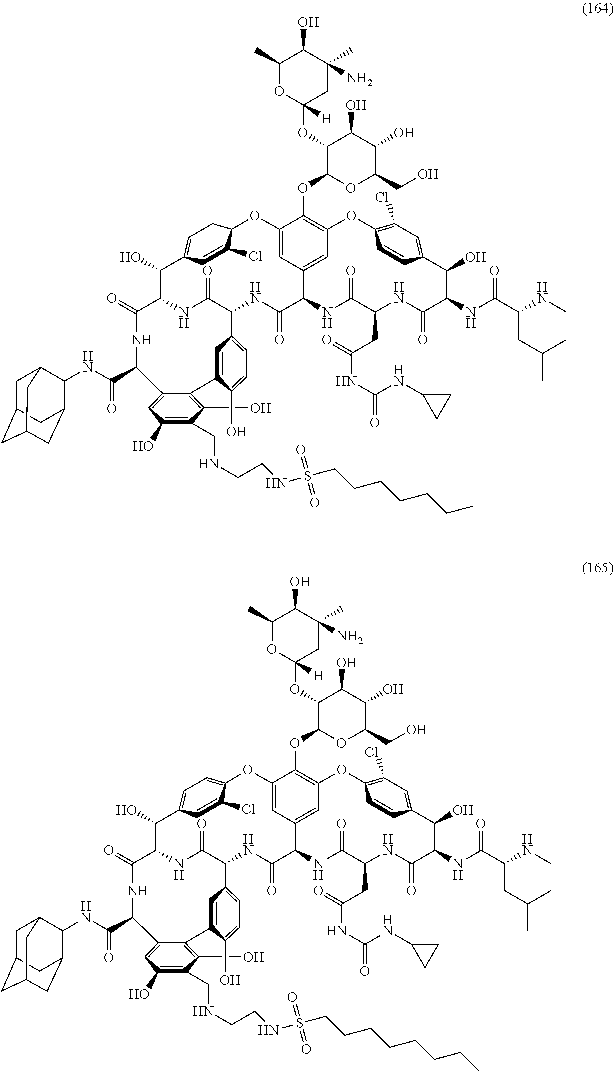

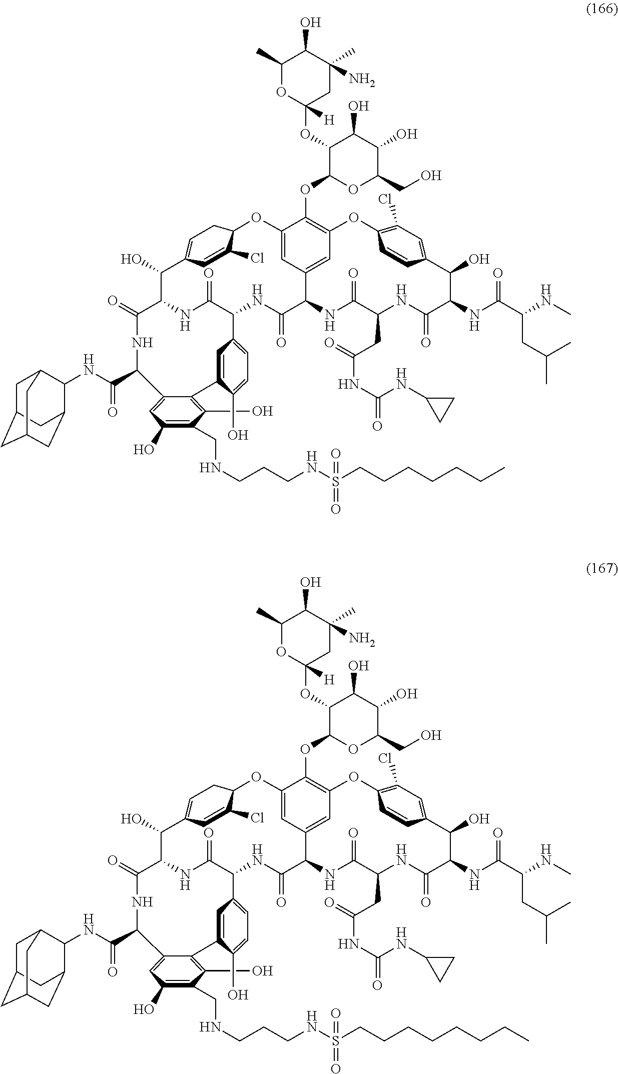

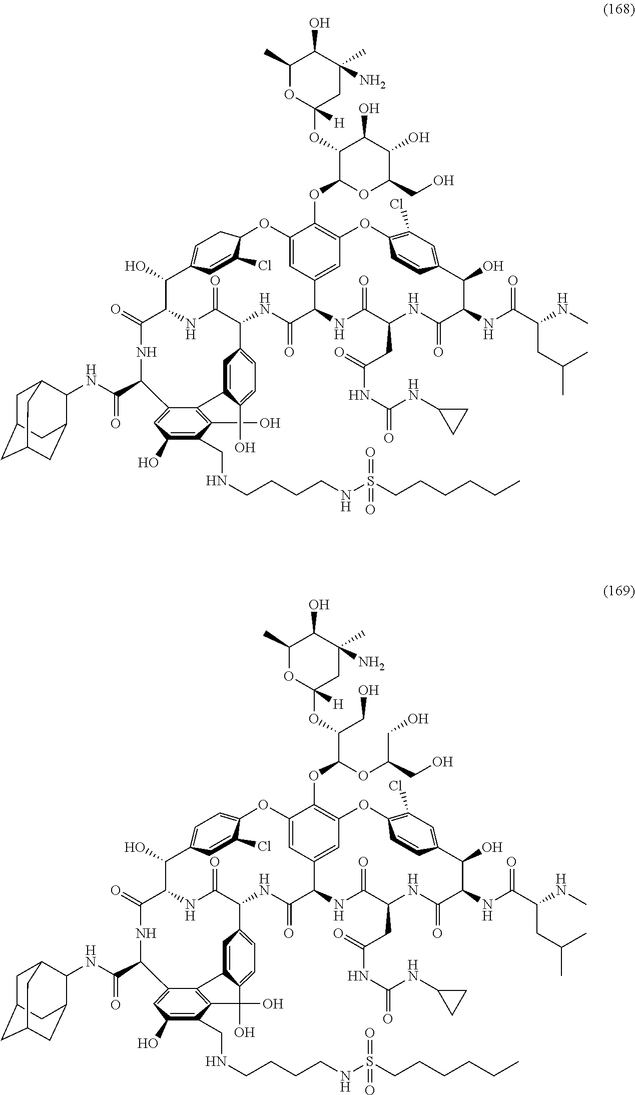

Using a procedure similar to the preparation of Compound (162) as in Example 97 and replacing Compound (139) with Compound (146), and N-(2-aminoethyl)-4-(pentyloxy)benzenesulfonamide with various aminoalkyl sulfonamide, Compounds (164), (165), (166), (167), (168) and (169) are prepared.

### Example 100

### Synthesis of Compound (170)

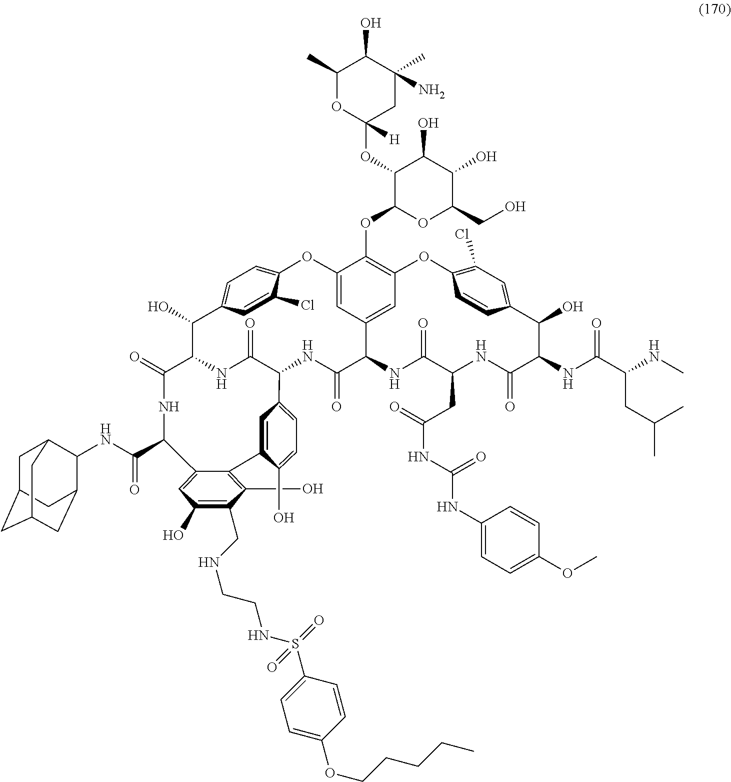

Using a procedure similar to the preparation of Compound (162) as in Example 97 and replacing Compound (139) with Compound (140), and Compound (170) is made.

### Example 101

### Synthesis of Compounds (171), (17), (173), (174), (175) (176), (177), (178), (179), (180) and (181)

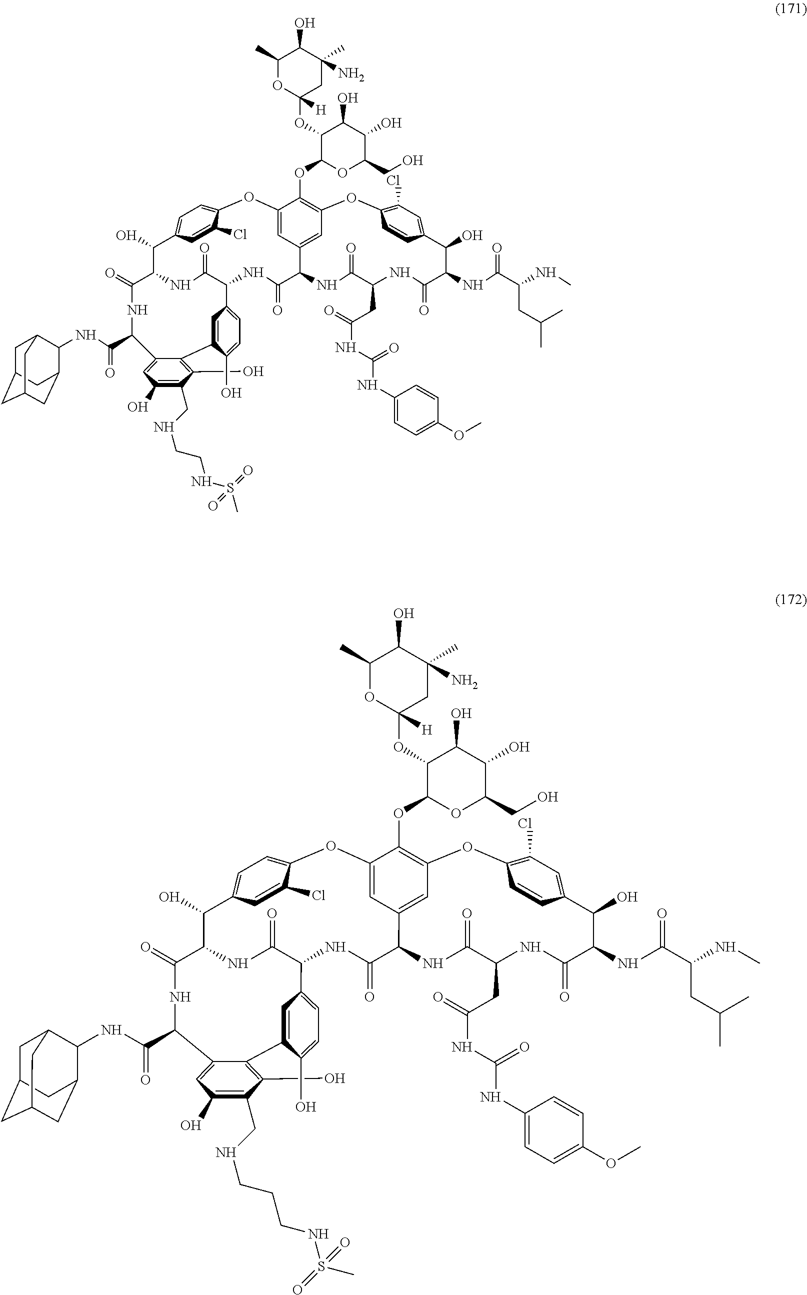

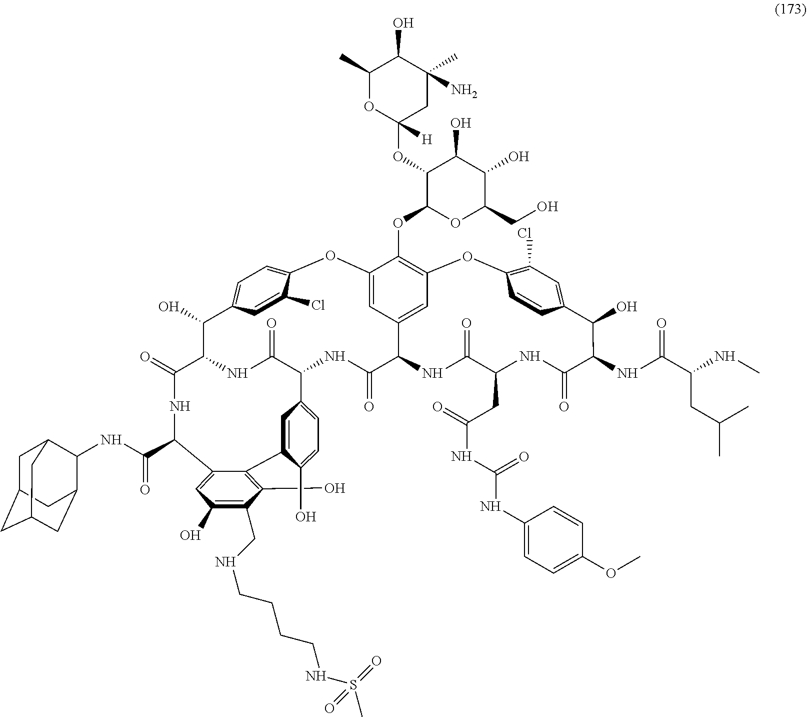

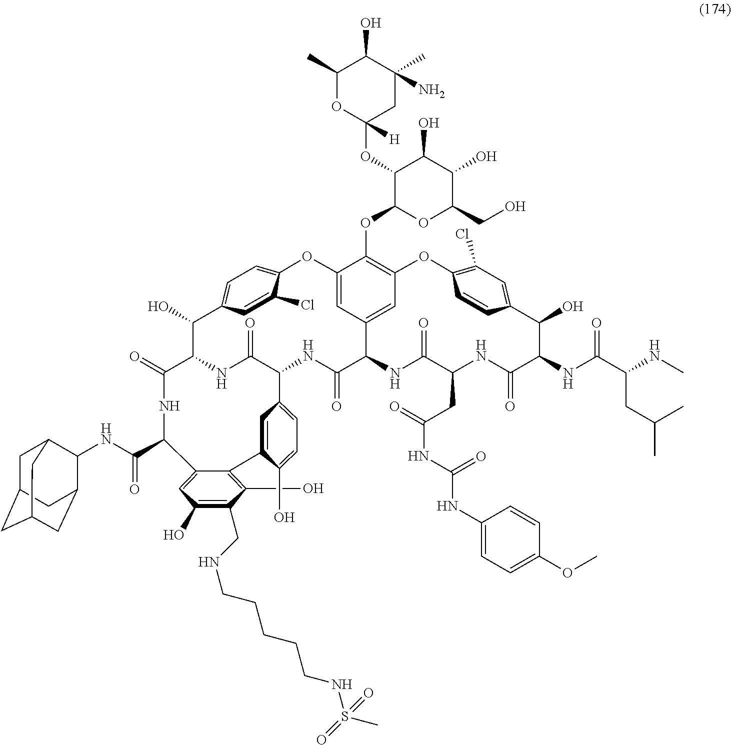

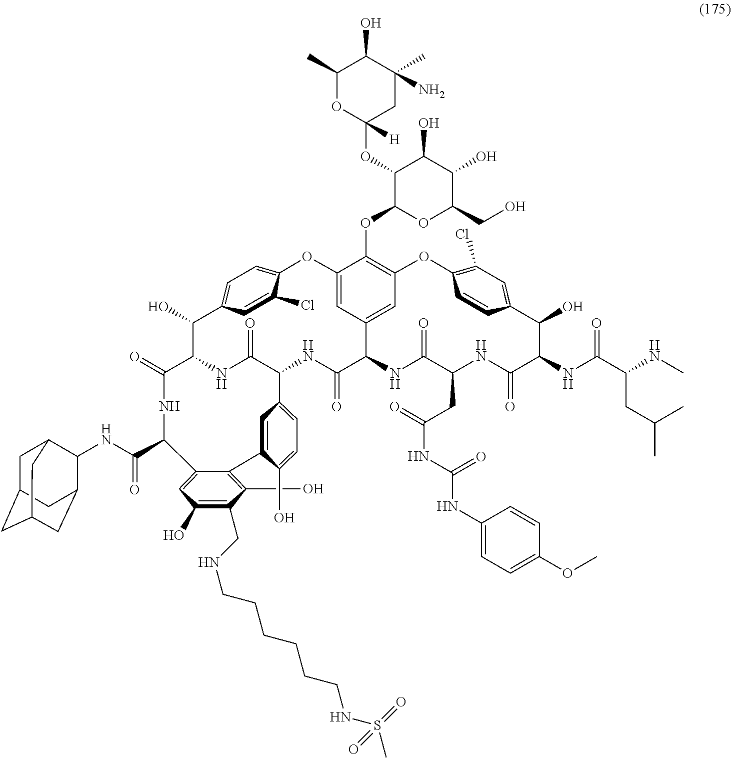

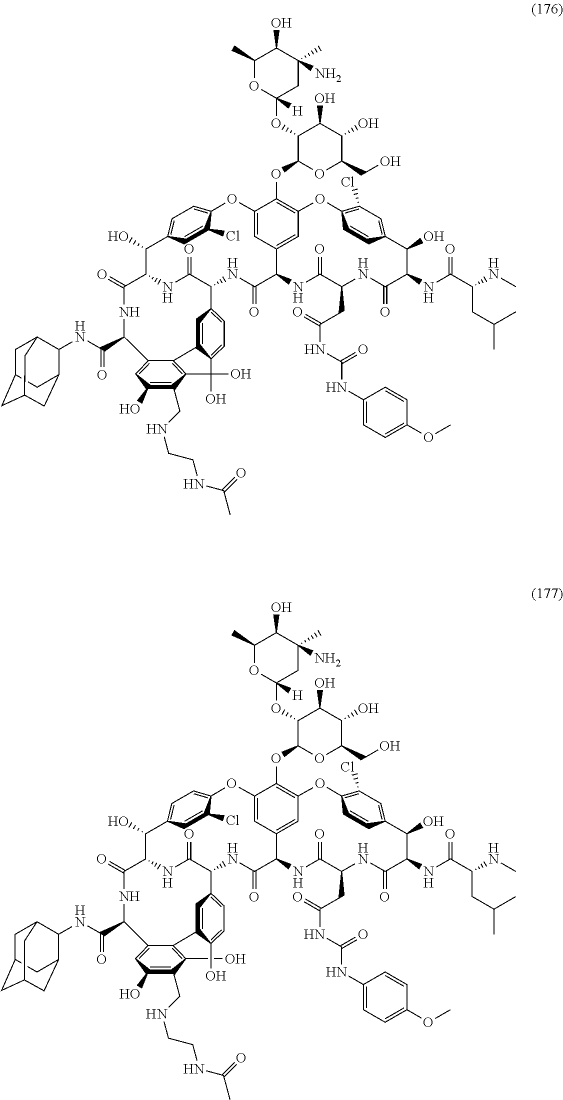

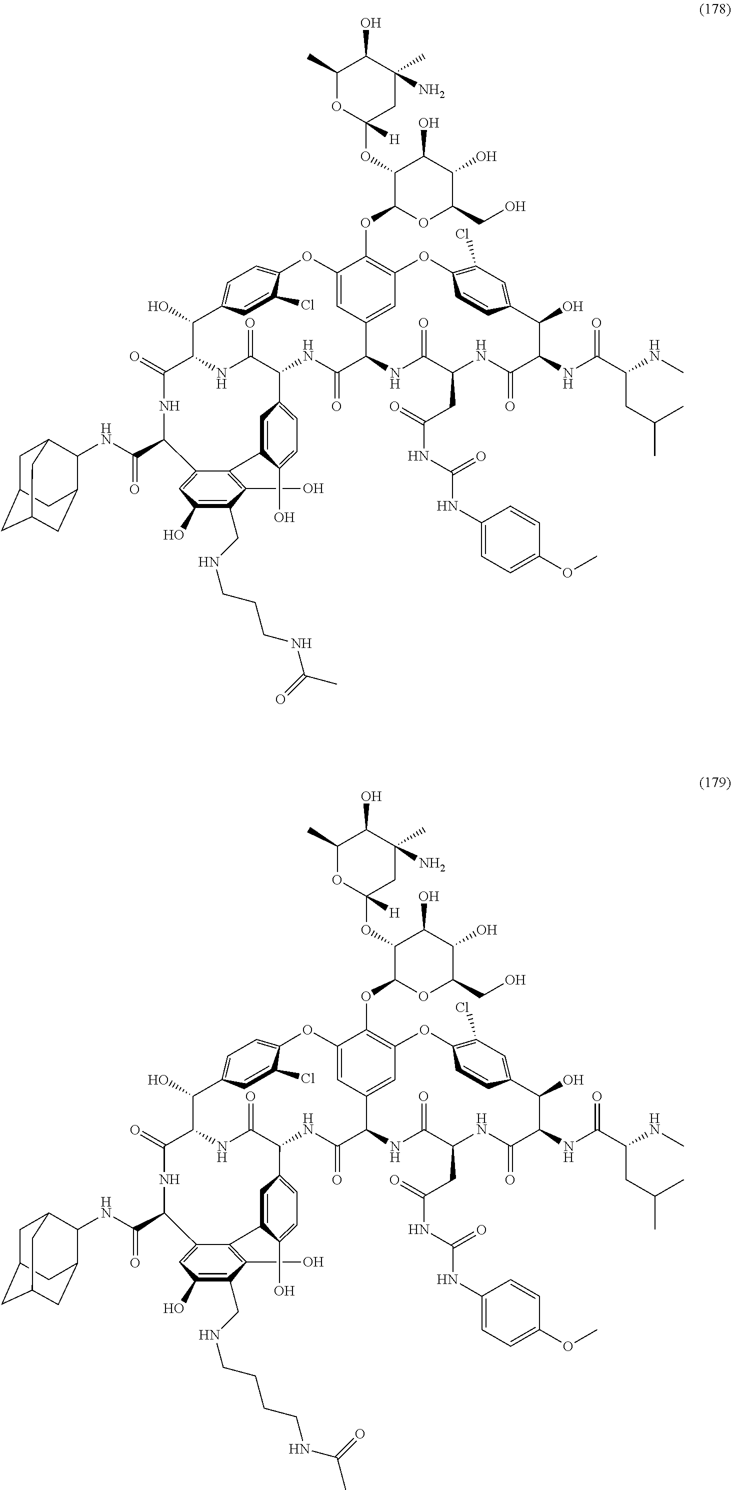

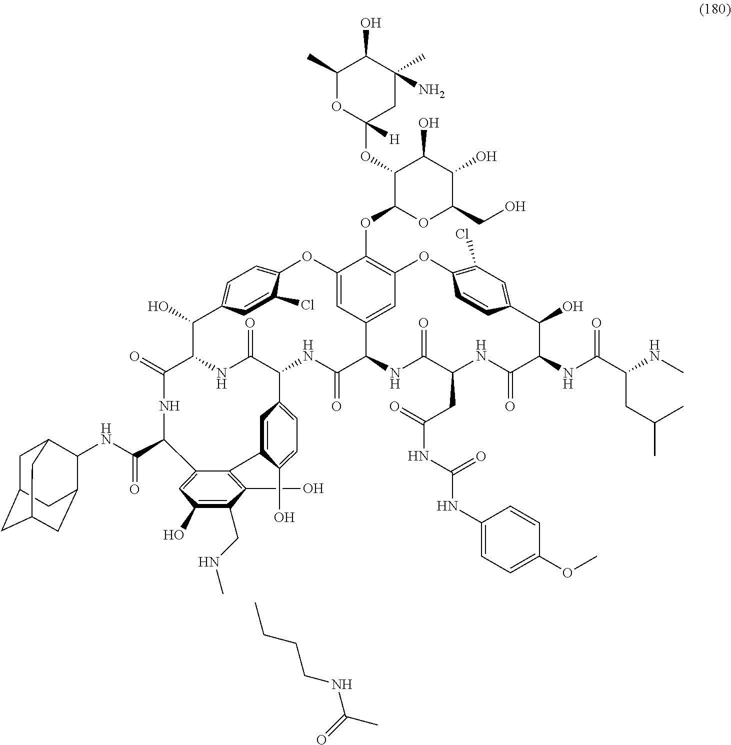

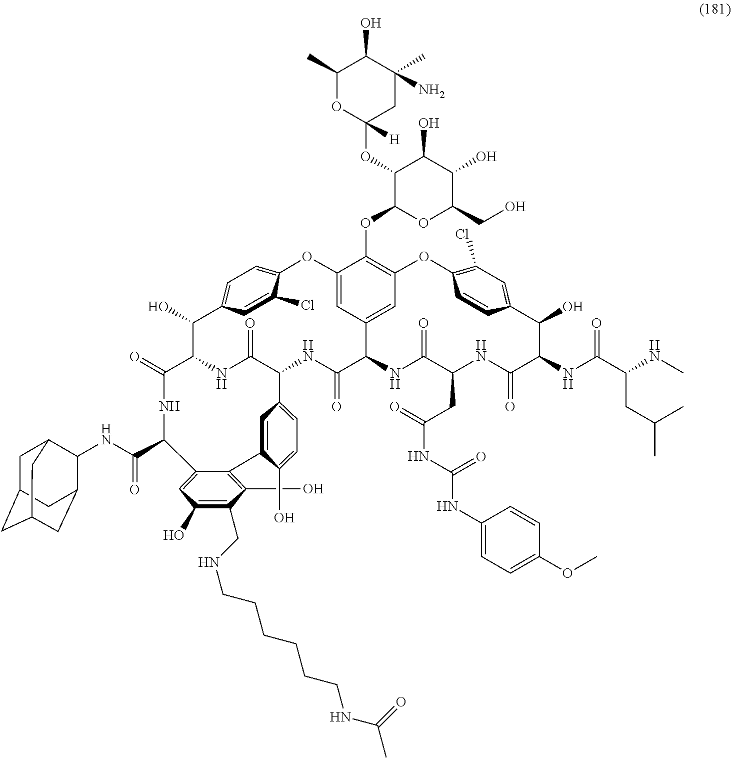

Using a procedure similar to the preparation of Compound (162) as in Example 97 and replacing Compound (139) with Compound (140), and N-(2-aminoethyl)-4-(pentyloxy)benzenesulfonamide with various aminoalkyl sulfonamide or aminoalkylacetamide, Compounds (171), (172), (173), (174), (175) (176), (177, (178), (179), (180) and (181) are prepared.

### Antibacterial Evaluation

Antibacterial activity in vitro is investigated by broth microdilution method in Meuller-Hinton broth as recommended by NCCLS. All strains tested are clinical isolates either sensitive or resistant to natural glycopeptides. MIC values were determined using the CLSI-recommended broth microdilution procedure (Clinical and Laboratory Standards Institute, Methods for Dilution Antimicrobial Susceptibility Tests for Bacteria That Grow Aerobically; Approved Standard-Seventh Edition). Automated liquid handlers (Multidrop 384, Labsystems, Helsinki, Finland; Biomek 2000 and Multimek 96, Beckman Coulter, Fullerton Calif.) were used to conduct serial dilutions and liquid transfers.

Biological data

| | SA | SA | SA | SE | SE | E FC | E FC | E FCM | E FCM | S PNE | S PYO |
|---|---|---|---|---|---|---|---|---|---|---|---|
| # | 100 | 757 | 2012 | 835 | 831 | 101 | 848 | 750 | 752 | 1195 | 712 |
| 29 | 2 | 1 | 1 | 1 | 0.5 | 1 | 2 | 0.5 | 2 | 0.12 | 0.12 |
| 75 | 4 | 2 | 4 | 2 | 1 | 2 | 2 | 0.5 | 2 | 1 | 0.25 |
| 76 | 2 | 2 | 4 | 1 | 1 | 2 | 8 | 0.5 | 8 | 0.25 | 0.06 |

SA 100 = Staphylococcus aureus 100 (MSSA); SA 757 = Staphylococcus aureus 757 (MRSA); SA2012 = Staphylococcus aureus 2012 (VISA); SE 835 = Staphylococcus epidermidis 835 (MSSE); SE 831 = Staphylococcus epidermidis 831 (MRSE); EFC 101 = Enterococcus faecalis 101 (vancomycin sensitive); EFC 848 = Enterococcus faecalis 848 (VRE); EFCM 750 = Enterococcus faecium 750 (vancomycin sensitive); EFCM 752 = Enterococcus faecium 752 (VRE); SPNE 1195 = Streptococcus pneumoniae 1195 (penicillin sensitive); SPYO 712 = Streptococcus pyogenes 712 (penicillin sensitive).

Clinical Trial of the Safety and Efficacy of Compounds of Formula (I)-(XII) in Patients with C. difficile-Associated Diarrhea

Purpose: This study aims to determine the safety and efficacy of glycopeptide compounds presented herein for the treatment of symptoms of C. difficile-associated diarrhea and lowering the risk of repeat episodes of diarrhea. The compounds are evaluated in comparison to current standard antibiotic treatment, so all patients will receive active medication. All study-related care is provided including doctor visits, physical exams, laboratory tests and study medication. Total length of participation is approximately 10 weeks.

Patients: Eligible subjects will be men and women 18 years and older.

### Criteria:

Inclusion Criteria:

Be at least 18 years old;

Have active mild to moderate C. difficile-Associated Diarrhea (CDAD);

Be able to tolerate oral medication;

Not be pregnant or breast-feeding; and

Sign and date an informed consent form.

Study Design: This is a randomized, double-blind, active control study of the efficacy, safety, and tolerability of a compound of Formula (I)-(XII) in patients with C. difficile-associated diarrhea.

Clinical Trial Comparing a Compound of Formula (I)-(XII) with Vancomycin for the Treatment of MRSA Osteomyleitis

Purpose: This study aims to determine the efficacy of glycopeptide compounds presented herein as compared to vancomycin for the treatment of methicillin-resistant Staphylococcus aureus (MRSA) osteomyelitis.

Patients: Eligible subjects will be men and women 18 years and older.

### Criteria:

Inclusion Criteria:

Culture-proven MRSA, obtained in operating room or sterile biopsy procedure from bone site. The infection and sampling site is either within the bone or a deep soft-tissue site that is contiguous with bone; OR radiographic abnormality consistent with osteomyelitis in conjunction with a positive blood culture for MRSA;

Surgical debridement of infection site, as needed;

Subject is capable of providing written informed consent; and

Subject capable of receiving outpatient parenteral therapy for 12 weeks.

Exclusion Criteria:

Hypersensitivity to a compound of Formula (I)-(XII) or vancomycin;

S. aureus resistant to a compound of Formula (I)-(XII) or vancomycin;

Osteomyelitis that develops directly from a chronic, open wound;

Polymicrobial culture (the only exception is if coagulase-negative staphylococcus is present in the culture and the clinical assessment is that it is a contaminant);

Subject has a positive pregnancy test at study enrollment;

Baseline renal or hepatic insufficiency that would preclude administration of study drugs;

Active injection drug use without safe conditions to administer intravenous antibiotics for 3 months; and

Anticipated use of antibiotics for greater than 14 days for an infection other than osteomyelitis.

Study Design: This is a randomized, open-label, active control, efficacy trial comparing vancomycin with a compound of Formula (I)-(XII) for the treatment of MRSA Osteomyelitis.

### Clinical Trial Evaluating a Compound of Formula (I)-(XII) in Selected Serious Infections Caused by Vancomycin-Resistant Enterococcus (VRE)

Purpose: This study aims to determine the safety and efficacy of a compound of Formula (I)-(XII) in the treatment of selected serious infections caused by VRE.

Patients: Eligible subjects will be men and women 18 years and older.

### Criteria:

Inclusion Criteria:

Isolation of one of the following multi-antibiotic resistant bacteria: vancomycin-resistant Enterococcus faecium, vancomycin-resistant Enterococcus faecalis alone or as part of a polymicrobial infection; and

Have a confirmed diagnosis of a serious infection (eg, bacteremia [unless due to an excluded infection], complicated intra-abdominal infection, complicated skin and skin structure infection, or pneumonia) requiring administration of intravenous (IV) antibiotic therapy.

Exclusion Criteria:

Subjects with any concomitant condition or taking any concomitant medication that, in the opinion of the investigator, could preclude an evaluation of a response or make it unlikely that the contemplated course of therapy or follow-up assessment will be completed or that will substantially increase the risk associated with the subject's participation in this study

Anticipated length of antibiotic therapy less than 7 days

Study Design: This is a randomized, double-blind, safety and efficacy study of a compound of Formula (I)-(XII) in the treatment of selected serious infections caused by VRE.

Although the foregoing embodiments have been described in some detail for purposes of clarity of understanding, it will be apparent that in some embodiments, certain changes and modifications are practiced within the scope of the appended claims. It should be noted that there are many alternative ways of implementing both the processes and compositions described herein. Accordingly, the present embodiments are to be considered as illustrative and not restrictive, and the aspects described herein are not to be limited to the details given herein, but in some embodiments are modified within the scope and equivalents of the appended claims.

What is claimed is:

1. A compound having a structure selected from the group consisting of Formulas (I-XII): 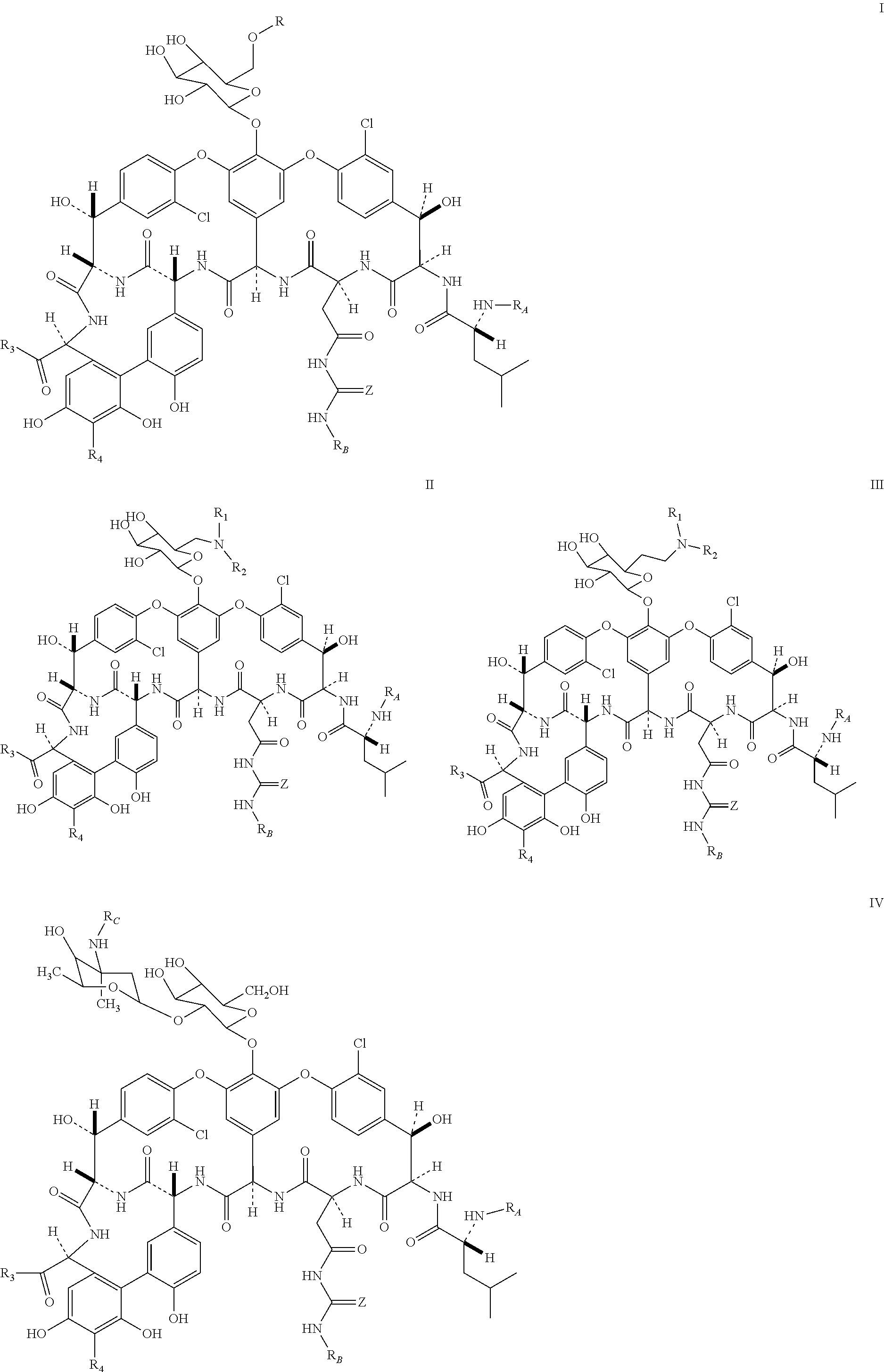 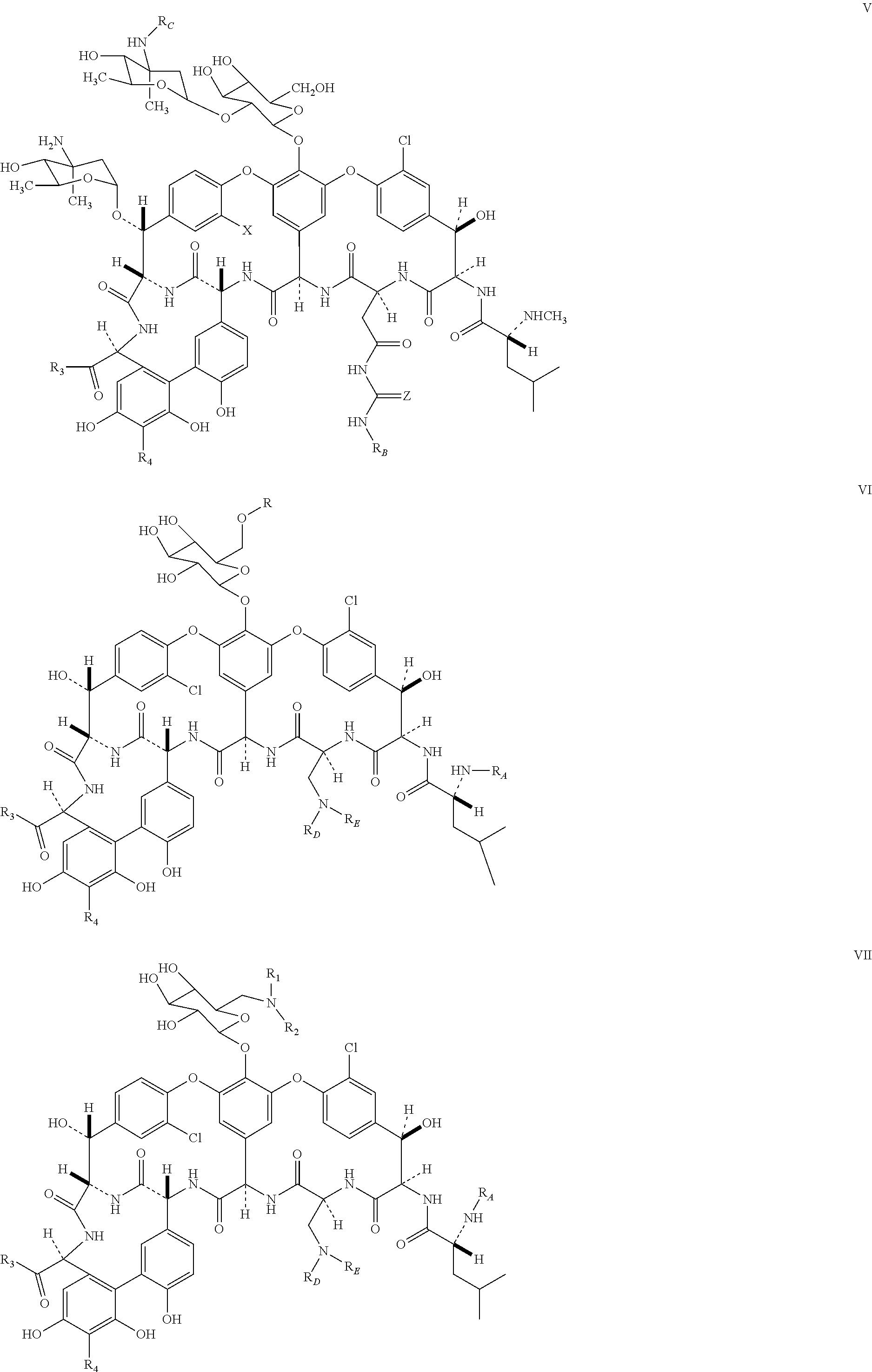 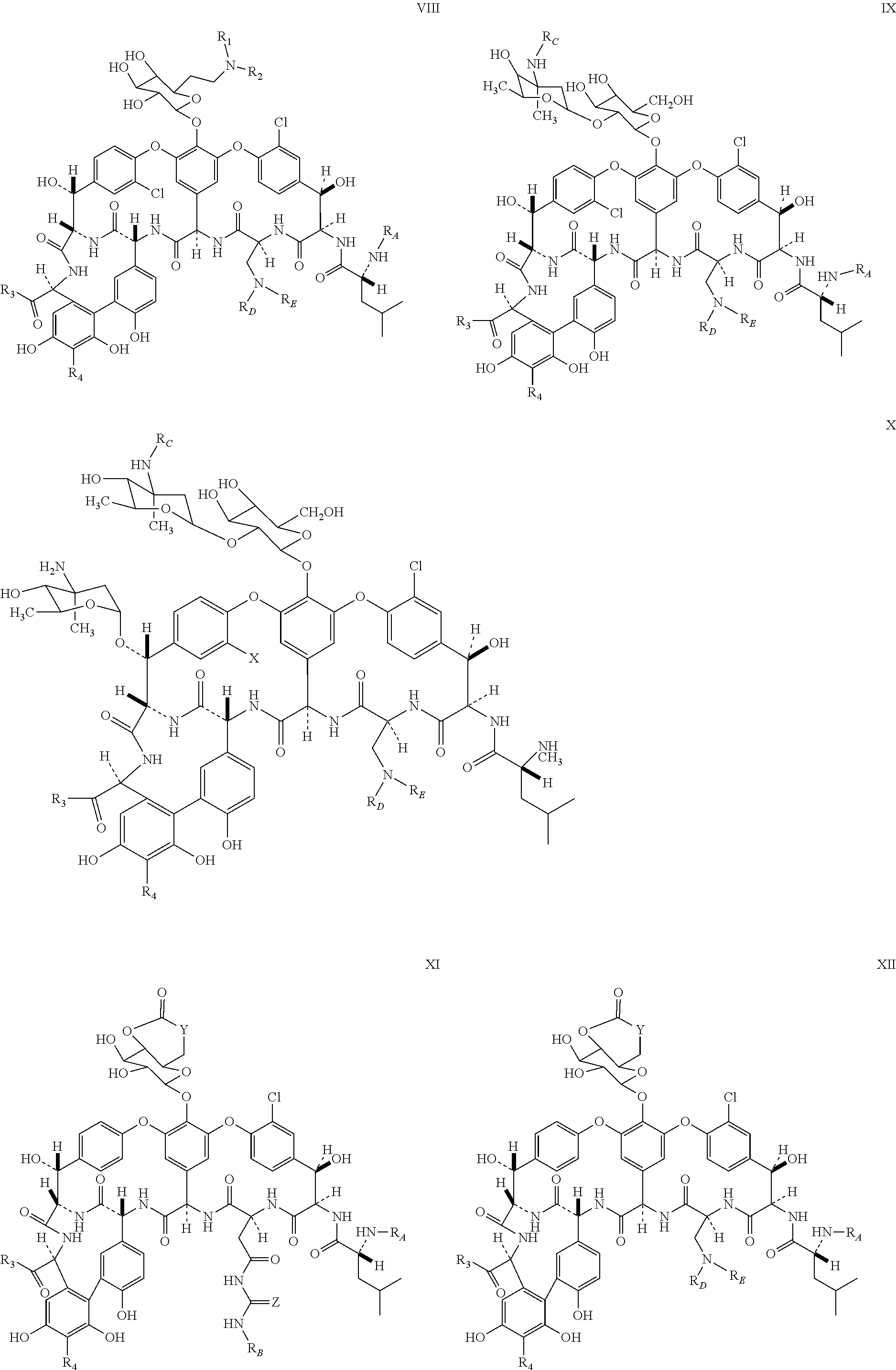
wherein,
R_{A} is selected from the group consisting of
a) hydrogen,
b) methyl,
c) C₂-C₁₂-alkyl;
R₁ and R₂ are each independently selected from the group consisting of
a) hydrogen,
b) C₁-C₁₂-alkyl,
c) C₁-C₁₂-alkyl substituted with one or more substituents selected from the group consisting of
(a) halogen,
(b) hydroxy,
(c) C₁-C₁₂-alkoxy,
(d) C₁-C₃-alkoxy-C₁-C₃-alkoxy,
(e) amino,
(f) C₁-C₁₂-alkylamino,
(g) C₁-C₁₂-dialkylamino,
(h) alkenyl,
(i) alkynyl,
(j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
or
R₁ and R₂ taken together with the atom to which they are attached form a substituted heteroaryl or 3-10 membered heterocycloalkyl ring which optionally contains one to two hetero functionalities selected from the group consisting of —O—, —N—, —NH, —N(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)ₙ— wherein n is 1 or 2 and the 3-10 membered heterocycloalkyl ring is optionally substituted with one or more substituents independently selected from the group consisting of
(a) halogen,
(b) hydroxyl,
(c) C₁-C₃-alkoxy,
(d) C₁-C₃-alkoxy-C₁-C₃-alkoxy,
(e) oxo,
(f) C₁-C₃-alkyl,
(g) halo-C₁-C₃-alkyl,
(h) C₁-C₃-alkoxy-C₁-C₃-alkyl,
and
k) C(═O) R₇,
l) C(═O) CH R₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl,
or
R₈ and R₁₀ or R₉ and R₁₀ taken together with the atom to which they are attached form a 3-10 membered heterocycloalkyl ring which is optionally substituted with one or more substituents independently selected from the group consisting of
(a) halogen,
(b) hydroxyl,
(c) C₁-C₃-alkoxy,
(d) C₁-C₃-alkoxy-C₁-C₃-alkoxy,
(e) oxo,
(f) C₁-C₃-alkyl,
(g) halo-C₁-C₃-alkyl,
(h) C₁-C₃-alkoxy-C₁-C₃-alkyl;
R₇ is selected from the group consisting of
a) hydrogen,
b) C₁-C₁₂-alkyl,
c) C₁-C₁₂-alkyl substituted with one or more substituents selected from the group consisting of
(a) halogen,
(b) hydroxy,
(c) C₁-C₁₂-alkoxy,
(d) C₁-C₃-alkoxy-C₁-C₃-alkoxy,
(e) amino,
(f) C₁-C₁₂-alkylamino,
(g) C₁-C₁₂-dialkylamino,
(h) alkenyl,
(i) alkynyl,
(j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
k) C₁-C₁₂-alkylamino;
X is selected from the group consisting of
(1) hydrogen,
(2) chlorine;
Y is selected from the group consisting of
(1) oxygen,
(2) NR₁, wherein R₁ is as previously defined;
Z is selected from the group consisting of
(1) oxygen,
(2) sulfur;
R is selected from the group consisting of
(1) hydrogen,
(2) cycloalkyl,
(3) cycloalkenyl,
(4) C₁-C₁₂-alkyl,
(5) C₁-C₁₂-alkyl substituted with one or more substituents selected from the group consisting of
(a) halogen,
(b) hydroxy,
(c) C₁-C₁₂-alkoxy,
(d) C₁-C₃-alkoxy-C₁-C₃-alkoxy,
(e) —COOR₅ wherein R₅ is hydrogen or loweralkyl,
(f) —C(O)NR₅R₆ wherein R₅ is as previously defined and R₆ is hydrogen or loweralkyl,
(g) amino,
(h) —NR₅R₆ wherein R₅ and R₆ are as previously defined,
or
R₅ and R₆ are taken together with the atom to which they are attached form a 3-10 membered heterocycloalkyl ring which is optionally substituted with one or more substituents independently selected from the group consisting of
(i) halogen,
(ii) hydroxy,
(iii) C₁-C₃-alkoxy,
(iv) C₁-C₃-alkoxy-C₁-C₃-alkoxy,
(v) oxo,
(vi) C₁-C₁₂-alkyl,
(vii) halo-C₁-C₁₂-alkyl,
and
(viii) C₁-C₃-alkoxy-C₁-C₁₂-alkyl,
(i) aryl,
(j) substituted aryl,
(k) heteroaryl,
(l) substituted heteroaryl,
(m) mercapto,
(n) C₁-C₁₂-thioalkoxy,
(6) C(═O)O R₁₁, wherein R₁₁ is hydrogen, loweralkyl, substituted loweralkyl, aryl, substituted aryl, heteroaryl or substituted heteroaryl,
(7) C(═O)N R₁₁ R₁₂, wherein R₁₁ is as previously defined and R₁₂ is hydrogen, loweralkyl, substituted loweralkyl, aryl, substituted aryl, heteroaryl or substituted heteroaryl,
or
R₁₁ and R₁₂ together with the atom to which they are attached form a 3-10 membered heterocycloalkyl ring, which is optionally substituted with one or more substituents independently selected from the group consisting of
(a) halogen,
(b) hydroxy,
(c) C₁-C₃-alkoxy,
(d) C₁-C₃-alkoxy-C₁-C₃-alkoxy,
(e) oxo,
(f) C₁-C₁₂-alkyl,
(g) substituted loweralkyl,
(h) halo-C₁-C₁₂-alkyl,
(i) amino,
(j) alkylamino,
(k) dialkylamino,
and
(l) C₁-C₃-alkoxy-C₁-C₁₂-alkyl,
or
R and its connected oxygen atom taken together is halogen;
R₃ is selected from the group consisting of
(1) OH,
(2) 1-adamantanamino,
(3) 2-adamantanamino,
(4) 3-amino-1-adamantanamino,
(5) 1-amino-3-adamantanamino,
(6) 3-loweralkylamino-1-adamantanamino,
(7) 1-loweralkylamino-3-adamantanamino,
(8) amino
(9) NR₁₃R₁₄ wherein R₁₃ and R₁₄ are each independently selected from the group consisting of hydrogen, loweralkyl, substituted loweralkyl, cycloalkyl, substituted cycloalkyl, aminoloweralkyl wherein the amino portion of the aminoloweralkyl group is further substituted with unsubstituted or substituted alkyl, alkenyl, cycloalkyl, cycloalkenyl, arylaryl, alkoxy, aryloxy, substituted alkoxy, and substituted aryloxy
or
R₁₃ and R₁₄ together with the atom to which they are attached form a 3-10 membered heterocycloalkyl ring, which is optionally substituted with one or more substituents independently selected from the group consisting of
(a) halogen,
(b) hydroxy,
(c) C₁-C₃-alkoxy,
(d) C₁-C₃-alkoxy-C₁-C₃-alkoxy,
(e) oxo,
(f) C₁-C₁₂-alkyl,
(g) substituted loweralkyl,
(h) halo-C₁-C₁₂-alkyl,
(i) amino,
(j) alkylamino,
(k) dialkylamino,
and
(l) C₁-C₃-alkoxy-C₁-C₁₂-alkyl;
R₄ is selected from the group consisting of
(1) CH₂NH—CHR₁₅—(CH₂)ₘ—NHSO₂R_{B}, wherein m is 1 to 6 and R₁₅ is H or loweralkyl,
(2) CH₂NH—CHR₁₅—(CH₂)ₚ—CONHSO₂R_{B}, wherein p is 0 to 6 and R₁₅ is H or loweralkyl,
(3) CH₂NH—CHR₁₅—(CH₂)ₚ—COOH, wherein p is 0 to 6 and R₁₅ is H or loweralkyl,
(4) CH₂NR_{F}—CHR₁₅—(CH₂)_{q}—NR_{G}SO₂R_{B}, wherein q is 2 to 4 and R₁₅ is H or loweralkyl, R_{F} and R_{G} are independently hydrogen, lower alkyl or taken together represents a —CH₂—,
(5) H,
(6) CH₂NHCH₂PO₃H₂,
(7) aminoloweralkyl wherein the amino portion of the aminoloweralkyl group is further substituted with unsubstituted or substituted alkyl, alkenyl, cycloalkyl, cycloalkenyl, arylaryl, alkoxy, aryloxy, substituted alkoxy, and substituted aryloxy;
R_{B} is selected from the group consisting of
a) aryl,
b) C₁-C₁₂-alkyl,
c) C₁-C₁₂-alkyl substituted with one or more substituents selected from the group consisting of
(a) halogen,
(b) hydroxy,
(c) C₁-C₁₂-alkoxy,
(d) C₁-C₃-alkoxy-C₁-C₃-alkoxy,
(e) amino,
(f) C₁-C₁₂-alkylamino,
(g) C₁-C₁₂-dialkylamino,
(h) alkenyl,
(i) alkynyl,
(j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) heteroaryl,
j) heterocycloalkyl,
k) aryl substituted with one or more substituents selected from the group consisting of
(a) halogen,
(b) hydroxy,
(c) C₁-C₁₂-alkoxy,
(d) C₁-C₆-alkoxy-C₁-C₆-alkoxy,
(e) amino,
(f) amino-C₁-C₆-alkoxy,
(g) C₁-C₁₂-alkylamino,
(h) C₁-C₁₂-alkylamino-C₁-C₆-alkoxy,
(i) C₁-C₁₂-dialkylamino,
(j) C₁-C₁₂-dialkylamino-C₁-C₆-alkoxy,
(k) alkenyl,
(l) alkynyl,
(m) C₁-C₁₂-thioalkoxy,
(n) C₁-C₁₂-alkyl,
l) heteroaryl substituted with one or more substituents selected from the group consisting of
(a) halogen,
(b) hydroxy,
(c) C₁-C₁₂-alkoxy,
(d) C₁-C₆-alkoxy-C₁-C₆-alkoxy,
(e) amino,
(f) amino-C₁-C₆-alkoxy,
(g) C₁-C₁₂-alkylamino,
(h) C₁-C₁₂-alkylamino-C₁-C₆-alkoxy,
(i) C₁-C₁₂-dialkylamino,
(j) C₁-C₁₂-dialkylamino-C₁-C₆-alkoxy,
(k) alkenyl,
(l) alkynyl,
(m) C₁-C₁₂-thioalkoxy,
(n) C₁-C₁₂-alkyl;
R_{C} is each selected from the group consisting of
a) hydrogen,
b) C₁-C₁₂-alkyl,
c) C₁-C₁₂-alkyl substituted with one or more substituents selected from the group consisting of
(a) halogen,
(b) hydroxy,
(c) C₁-C₁₂-alkoxy,
(d) C₁-C₃-alkoxy-C₁-C₃-alkoxy,
(e) amino,
(f) C₁-C₁₂-alkylamino,
(g) C₁-C₁₂-dialkylamino,
(h) alkenyl,
(i) alkynyl,
(j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
k) C(═O) R₇ wherein R₇ is previously defined,
l) C(═O) CHR₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl,
or
R₈ and R₁₀ or R₉ and R₁₀ R₈ and R₁₀ or R₉ and R₁₀ taken together with the atom to which they are attached form a 3-10 membered heterocycloalkyl ring which is optionally substituted with one or more substituents independently selected from the group consisting of
(a) halogen,
(b) hydroxyl,
(c) C₁-C₃-alkoxy,
(d) C₁-C₃-alkoxy-C₁-C₃-alkoxy,
(e) oxo,
(f) C₁-C₃-alkyl,
(g) halo-C₁-C₃-alkyl,
(h) C₁-C₃-alkoxy-C₁-C₃-alkyl;
R_{D} and R_{E} are each independently selected from the group consisting of
a) hydrogen,
b) C₁-C₁₂-alkyl,
c) C₁-C₁₂-alkyl substituted with one or more substituents selected from the group consisting of
(a) halogen,
(b) hydroxy,
(c) C₁-C₁₂-alkoxy,
(d) C₁-C₃-alkoxy-C₁-C₃-alkoxy,
(e) amino,
(f) C₁-C₁₂-alkylamino,
(g) C₁-C₁₂-dialkylamino,
(h) alkenyl,
(i) alkynyl,
(j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
or
R_{D} and R_{E} taken together with the atom to which they are attached form a 3-10 membered heterocycloalkyl ring which optionally contains one to two hetero functionalities selected from the group consisting of —O—, —N—, —NH, —N(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)ₘ— wherein n is 1 or 2 and the 3-10 membered heterocycloalkyl ring is optionally substituted with one or more substituents independently selected from the group consisting of
(a) halogen,
(b) hydroxyl,
(c) C₁-C₃-alkoxy,
(d) C₁-C₃-alkoxy-C₁-C₃-alkoxy,
(e) oxo,
(f) C₁-C₃-alkyl,
(g) halo-C₁-C₃-alkyl,
(h) C₁-C₃-alkoxy-C₁-C₃-alkyl,
and
k) C(═O) R₇ wherein R₇ is previously defined,
l) C(═O) CH R₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl,
or
R₈ and R₁₀ or R₉ and R₁₀ taken together with the atom to which they are attached form a 3-10 membered heterocycloalkyl ring which is optionally substituted with one or more substituents independently selected from the group consisting of
(a) halogen,
(b) hydroxyl,
(c) C₁-C₃-alkoxy,
(d) C₁-C₃-alkoxy-C₁-C₃-alkoxy,
(e) oxo,
(f) C₁-C₃-alkyl,
(g) halo-C₁-C₃-alkyl,
(h) C₁-C₃-alkoxy-C₁-C₃-alkyl,
m) C(═O) CH R₈NR₉R₇ wherein R₇, R₈ and R₉ are as previously defined;
or a pharmaceutically acceptable salt, ester, solvate, alkylated quaternary ammonium salt, stereoisomer, tautomer or prodrug thereof.

2. The compound of claim 1 wherein R_{D} and R_{E} are each independently selected from the group consisting of
a) hydrogen,
b) C₁-C₁₂-alkyl,
c) C₁-C₁₂-alkyl substituted with one or more substituents selected from the group consisting of
(a) halogen,
(b) hydroxy,
(c) C₁-C₁₂-alkoxy,
(d) C₁-C₃-alkoxy-C₁-C₃-alkoxy,
(e) amino,
(f) C₁-C₁₂-alkylamino,
(g) C₁-C₁₂-dialkylamino,
(h) alkenyl,
(i) alkynyl,
(j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
or
R_{D} and R_{E} taken together with the atom to which they are attached form a 3-10 membered heterocycloalkyl ring which optionally contains one to two hetero functionalities selected from the group consisting of —O—, —N—, —NH, —N(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)ₙ— wherein n is 1 or 2 and the 3-10 membered heterocycloalkyl ring is optionally substituted with one or more substituents independently selected from the group consisting of
(a) halogen,
(b) hydroxyl,
(c) C₁-C₃-alkoxy,
(d) C₁-C₃-alkoxy-C₁-C₃-alkoxy,
(e) oxo,
(f) C₁-C₃-alkyl,
(g) halo-C₁-C₃-alkyl,
(h) C₁-C₃-alkoxy-C₁-C₃-alkyl,
and
k) C(═O) R₇ wherein R₇ is previously defined,
l) C(═O) CH R₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl,
or
R₈ and R₁₀ or R₉ and R₁₀ taken together with the atom to which they are attached form a 3-10 membered heterocycloalkyl ring which is optionally substituted with one or more substituents independently selected from the group consisting of
(a) halogen,
(b) hydroxyl,
(c) C₁-C₃-alkoxy,
(d) C₁-C₃-alkoxy-C₁-C₃-alkoxy,
(e) oxo,
(f) C₁-C₃-alkyl,
(g) halo-C₁-C₃-alkyl,
(h) C₁-C₃-alkoxy-C₁-C₃-alkyl,
m) C(═O) CH R₈NR₉R₇ wherein R₇, R₈ and R₉ are as previously defined.

3. The compound of claim 1 wherein the compound is of Formula I and R is selected from the group consisting of
(1) hydrogen,
(2) cycloalkyl,
(3) cycloalkenyl,
(4) C₁-C₁₂-alkyl,
(5) C₁-C₁₂-alkyl substituted with one or more substituents selected from the group consisting of
(a) halogen,
(b) hydroxy,
(c) C₁-C₁₂-alkoxy,
(d) C₁-C₃-alkoxy-C₁-C₃-alkoxy,
(e) —COOR₅ wherein R₅ is hydrogen or loweralkyl,
(f) —C(O)NR₅R₆ wherein R₅ is as previously defined and R₆ is hydrogen or loweralkyl,
(g) amino,
(h) —NR₅R₆ wherein R₅ and R₆ are as previously defined,
or
R₅ and R₆ are taken together with the atom to which they are attached form a 3-10 membered heterocycloalkyl ring which is optionally substituted with one or more substituents independently selected from the group consisting of
(i) halogen,
(ii) hydroxy,
(iii) C₁-C₃-alkoxy,
(iv) C₁-C₃-alkoxy-C₁-C₃-alkoxy,
(v) oxo,
(vi) C₁-C₁₂-alkyl,
(vii) halo-C₁-C₁₂-alkyl,
and
(viii) C₁-C₃-alkoxy-C₁-C₁₂-alkyl,
(i) aryl,
(j) substituted aryl,
(k) heteroaryl,
(l) substituted heteroaryl,
(m) mercapto,
(n) C₁-C₁₂-thioalkoxy,
(6) C(═O)O R₁₁, wherein R₁₁ is hydrogen, loweralkyl, substituted loweralkyl, aryl, substituted aryl, heteroaryl or substituted heteroaryl,
(7) C(═O)N R₁₁ R₁₂, wherein R₁₁ is as previously defined and R₁₂ is hydrogen, loweralkyl, substituted loweralkyl, aryl, substituted aryl, heteroaryl or substituted heteroaryl,
or
R₁₁ and R₁₂ together with the atom to which they are attached form a 3-10 membered heterocycloalkyl ring, which is optionally substituted with one or more substituents independently selected from the group consisting of
(a) halogen,
(b) hydroxy,
(c) C₁-C₃-alkoxy,
(d) C₁-C₃-alkoxy-C₁-C₃-alkoxy,
(e) oxo,
(f) C₁-C₁₂-alkyl,
(g) substituted loweralkyl,
(h) halo-C₁-C₁₂-alkyl,
(i) amino,
(j) alkylamino,
(k) dialkylamino,
and
(l) C₁-C₃-alkoxy-C₁-C₁₂-alkyl,
or
R and its connected oxygen atom taken together is halogen.

4. The compound of claim 1 where the compound is according to Formula IX and R_{C} is selected from the group consisting of
a) hydrogen,
b) C₁-C₁₂-alkyl,
c) C₁-C₁₂-alkyl substituted with one or more substituents selected from the group consisting of
(a) halogen,
(b) hydroxy,
(c) C₁-C₁₂-alkoxy,
(d) C₁-C₃-alkoxy-C₁-C₃-alkoxy,
(e) amino,
(f) C₁-C₁₂-alkylamino,
(g) C₁-C₁₂-dialkylamino,
(h) alkenyl,
(i) alkynyl,
(j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
k) C(═O) R₇ wherein R₇ is previously defined,
l) C(═O) CHR₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl,
or
R₈ and R₁₀ or R₉ and R₁₀ taken together with the atom to which they are attached form a 3-10 membered heterocycloalkyl ring which is optionally substituted with one or more substituents independently selected from the group consisting of
(a) halogen,
(b) hydroxyl,
(c) C₁-C₃-alkoxy,
(d) C₁-C₃-alkoxy-C₁-C₃-alkoxy,
(e) oxo,
(f) C₁-C₃-alkyl,
(g) halo-C₁-C₃-alkyl,
(h) C₁-C₃-alkoxy-C₁-C₃-alkyl.

5. The compound of claim 1 wherein the compound is according to Formula X and R_{C} is selected from the group consisting of
a) hydrogen,
b) C₁-C₁₂-alkyl,
c) C₁-C₁₂-alkyl substituted with one or more substituents selected from the group consisting of
(a) halogen,
(b) hydroxy,
(c) C₁-C₁₂-alkoxy,
(d) C₁-C₃-alkoxy-C₁-C₃-alkoxy,
(e) amino,
(f) C₁-C₁₂-alkylamino,
(g) C₁-C₁₂-dialkylamino,
(h) alkenyl,
(i) alkynyl,
(j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
k) C(═O) R₇ wherein R₇ is previously defined,
l) C(═O) CHR₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl,
or
R₈ and R₁₀ or R₉ and R₁₀ taken together with the atom to which they are attached form a 3-10 membered heterocycloalkyl ring which is optionally substituted with one or more substituents independently selected from the group consisting of
(a) halogen,
(b) hydroxyl,
(c) C₁-C₃-alkoxy,
(d) C₁-C₃-alkoxy-C₁-C₃-alkoxy,
(e) oxo,
(f) C₁-C₃-alkyl,
(g) halo-C₁-C₃-alkyl,
(h) C₁-C₃-alkoxy-C₁-C₃-alkyl.

6. The compound of claim 1 wherein the compound is according to Formula II and R₁ and R₂ are each independently selected from the group consisting of
a) hydrogen,
b) C₁-C₁₂-alkyl,
c) C₁-C₁₂-alkyl substituted with one or more substituents selected from the group consisting of
(a) halogen,
(b) hydroxy,
(c) C₁-C₁₂-alkoxy,
(d) C₁-C₃-alkoxy-C₁-C₃-alkoxy,
(e) amino,
(f) C₁-C₁₂-alkylamino,
(g) C₁-C₁₂-dialkylamino,
(h) alkenyl,
(i) alkynyl,
(j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
or
R₁ and R₂ taken together with the atom to which they are attached form a substituted heteroaryl or 3-10 membered heterocycloalkyl ring which optionally contains one to two hetero functionalities selected from the group consisting of —O—, —N—, —NH, —N(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)ₙ— wherein n is 1 or 2 and the 3-10 membered heterocycloalkyl ring is optionally substituted with one or more substituents independently selected from the group consisting of
(a) halogen,
(b) hydroxyl,
(c) C₁-C₃-alkoxy,
(d) C₁-C₃-alkoxy-C₁-C₃-alkoxy,
(e) oxo,
(f) C₁-C₃-alkyl,
(g) halo-C₁-C₃-alkyl,
(h) C₁-C₃-alkoxy-C₁-C₃-alkyl,
and
k) C(═O) R₇,
l) C(═O) CH R₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl,
or
R₈ and R₁₀ or R₉ and R₁₀ taken together with the atom to which they are attached form a 3-10 membered heterocycloalkyl ring which is optionally substituted with one or more substituents independently selected from the group consisting of
(a) halogen,
(b) hydroxyl,
(c) C₁-C₃-alkoxy,
(d) C₁-C₃-alkoxy-C₁-C₃-alkoxy,
(e) oxo,
(f) C₁-C₃-alkyl,
(g) halo-C₁-C₃-alkyl,
(h) C₁-C₃-alkoxy-C₁-C₃-alkyl.

7. The compound of claim 1 wherein the compound is according to Formula II and R₁ and R₂ are each independently selected from the group consisting of
a) hydrogen,
b) C₁-C₁₂-alkyl,
c) C₁-C₁₂-alkyl substituted with one or more substituents selected from the group consisting of
(a) halogen,
(b) hydroxy,
(c) C₁-C₁₂-alkoxy,
(d) C₁-C₃-alkoxy-C₁-C₃-alkoxy,
(e) amino,
(f) C₁-C₁₂-alkylamino,
(g) C₁-C₁₂-dialkylamino,
(h) alkenyl,
(i) alkynyl,
(j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
or
R₁ and R₂ taken together with the atom to which they are attached form a substituted heteroaryl or 3-10 membered heterocycloalkyl ring which optionally contains one to two hetero functionalities selected from the group consisting of —O—, —N—, —NH, —N(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)ₙ— wherein n is 1 or 2 and the 3-10 membered heterocycloalkyl ring is optionally substituted with one or more substituents independently selected from the group consisting of
(a) halogen,
(b) hydroxyl,
(c) C₁-C₃-alkoxy,
(d) C₁-C₃-alkoxy-C₁-C₃-alkoxy,
(e) oxo,
(f) C₁-C₃-alkyl,
(g) halo-C₁-C₃-alkyl,
(h) C₁-C₃-alkoxy-C₁-C₃-alkyl,
and
k) C(═O) R₇,
l) C(═O) CH R₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl,
or
R₈ and R₁₀ or R₉ and R₁₀ taken together with the atom to which they are attached form a 3-10 membered heterocycloalkyl ring which is optionally substituted with one or more substituents independently selected from the group consisting of
(a) halogen,
(b) hydroxyl,
(c) C₁-C₃-alkoxy,
(d) C₁-C₃-alkoxy-C₁-C₃-alkoxy,
(e) oxo,
(f) C₁-C₃-alkyl,
(g) halo-C₁-C₃-alkyl,
(h) C₁-C₃-alkoxy-C₁-C₃-alkyl.

8. The compound of claim 1 wherein the compound is according to Formula VII and R₁ and R₂ are each independently selected from the group consisting of
a) hydrogen,
b) C₁-C₁₂-alkyl,
c) C₁-C₁₂-alkyl substituted with one or more substituents selected from the group consisting of
(a) halogen,
(b) hydroxy,
(c) C₁-C₁₂-alkoxy,
(d) C₁-C₃-alkoxy-C₁-C₃-alkoxy,
(e) amino,
(f) C₁-C₁₂-alkylamino,
(g) C₁-C₁₂-dialkylamino
(h) alkenyl,
(i) alkynyl,
(j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
or
R₁ and R₂ taken together with the atom to which they are attached form a substituted heteroaryl or 3-10 membered heterocycloalkyl ring which optionally contains one to two hetero functionalities selected from the group consisting of —O—, —N—, —NH, —N(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)ₙ— wherein n is 1 or 2 and the 3-10 membered heterocycloalkyl ring is optionally substituted with one or more substituents independently selected from the group consisting of
(a) halogen,
(b) hydroxyl,
(c) C₁-C₃-alkoxy,
(d) C₁-C₃-alkoxy-C₁-C₃-alkoxy,
(e) oxo,
(f) C₁-C₃-alkyl,
(g) halo-C₁-C₃-alkyl,
(h) C₁-C₃-alkoxy-C₁-C₃-alkyl,
and
k) C(═O) R₇,
l) C(═O) CH R₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl,
or
R₈ and R₁₀ or R₉ and R₁₀ taken together with the atom to which they are attached form a 3-10 membered heterocycloalkyl ring which is optionally substituted with one or more substituents independently selected from the group consisting of
(a) halogen,
(b) hydroxyl,
(c) C₁-C₃-alkoxy,
(d) C₁-C₃-alkoxy-C₁-C₃-alkoxy,
(e) oxo,
(f) C₁-C₃-alkyl,
(g) halo-C₁-C₃-alkyl,
(h) C₁-C₃-alkoxy-C₁-C₃-alkyl.

9. The compound of claim 1 wherein the compound is according to Formula VIII and R₁ and R₂ are each independently selected from the group consisting of
a) hydrogen,
b) C₁-C₁₂-alkyl,
c) C₁-C₁₂-alkyl substituted with one or more substituents selected from the group consisting of
(a) halogen,
(b) hydroxy,
(c) C₁-C₁₂-alkoxy,
(d) C₁-C₃-alkoxy-C₁-C₃-alkoxy,
(e) amino,
(f) C₁-C₁₂-alkylamino,
(g) C₁-C₁₂-dialkylamino,
(h) alkenyl,
(i) alkynyl,
C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
or
R₁ and R₂ taken together with the atom to which they are attached form a substituted heteroaryl or 3-10 membered heterocycloalkyl ring which optionally contains one to two hetero functionalities selected from the group consisting of —O—, —N—, —NH, —N(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)ₙ— wherein n is 1 or 2 and the 3-10 membered heterocycloalkyl ring is optionally substituted with one or more substituents independently selected from the group consisting of
(a) halogen,
(b) hydroxyl,
(c) C₁-C₃-alkoxy,
(d) C₁-C₃-alkoxy-C₁-C₃-alkoxy,
(e) oxo,
(f) C₁-C₃-alkyl,
(g) halo-C₁-C₃-alkyl,
(h) C₁-C₃-alkoxy-C₁-C₃-alkyl,
and
k) C(═O) R₇,
l) C(═O) CH R₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl,
or
R₈ and R₁₀ or R₉ and R₁₀ taken together with the atom to which they are attached form a 3-10 membered heterocycloalkyl ring which is optionally substituted with one or more substituents independently selected from the group consisting of
(a) halogen,
(b) hydroxyl,
(c) C₁-C₃-alkoxy,
(d) C₁-C₃-alkoxy-C₁-C₃-alkoxy,
(e) oxo,
(f) C₁-C₃-alkyl,
(g) halo-C₁-C₃-alkyl,
(h) C₁-C₃-alkoxy-C₁-C₃-alkyl.

10. The compound of claim 1 wherein Z is selected from the group consisting of oxygen and sulfur.

11. A compound selected from the group consisting of:

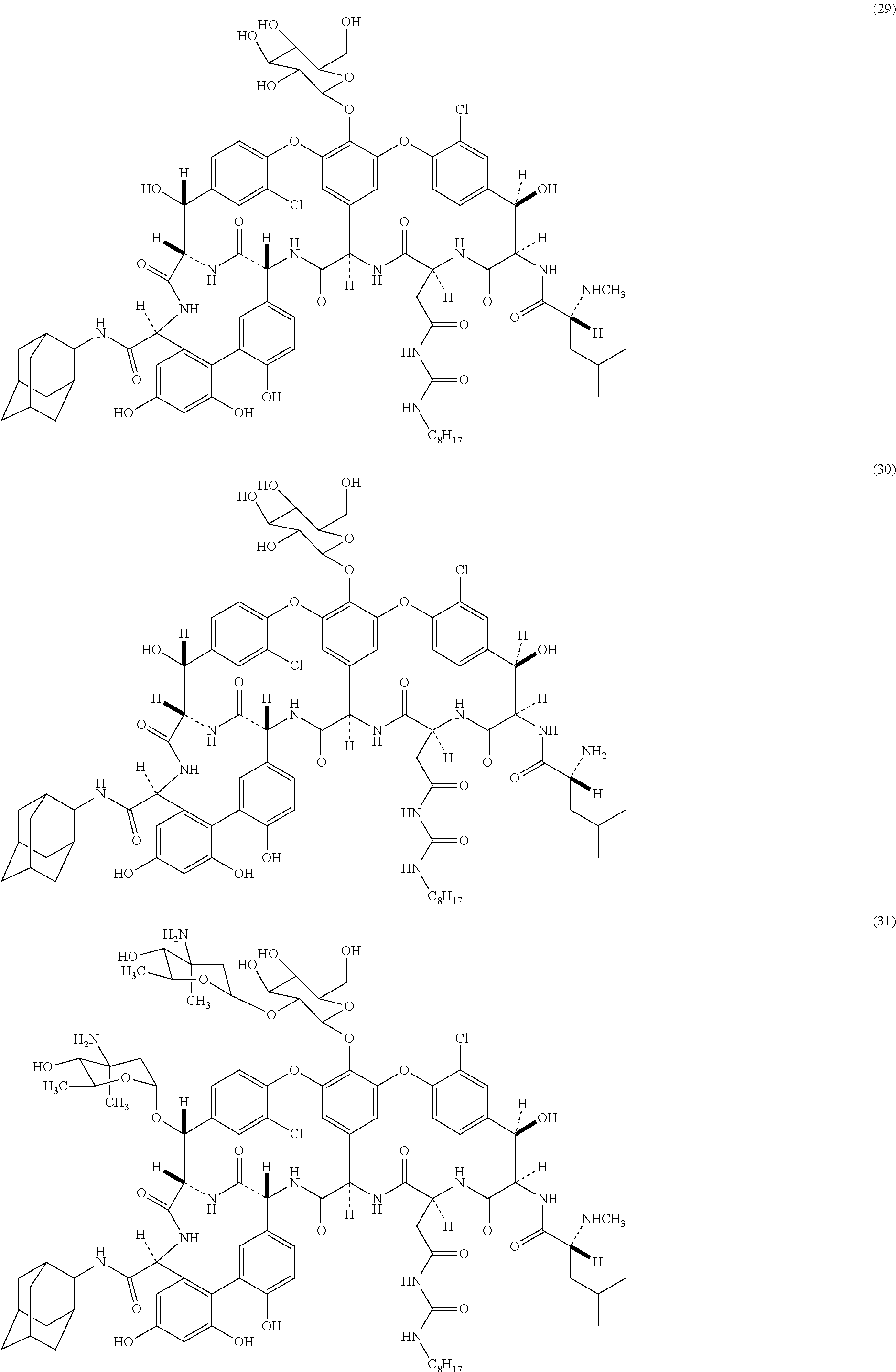

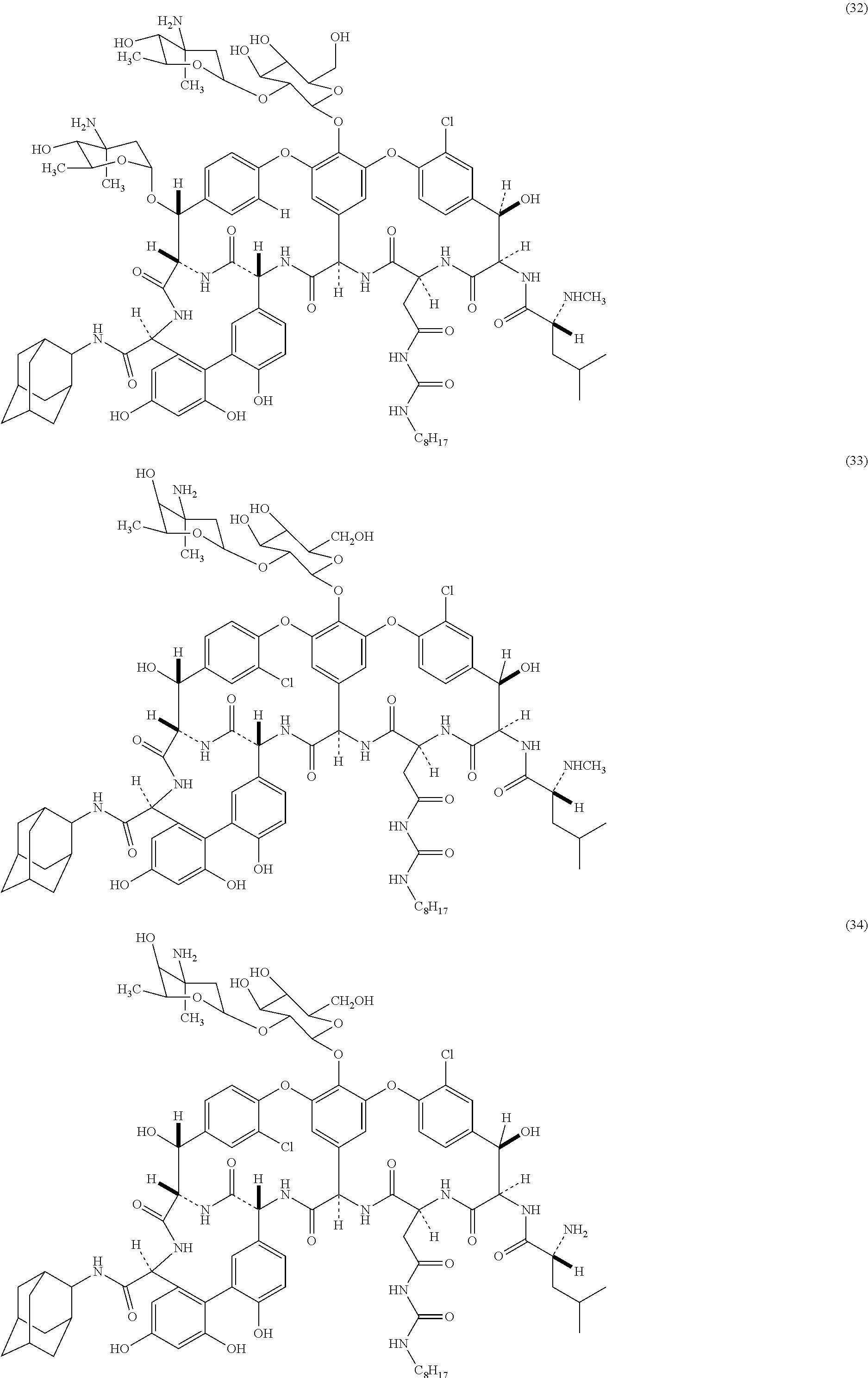

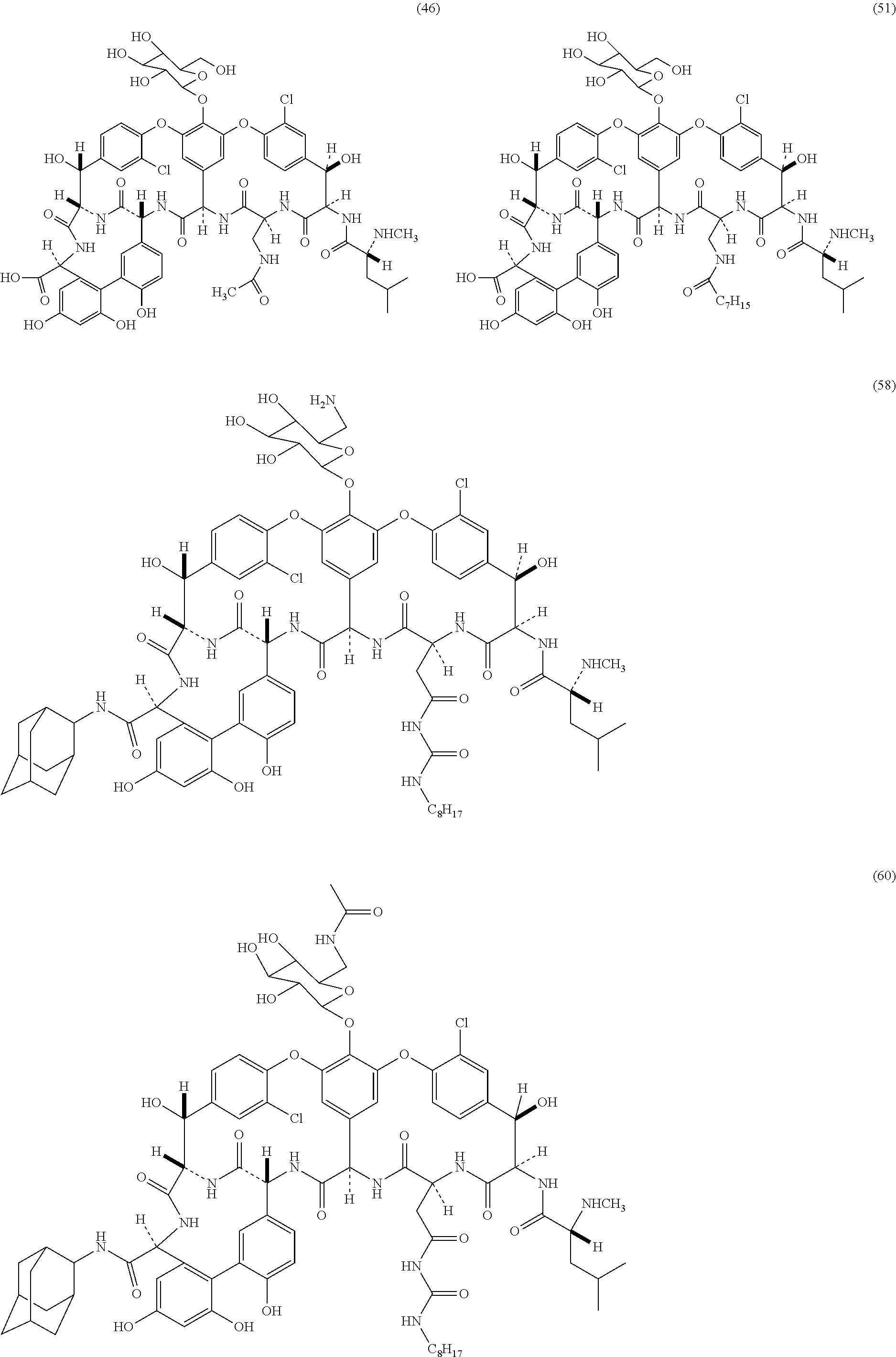

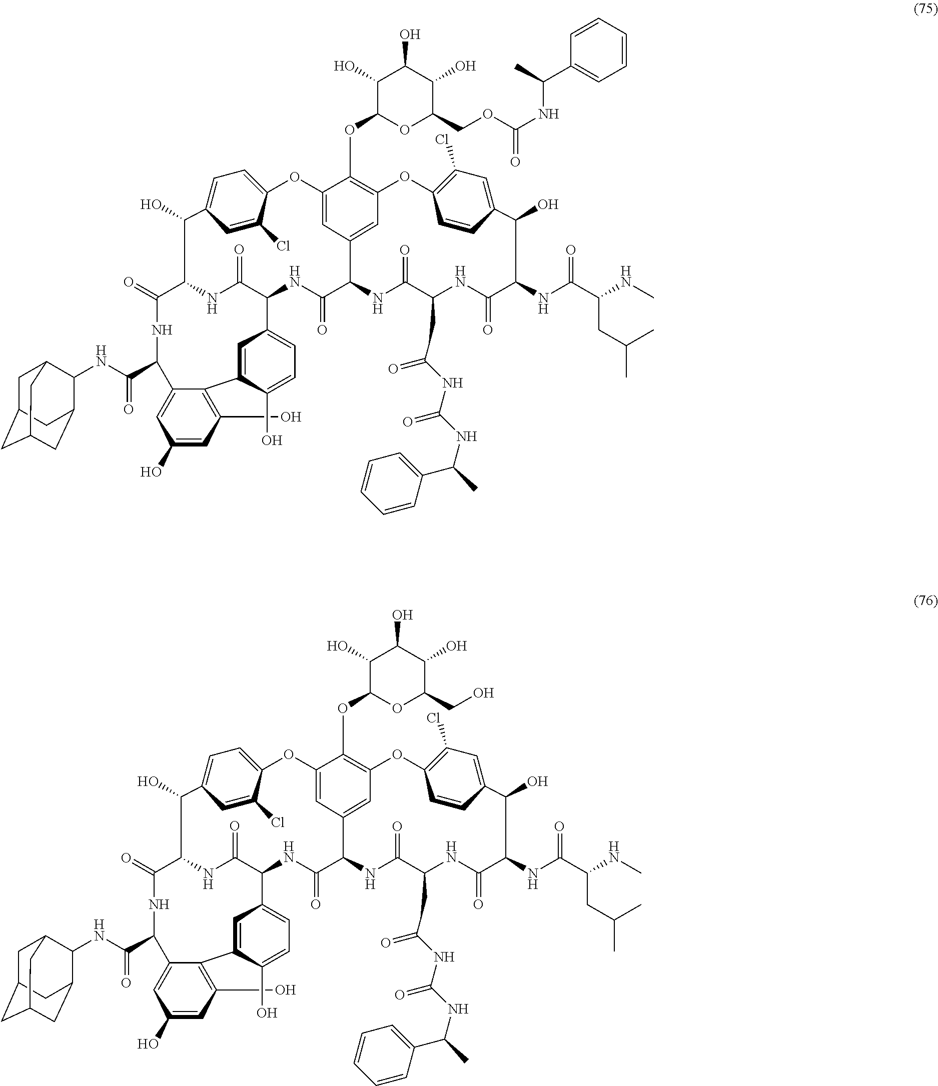

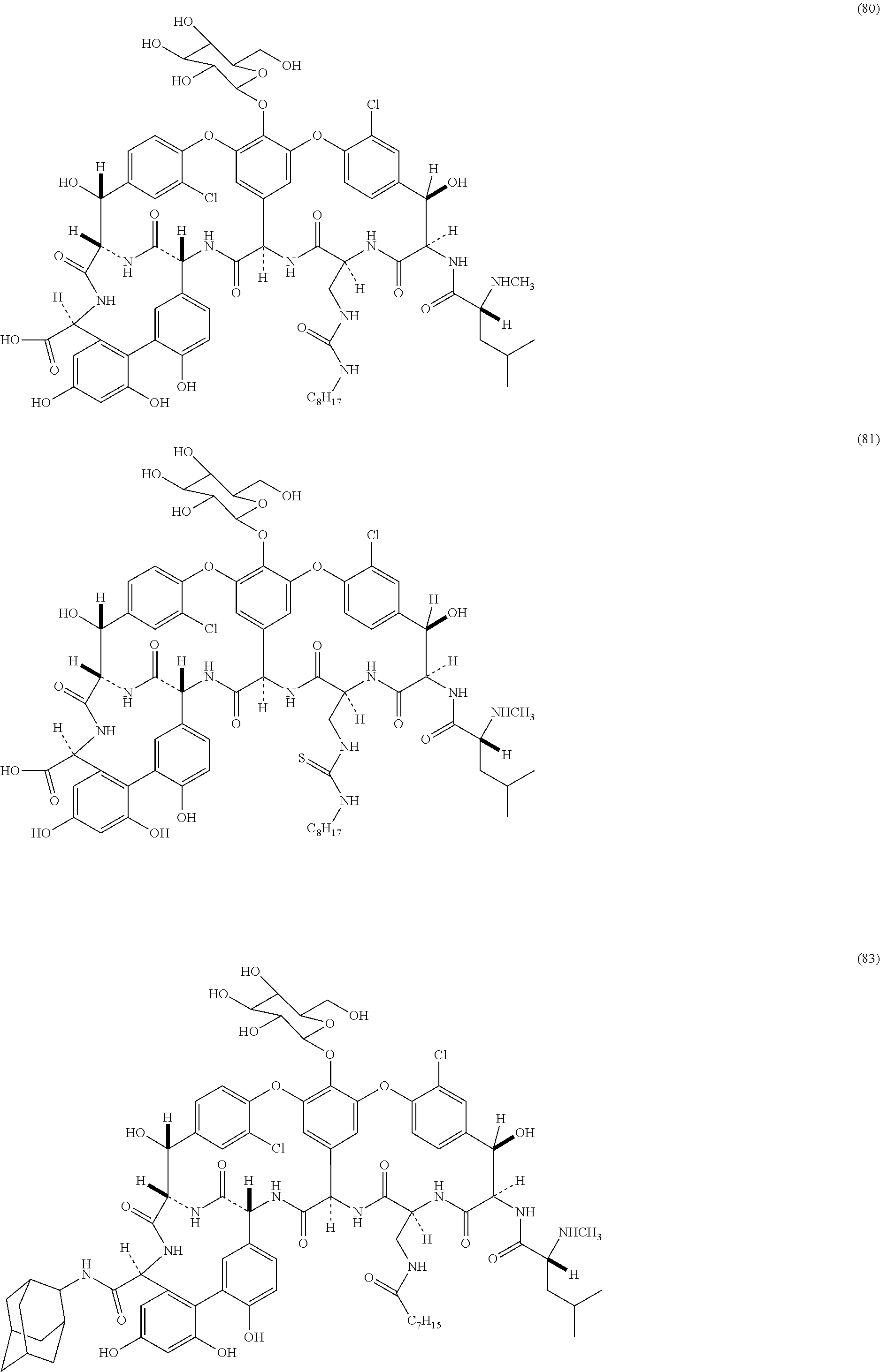

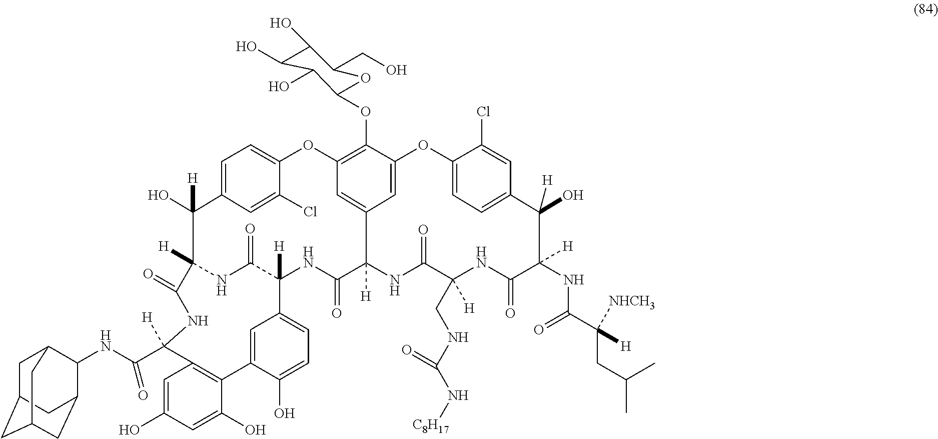

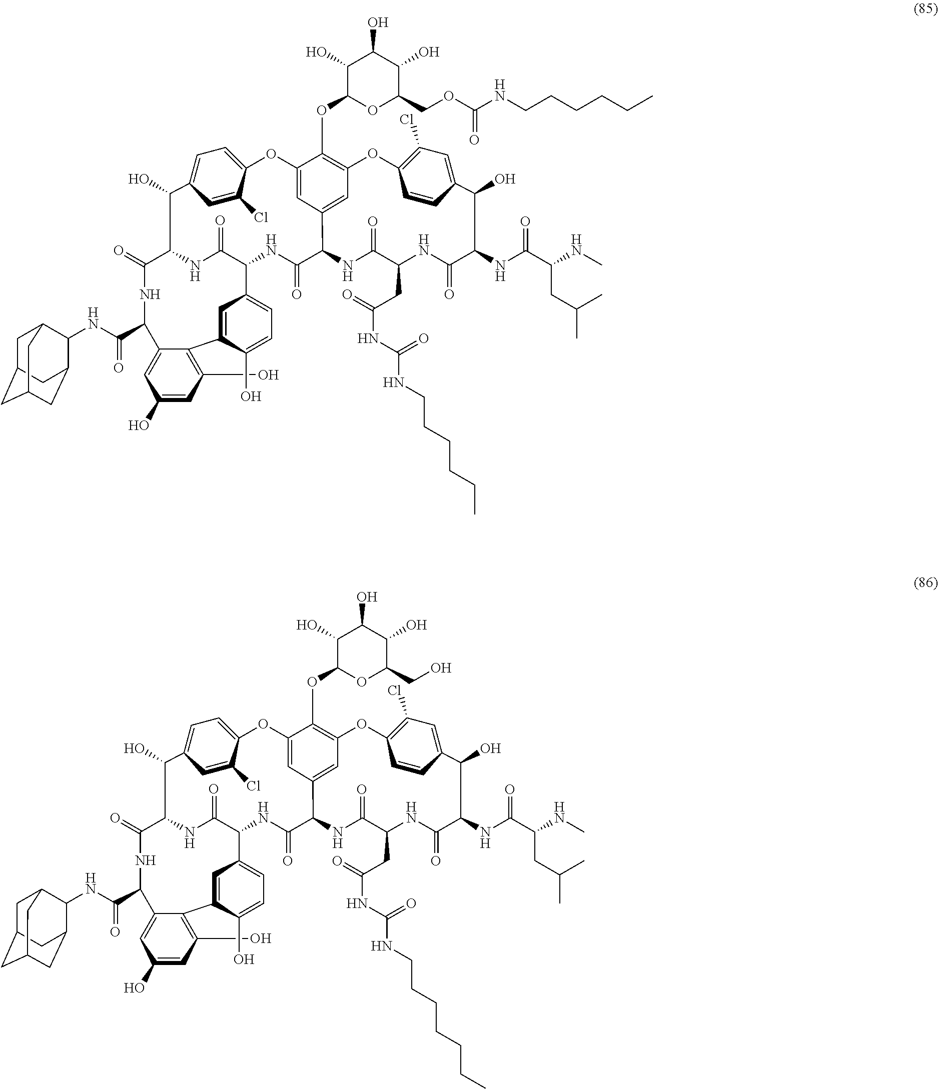

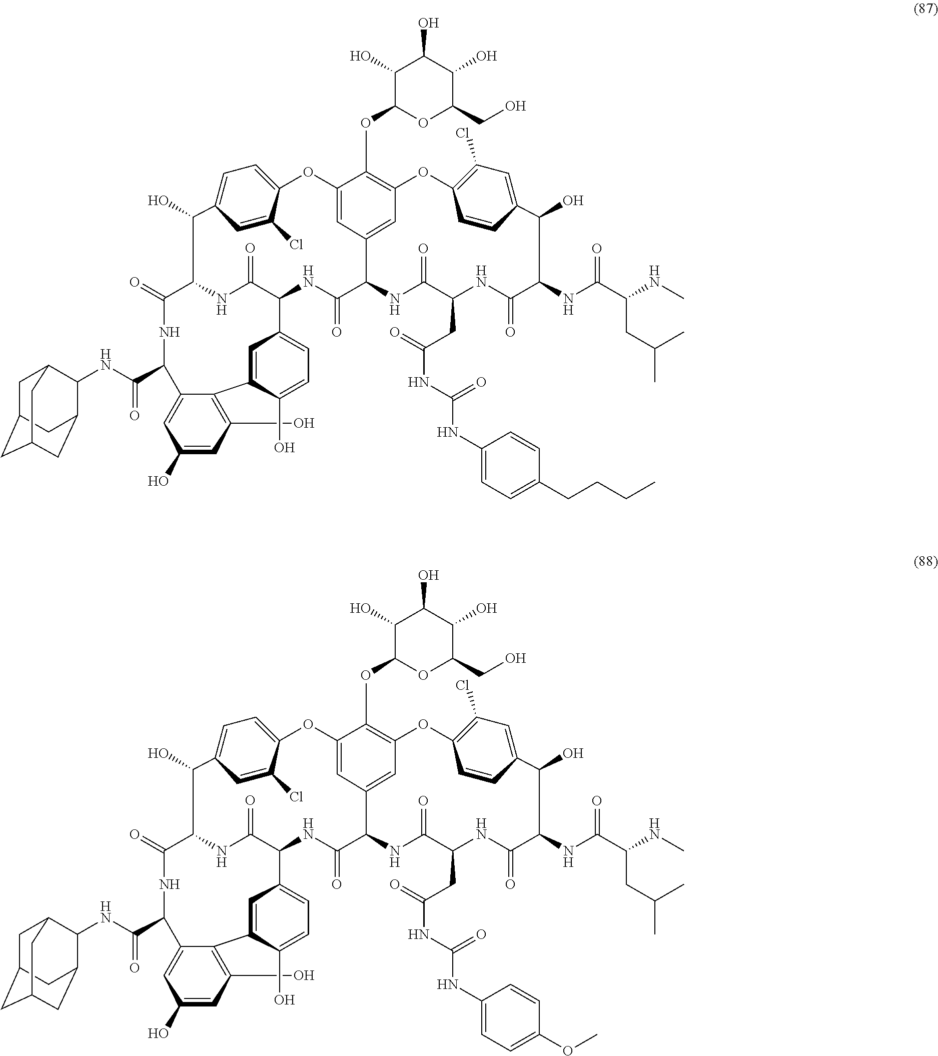

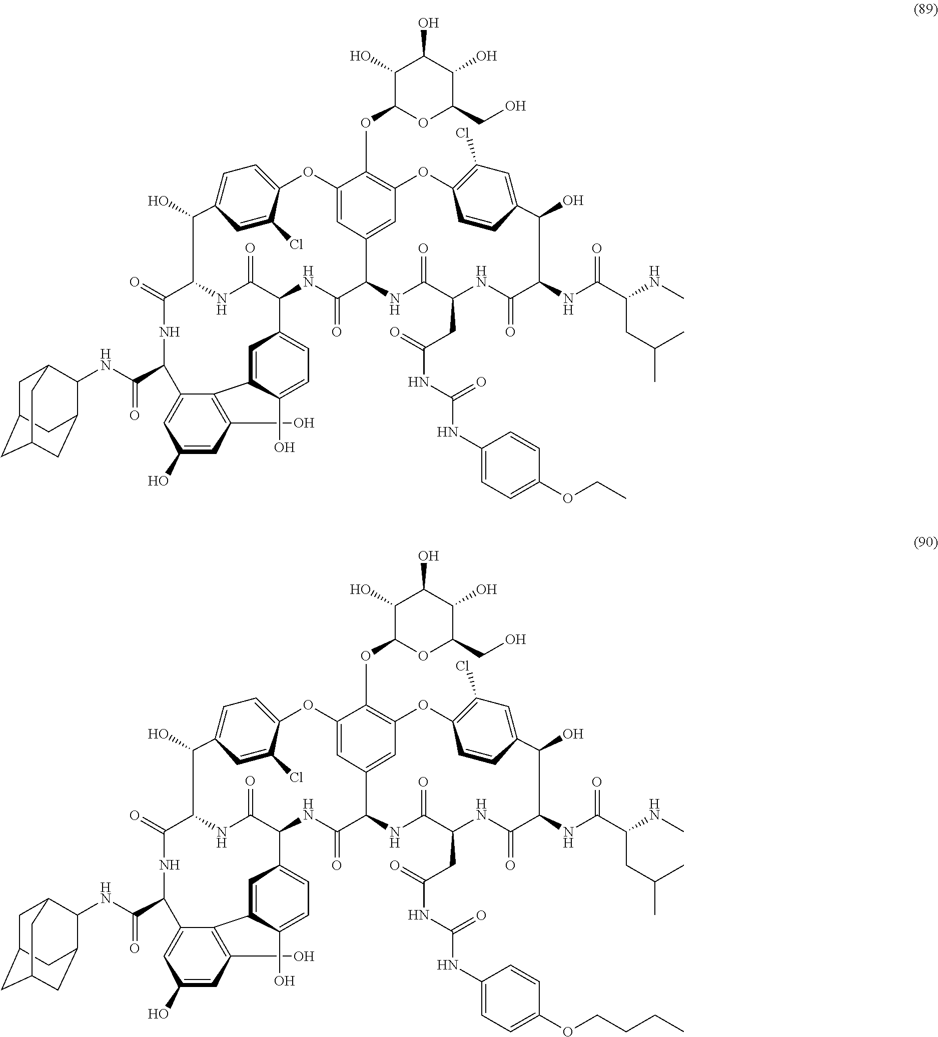

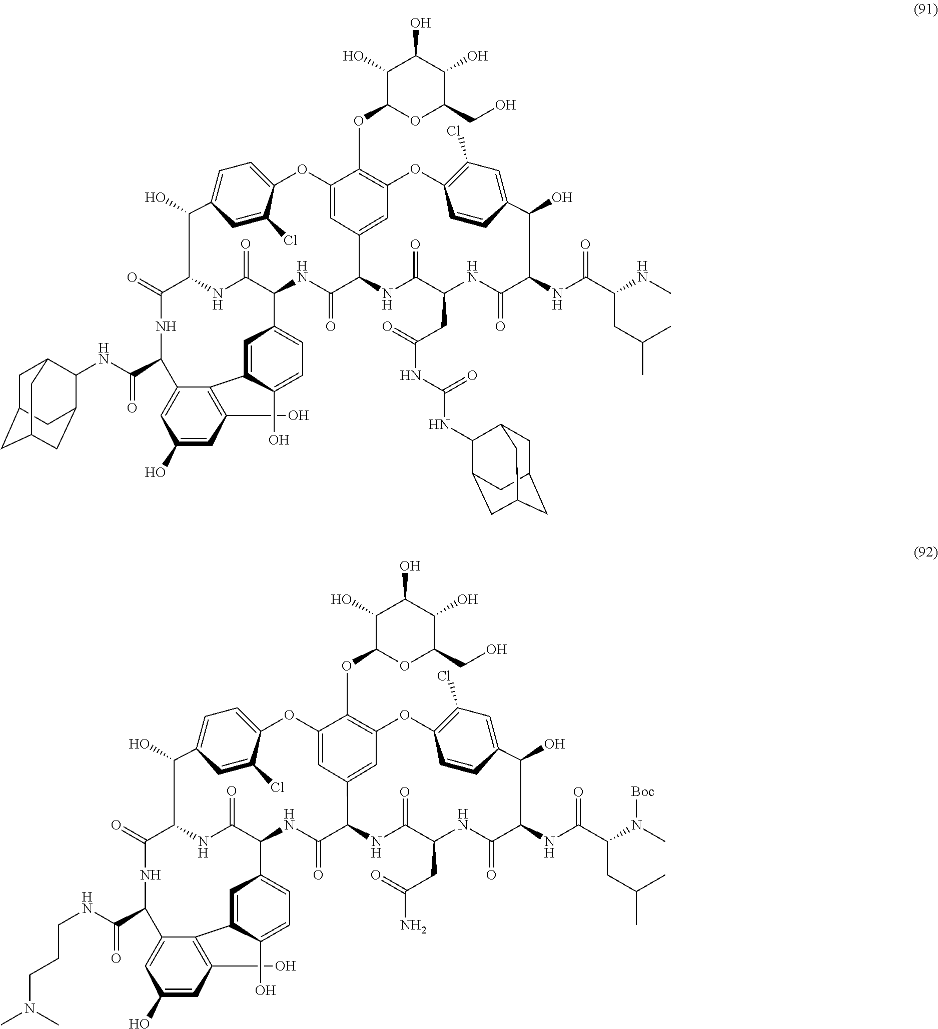

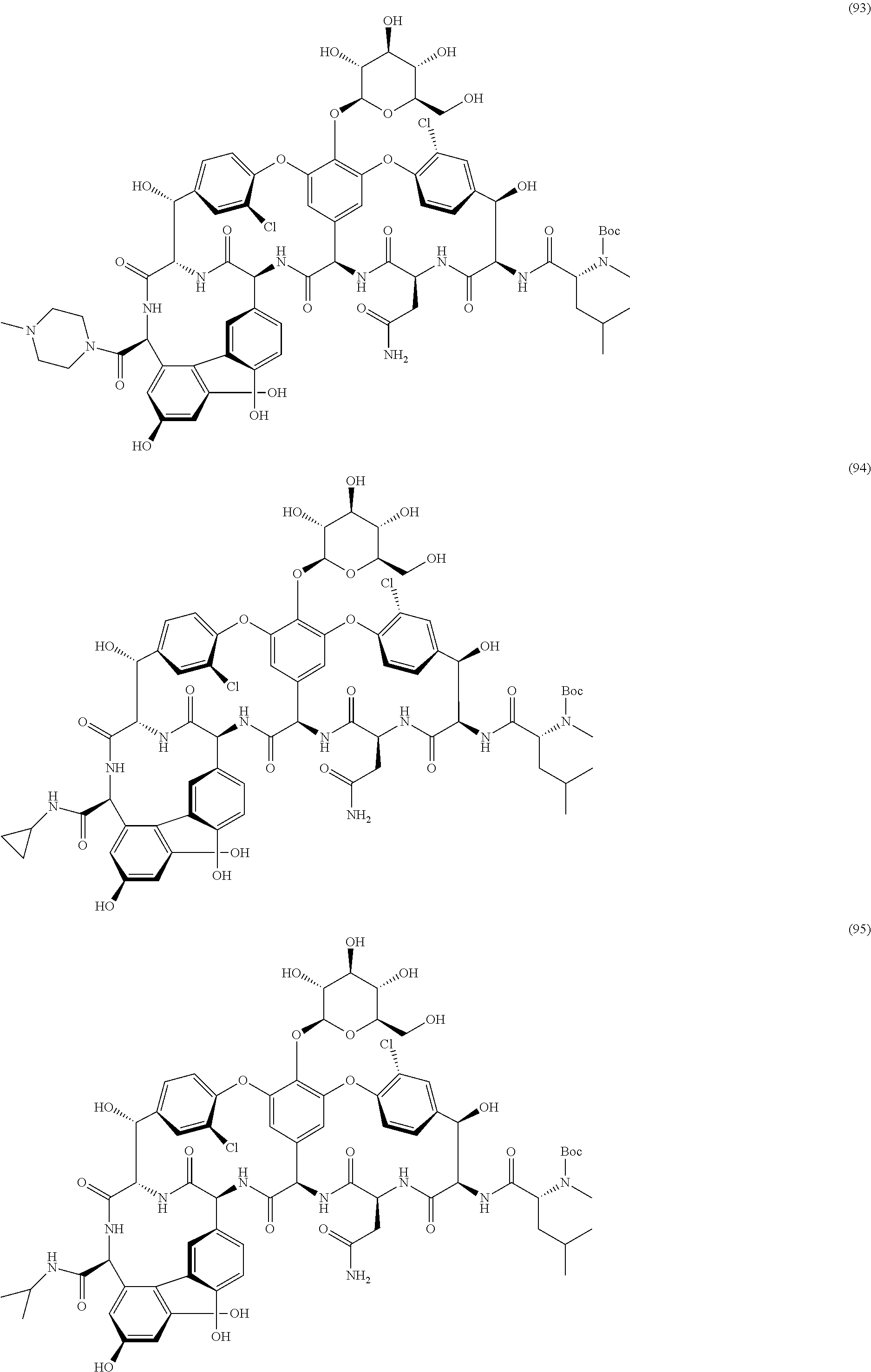

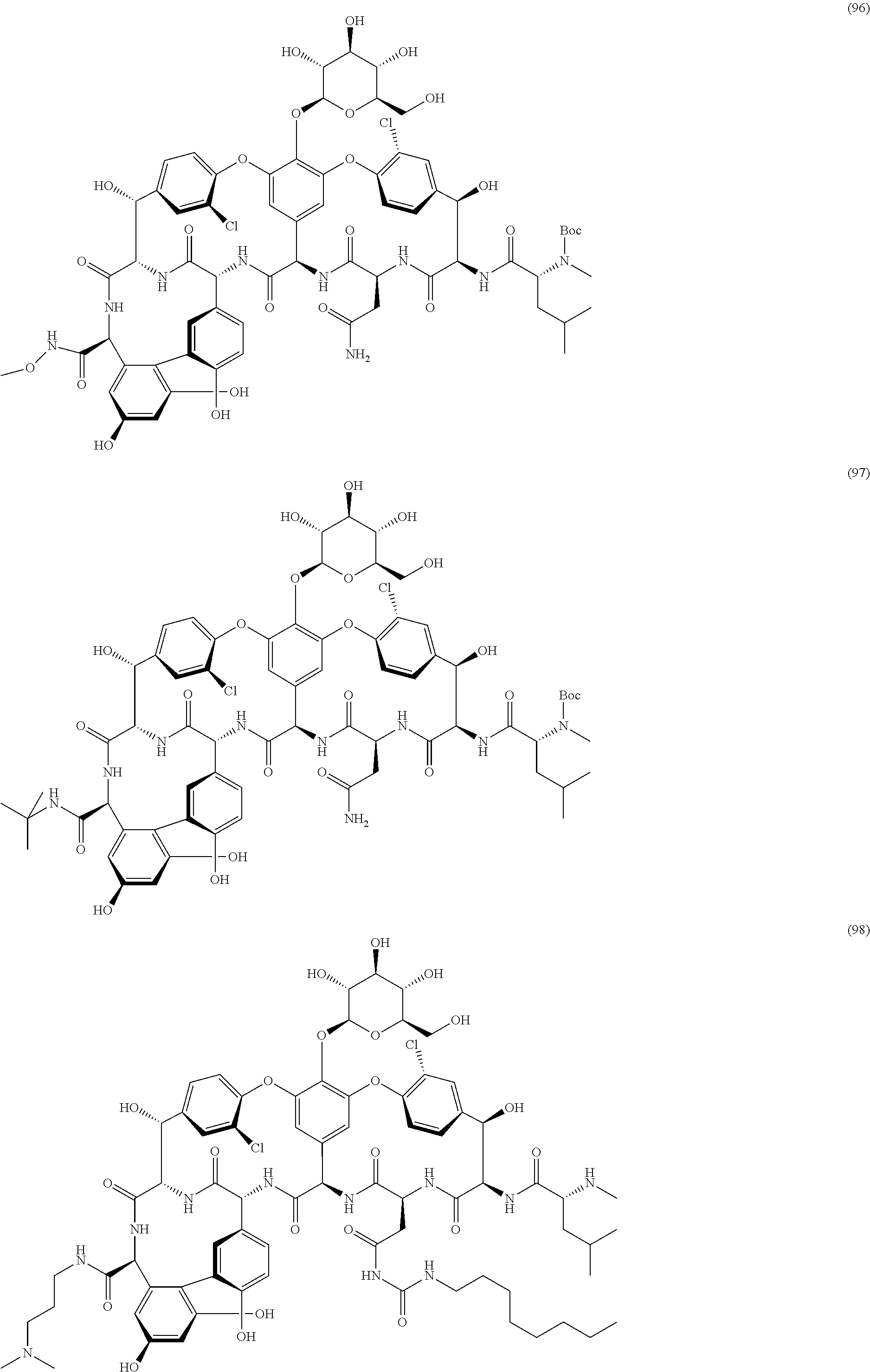

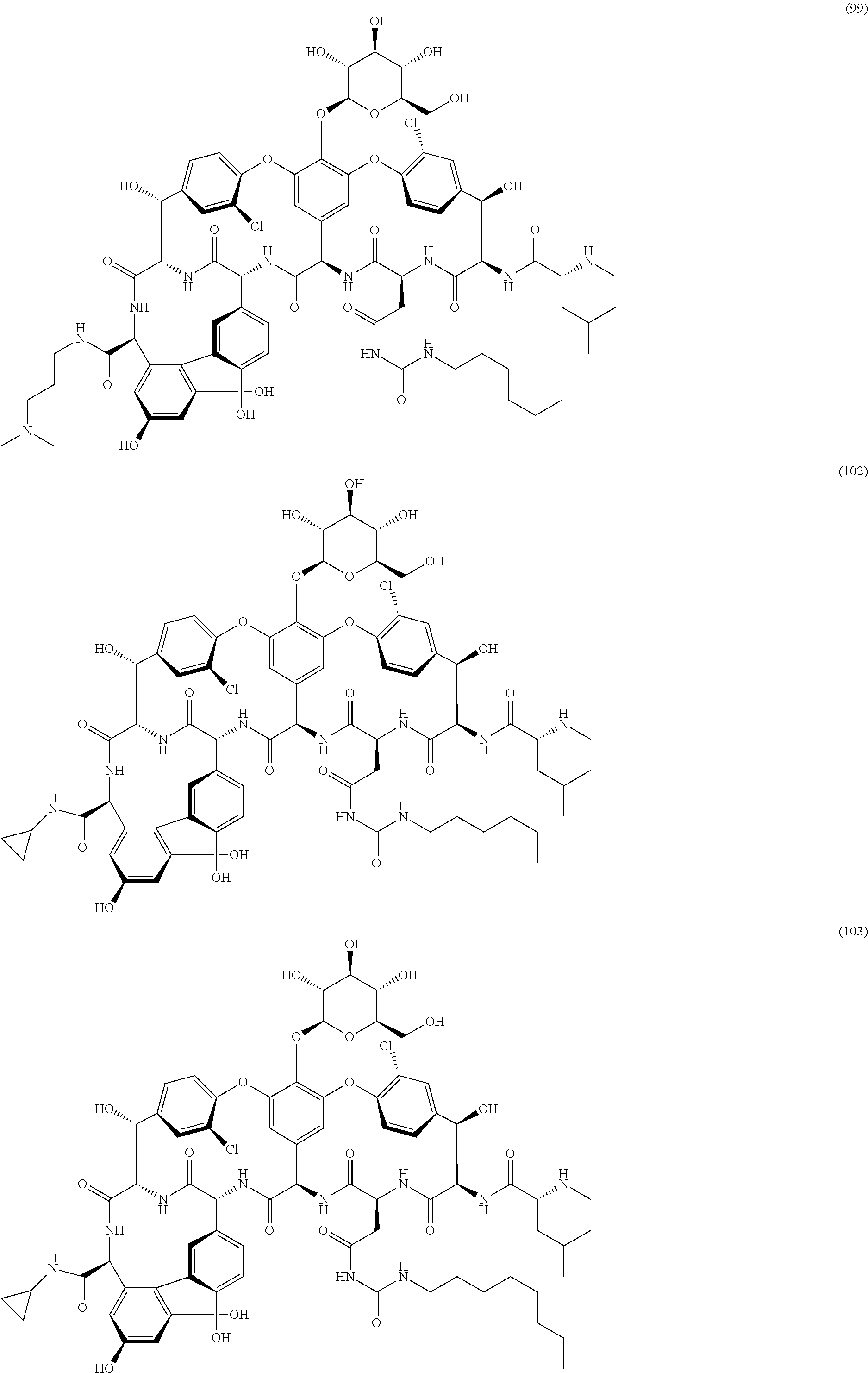

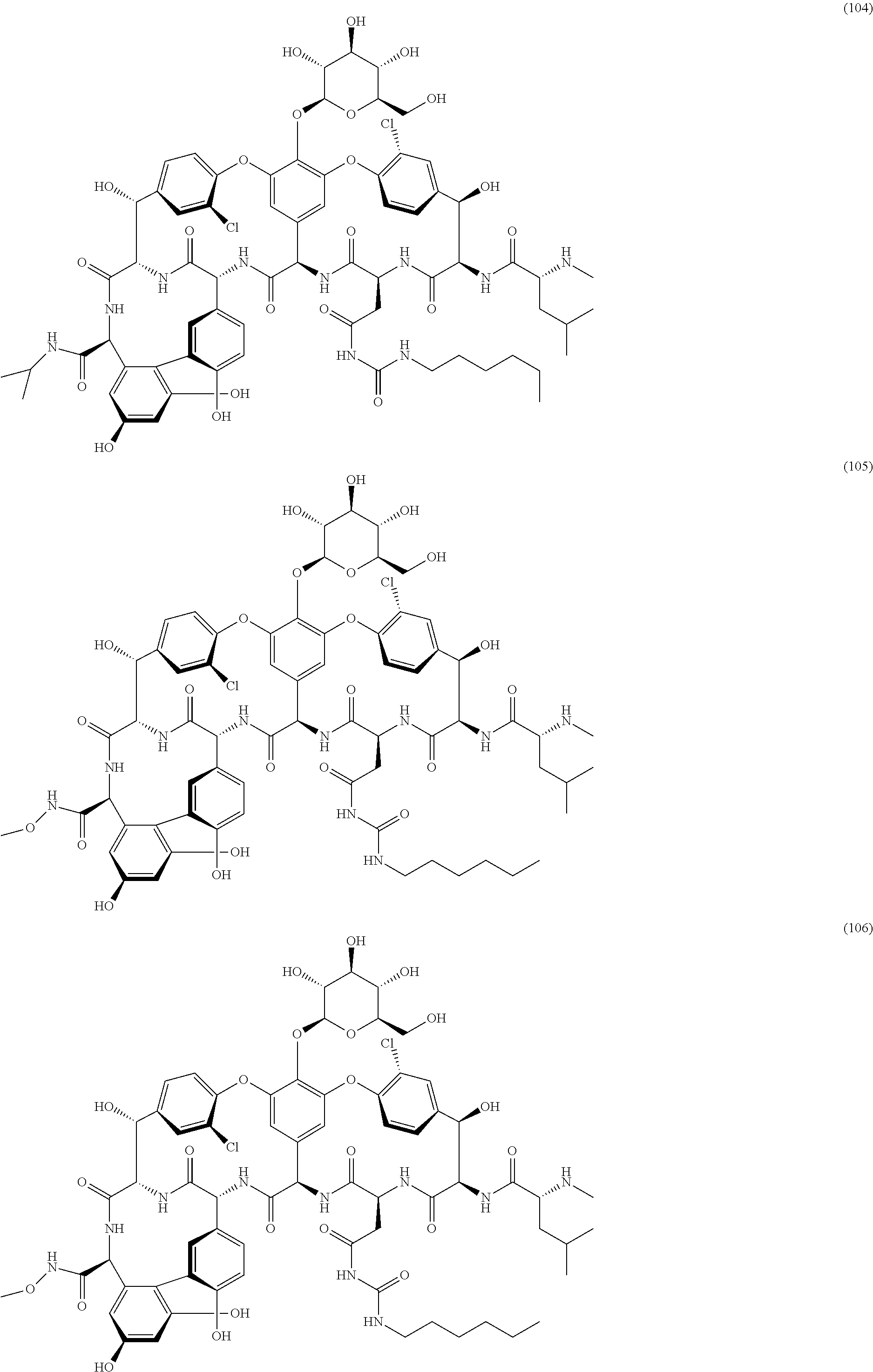

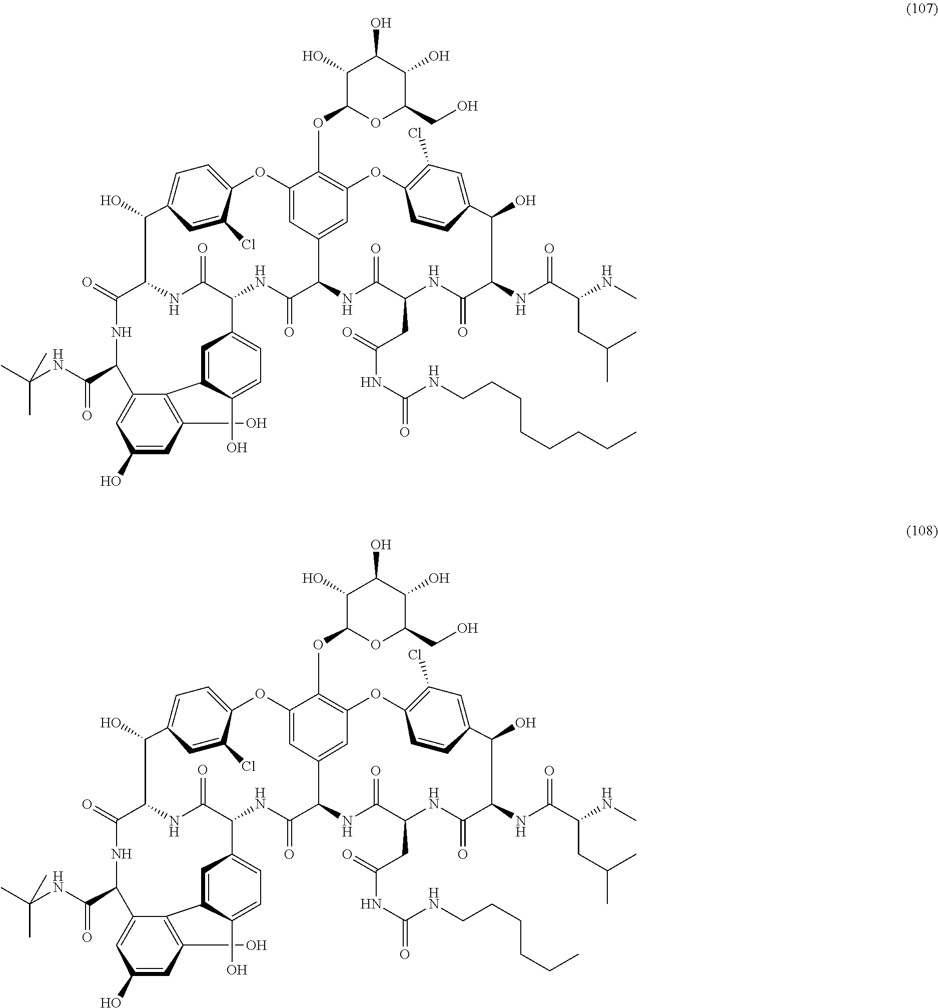

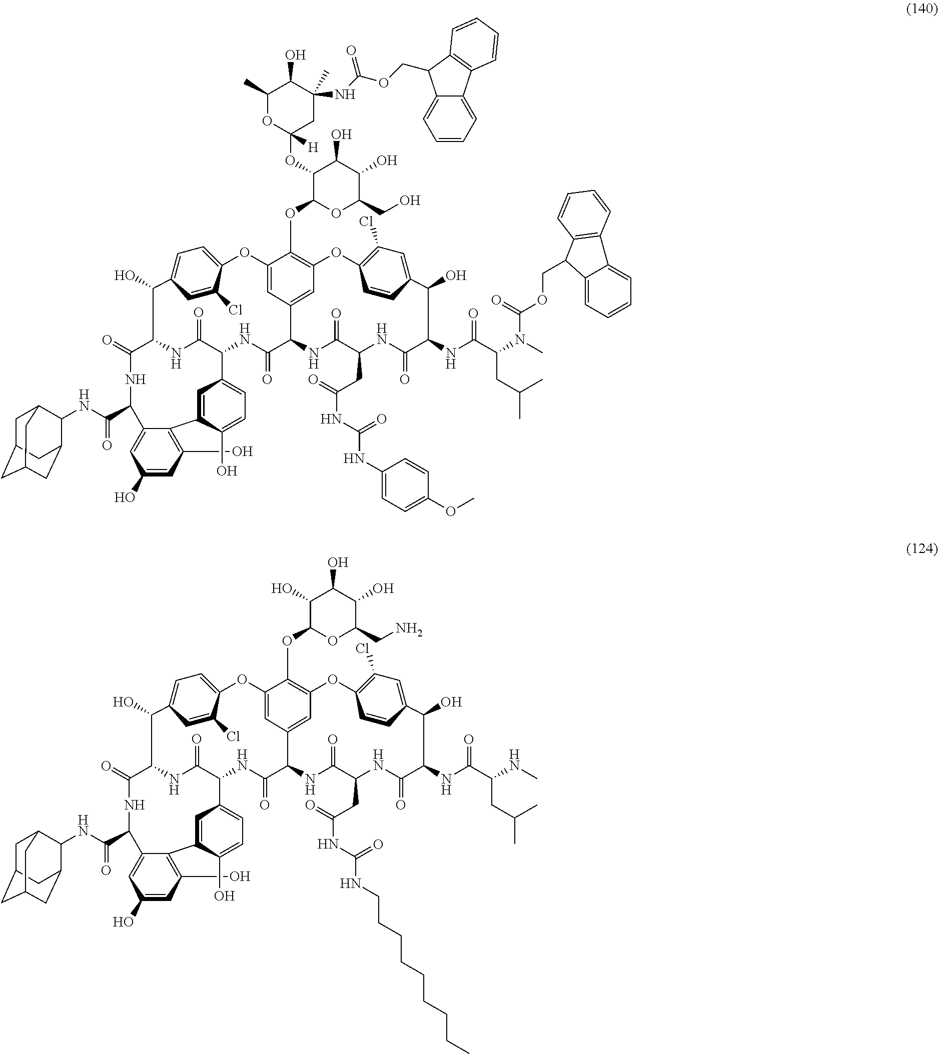

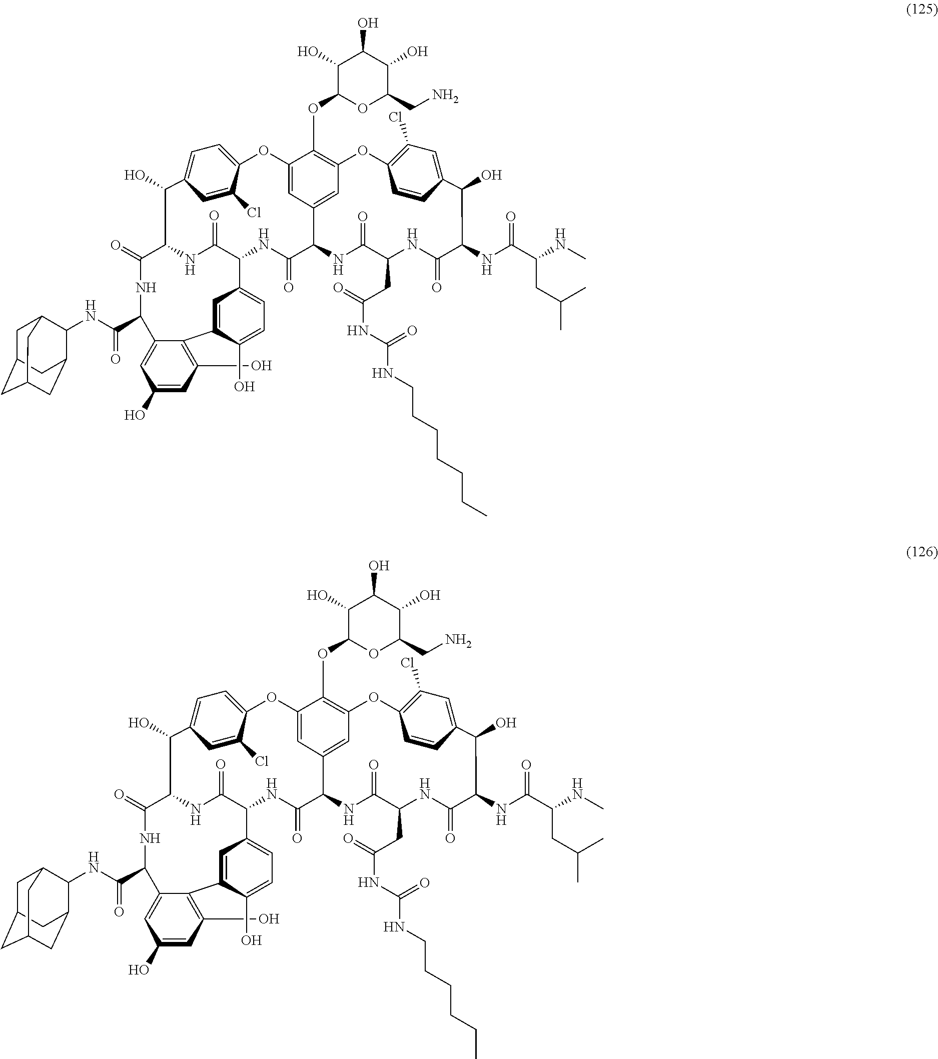

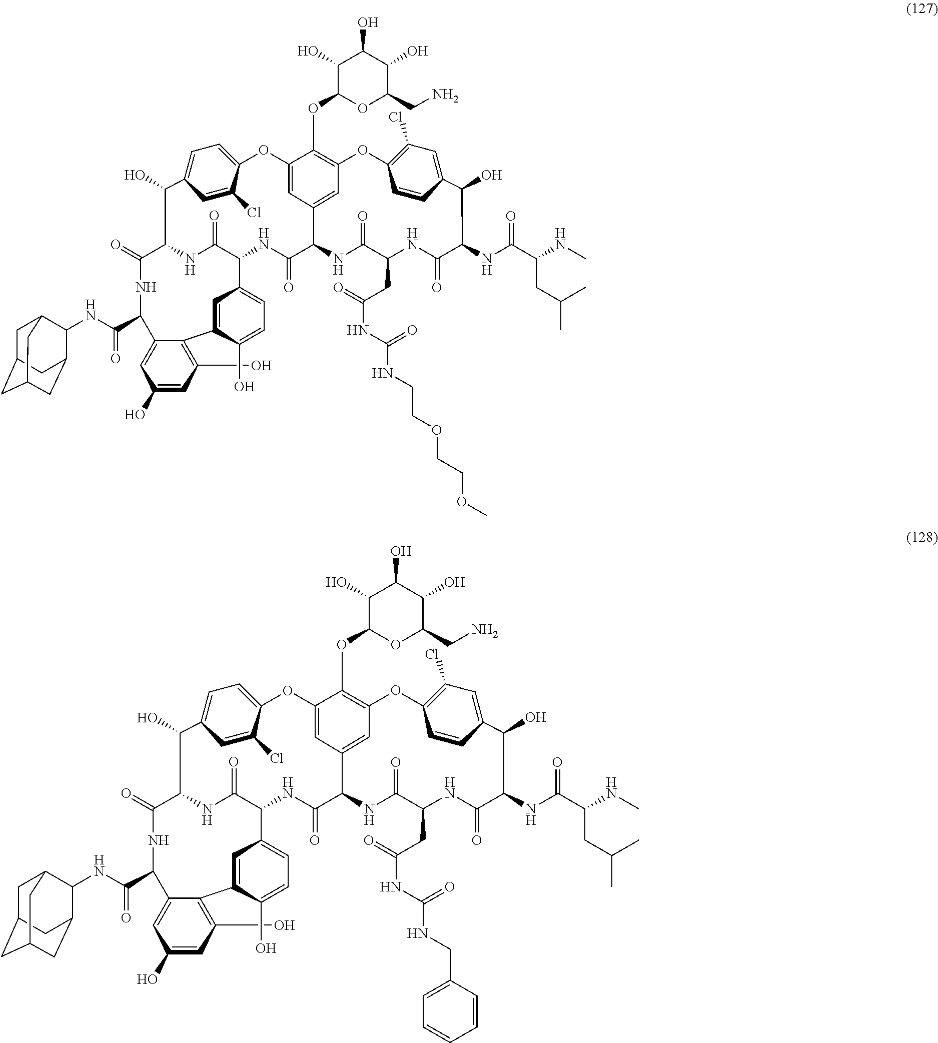

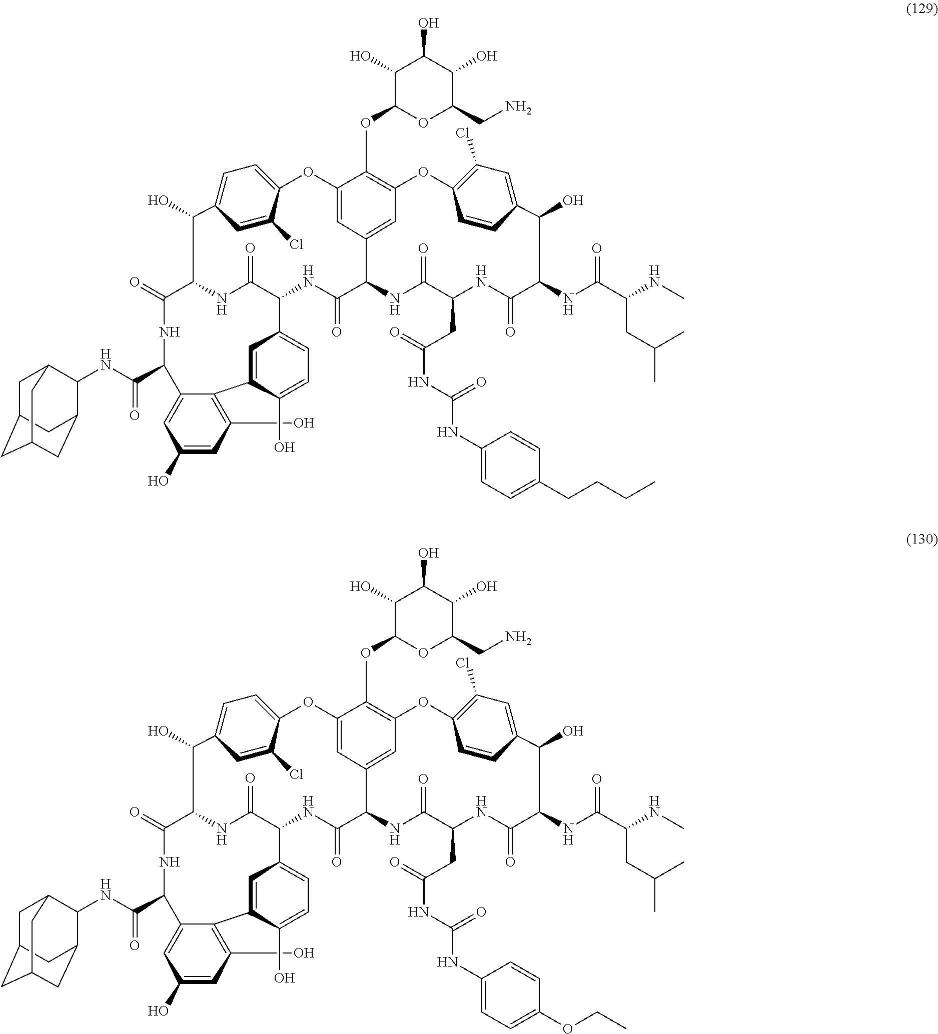

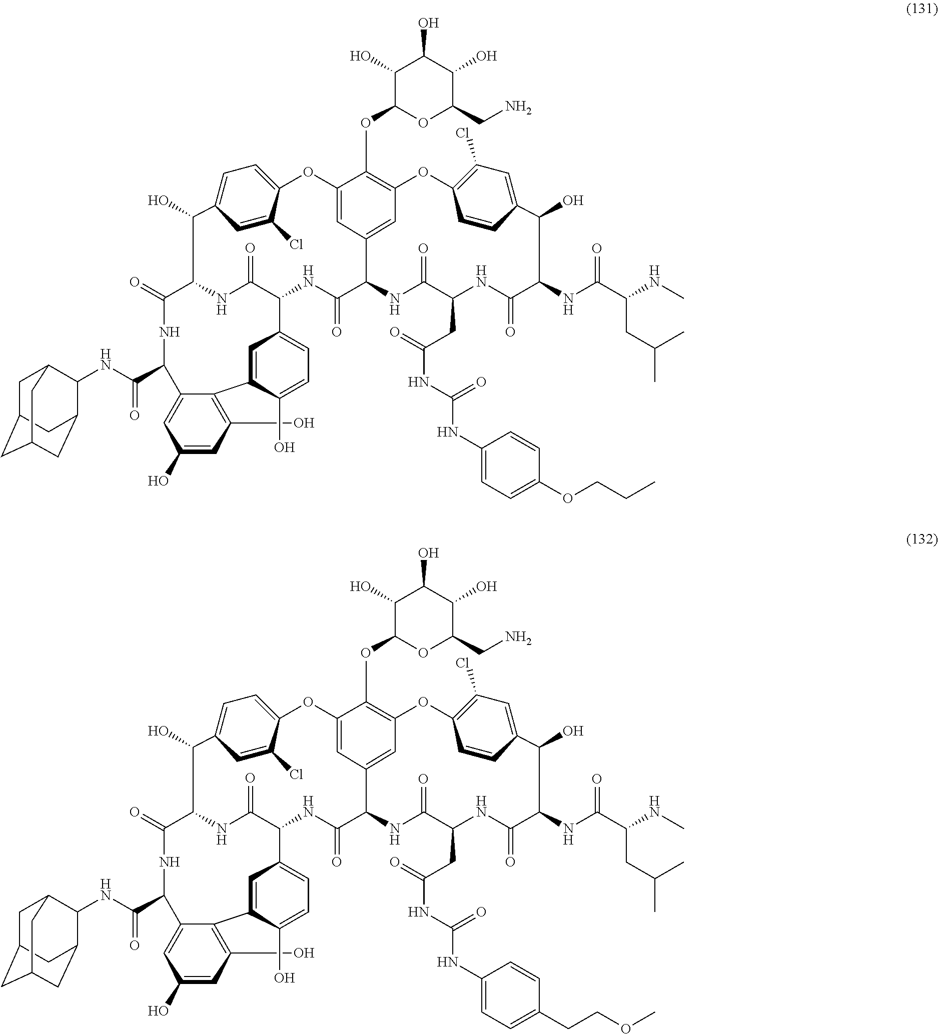

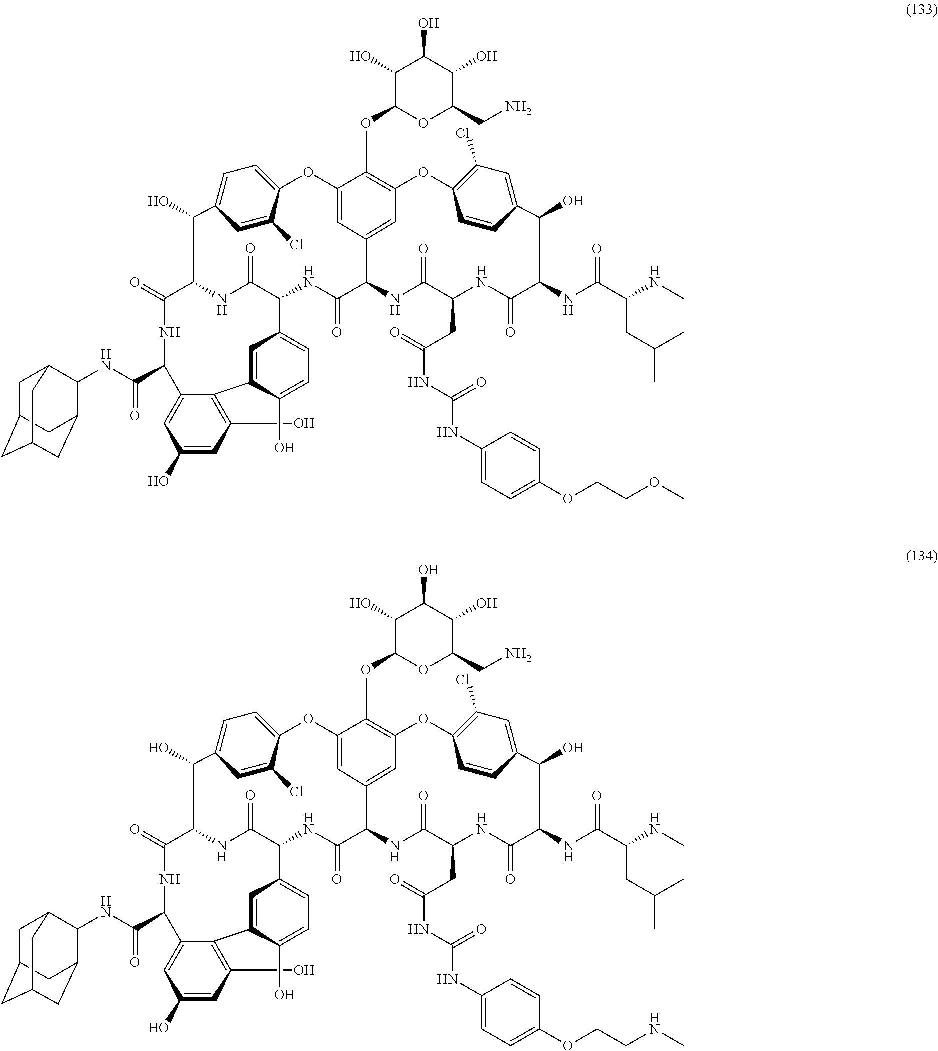

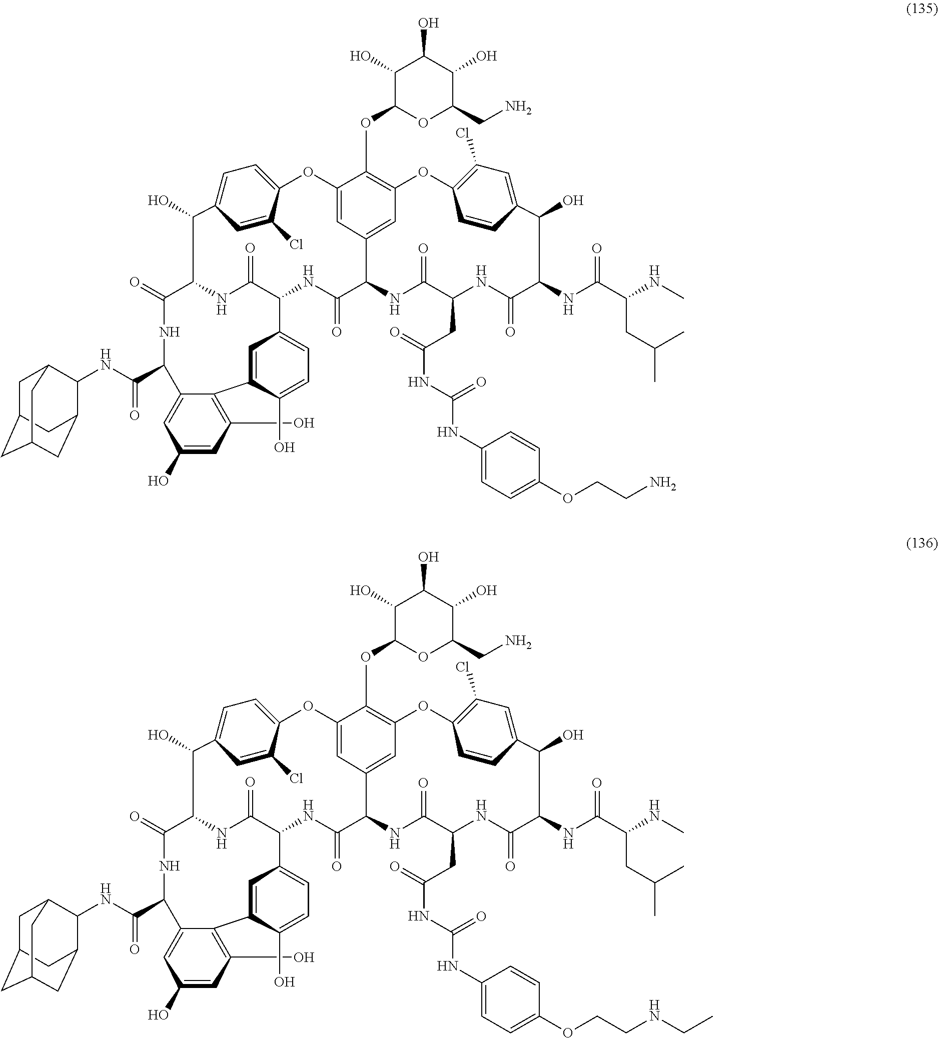

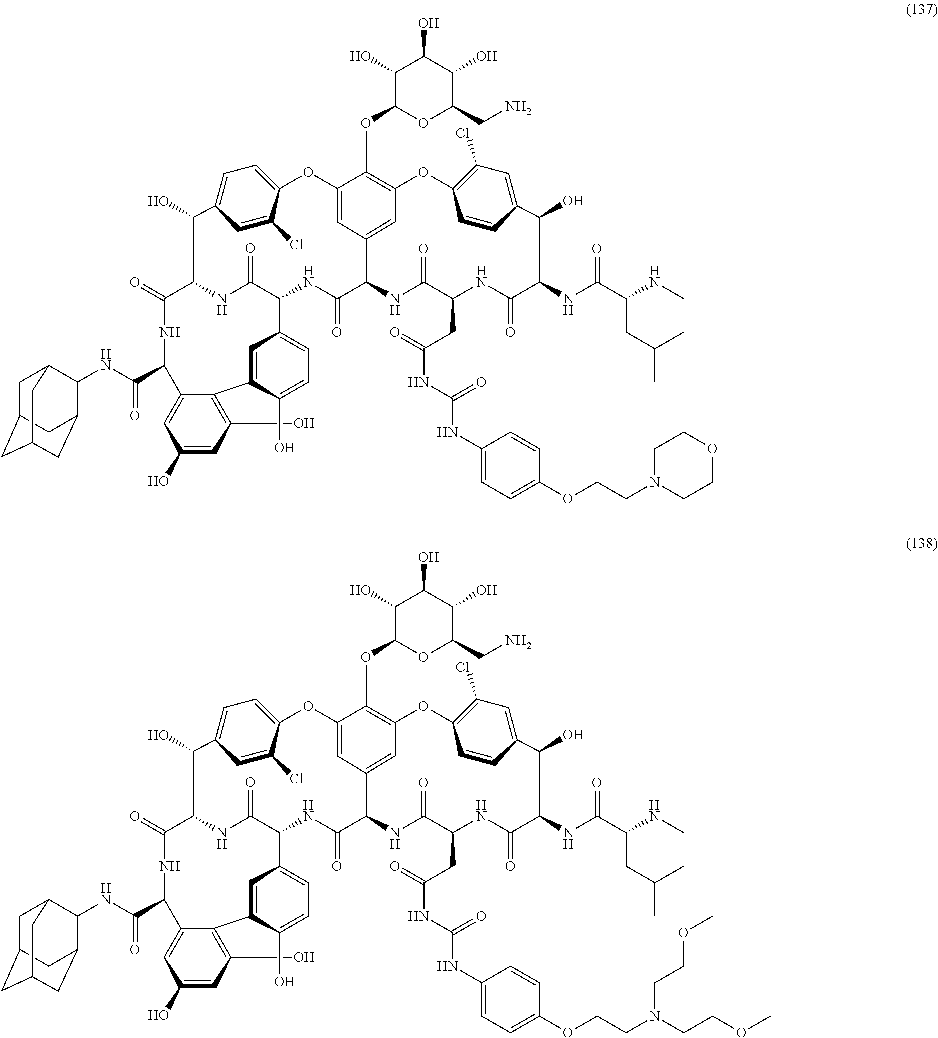

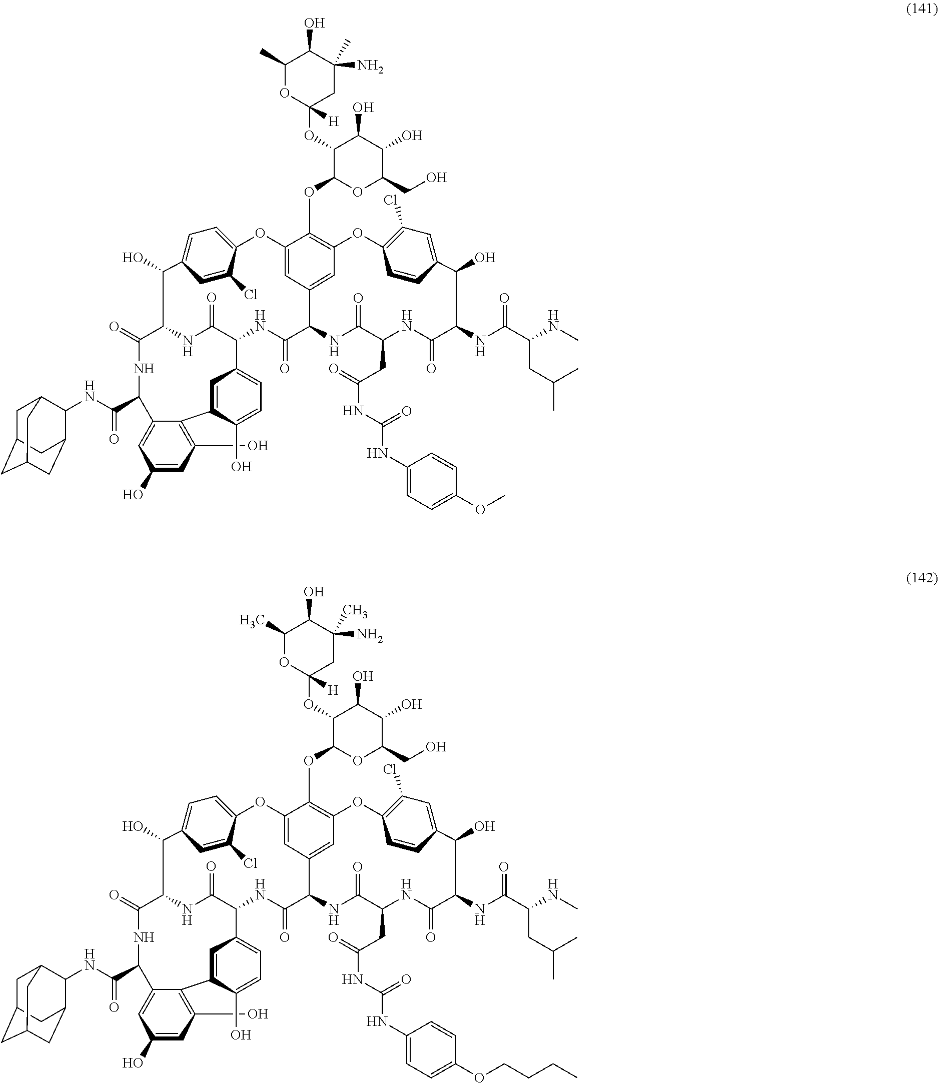

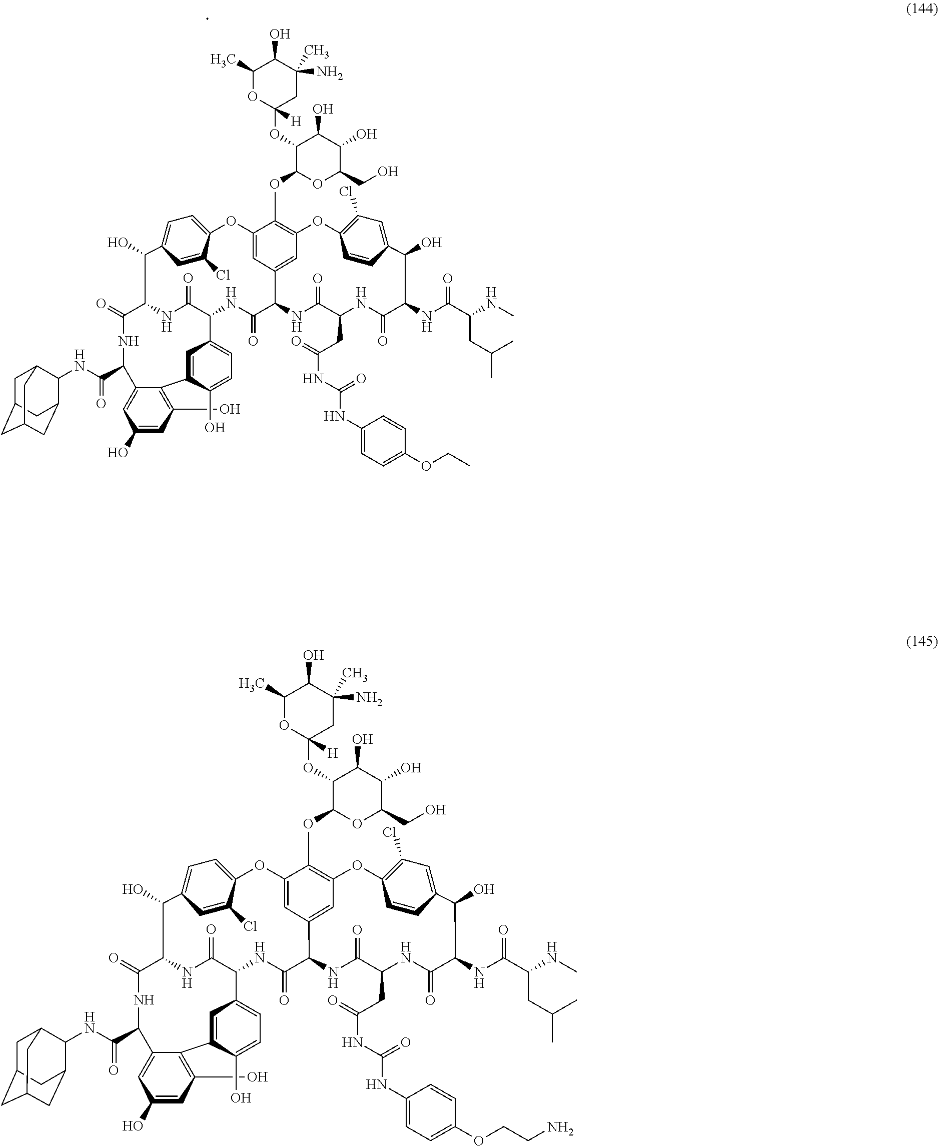

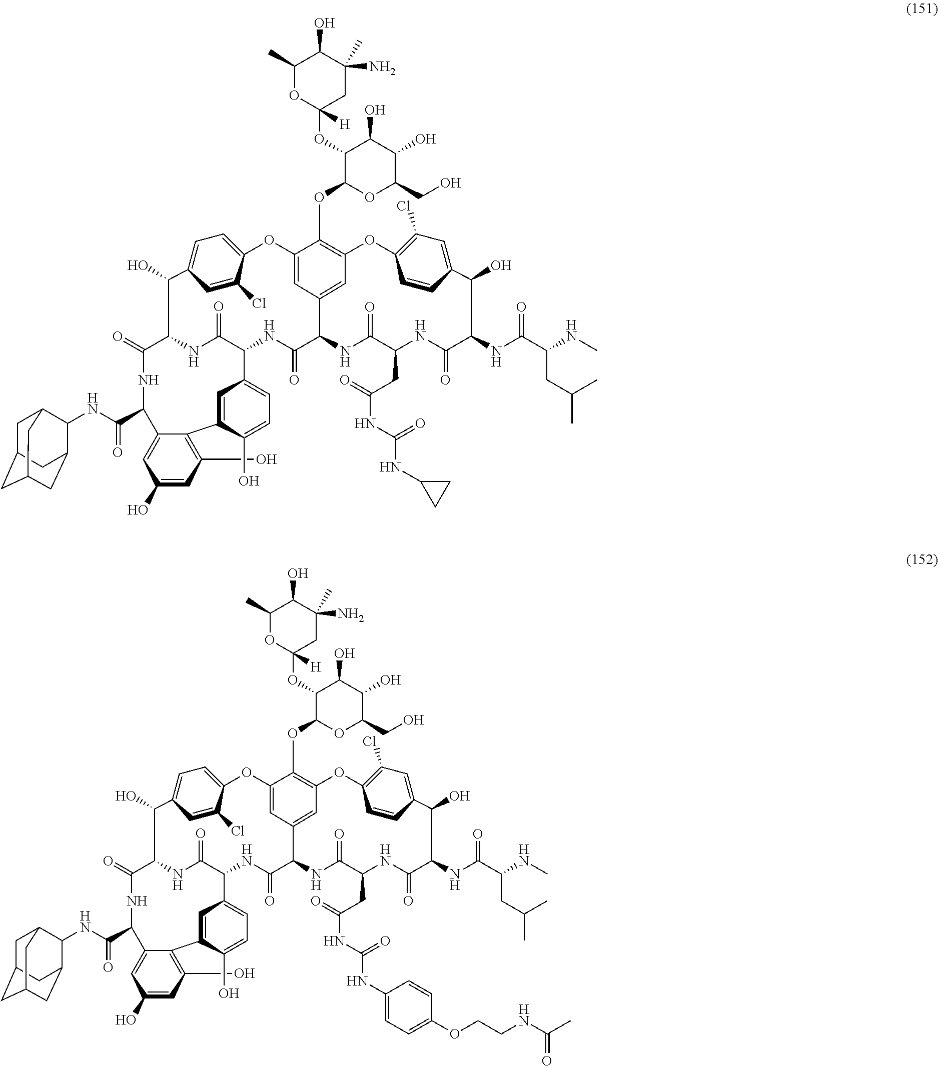

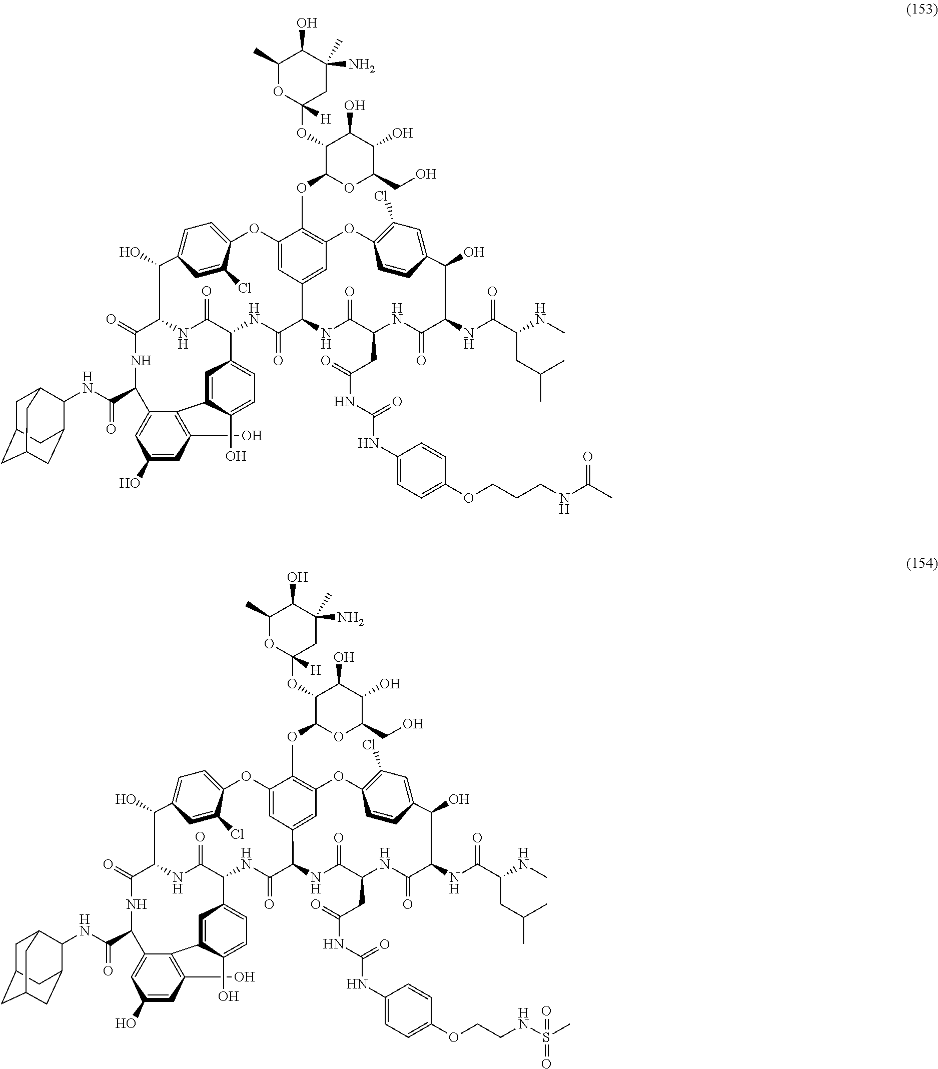

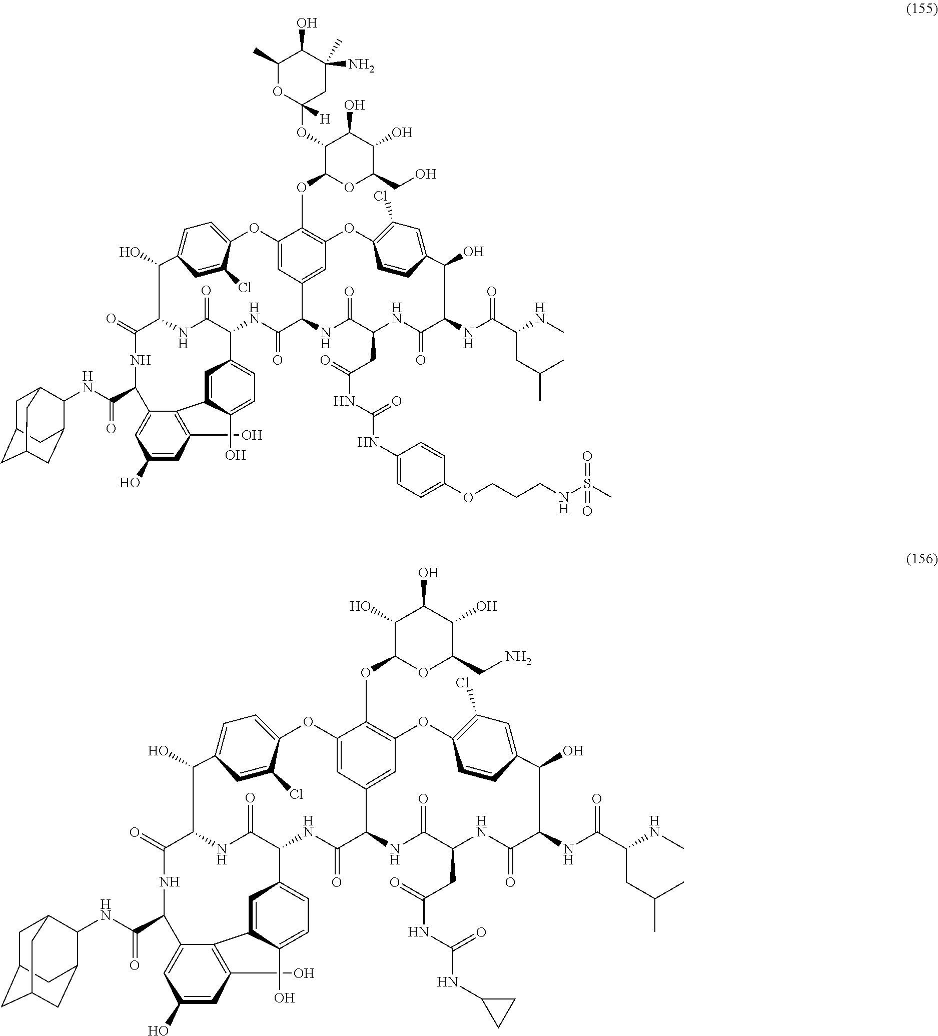

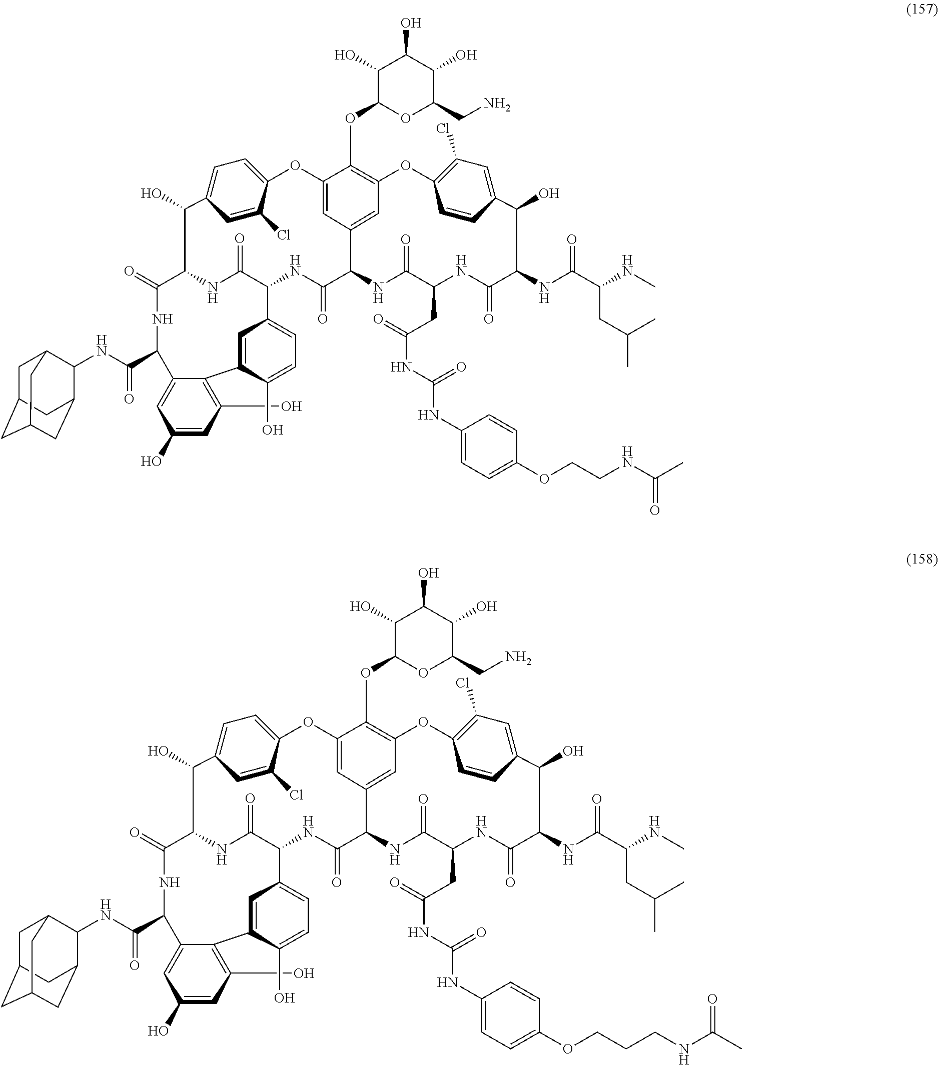

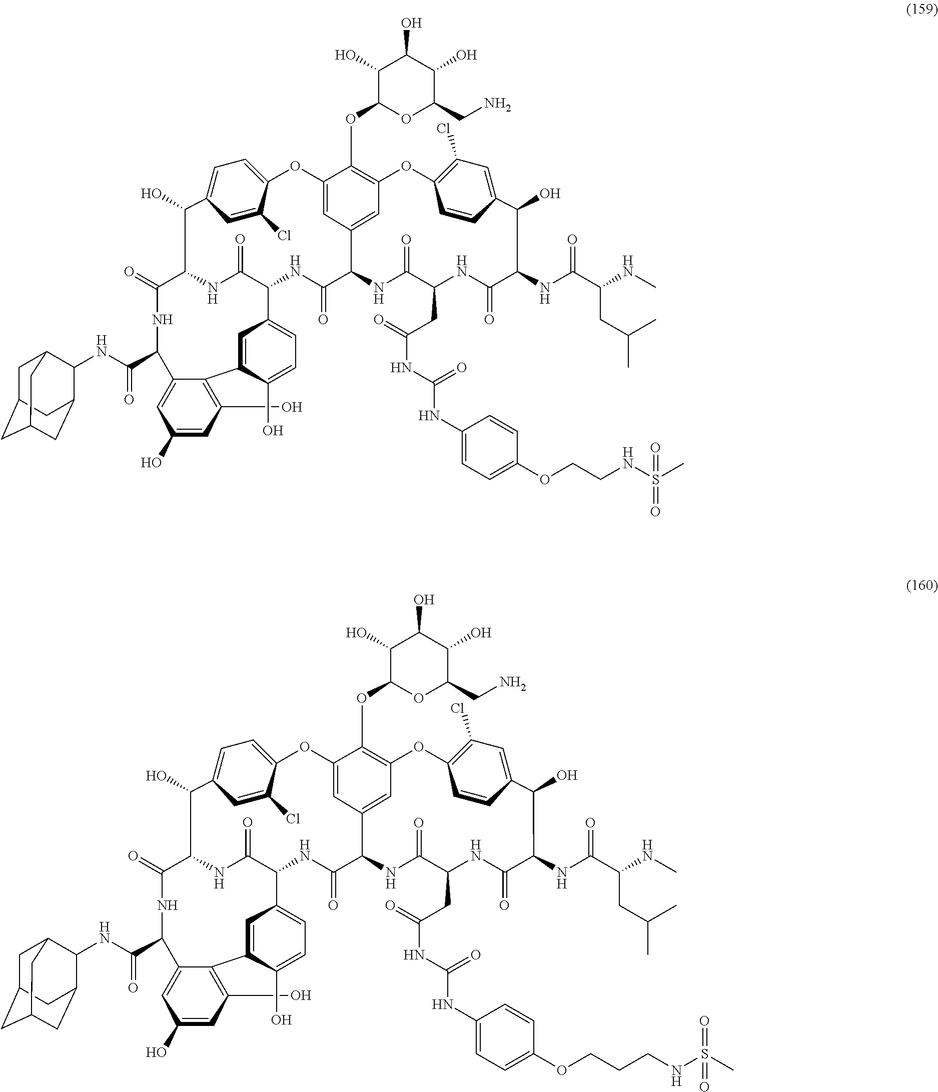

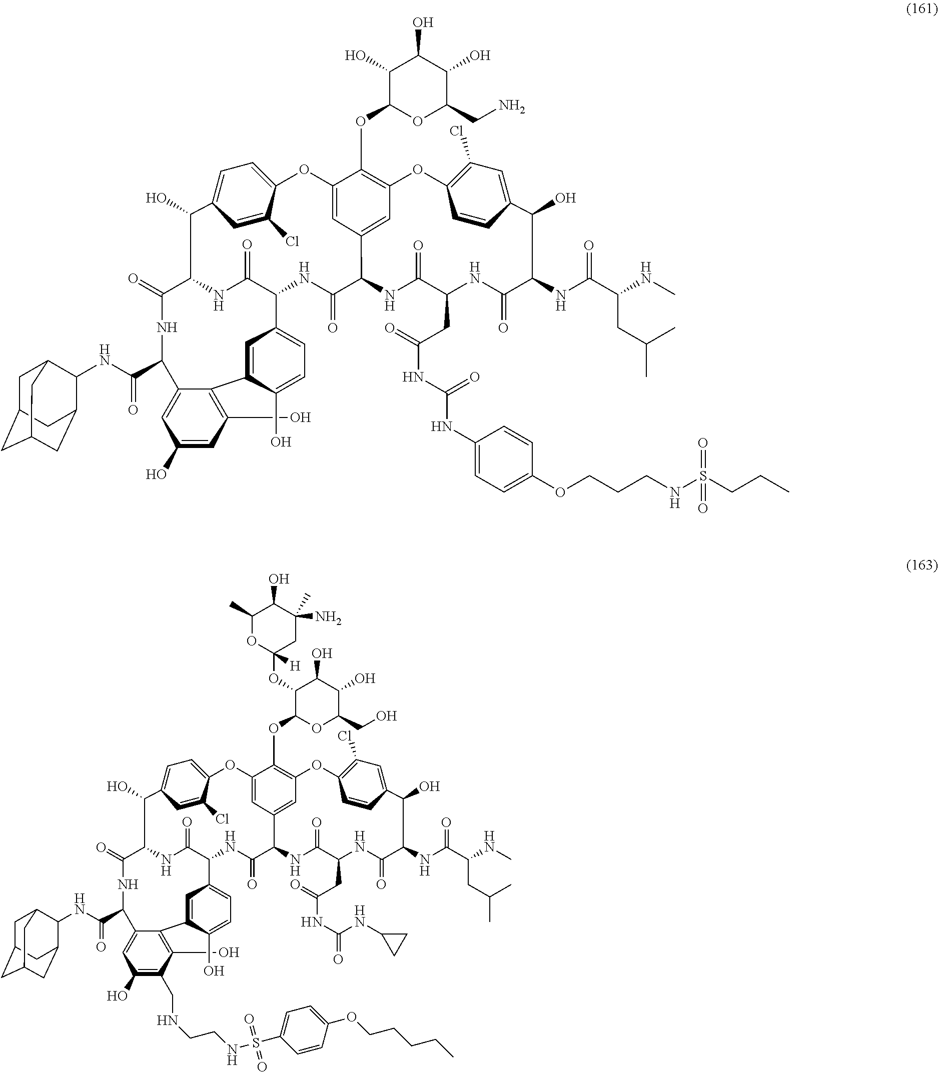

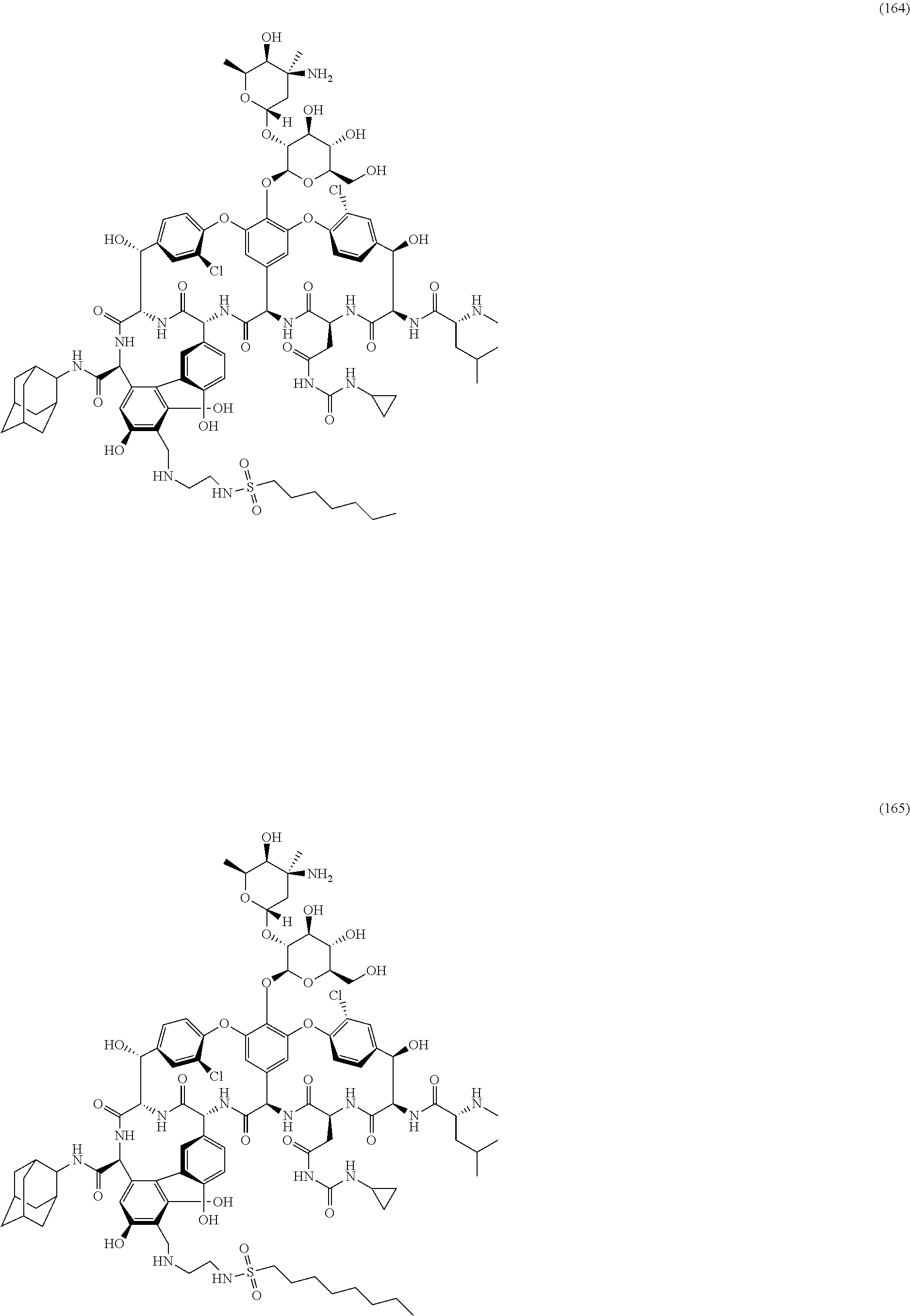

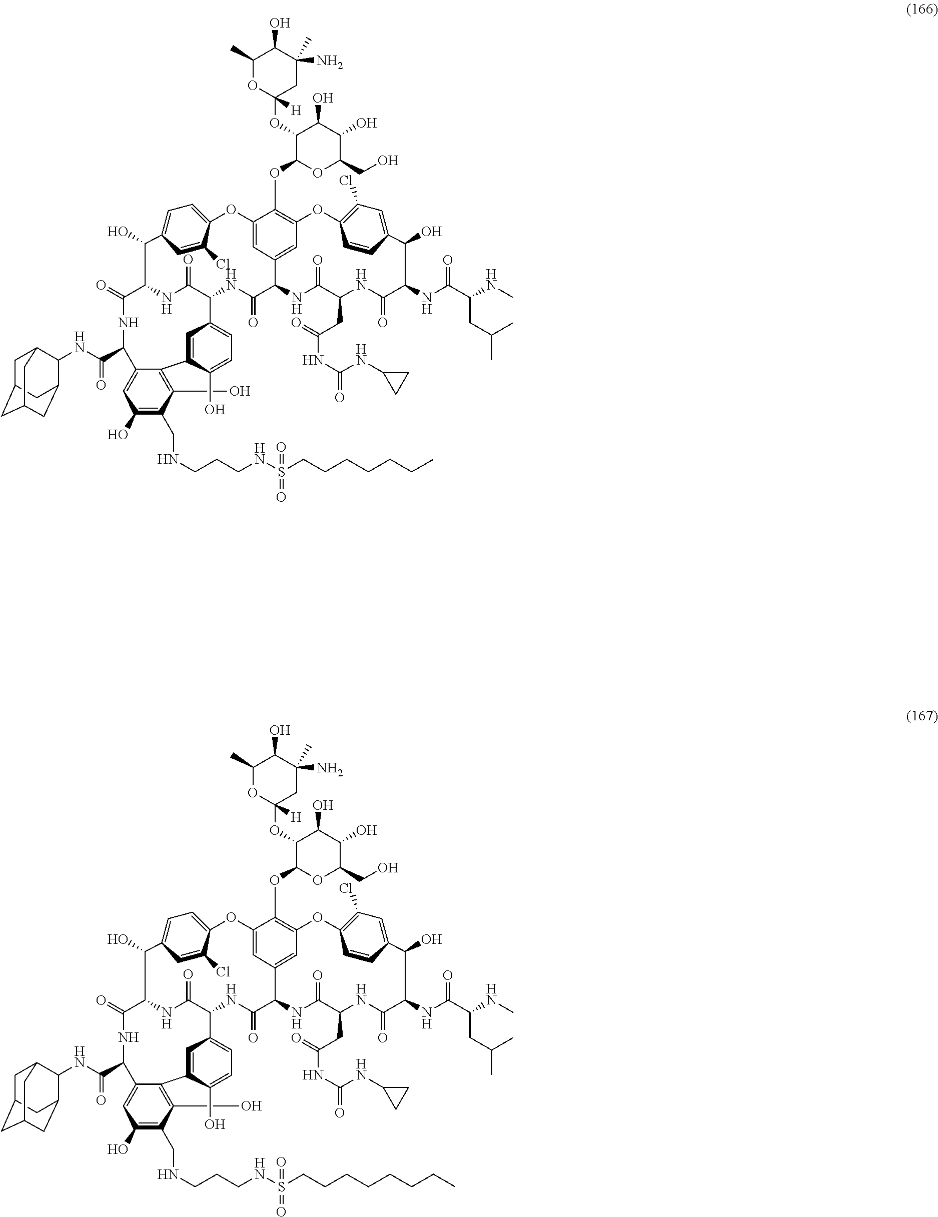

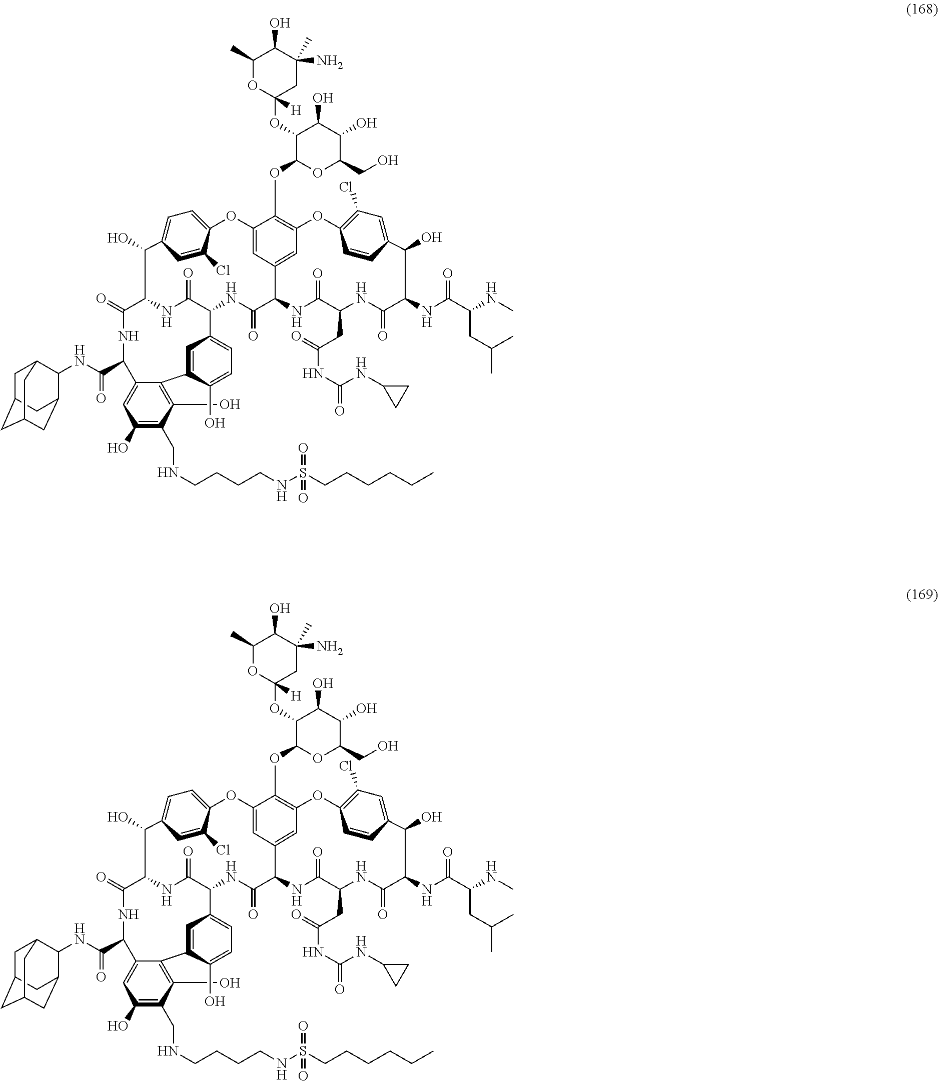

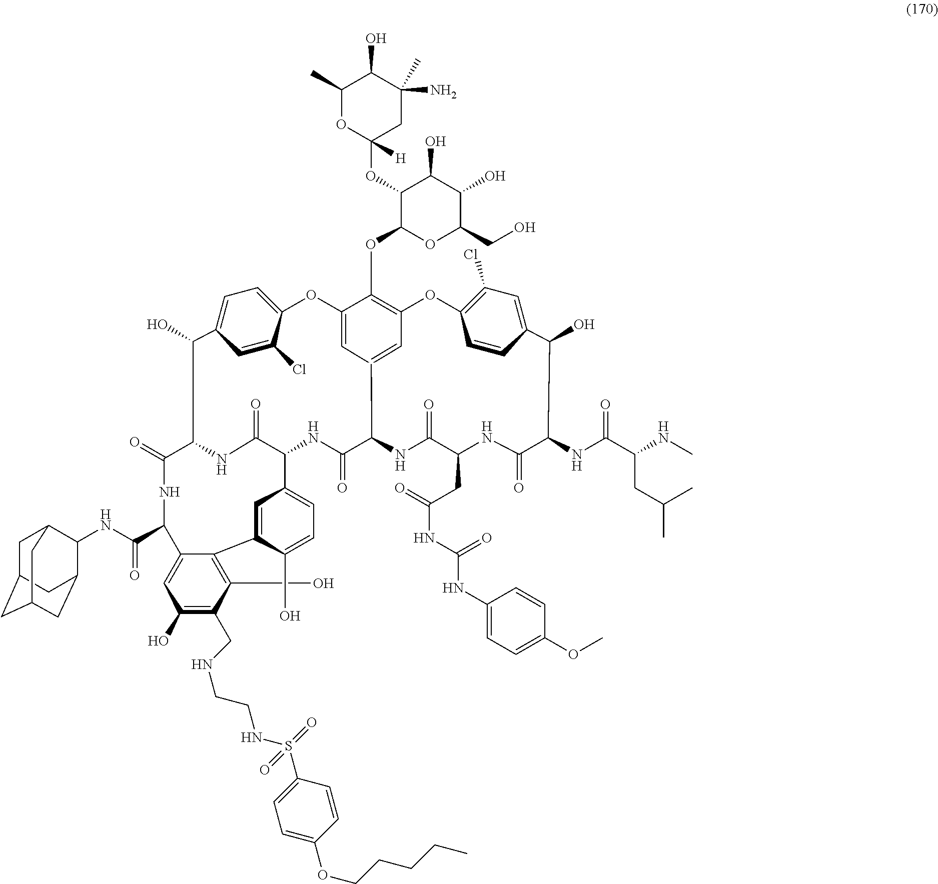

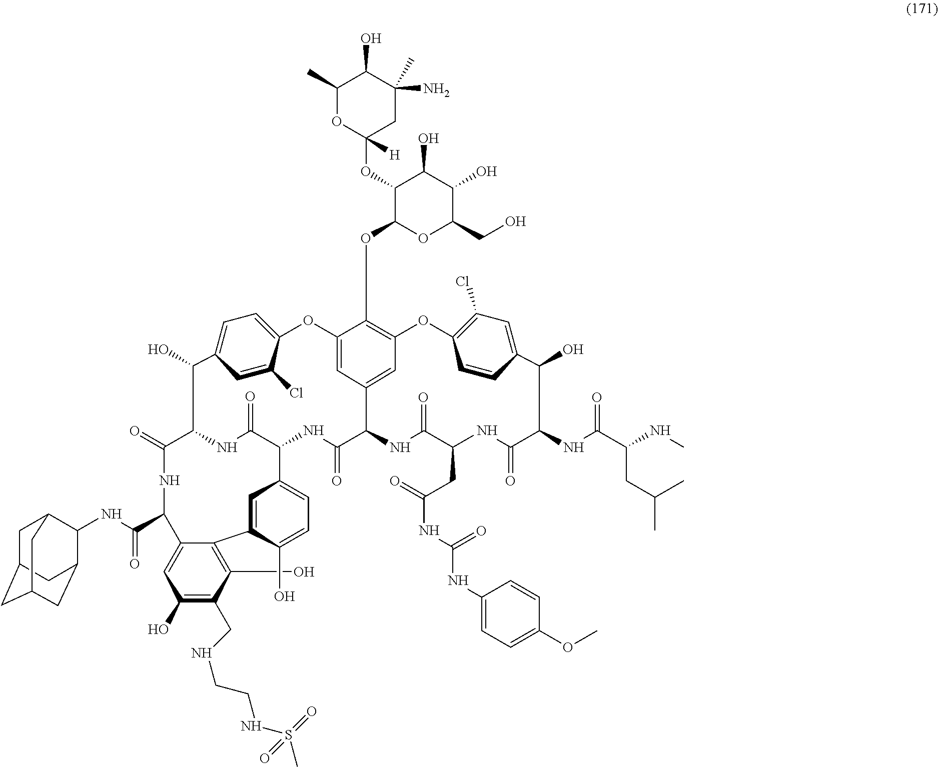

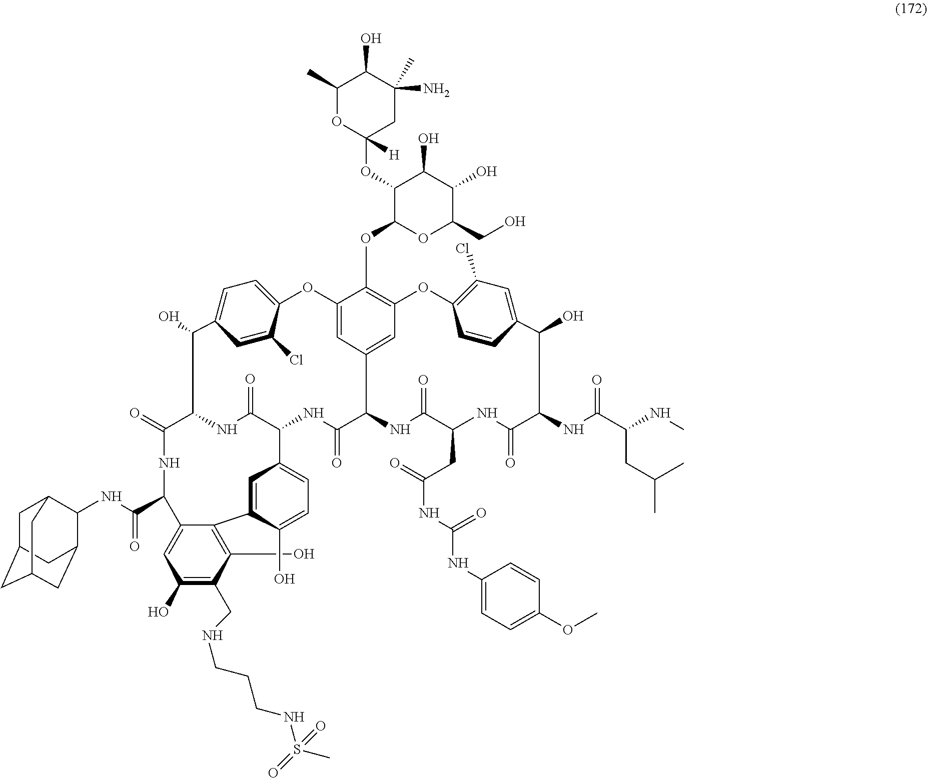

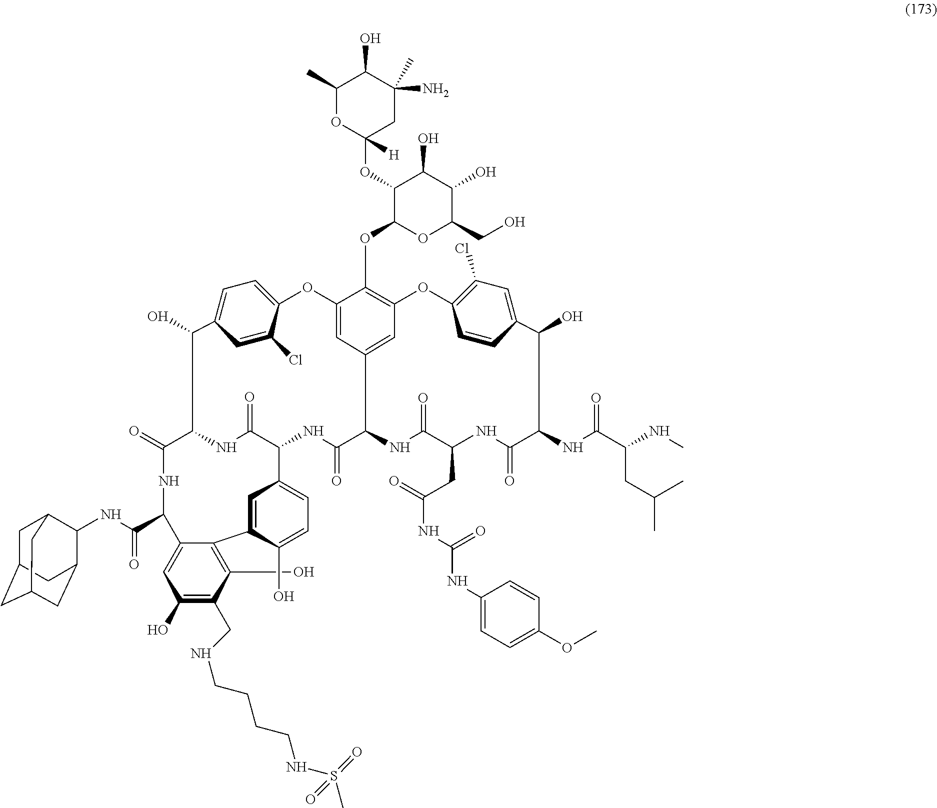

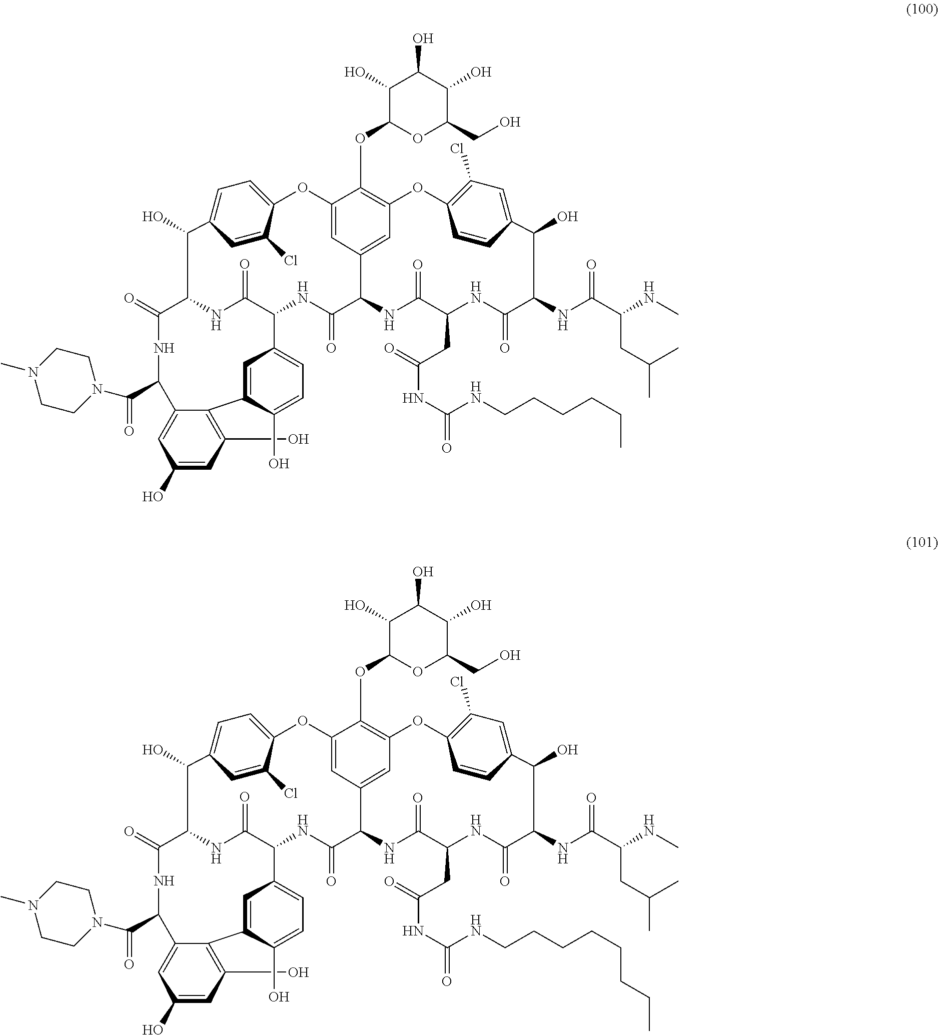

12. A compound obtainable by
(i) reacting a compound selected from the group consisting of Formulas 11, 12, 13, 14, 15 and 16: 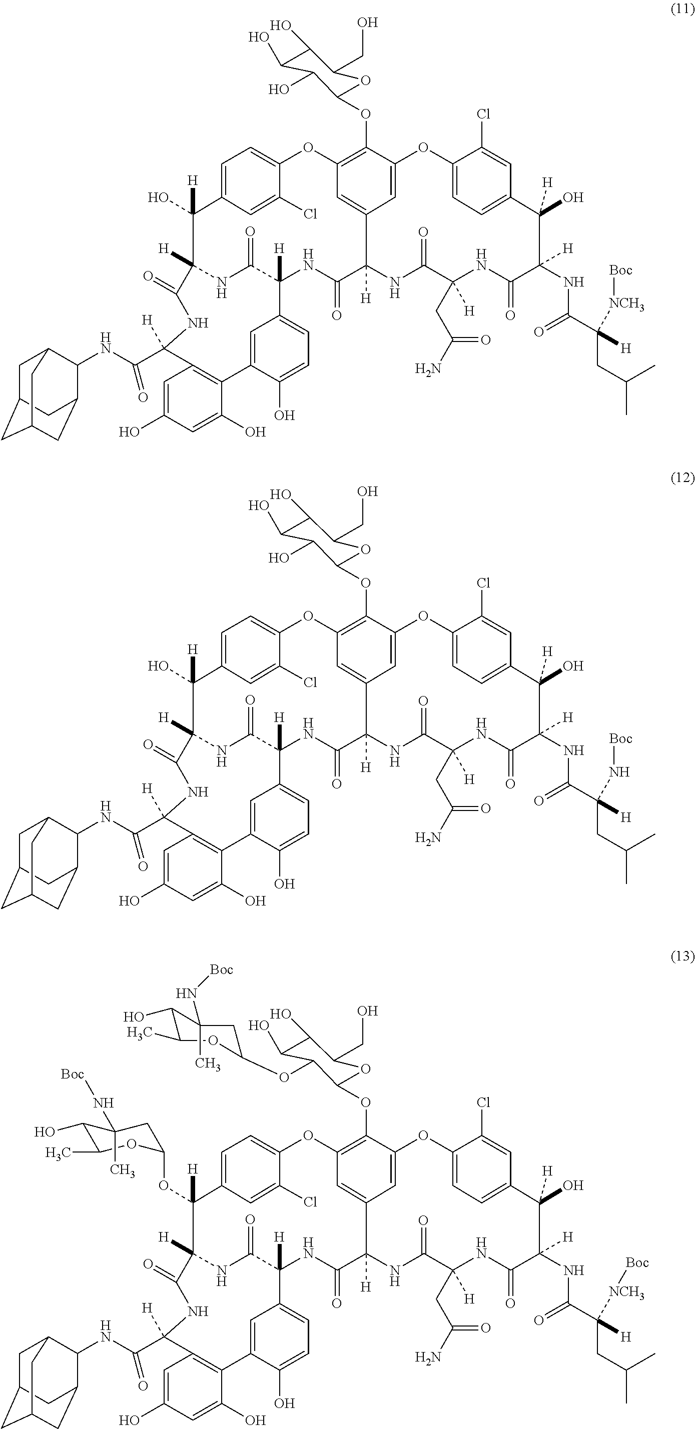 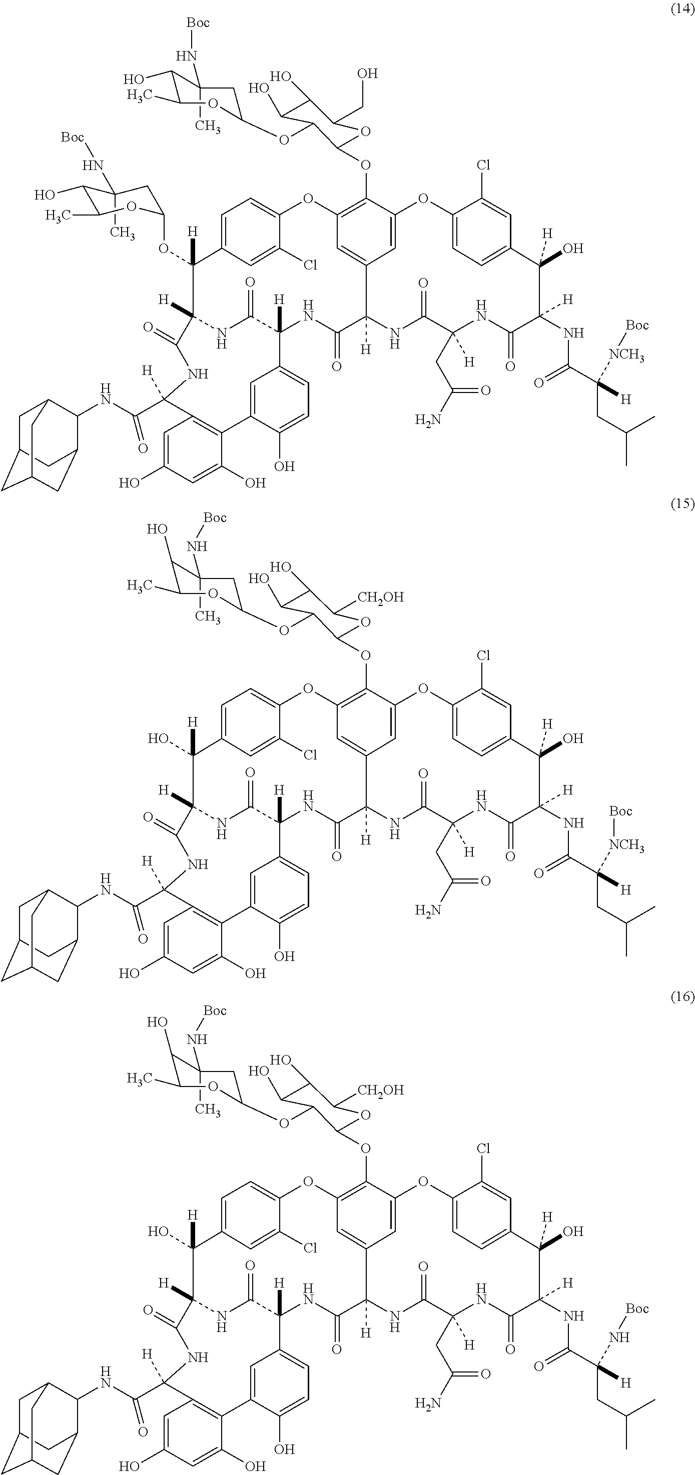
with dimethylaminopyridine and R_{B}—NCZ in dimethylformamide at room temperature,
wherein R_{B} is loweralkyl, substituted loweralkyl, pheny, pyridyl,
substituted aryl or substituted heteroaryl, and Z is O or S; and
(ii) reacting the reaction product of (i) with trifluoroacetic acid in methylene chloride at 0° C.

13. The compound of claim 12, wherein R₁₃ is C₈H₁₇ and Z is O.

14. A pharmaceutical composition comprising a therapeutically effective amount of a compound of claim 12, together with a pharmaceutically acceptable carrier, diluent or excipient.

15. A method of treating a mammal with a bacterial infection comprising administering to the mammal an antibacterial effective amount of a compound of claim 12 or a pharmaceutically acceptable carrier, diluent or excipient thereof.

16. The method of claim 15 where the bacterial infection is gram-positive.

17. The method of claim 15 where the bacterial infection is bacteremia, complicated intra-abdominal infection, complicated skin and skin structure infection, or bacterial pneumonia.

18. The method of claim 15 or 16 where the bacterium to be treated is resistant or refractory to a beta-lactam antibiotic, vancomycin, desmethylvancomycin, eremomycin, teicoplanin, dalbavancin, oritavancin, telavancin, or A82846B (LY264826).

19. The method of claim 15 or 16 where the bacterium to be treated is resistant or refractory to methicillin, flucloxacillin, dicloxacillin, a first-generation cephalosporin agent, a second-generation cephalosporin agent, a third-generation cephalosporin agent, a fourth-generation cephalosporin agent, carbapenem, imipenem, meropenem, ertapenem, faropenem, doripenem, panipenem, biapenem, vancomycin, desmethylvancomycin, eremomycin, teicoplanin, dalbavancin, oritavancin, telavancin, or A828468 (LY264826).
